(19) United States
(12) Patent Application Publication
(10) Pub. No.: US 2025/0099614 A1
(43) Pub. Date: Mar. 27, 2025

(54) NUCLEIC ACID ENCODED TRANSCRIPTION FACTOR INHIBITORS

(71) Applicants: CureVac SE, Tübingen (DE); The Schepens Eye Research Institute, Inc., Boston, MA (US)

(72) Inventors: Joanna REJMAN, Tübingen (DE); Tim SONNTAG, Tübingen (DE); Markus CONZELMANN, Tübingen (DE); Joseph ARBOLEDA-VELASQUEZ, Newton, MA (US); Leo A. KIM, Brookline, MA (US)

(73) Assignees: CureVac SE, Tübingen (DE); The Schepens Eye Research Institute, Inc., Boston, MA (US)

(21) Appl. No.: 18/833,889

(22) PCT Filed: Jan. 27, 2023
(86) PCT No.: PCT/EP2023/052056

§ 371 Date: Jul. 27, 2024

Related U.S. Application Data
(60) Provisional application No. 63/304,188, filed on Jan. 28, 2022.

(51) Int. Cl. A61K 48/00; A61K 9/127; A61P 27/02; C07K 14/47

(52) U.S. Cl. CPC A61K 48/005; A61K 9/1275; A61P 27/02; C07K 14/4703

(57) ABSTRACT

The present invention is inter alia directed to artificial nucleic acid constructs, preferably RNA, comprising at least one coding sequence encoding at least one transcription factor inhibitor for reducing or inhibiting the activity of a target transcription factor in a cell. A preferred transcription factor inhibitor according to the invention is a Runt-related transcription factor (RUNX) inhibitor, for example a RUNX trap comprising at least one amino acid sequence for binding a RUNX transcription factor and at least one amino acid sequence for capturing or trapping RUNX. Further provided are pharmaceutical compositions comprising the artificial nucleic acid, preferably formulated in polyethylene glycol/peptide polymers, polymeric carriers, or lipid-based carriers. Also provided are methods of treating or preventing disorders, diseases, or conditions, and medical uses.

Transcription factor malfunctions play a crucial role in the development and progression of various different diseases. For example, increased RUNX1 function is a hallmark of pathological epithelial to mesenchymal transition (EMT), aberrant angiogenesis, degeneration, and fibrosis; processes underlying multiple prevalent conditions in the eye and elsewhere. Accordingly, transcription factors represent powerful therapeutic targets for treating or preventing numerous diseases.

However, developing effective medical treatments for transcription factor pathologies, for example pathologies relating to transcription factor overexpression, remains to be a challenge. For example, WO2019099560, WO2018093797, WO2019099595, and WO2021216378 describe small molecule inhibitors of RUNX1, inhibitory nucleic acids (e.g. siRNA), and also suggest the use of protein-based inhibitors (e.g., CBFB-MYH11). However, using protein-based inhibitors for transcription inhibiting factors is hampered by the fact that transcription factors are intracellular protein, thus making delivery of these into the cell challenging.

Nucleic acid-based sequences, for example RNA, may represent a promising class of molecules to provide the information for expressing intracellular proteins such as transcription factor inhibitors. So far, the use of RNA technologies for clinical applications has mainly focused on immunotherapeutics for multiple clinical applications. Pathologies caused by increased function or activity of a gene (e.g. a transcription factor) are more difficult to address directly with RNA-based therapeutics (Sahin et al 2014; Nature Reviews Drug Discovery. 2014; 13 (10): 759-80).

Thus, the underlying object of the invention is to provide nucleic acid-based therapeutics for producing transcription factor inhibitors suitable for reducing or inhibiting the activity of a target transcription factor in a cell or a subject.

The objects mentioned above are solved by the underlying description and the accompanying claims.

### Short Description of the Invention

The present invention is inter alia directed to artificial nucleic acid constructs, preferably RNA, comprising at least one coding sequence encoding at least one transcription factor inhibitor for reducing or inhibiting the activity of a target transcription factor in a cell. A preferred transcription factor inhibitor according to the invention is a Runt-related transcription factor (RUNX) inhibitor, for example a RUNX trap comprising at least one amino acid sequence for binding a RUNX transcription factor and at least one amino acid sequence for capturing or trapping RUNX. Further provided are pharmaceutical compositions comprising the artificial nucleic acid, preferably formulated in polyethylene glycol/peptide polymers, polymeric carriers, or lipid-based carriers. Also provided are methods of treating or preventing disorders, diseases, or conditions, and medical uses.

As shown in the Example section, the invention is inter alia based on the surprising finding that artificial nucleic acid molecules, e.g. RNA molecules, that encode transcription factor inhibitors can be used as specific inhibitors of cellular transcription factors, in particular transcription factors that have a pathologic transcription factor activity, e.g. transcription factors that are overexpressed or overactive in a disease, disorder, or condition.

As exemplified for a Runt-related transcription factor (RUNX), a transient, localized expression of RUNX transcription factor inhibitors using artificial nucleic acid constructs led to a reduction or inhibition of the activity of the RUNX target transcription factor (RUNX1) (see Examples). The inventors generated an effective dominant negative inhibitor for RUNX (also herein referred to as RUNX-Trap), by transiently expressing a fusion protein comprising a fragment of Core Factor Binding β (CBFbeta, CBFβ, CBFB) and Smooth Muscle Myosin Heavy Chain (SMMHC, MYH11) using an artificial nucleic acid construct, in particular an RNA construct. Without wishing to be bound to theory, the protein fusion of CBFbeta-SMMHC inhibited RUNX1 transcription factor activity inter alia by preventing its nuclear translocation and by reducing the interaction with the cellular transcription co-factor CBFbeta (see FIG. 1). Surprisingly, after administration of the artificial nucleic acid, the produced RUNX inhibitor sequestered RUNX1 from the cell nucleus and strongly reduced proliferation in primary human cell cultures derived from surgically excised membranes from eyes of patients with proliferative vitreoretinopathy (PVR). PVR is a blinding, relatively common complication of retinal detachment often associated with eye trauma driven by RUNX1-mediated epithelial-mesenchymal transition (EMT) that currently lacks medical treatment. PVR is characterized by the development of membranous intraocular scar tissue (membranes that consist of proliferating cells and extracellular matrix) and is the most common cause of failure after retinal detachment surgery. In addition, it has been shown that alternative RUNX inhibitors were effective as well. As further shown herein, RUNX transcription factor inhibitors or traps (formulated in LNPs) were effective in a choroidal neovascularization (CNV) in vivo model.

As demonstrated herein, treatment using the nucleic acid encoded RUNX inhibitor blunted the expression of RUNX1 and shifted gene expression from a mesenchymal phenotype towards an epithelial profile across the EMT continuum. Further, the inventors showed that intraocular administration of the nucleic acid encoded RUNX inhibitor strongly reduced proliferation and ocular pathology triggered by injection of human PVR cells in a rabbit eye.

The present invention therefore demonstrates that artificial nuclei acid, in particular RNA, can be leveraged to provide transcription factor inhibitors for reducing or inhibiting the activity of a target transcription factor in a cell.

Therefore, the present invention forms the foundation for a plethora of various potential clinical applications that require an inhibition of intracellular target molecules such as intracellular transcription factors.

In a first aspect, the present invention provides an artificial nucleic acid comprising at least one coding sequence encoding at least one transcription factor inhibitor for reducing or inhibiting the activity of a target transcription factor in a cell.

In preferred embodiments, the transcription factor inhibitor is a Runt-related transcription factor inhibitor (e.g. RUNX inhibitor) or a Runt-related transcription factor trap (e.g. RUNX trap).

In particularly preferred embodiments, the Runt-related transcription factor (RUNX) inhibitor comprises or consists of a fusion protein comprising a CBFbeta amino acid sequence element and a SMMHC amino acid sequence element. Such a RUNX inhibitor sequester cellular RUNX by binding to a RUNX protein in the cytosol and preferably trapping the RUNX protein in the cytosol. Accordingly, said RUNX inhibitor can reduce or prevent the translocation of cellular CBFbeta from the cytosol to the nucleus by reducing or preventing its interaction with cellular RUNX (e.g. RUNX1).

In preferred embodiments, the artificial nucleic acid is an RNA, more preferably an mRNA.

In a second aspect, the present invention provides a pharmaceutical composition comprising at least one artificial nucleic acid comprising at least one coding sequence encoding at least one transcription factor inhibitor as defined in the first aspect.

In preferred embodiments, the artificial nucleic acid of the pharmaceutical composition is formulated in polyethylene glycol/peptide polymers, polymeric carriers, or lipid-based carriers (e.g. LNPs). Suitably, the formulation is selected from LNPs.

In a third aspect, the present invention provides a kit or kit of parts comprising at least one artificial nucleic acid of the first aspect or at least one pharmaceutical composition of the second aspect.

In further aspects, the present invention provides methods of treating or preventing disease, disorder or condition and first and further medical uses of the artificial nucleic acid, the pharmaceutical composition, or the kit or kit of parts.

In preferred embodiments, the disease, disorder or condition is an ocular disease, disorder, or condition, preferably proliferative vitreoretinopathy (PVR).

A further aspect relates to a method of reducing the activity of a transcription factor in a cell or a subject.

### Definitions

For the sake of clarity and readability the following definitions are provided. Any technical feature mentioned for these definitions may be read on each and every embodiment of the invention. Additional definitions and explanations may be specifically provided in the context of these embodiments.

Percentages in the context of numbers should be understood as relative to the total number of the respective items. In other cases, and unless the context dictates otherwise, percentages should be understood as percentages by weight (wt.-%).

About: The term “about” is used when determinants or values do not need to be identical, i.e. 100% the same. Accordingly, “about” means, that a determinant or values may diverge by 1% to 20% preferably by 1% to 10%; in particular, by 1%, 2%, 3%, 4%, 5%, 6%, 7%, 8%, 9%, 10%, 11%, 12%, 13%, 14%, 15%, 16%, 17%, 18%, 19%, 20%. Preferably, “about” means, that a determinant or values may diverge by +/−1%, +/−2%, +/−3%, +/−4%, +/−5%, +/−6%, +/−7%, +/−8%, +/−9%, +/−10%. The skilled person knows that e.g. certain parameters or determinants can slightly vary based on the method how the parameter has been determined.

Angiogenesis: As used herein, “angiogenesis” means the physiological process through which new blood vessels form from pre-existing vessels. Angiogenesis is particularly relevant to aberrant vessel growth in infants, children, adults, such as during tumor growth, and tumor-like growth, and e.g. in wet age-related macular degeneration, and proliferative diabetic retinopathy.

Blood vessel growth may occur via the process of angiogenesis and/or vasculogenesis. The processes are distinct, and the involvement of a protein or pathway in vasculogenesis (e.g., during embryonic development) does not necessarily indicate that the protein or pathway is relevant to angiogenesis, much less aberrant angiogenesis. Moreover, the involvement of a protein or pathway in embryonic angiogenesis does not indicate that targeting the protein or pathway would be capable of reducing the aberrant angiogenesis, much less sufficient for inhibiting aberrant angiogenesis or safe for targeting in an infant, child, or adult.

“Vasculogenesis” means the process of blood vessel formation occurring by a de novo production of endothelial cells. Vasculogenesis is particularly relevant to embryonic blood vessel formation. Vasculogenesis and angiogenesis are distinct from each other in that angiogenesis relates to the development of new blood vessels from (e.g., sprouting or extending from) pre-existing blood vessels, whereas vasculogenesis relates to the formation of new blood vessels that have not extended/sprouted from pre-existing blood vessels (e.g., where there are no pre-existing vessels). For example, if a monolayer of endothelial cells begins sprouting to form capillaries, angiogenesis is occurring. Vasculogenesis, in contrast, is when endothelial precursor cells (angioblasts) migrate and differentiate in response to local cues (such as growth factors and extracellular matrices) to form new blood vessels. These new blood vessels formed by vasculogenesis are then pruned and extended through angiogenesis.

Cationic: The term “cationic” means that the respective structure bears a positive charge, either permanently or not permanently, for example in response to certain conditions such as pH. Thus, the term “cationic” covers both “permanently cationic” and “cationisable”. The term “permanently cationic” means, e.g., that the respective compound, or group, or atom, is positively charged at any pH value or hydrogen ion activity of its environment. Typically, the positive charge results from the presence of a quaternary nitrogen atom.

Cationisable: The term “cationisable” as used herein means that a compound, or group or atom, is positively charged at a lower pH and uncharged at a higher pH of its environment. Also in non-aqueous environments where no pH value can be determined, a cationisable compound, group or atom is positively charged at a high hydrogen ion concentration and uncharged at a low concentration or activity of hydrogen ions. It depends on the individual properties of the cationisable or polycationisable compound, in particular the pKa of the respective cationisable group or atom, at which pH or hydrogen ion concentration it is charged or uncharged. In diluted aqueous environments, the fraction of cationisable compounds, groups or atoms bearing a positive charge may be estimated using the so-called Henderson-Hasselbalch equation which is well-known to a person skilled in the art. E.g., in some embodiments, if a compound or moiety is cationisable, it is preferred that it is positively charged at a pH value of about 1 to 9, preferably 4 to 9, 5 to 8 or even 6 to 8, more preferably of a pH value of or below 9, of or below 8, of or below 7, most preferably at physiological pH values, e.g. about 7.3 to 7.4, i.e. under physiological conditions, particularly under physiological salt conditions. In other embodiments, it is preferred that the cationisable compound or moiety is predominantly neutral at physiological pH values, e.g. about 7.0-7.4, but becomes positively charged at lower pH values. In some embodiments, the preferred range of pKa for the cationisable compound or moiety is about 5 to about 7.

Coding sequence, coding region, cds: The terms “coding sequence” or “coding region” and the corresponding abbreviation “cds” as used herein is intended to refer to a sequence of several nucleotide triplets, which may be translated into a peptide or protein. A coding sequence in the context of the present invention may be a DNA or RNA sequence consisting of a number of nucleotides that may be divided by three, which starts with a start codon and which preferably terminates with a stop codon. Suitably in the context of the invention, the coding sequence encodes at least one transcription factor, accordingly, the coding sequence provides the information that is translated into least one transcription factor inhibitor.

Derived from: The term “derived from” as used herein in the context of a nucleic acid, i.e. for a nucleic acid “derived from” (another) nucleic acid, means that the nucleic acid, which is derived from (another) nucleic acid, shares e.g. at least 60%, 70%, 80%, 81%, 82%, 83%, 84%, 85%, 86%, 87%, 88%, 89%, 90%, 91%, 92%, 93%, 94%, 95%, 96%, 97%, 98%, or 99% sequence identity or are identical with the nucleic acid from which it is derived. The skilled person is aware that sequence identity is typically calculated for the same types of nucleic acids, i.e. for DNA sequences or for RNA sequences. Thus, it is understood, if a DNA is “derived from” an RNA or if an RNA is “derived from” a DNA, in a first step the RNA sequence is converted into the corresponding DNA sequence (in particular by replacing the uracils (U) by thymidines (T) throughout the sequence) or, vice versa, the DNA sequence is converted into the corresponding RNA sequence (in particular by replacing the T by U throughout the sequence). Thereafter, the sequence identity of the DNA sequences or the sequence identity of the RNA sequences is determined. Preferably, a nucleic acid “derived from” a nucleic acid also refers to nucleic acid, which is modified in comparison to the nucleic acid from which it is derived, e.g. in order to increase RNA stability even further and/or to prolong and/or increase protein production. In the context of amino acid sequences the term “derived from” means that the amino acid sequence, which is derived from (another) amino acid sequence, shares e.g. at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity or are identical with the amino acid sequence from which it is derived.

Epithelial-mesenchymal transition (EMT): The term epithelial-mesenchymal transition and the corresponding abbreviation “EMT” as used herein will be recognized and understood by the person of ordinary skill in the art, and is e.g. characterized by a loss of cell adhesion, which leads to constriction and extrusion of new mesenchymal cells. EMT is a process by which epithelial cells lose their cell polarity, which leads to cell-cell adhesion loss, and gain of migratory and invasive properties to become mesenchymal stem cells (which are multipotent stromal cells that can differentiate into a variety of cell types). EMT is essential for numerous developmental processes including mesoderm formation and neural tube formation. EMT has also been shown to occur in wound healing, in organ fibrosis and in the initiation of metastasis in cancer progression. EMT, and its reverse process, MET (mesenchymal-epithelial transition) are critical for development of many tissues and organs in the developing embryo, and numerous embryonic events such as gastrulation, neural crest formation, heart valve formation, palatogenesis and myogenesis. Epithelial cells are closely connected to each other by tight junctions, gap junctions and adherens junctions, have an apico-basal polarity, polarization of the actin cytoskeleton and are bound by a basal lamina at their basal surface. Mesenchymal cells, on the other hand, lack this polarization, have a spindle-shaped morphology and interact with each other only through focal points. Epithelial cells express high levels of E-cadherin, whereas mesenchymal cells express those of N-cadherin, fibronectin and vimentin. Thus, EMT entails profound morphological and phenotypic changes to a cell. Based on the biological context, EMT has been categorized into 3 types: developmental (Type I), fibrosis and wound healing (Type II), and cancer (Type III). Loss of E-cadherin is a fundamental event in EMT. Many transcription factors (TFs) that can repress E-cadherin directly or indirectly are considered as EMT-TF (EMT inducing TFs). SNAII/Snail 1, SNAI2/Snail 2 (also known as Slug or Zinc finger protein), Zinc finger E-box binding homeobox 1 and 2 (ZEB1 and ZEB2), transcription factor 3 (TCF3) and krueppel-like factor 8 (KLF8) can bind to the E-cadherin promoter and repress its transcription, whereas factors such as Twist (also referred to as class A basic helix-loop-helix protein 38; bHLHa38), Goosecoid, transcription factor 4 (TCF4), homeobox protein Sineoculis homeobox homolog 1 (SIX1) and fork-head box protein C2 (FOXC2) repress E-cadherin indirectly. Several signaling pathways (transforming growth factor beta (TGFbeta), fibroblast growth factor (FGF), epidermal growth factor (EGF), hepatocyte growth factor (HGF), Wnt/beta-catenin and Notch) and hypoxia may induce EMT. In particular, Ras-MAPK (mitogen-activated protein kinases) activates Snail and Slug. Slug triggers the steps of desmosomal disruption, cell spreading, and partial separation at cell-cell borders, which comprise the first and necessary phase of the EMT process. Wnt signaling pathway regulates EMT in gastrulation, cardiac valve formation and cancer. Activation of Wnt pathway in breast cancer cells induces the EMT regulator SNAIL and upregulates the mesenchymal marker, vimentin. Also, active Wnt/beta-catenin pathway correlates with poor prognosis in breast cancer patients in the clinic. Similarly, TGFbeta activates the expression of SNAIL and ZEB to regulate EMT in heart development, palatogenesis, and cancer. The breast cancer bone metastasis has activated TGF-b signaling, which contributes to the formation of these lesions. However, on the other hand, tumor protein 53 (p53), a well-known tumor suppressor, represses EMT by activating the expression of various microRNAs-miR-200 and miR-34 that inhibit the production of protein ZEB and SNAIL, and thus maintain the epithelial phenotype. After the initial stage of embryogenesis, the implantation of the embryo and the initiation of placenta formation are associated with EMT. The trophoectoderm cells undergo EMT to facilitate the invasion of endometrium and appropriate placenta placement, thus enabling nutrient and gas exchange to the embryo. Later in embryogenesis, during gastrulation, EMT allows the cells to ingress in a specific area of the embryo—the primitive streak in amniotes, and the ventral furrow in Drosophila. The cells in this tissue express E-cadherin and apical-basal polarity. During wound healing, keratinocytes at the border of the wound undergo EMT and undergo re-epithelialization or MET when the wound is closed. Snail2 expression at the migratory front influences this state, as its overexpression accelerates wound healing. Similarly, in each menstrual cycle, the ovarian surface epithelium undergoes EMT during post-ovulatory wound healing. Initiation of metastasis requires invasion, which is enabled by EMT. Carcinoma cells in a primary tumor lose cell-cell adhesion mediated by E-cadherin repression and break through the basement membrane with increased invasive properties and enter the bloodstream through intravasation. Later, when these circulating tumor cells (CTCs) exit the bloodstream to form micro-metastases, they undergo MET for clonal outgrowth at these metastatic sites. Thus, EMT and MET form the initiation and completion of the invasion-metastasis cascade. At this new metastatic site, the tumor may undergo other processes to optimize growth. For example, EMT has been associated with programmed death ligand 1 (PD-L1) expression, particularly in lung cancer. Increased levels of PD-L1 suppresses the immune system which allows the cancer to spread more easily. EMT has been shown to be induced by androgen deprivation therapy in metastatic prostate cancer. Activation of EMT programs via inhibition of the androgen axis provides a mechanism by which tumor cells can adapt to promote disease recurrence and progression. Brachyury, Axl (tyrosine protein kinase receptor UFO), MEK, and Aurora kinase A are molecular drivers of these programs, and inhibitors are currently in clinical trials to determine therapeutic applications. Oncogenic protein kinase C iota type (PKC-iota) can promote melanoma cell invasion by activating Vimentin during EMT. PKC-iota inhibition or knockdown resulted an increase E-cadherin and ras homolog gene family, member A (RhoA) levels while decreasing total Vimentin, phophorylated Vimentin (S39) and partitioning defective 6 homolog alpha (Par6) in metastatic melanoma cells. Cells that undergo EMT gain stem cell-like properties, thus giving rise to Cancer Stem Cells (CSCs).

Fibrosis: The term “fibrosis” will be recognized and understood by the person of ordinary skill in the art, and inter alia relates to pathological wound healing in which e.g. connective tissue replaces normal parenchymal tissue to the extent that it goes unchecked, leading to considerable tissue remodelling and the formation of permanent scar tissue. Repeated injuries, chronic inflammation and repair are typically susceptible to fibrosis where an accidental excessive accumulation of extracellular matrix components, such as the collagen is produced by fibroblasts, leading to the formation of a permanent fibrotic scar. In response to injury, this is called scarring, and if fibrosis arises from a single cell line, this is called a fibroma. Physiologically, fibrosis acts to deposit connective tissue, which can interfere with or totally inhibit the normal architecture and function of the underlying organ or tissue. Fibrosis can be used to describe the pathological state of excess deposition of fibrous tissue, as well as the process of connective tissue deposition in healing. Defined by the pathological accumulation of extracellular matrix (ECM) proteins, fibrosis results in scarring and thickening of the affected tissue. It is in essence an exaggerated wound healing response which interferes with normal organ function. From the physiological perspective, fibrosis is similar to the process of scarring, in that both involve stimulated fibroblasts laying down connective tissue, including collagen and glycosaminoglycans. The process is initiated when immune cells such as macrophages release soluble factors that stimulate fibroblasts. The most well characterized pro-fibrotic mediator is TGFbeta, which is released by macrophages as well as any damaged tissue between surfaces called interstitium. Other soluble mediators of fibrosis include CTGF, platelet-derived growth factor (PDGF), and interleukin 10 (IL-10). These initiate signal transduction pathways such as the AKT/mTOR and SMAD pathways that ultimately lead to the proliferation and activation of fibroblasts, which deposit extracellular matrix into the surrounding connective tissue. This process of tissue repair is a complex one, with tight regulation of extracellular matrix (ECM) synthesis and degradation ensuring maintenance of normal tissue architecture. However, the entire process, although necessary, can lead to a progressive irreversible fibrotic response if tissue injury is severe or repetitive, or if the wound healing response itself becomes deregulated. Fibrosis can occur in many tissues within the body, typically as a result of inflammation or damage, and examples include pathologies in the lung (e.g. cystic fibrosis, idiopathic pulmonary fibrosis), pathologies in the liver (e.g. cirrhosis), or pathologies in the heart (e.g. myocardial fibrosis).

Fragment: The term “fragment” as used herein in the context of a nucleic acid sequence (e.g. RNA or a DNA) or an amino acid sequence may typically be a shorter portion of a full-length sequence of e.g. a nucleic acid sequence or an amino acid sequence. Accordingly, a fragment typically consists of a sequence that is identical to the corresponding stretch within the full-length sequence. A preferred fragment of a sequence in the context of the present invention, consists of a continuous stretch of entities, such as nucleotides or amino acids corresponding to a continuous stretch of entities in the molecule the fragment is derived from, which represents at least 40%, 50%, 60%, 70%, 80%, 90%, 95% of the total (i.e. full-length) molecule from which the fragment is derived. The term “fragment” as used throughout the present specification in the context of proteins or peptides may, typically, comprise a sequence of a protein or peptide as defined herein, which is, with regard to its amino acid sequence, N-terminally and/or C-terminally truncated compared to the amino acid sequence of the original protein. The term “fragment” as used throughout the present specification in the context of RNA sequences may, typically, comprise an RNA sequence that is 5′-terminally and/or 3′-terminally truncated compared to the reference RNA sequence. Such truncation may thus occur either on the amino acid level or correspondingly on the nucleic acid level. A sequence identity with respect to such a fragment as defined herein may therefore preferably refer to the entire protein or peptide as defined herein or to the entire (coding) nucleic acid molecule of such a protein or peptide.

Identity (of a sequence): The term “identity” as used herein in the context of a nucleic acid sequence or an amino acid sequence will be recognized and understood by the person of ordinary skill in the art, and is e.g. intended to refer to the percentage to which two sequences are identical. To determine the percentage to which two sequences are identical, e.g. nucleic acid sequences or amino acid (aa) sequences, preferably the aa sequences encoded by the nucleic acid sequence as defined herein or the aa sequences themselves, the sequences can be aligned in order to be subsequently compared to one another. Therefore, e.g. a position of a first sequence may be compared with the corresponding position of the second sequence. If a position in the first sequence is occupied by the same residue as is the case at a position in the second sequence, the two sequences are identical at this position. If this is not the case, the sequences differ at this position. If insertions occur in the second sequence in comparison to the first sequence, gaps can be inserted into the first sequence to allow a further alignment. If deletions occur in the second sequence in comparison to the first sequence, gaps can be inserted into the second sequence to allow a further alignment. The percentage to which two sequences are identical is then a function of the number of identical positions divided by the total number of positions including those positions which are only occupied in one sequence. The percentage to which two sequences are identical can be determined using an algorithm, e.g. an algorithm integrated in the BLAST program.

Neovascularization: The term “neovascularization” has to be understood as the (natural) process of formation of new blood vessels. Typically, neovascularization is in the form of functional microvascular networks, capable of perfusion by red blood cells, that form to serve as collateral circulation in response to local poor perfusion or ischemia. Growth factors that inhibit neovascularization include those that affect endothelial cell division and differentiation. These growth factors often act in a paracrine or autocrine fashion; they include fibroblast growth factor, placental growth factor, insulin-like growth factor, hepatocyte growth factor, and platelet-derived endothelial growth factor. Typically, there are three different pathways that comprise neovascularization: (1) vasculogenesis, (2) angiogenesis, and (3) arteriogenesis. Several pathologies and diseases can be associated with aberrant neovascularization, including ocular pathologies such as corneal neovascularization, retinopathy of prematurity, diabetic retinopathy, age-related macular degeneration, and choroidal neovascularization. Aberrant neovascularization can also be associated with cardiovascular diseases e.g. Ischemic heart disease.

Nucleic acid, nucleic acid molecule: The terms “nucleic acid” or “nucleic acid molecule” will be recognized and understood by the person of ordinary skill in the art. The term “nucleic acid” or “nucleic acid molecule” as used herein preferably refers to DNA (molecules) or RNA (molecules). It is preferably used synonymously with the term polynucleotide. Preferably, a nucleic acid or a nucleic acid molecule is a polymer comprising or consisting of nucleotide monomers, which are covalently linked to each other by phosphodiester-bonds of a sugar/phosphate-backbone. The term “nucleic acid molecule” also encompasses modified nucleic acid molecules, such as base-modified, sugar-modified, or backbone-modified DNA or RNA molecules as defined herein.

Nucleic acid sequence, DNA sequence, RNA sequence: The terms “nucleic acid sequence”, “DNA sequence”, “RNA sequence” will be recognized and understood by the person of ordinary skill in the art, and e.g. refer to a particular and individual order of the succession of its nucleotides.

Nucleic species: In the context of the invention, the term “nucleic acid species” is not restricted to mean one single molecule but is understood to comprise an ensemble of essentially identical nucleic acid molecules. Accordingly, it may relate to a plurality of essentially identical nucleic acid molecules.

RNA species: In the context of the invention, the term “RNA species” is not restricted to mean one single molecule but is understood to comprise an ensemble of essentially identical RNA molecules. Accordingly, it may relate to a plurality of essentially identical RNA molecules.

RNA: The term “RNA” is the usual abbreviation for ribonucleic acid. It is a nucleic acid molecule, i.e. a polymer consisting of nucleotide monomers. These nucleotides are usually adenosine-monophosphate (AMP), uridine-monophosphate (UMP), guanosine-monophosphate (GMP) and cytidine-monophosphate (CMP) monomers or analogs thereof, which are connected to each other along a so-called backbone. The backbone is typically formed by phosphodiester bonds between the sugar, i.e. ribose, of a first and a phosphate moiety of a second, adjacent monomer. The specific order of the monomers, i.e. the order of the bases linked to the sugar/phosphate-backbone, is called the RNA sequence. RNA can be obtained by transcription of a DNA sequence, e.g., inside a cell or in vitro. In the context of the invention, the RNA may be obtained by RNA in vitro transcription. Alternatively, RNA may be obtained by chemical synthesis.

RNA in vitro transcription: The terms “RNA in vitro transcription” or “in vitro transcription” relate to a process wherein RNA is synthesized in a cell-free system in vitro. RNA may be obtained by DNA-dependent in vitro transcription of an appropriate DNA template, which is typically a linear DNA template (e.g. linearized plasmid DNA or PCR product). The promoter for controlling RNA in vitro transcription can be any promoter for any DNA-dependent RNA polymerase. Particular examples of DNA-dependent RNA polymerases are the T7, T3, SP6, or Syn5 RNA polymerases. In the context of the invention, the DNA template is typically linearized with a suitable restriction enzyme before it is subjected to RNA in vitro transcription. Reagents typically used in RNA in vitro transcription include: a DNA template (linearized plasmid DNA or PCR product) with a promoter sequence that has a high binding affinity for its respective RNA polymerase such as bacteriophage-encoded RNA polymerases (T7, T3, SP6, or Syn5); ribonucleotide triphosphates (NTPs) for the four bases (adenine, cytosine, guanine and uracil); optionally, a cap analogue as defined herein; optionally, modified nucleotides as defined herein; a DNA-dependent RNA polymerase capable of binding to the promoter sequence within the DNA template (e.g. 17, T3, SP6, or Syn5 RNA polymerase); optionally, a ribonuclease (RNase) inhibitor to inactivate any potentially contaminating RNase; optionally, pyrophosphatase; MgCl₂; a buffer (TRIS or HEPES) to maintain a suitable pH value, which can also contain antioxidants (e.g. DTT), and/or polyamines such as spermidine.

Variant (of a sequence); The term “variant” as used herein in the context of a nucleic acid sequence will be recognized and understood by the person of ordinary skill in the art, and is e.g. intended to refer to a variant of a nucleic acid sequence derived from another nucleic acid sequence. E.g., a variant of a nucleic acid sequence may exhibit one or more nucleotide deletions, insertions, additions and/or substitutions compared to the nucleic acid sequence from which the variant is derived. A variant of a nucleic acid sequence may at least 50%, 60%, 70%, 80%, 90%, or 95% identical to the nucleic acid sequence the variant is derived from. A variant may be a functional variant in the sense that the variant has retained at least 50%, 60%, 70%, 80%, 90%, or 95% or more of the function of the sequence where it is derived from. A “variant” of a nucleic acid sequence may have at least 70%, 75%, 80%, 85%, 90%, 95%, or 99% nucleotide identity over a stretch of at least 30, 50, 75 or 100 nucleotide of such nucleic acid sequence.

The term “variant” as used herein in the context of proteins or peptides is e.g. intended to refer to a proteins or peptide variant having an amino acid sequence which differs from the original sequence in one or more mutation(s)/substitution(s), such as one or more substituted, inserted and/or deleted amino acid(s). Preferably, these fragments and/or variants have the same, or a comparable specific property. Insertions and substitutions are possible, in particular, at those sequence positions which cause no modification to the three-dimensional structure or do not affect the binding region. Modifications to a three-dimensional structure by insertion(s) or deletion(s) can easily be determined e.g. using CD spectra (circular dichroism spectra). A variant of a protein may be a functional variant of the protein, which means that the variant exerts essentially the same, or at least 40%, 50%, 60%, 70%, 80%, 90% of the function of the protein it is derived from. A “variant” of a protein or peptide may have at least 70%, 75%, 80%, 85%, 90%, 95%, 98% or 99% amino acid identity over a stretch of at least 30, 50, 75 or 100 amino acids of such protein or peptide.

### DETAILED DESCRIPTION OF THE INVENTION

Where reference is made to “SEQ ID NOs” of other patent applications or patents, said sequences, e.g. amino acid sequences or nucleic acid sequences, are explicitly incorporated herein by reference. For “SEQ ID NOs” provided herein, information provided under “feature key”, i.e. “source” (for nucleic acids or proteins) or “misc_feature” (for nucleic acids) or “REGION” (for proteins). (in the sequence listing according to WIPO ST.26 Standard) is also explicitly included herein in its entirety. Where reference is made to “SEQ ID NOs” in the context of RNA sequences, the skilled person will be able to derive RNA sequences from the referenced SEQ ID NOs also in cases where DNA sequences are provided. Where reference is made to “SEQ ID NOs” in the context of DNA sequences, the skilled person will be able to derive respective DNA sequences from the referenced SEQ ID NOs also in cases where RNA sequences are provided.

### 1: Nucleic Acid Encoding at Least One Transcription Factor Inhibitor:

In a first aspect, the invention provides a nucleic acid encoding at least one transcription factor inhibitor.

It has to be noted that specific features and embodiments that are described in the context of the first aspect of the invention, that is the nucleic acid of the invention, are likewise applicable to the second aspect (pharmaceutical composition of the invention), the third aspect (kit or kit of parts of the invention), or further aspects including medical uses and method of treatments.

In preferred embodiments, the nucleic acid encoding the at least one transcription factor inhibitor is an artificial nucleic acid.

The term “artificial nucleic acid” as used herein is intended to refer to a nucleic acid that does not occur naturally. In other words, an artificial nucleic acid may be understood as a non-natural nucleic acid molecule. Such nucleic acid molecules may be non-natural due to its individual sequence (e.g. G/C content modified coding sequence, UTRs) and/or due to other modifications, e.g. structural modifications of nucleotides. Typically, artificial nucleic acid may be designed and/or generated by genetic engineering to correspond to a desired artificial sequence of nucleotides. In this context, an artificial nucleic acid is a sequence that may not occur naturally, i.e. a sequence that differs from the wild type sequence/the naturally occurring sequence by at least one nucleotide. The term “artificial nucleic acid” is not restricted to mean “one single molecule” but is understood to comprise an ensemble of essentially identical nucleic acid molecules. The term “artificial nucleic acid” as used herein may relate to artificial DNA or, preferably, to artificial RNA. Preferably, the artificial nucleic is selected from an artificial DNA or an artificial RNA.

In preferred embodiments, the artificial nucleic acid comprises at least one coding sequence encoding at least one transcription factor inhibitor.

The term “target transcription factor” as used herein is intended to refer to the cellular transcription factor that is intended to be inhibited by the at least one transcription factor inhibitor (encoded by the artificial nucleic acid). In various embodiments, inhibiting the “target transcription factor” is associated with advantageous cellular or physiological effects as further outlined herein.

“Cellular” in the context of the invention e.g. in the context of a protein (e.g. “cellular target transcription factor” or “cellular transcription co-factor”) relates to the respective protein that is present in a cellular environment. Accordingly, the term refers to the respective physiological protein and not to the protein that is provided by the artificial nucleic acid of the invention.

The inhibition of the target transcription factor can be a direct inhibition (e.g. via an interaction of the transcription factor inhibitor with the cellular target transcription factor) resulting in reduced transcriptional activity of the target transcription factor in a cell. An example is the direct inhibition of the cellular target transcription factor RUNX e.g. via a direct interaction of the provided transcription factor inhibitor with the cellular target transcription factor RUNX, resulting in reduced transcriptional activity of the target transcription factor RUNX in a cell.

Alternatively, the inhibition of the target transcription factor can be an indirect inhibition (e.g. via an interaction of the transcription factor inhibitor with a co-factor of the target transcription factor) which may also result in a reduced transcriptional activity of the target transcription factor in a cell. An example is the indirect inhibition of the cellular target transcription factor RUNX e.g. via interaction of the provided transcription factor inhibitor with its co-factor CBFbeta which may also result in reduced transcriptional activity of the target transcription factor RUNX in a cell as e.g. the interaction of cellular CBFbeta with cellular RUNX is disturbed.

Preferably, the artificial nucleic acid additionally comprises at least one heterologous nucleic acid sequence element. A preferred heterologous nucleic acid sequence may be selected from at least one heterologous untranslated region (UTR).

The term “heterologous” sequence as used herein is intended to refer to a nucleic acid sequence that is not from the same gene or the same genomic fusion. Accordingly, heterologous sequences may be derivable from the same organism (e.g. human) or from a different organism. Heterologous sequences do naturally (in nature) not occur in the same nucleic acid molecule.

In preferred embodiments, the artificial nucleic acid comprises at least one coding sequence encoding at least one transcription factor inhibitor, wherein the transcription factor inhibitor reduces or inhibits the activity of a target transcription factor in a cell, wherein the nucleic acid comprises at least one heterologous untranslated region (UTR).

In some embodiments, the encoded transcription factor inhibitor is not an intrabody.

In preferred embodiments, the transcription factor inhibitor (that is provided by the artificial nucleic acid) is produced in the cytosol upon administration of the artificial nucleic acid to a cell, tissue, or subject.

Accordingly, the administration of the artificial nucleic acid (e.g. RNA) to a cell, tissue, or subject leads to a translation of the at least one coding sequence into at least one transcription factor inhibitor protein. Accordingly, whenever reference is made to a “produced transcription factor inhibitor”, the term relates to the protein product that is generated from the artificial nucleic acid of the invention by translating the coding sequence of the nucleic acid into a protein. Accordingly, functional and structural features and embodiments that are described herein relating to the “transcription factor inhibitor” or relating to the “(produced) transcription factor inhibitor” should be understood to refer to transcription factor inhibitor proteins that are produced/translated in the cytosol upon administration of the artificial nucleic acid of the invention to a cell, tissue, or subject.

In preferred embodiments, upon administration of the artificial nucleic acid to a cell or subject, the produced transcription factor inhibitor is a dominant negative inhibitor of the target transcription factor. In preferred embodiments, the produced transcription factor inhibitor is a dominant negative inhibitor of the target transcription factor and/or its transcription co-factor.

In general, a dominant negative inhibition is a phenomenon in which the function of a wild-type gene product (e.g. a transcription factor protein or its transcription co-factor protein) is impaired by a co-expressed mutant or variant of the same gene product or a related gene product.

In preferred embodiments, upon administration of the artificial nucleic acid to a cell or subject, the produced transcription factor inhibitor binds to the cellular target transcription factor.

Accordingly, the transcription factor inhibitor comprises at least one amino acid sequence that facilitates binding to the target transcription factor (e.g. RUNX). Advantageously, the binding of the (produced) transcription factor inhibitor to the cellular target transcription factor is strong enough to reduce the activity of a target transcription factor in a cell.

In preferred embodiments, upon administration of the artificial nucleic acid to a cell or subject, the produced transcription factor inhibitor binds to at least one transcription co-factor of the target transcription factor. Preferably, the transcription co-factor is selected from a transcription co-activator.

The term “transcription co-factor of the target transcription factor” relates to any factor (e.g. co-factor protein, non-coding nucleic acid e.g. a non-coding RNA) that is able to modulate the activity or function of the target transcription factor. Typically, a transcription co-factor can interact with a transcription factor to promote the formation of transcription complexes, or a transcription co-factor can influence the affinity of a transcription factor to its target DNA (e.g. promoter sequence). Most transcription factors require transcription co-factors to be fully functional. For gene transcription to occur, a number of transcription factors must bind to DNA regulatory sequences. This collection of transcription factors, in turn, inter alia recruit intermediary proteins such as cofactors that allow efficient recruitment of the preinitiation complex and RNA polymerase. Thus, for a single transcription factor to initiate transcription, all of these other proteins must typically also be present, and the transcription factor must be in a state where it can bind to them if necessary. Typically, a transcription co-factor can have inhibitory or activatory function. In case of activatory functions, such transcription co-factors are also called co-activators. An example of a transcription co-factor is CBFbeta which is a co-activator of the transcription factor RUNX (e.g. RUNX1).

In preferred embodiments, upon administration of the artificial nucleic acid to a cell or subject, the produced transcription factor inhibitor reduces or prevents interaction of the cellular target transcription factor with its target DNA.

The term “target DNA” relates to the DNA sequence in the nucleus to which a transcription factor binds to. Typically, a transcription factor comprises at least one DNA-binding domain, which attaches to a specific sequence of DNA adjacent to the genes that they regulate. Target DNA sequences may comprise DNA regulatory sequences, for example DNA promoter sequences or enhancer sequences. In the context of the invention, the interaction of the target transcription factor with its target DNA can be reduced by e.g. reducing or preventing translocation of the target transcription factor into the nucleus and/or by reducing translocation of a co-factor of the target transcription factor into the nucleus. It can also be reduced by mutations in the transcription factor that reduce the affinity of binding to DNA.

In preferred embodiments, upon administration of the artificial nucleic acid to a cell or subject, the produced transcription factor inhibitor reduces or prevents interaction of the cellular target transcription factor with at least one of its cellular transcription co-factors. Suitably, the transcription co-factor is selected from a transcription co-activator.

For example, the interaction of the target transcription factor (e.g. RUNX) with at least one of its transcription co-factors (e.g. CBFbeta) may be reduced by binding of the produced transcription factor inhibitor to the cellular target transcription factor (which prevents interaction of the transcription factor with its co-factor). Alternatively, the interaction of the target transcription factor with at least one of its transcription co-factors may be reduced by binding of the produced transcription factor inhibitor to the cellular target transcription co-factor (which prevents interaction of the transcription factor with its co-factor).

In preferred embodiments, upon administration of the artificial nucleic acid to a cell or subject, the produced transcription factor inhibitor reduces or prevents nuclear translocation of the cellular target transcription factor and/or its transcription co-factor. In particularly preferred embodiments, the produced transcription factor inhibitor reduces or prevents nuclear translocation of the cellular target transcription factor and its transcription co-factor.

The term “nuclear translocation” relates to the transport of a protein from the cytosol into the nucleus. Proteins are typically translocated into the nucleus through nuclear pore complexes (NPCs) by receptor-mediated import pathways. For getting transported into the nucleus, proteins typically comprise specific amino acid sequences (e.g. nuclear localization signal, NLS) that promote nuclear translocation or they have to interact with proteins that comprise such specific amino acid sequences (e.g. NLS).

In the context of the invention, the transcription factor inhibitor (that is provided by the nucleic acid) may be configured to reduce or prevent nuclear translocation of the cellular target transcription factor (e.g., RUNX). The reduction or prevention of nuclear translocation may be achieved by binding of the produced transcription factor inhibitor to the cellular target transcription factor in the cytosol. Alternatively or in addition, the reduction or prevention of nuclear translocation of the target transcription factor (e.g., RUNX) may lead to a reduction or prevention of nuclear translocation of proteins that interact with said target transcription factor (e.g. transcription co-factors of RUNX such as CBFbeta).

In preferred embodiments, upon administration of the artificial nucleic acid to a cell or subject, the produced transcription factor inhibitor reduces the activity of the target transcription factor.

The “reducing the activity of the target transcription factor” can be a direct e.g. via a direct interaction of the produced transcription factor inhibitor with the cellular target transcription factor (e.g. RUNX), or indirect e.g. via interaction of the produced transcription factor inhibitor with at least one cellular transcription co-factor (e.g. CBFbeta) of the respective target transcription factor (e.g. RUNX).

In preferred embodiments, upon administration of the artificial nucleic acid to a cell or subject, the produced transcription factor inhibitor reduces the cellular expression of the target transcription factor.

In cells, the expression of transcription factors is often regulated by self-regulatory feedback loops. That means that e.g. transcription factors proteins can activate their own expression (self-activation). As the transcription factor inhibitor of the present invention may reduce or inhibit the activity of the target transcription factor in a cell (e.g. RUNX), that can also lead to a reduced expression of the target transcription factor as such. Accordingly, a further reduction of the cellular expression of the target transcription factor may increase or enhance advantageous cellular or physiological effects of the transcription factor inhibitor that is provided by the artificial nucleic acid.

Typically, transcription factors bind to either enhancer or promoter regions of DNA adjacent to the genes that they regulate. Depending on the transcription factor, the transcription of the adjacent gene is either up- or down-regulated. Transcription factors use a variety of mechanisms for the regulation of gene expression. Typically, transcription factors directly control or regulate the expression of various different proteins in a cell e.g. by directly activating the transcription of genes. In addition, these directly regulated gene-products can also be involved in the regulation and expression of other gene-products. Accordingly, the transcription factor inhibitor may reduce (or alternatively increase) the cellular expression of proteins that are directly controlled or regulated by the target transcription factor and, additionally, reduce or increase the cellular expression of further proteins that are indirectly controlled or regulated by the target transcription factor (e.g. via the above described gene-products that are directly controlled by the target transcription factor).

In preferred embodiments, upon administration of the artificial nucleic acid to a cell or subject, the produced transcription factor inhibitor reduces the cellular expression of proteins that are controlled or regulated by the target transcription factor. Notably, “controlled or regulated” in that context may be directly or indirectly, preferably directly.

Alternatively or additionally, the produced transcription factor inhibitor increases the cellular expression of proteins that are controlled or regulated by the target transcription factor. Notably, “controlled or regulated” in that context may be directly or indirectly, preferably directly.

In preferred embodiments, the encoded transcription factor inhibitor is a transcription factor trap preferably configured to bind and trap the target transcription factor in the cytosol.

The term “transcription factor trap” in the context of the invention has to be understood as a protein that is configured to bind to a cellular target transcription factor (e.g. via an amino acid sequence that interacts or binds to the target transcription factor) and is additionally capable of capturing or trapping said target transcription factor in the cytosol (e.g. via an amino acid sequence that blocks nuclear translocation of the target transcription factor). One example of a transcription factor trap in the context of the invention is a RUNX trap, in particular a RUNX1 trap. A “transcription factor trap” is particularly suitable in the context of the invention as the target transcription factor is blocked from entering into the nucleus where transcription factors typically act. Blocking of the target transcription factor to enter into the nucleus (e.g. RUNX) may also be associated with a reduced or blocked transport of its transcription co-factors (e.g. CBFbeta), in particular in cases where the transcription co-factors of the target transcription factor is transported via the transcription factor into the nucleus. Of course, in such a case, a capturing of the target transcription factor in the cytosol (e.g. RUNX) would, at the same time, prevent or reduce the transport of its transcription co-factors (e.g. CBFbeta) into the nucleus.

In preferred embodiments, the target transcription factor is selected from a transcription factor that has an aberrant transcription factor activity or pathologic transcription factor activity.

Suitably, the aberrant or pathologic transcription factor activity is an overexpression and/or an overactivation.

In preferred embodiments, the target transcription factor is selected from a transcription factor that has an aberrant or pathologic transcription factor activity (e.g. overexpression and/or an overactivation) associated with epithelial to mesenchymal transition (EMT), pathological cell proliferation, aberrant angiogenesis, aberrant neovascularization, degeneration, and/or fibrosis.

An example of a target transcription factor that shows pathologic transcription factor activity (e.g. overexpression and/or an overactivation) associated with EMT, pathological cell proliferation, aberrant angiogenesis, aberrant neovascularization, degeneration, and/or fibrosis is the Runt-related transcription factor RUNX1.

In preferred embodiments, the target transcription factor is selected from a transcription factor that is overexpressed and/or overactive in a disease, disorder, or condition.

In preferred embodiments, the target transcription factor is selected from a transcription factor that is overexpressed or overactive in an ocular disease, disorder, or condition.

In preferred embodiments, the target transcription factor is selected from a human transcription factor, preferably a member of the following list (List A): AC008770.3; AC023509.3; AC092835.1; AC138696.1; ADNP; ADNP2; AEBP1; AEBP2; AHCTF1; AHDC1; AHR; AHRR; AIRE; AKAP8; AKAP8L; AKNA; ALX1; ALX3; ALX4; ANHX; ANKZF1; AP1; AR; ARGFX; ARHGAP35; ARID2; ARID3A; ARID3B; ARID3C; ARID5A; ARID5B; ARNT; ARNT2; ARNTL; ARNTL2; ARX; ASCL1; ASCL2; ASCL3; ASCL4; ASCL5; ASH1L; ATF1; ATF2; ATF3; ATF4; ATF5; ATF6; ATF6B; ATF7; ATMIN; ATOH1; ATOH7; ATOH8; BACH1; BACH2; BARHL1; BARHL2; BARX1; BARX2; BATF; BATF2; BATF3; BAZ2A; BAZ2B; BBX; BCL11A; BCL11B; BCL6; BCL6B; BHLHA15; BHLHA9; BHLHE22; BHLHE23; BHLHE40; BHLHE41; BNC1; BNC2; BORCS8-MEF2B; BPTF; BRF2; BSX; C11orf95; CAMTA1; CAMTA2; CARF; CASZ1; CBX2; CC2D1A; CCDC169-SOHLH2; CCDC17; CDC5L; CDX1; CDX2; CDX4; CEBPA; CEBPB; CEBPD; CEBPE; CEBPG; CEBPZ; CENPA; CENPB; CENPBD1; CENPS; CENPT; CENPX; CGGBP1; CHAMP1; CHCHD3; CIC; CLOCK; CPEB1; CPXCR1; CREB1; CREB3; CREB3L1; CREB3L2; CREB3L3; CREB3L4; CREB5; CREBL2; CREBZF; CREM; CRX; CSRNP1; CSRNP2; CSRNP3; CTCF; CTCFL; CUX1; CUX2; CXXC1; CXXC4; CXXC5; DACH1; DACH2; DBP; DBX1; DBX2; DDIT3; DEAF1; DLX1; DLX2; DLX3; DLX4; DLX5; DLX6; DMBX1; DMRT1; DMRT2; DMRT3; DMRTA1; DMRTA2; DMRTB1; DMRTC2; DMTF1; DNMT1; DNTTIP1; DOT1L; DPF1; DPF3; DPRX; DR1; DRAP1; DRGX; DUX1; DUX3; DUX4; DUXA; DZIP1; E2F1; E2F2; E2F3; E2F4; E2F5; E2F6; E2F7; E2F8; E4F1; EBF1; EBF2; EBF3; EBF4; EEA1; EGR1; EGR2; EGR3; EGR4; EHF; ELF1; ELF2; ELF3; ELF4; ELF5; ELK1; ELK3; ELK4; EMX1; EMX2; EN1; EN2; EOMES; EPAS1; ERF; ERG; ESR1; ESR2; ESRRA; ESRRB; ESRRG; ESX1; ETS1; ETS2; ETV1; ETV2; ETV3; ETV3L; ETV4; ETV5; ETV6; ETV7; EVX1; EVX2; FAM170A; FAM200B; FBXL19; FERD3L; FEV; FEZF1; FEZF2; FIGLA; FIZ1; FLI1; FLYWCH1; FOS; FOSB; FOSL1; FOSL2; FOXA1; FOXA2; FOXA3; FOXB1; FOXB2; FOXC1; FOXC2; FOXD1; FOXD2; FOXD3; FOXD4; FOXD4L1; FOXD4L3; FOXD4L4; FOXD4L5; FOXD4L6; FOXE1; FOXE3; FOXF1; FOXF2; FOXG1; FOXH1; FOXI1; FOXI2; FOXI3; FOXJ1; FOXJ2; FOXJ3; FOXK1; FOXK2; FOXL1; FOXL2; FOXM1; FOXN1; FOXN2; FOXN3; FOXN4; FOXO1; FOXO3; FOXO4; FOX06; FOXP1; FOXP2; FOXP3; FOXP4; FOXQ1; FOXR1; FOXR2; FOXS1; GABPA; GATA1; GATA2; GATA3; GATA4; GATA5; GATA6; GATAD2A; GATAD2B; GBX1; GBX2; GCM1; GCM2; GFI1; GFI1B; GLI1; GLI2; GLI3; GLI4; GLIS1; GLIS2; GLIS3; GLMP; GLYR1; GMEB1; GMEB2; GPBP1; GPBP1L1; GRHL1; GRHL2; GRHL3; GSC; GSC2; GSX1; GSX2; GTF2B; GTF21; GTF2IRD1; GTF2IRD2; GTF2IRD2B; GTF3A; GZF1; HAND1; HAND2; HBP1; HDX; HELT; HES1; HES2; HES3; HES4; HES5; HES6; HES7; HESX1; HEY1; HEY2; HEYL; HHEX; HIC1; HIC2; HIF1A; HIF3A; HINFP; HIVEP1; HIVEP2; HIVEP3; HKR1; HLF; HLX; HMBOX1; HMG20A; HMG20B; HMGA1; HMGA2; HMGN3; HMX1; HMX2; HMX3; HNF1A; HNF1B; HNF4A; HNF4G; HOMEZ; HOXA1; HOXA10; HOXA11; HOXA13; HOXA2; HOXA3; HOXA4; HOXA5; HOXA6; HOXA7; HOXA9; HOXB1; HOXB13; HOXB2; HOXB3; HOXB4; HOXB5; HOXB6; HOXB7; HOXB8; HOXB9; HOXC10; HOXC11; HOXC12; HOXC13; HOXC4; HOXC5; HOXC6; HOXC8; HOXC9; HOXD1; HOXD10; HOXD11; HOXD12; HOXD13; HOXD3; HOXD4; HOXD8; HOXD9; HSF1; HSF2; HSF4; HSF5; HSFX1; HSFX2; HSFY1; HSFY2; IKZF1; IKZF2; IKZF3; IKZF4; IKZF5; INSM1; INSM2; IRF1; IRF2; IRF3; IRF4; IRF5; IRF6; IRF7; IRF8; IRF9; IRX1; IRX2; IRX3; IRX4; IRX5; IRX6; ISL1; ISL2; ISX; JAZF1; JDP2; JRK; JRKL; JUN; JUNB; JUND; KAT7; KCMF1; KCNIP3; KDM2A; KDM2B; KDM5B; KIN; KLF1; KLF10; KLF11; KLF12; KLF13; KLF14; KLF15; KLF16; KLF17; KLF2; KLF3; KLF4; KLF5; KLF6; KLF7; KLF8; KLF9; KMT2A; KMT2B; L3MBTL1; L3MBTL3; L3MBTL4; LBX1; LBX2; LCOR; LCORL; LEF1; LEUTX; LHX1; LHX2; LHX3; LHX4; LHX5; LHX6; LHX8; LHX9; LIN28A; LIN28B; LIN54; LMX1A; LMX1B; LTF; LYL1; MAF; MAFA; MAFB; MAFF; MAFG; MAFK; MAX; MAML1; MAML2; MAZ; MBD1; MBD2; MBD3; MBD4; MBD6; MBNL2; MECOM; MECP2; MEF2A; MEF2B; MEF2C; MEF2D; MEIS1; MEIS2; MEIS3; MEOX1; MEOX2; MESP1; MESP2; MGA; MITF; MIXL1; MKX; MLX; MLXIP; MLXIPL; MNT; MNX1; MSANTD1; MSANTD3; MSANTD4; MSC; MSGN1; MSX1; MSX2; MTERF1; MTERF2; MTERF3; MTERF4; MTF1; MTF2; MXD1; MXD3; MXD4; MXI1; MYB; MYBL1; MYBL2; MYC; MYCL; MYCN; MYF5; MYF6; MYNN; MYOD1; MYOG; MYPOP; MYRF; MYRFL; MYSM1; MYT1; MYT1L; MZF1; NACC2; NAIF1; NANOG; NANOGNB; NANOGP8; NCOA1; NCOA2; NCOA3; NEUROD1; NEUROD2; NEUROD4; NEUROD6; NEUROG1; NEUROG2; NEUROG3; NF-kappaB; NFAT5; NFAT5A; NFATC1; NFATC2; NFATC3; NFATC4; NFE2; NFE2L1; NFE2L2; NFE2L3; NFE4; NFIA; NFIB; NFIC; NFIL3; NFIX; NFKB1; NFKB2; NFX1; NFXL1; NFYA; NFYB; NFYC; NHLH1; NHLH2; NKRF; NKX1-1; NKX1-2; NKX2-1; NKX2-2; NKX2-3; NKX2-4; NKX2-5; NKX2-6; NKX2-8; NKX3-1; NKX3-2; NKX6-1; NKX6-2; NKX6-3; NME2; NOBOX; NOTO; NOTCH1; NOTCH2; NOTCH3; NOTCH4; NPAS1; NPAS2; NPAS3; NPAS4; NROB1; NR1D1; NR1D2; NR1H2; NR1H3; NR1H4; NR112; NR113; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRF1; NRL; OLIG1; OLIG2; OLIG3; ONECUT1; ONECUT2; ONECUT3; OSR1; OSR2; OTP; OTX1; OTX2; OVOL1; OVOL2; OVOL3; PA2G4; PATZ1; PAX1; PAX2; PAX3; PAX4; PAX5; PAX6; PAX7; PAX8; PAX9; PBX1; PBX2; PBX3; PBX4; PCGF2; PCGF6; PDX1; PEG3; PGR; PHF1; PHF19; PHF20; PHF21A; PHOX2A; PHOX2B; PIN1; PITX1; PITX2; PITX3; PKNOX1; PKNOX2; PLAG1; PLAGL1; PLAGL2; PLSCR1; POGK; POU1F1; POU2AF1; POU2F1; POU2F2; POU2F3; POU3F1; POU3F2 (BRN2); POU3F3; POU3F4; POU4F1; POU4F2; POU4F3; POU5F1; POU5F1B; POU5F2; POU6F1; POU6F2; PPARA; PPARD; PPARG; PRDM1; PRDM10; PRDM12; PRDM13; PRDM14; PRDM15; PRDM16; PRDM2; PRDM4; PRDM5; PRDM6; PRDM8; PRDM9; PREB; PRMT3; PROP1; PROX1; PROX2; PRR12; PRRX1; PRRX2; PTF1A; PURA; PURB; PURG; RAG1; RARA; RARB; RARG; RAX; RAX2; RBAK; RBCK1; RBPJ; RBPJL; RBSN; REL; RELA; RELB; REPIN1; REST; REXO4; RFX1; RFX2; RFX3; RFX4; RFX5; RFX6; RFX7; RFX8; RHOXF1; RHOXF2; RHOXF2B; RLF; RORA; RORB; RORC; RREB1; RUNX1; RUNX2; RUNX3; RXRA; RXRB; RXRG; SAFB; SAFB2; SALL1; SALL2; SALL3; SALL4; SATB1; SATB2; SCMH1; SCML4; SCRT1; SCRT2; SCX; SEBOX; SETBP1; SETDB1; SETDB2; SGSM2; SHOX; SHOX2; SIM1; SIM2; SIX1; SIX2; SIX3; SIX4; SIX5; SIX6; SKI; SKIL; SKOR1; SKOR2; SLC2A4RG; SMAD1; SMAD3; SMAD4; SMAD5; SMAD9; SMYD3; SNAI1; SNAI2; SNAI3; SNAPC2; SNAPC4; SNAPC5; SOHLH1; SOHLH2; SON; SOX1; SOX10; SOX11; SOX12; SOX13; SOX14; SOX15; SOX17; SOX18; SOX2; SOX21; SOX3; SOX30; SOX4; SOX5; SOX6; SOX7; SOX8; SOX9; SP1; SP100; SP110; SP140; SP140L; SP2; SP3; SP4; SP5; SP6; SP7; SP8; SP9; SPDEF; SPEN; SPI1; SPIB; SPIC; SPZ1; SRCAP; SREBF1; SREBF2; SRF; SRY; ST18; STAT1; STAT2; STAT3; STAT4; STAT5A; STAT5B; STAT6; T; TAZ; TAL1; TAL2; TBP; TBPL1; TBPL2; TBR1; TBX1; TBX10; TBX15; TBX18; TBX19; TBX2; TBX20; TBX21; TBX22; TBX3; TBX4; TBX5; TBX6; TCF12; TCF15; TCF20; TCF21; TCF23; TCF24; TCF3; TCF4; TCF7; TCF7L1; TCF7L2; TCFL5; TEAD1; TEAD2; TEAD3; TEAD4; TEF; TERB1; TERF1; TERF2; TET1; TET2; TET3; TFAP2A; TFAP2B; TFAP2C; TFAP2D; TFAP2E; TFAP4; TFCP2; TFCP2L1; TFDP1; TFDP2; TFDP3; TFE3; TFEB; TFEC; TGIF1; TGIF2; TGIF2LX; TGIF2LY; THAP1; THAP10; THAP11; THAP12; THAP2; THAP3; THAP4; THAP5; THAP6; THAP7; THAP8; THAP9; THRA; THRB; THYN1; TIGD1; TIGD2; TIGD3; TIGD4; TIGD5; TIGD6; TIGD7; TLX1; TLX2; TLX3; TMF1; TOPORS; TP53; TP63; TP73; TPRX1; TRAFD1; TRERF1; TRPS1; TSC22D1; TSHZ1; TSHZ2; TSHZ3; TTF1; TWIST1; TWIST2; UBP1; UNCX; USF1; USF2; USF3; VAX1; VAX2; VDR; VENTX; VEZF1; VSX1; VSX2; WIZ; WT1; XBP1; XPA; YBX1; YAP; YBX2; YBX3; YY1; YY2; ZBED1; ZBED2; ZBED3; ZBED4; ZBED5; ZBED6; ZBED9; ZBTB1; ZBTB10; ZBTB11; ZBTB12; ZBTB14; ZBTB16; ZBTB17; ZBTB18; ZBTB2; ZBTB20; ZBTB21; ZBTB22; ZBTB24; ZBTB25; ZBTB26; ZBTB3; ZBTB32; ZBTB33; ZBTB34; ZBTB37; ZBTB38; ZBTB39; ZBTB4; ZBTB40; ZBTB41; ZBTB42; ZBTB43; ZBTB44; ZBTB45; ZBTB46; ZBTB47; ZBTB48; ZBTB49; ZBTB5; ZBTB6; ZBTB7A; ZBTB7B; ZBTB7C; ZBTB8A; ZBTB8B; ZBTB9; ZC3H8; ZEB1; ZEB2; ZFAT; ZFHX2; ZFHX3; ZFHX4; ZFP1; ZFP14; ZFP2; ZFP28; ZFP3; ZFP30; ZFP37; ZFP41; ZFP42; ZFP57; ZFP62; ZFP64; ZFP69; ZFP69B; ZFP82; ZFP90; ZFP91; ZFP92; ZFPM1; ZFPM2; ZFX; ZFY; ZGLP1; ZGPAT; ZHX1; ZHX2; ZHX3; ZIC1; ZIC2; ZIC3; ZIC4; ZIC5; ZIK1; ZIM2; ZIM3; ZKSCAN1; ZKSCAN2; ZKSCAN3; ZKSCAN4; ZKSCAN5; ZKSCAN7; ZKSCAN8; ZMAT1; ZMAT4; ZNF10; ZNF100; ZNF101; ZNF107; ZNF112; ZNF114; ZNF117; ZNF12; ZNF121; ZNF124; ZNF131; ZNF132; ZNF133; ZNF134; ZNF135; ZNF136; ZNF138; ZNF14; ZNF140; ZNF141; ZNF142; ZNF143; ZNF146; ZNF148; ZNF154; ZNF155; ZNF157; ZNF16; ZNF160; ZNF165; ZNF169; ZNF17; ZNF174; ZNF175; ZNF177; ZNF18; ZNF180; ZNF181; ZNF182; ZNF184; ZNF189; ZNF19; ZNF195; ZNF197; ZNF2; ZNF20; ZNF200; ZNF202; ZNF205; ZNF207; ZNF208; ZNF211; ZNF212; ZNF213; ZNF214; ZNF215; ZNF217; ZNF219; ZNF22; ZNF221; ZNF222; ZNF223; ZNF224; ZNF225; ZNF226; ZNF227; ZNF229; ZNF23; ZNF230; ZNF232; ZNF233; ZNF234; ZNF235; ZNF236; ZNF239; ZNF24; ZNF248; ZNF25; ZNF250; ZNF251; ZNF253; ZNF254; ZNF256; ZNF257; ZNF26; ZNF260; ZNF263; ZNF264; ZNF266; ZNF267; ZNF268; ZNF273; ZNF274; ZNF275; ZNF276; ZNF277; ZNF28; ZNF280A; ZNF280B; ZNF280C; ZNF280D; ZNF281; ZNF282; ZNF283; ZNF284; ZNF285; ZNF286A; ZNF286B; ZNF287; ZNF292; ZNF296; ZNF3; ZNF30; ZNF300; ZNF302; ZNF304; ZNF311; ZNF316; ZNF317; ZNF318; ZNF319; ZNF32; ZNF320; ZNF322; ZNF324; ZNF324B; ZNF326; ZNF329; ZNF331; ZNF333; ZNF334; ZNF335; ZNF337; ZNF33A; ZNF33B; ZNF34; ZNF341; ZNF343; ZNF345; ZNF346; ZNF347; ZNF35; ZNF350; ZNF354A; ZNF354B; ZNF354C; ZNF358; ZNF362; ZNF365; ZNF366; ZNF367; ZNF37A; ZNF382; ZNF383; ZNF384; ZNF385A; ZNF385B; ZNF385C; ZNF385D; ZNF391; ZNF394; ZNF395; ZNF396; ZNF397; ZNF398; ZNF404; ZNF407; ZNF408; ZNF41; ZNF410; ZNF414; ZNF415; ZNF416; ZNF417; ZNF418; ZNF419; ZNF420; ZNF423; ZNF425; ZNF426; ZNF428; ZNF429; ZNF43; ZNF430; ZNF431; ZNF432; ZNF433; ZNF436; ZNF438; ZNF439; ZNF44; ZNF440; ZNF441; ZNF442; ZNF443; ZNF444; ZNF445; ZNF446; ZNF449; ZNF45; ZNF451; ZNF454; ZNF460; ZNF461; ZNF462; ZNF467; ZNF468; ZNF469; ZNF470; ZNF471; ZNF473; ZNF474; ZNF479; ZNF48; ZNF480; ZNF483; ZNF484; ZNF485; ZNF486; ZNF487; ZNF488; ZNF490; ZNF491; ZNF492; ZNF493; ZNF496; ZNF497; ZNF500; ZNF501; ZNF502; ZNF503; ZNF506; ZNF507; ZNF510; ZNF511; ZNF512; ZNF512B; ZNF513; ZNF514; ZNF516; ZNF517; ZNF518A; ZNF518B; ZNF519; ZNF521; ZNF524; ZNF525; ZNF526; ZNF527; ZNF528; ZNF529; ZNF530; ZNF532; ZNF534; ZNF536; ZNF540; ZNF541; ZNF543; ZNF544; ZNF546; ZNF547; ZNF548; ZNF549; ZNF550; ZNF551; ZNF552; ZNF554; ZNF555; ZNF556; ZNF557; ZNF558; ZNF559; ZNF560; ZNF561; ZNF562; ZNF563; ZNF564; ZNF565; ZNF566; ZNF567; ZNF568; ZNF569; ZNF57; ZNF570; ZNF571; ZNF572; ZNF573; ZNF574; ZNF575; ZNF576; ZNF577; ZNF578; ZNF579; ZNF580; ZNF581; ZNF582; ZNF583; ZNF584; ZNF585A; ZNF585B; ZNF586; ZNF587; ZNF587B; ZNF589; ZNF592; ZNF594; ZNF595; ZNF596; ZNF597; ZNF598; ZNF599; ZNF600; ZNF605; ZNF606; ZNF607; ZNF608; ZNF609; ZNF610; ZNF611; ZNF613; ZNF614; ZNF615; ZNF616; ZNF618; ZNF619; ZNF620; ZNF621; ZNF623; ZNF624; ZNF625; ZNF626; ZNF627; ZNF628; ZNF629; ZNF630; ZNF639; ZNF641; ZNF644; ZNF645; ZNF646; ZNF648; ZNF649; ZNF652; ZNF653; ZNF654; ZNF655; ZNF658; ZNF66; ZNF660; ZNF662; ZNF664; ZNF665; ZNF667; ZNF668; ZNF669; ZNF670; ZNF671; ZNF672; ZNF674; ZNF675; ZNF676; ZNF677; ZNF678; ZNF679; ZNF680; ZNF681; ZNF682; ZNF683; ZNF684; ZNF687; ZNF688; ZNF689; ZNF69; ZNF691; ZNF692; ZNF695; ZNF696; ZNF697; ZNF699; ZNF7; ZNF70; ZNF700; ZNF701; ZNF703; ZNF704; ZNF705A; ZNF705B; ZNF705D; ZNF705E; ZNF705G; ZNF706; ZNF707; ZNF708; ZNF709; ZNF71; ZNF710; ZNF711; ZNF713; ZNF714; ZNF716; ZNF717; ZNF718; ZNF721; ZNF724; ZNF726; ZNF727; ZNF728; ZNF729; ZNF730; ZNF732; ZNF735; ZNF736; ZNF737; ZNF74; ZNF740; ZNF746; ZNF747; ZNF749; ZNF750; ZNF75A; ZNF75D; ZNF76; ZNF761; ZNF763; ZNF764; ZNF765; ZNF766; ZNF768; ZNF77; ZNF770; ZNF771; ZNF772; ZNF773; ZNF774; ZNF775; ZNF776; ZNF777; ZNF778; ZNF780A; ZNF780B; ZNF781; ZNF782; ZNF783; ZNF784; ZNF785; ZNF786; ZNF787; ZNF788; ZNF789; ZNF79; ZNF790; ZNF791; ZNF792; ZNF793; ZNF799; ZNF8; ZNF80; ZNF800; ZNF804A; ZNF804B; ZNF805; ZNF808; ZNF81; ZNF813; ZNF814; ZNF816; ZNF821; ZNF823; ZNF827; ZNF829; ZNF83; ZNF830; ZNF831; ZNF835; ZNF836; ZNF837; ZNF84; ZNF841; ZNF843; ZNF844; ZNF845; ZNF846; ZNF85; ZNF850; ZNF852; ZNF853; ZNF860; ZNF865; ZNF878; ZNF879; ZNF880; ZNF883; ZNF888; ZNF891; ZNF90; ZNF91; ZNF92; ZNF93; ZNF98; ZNF99; ZSCAN1; ZSCAN10; ZSCAN12; ZSCAN16; ZSCAN18; ZSCAN2; ZSCAN20; ZSCAN21; ZSCAN22; ZSCAN23; ZSCAN25; ZSCAN26; ZSCAN29; ZSCAN30; ZSCAN31; ZSCAN32; ZSCAN4; ZSCAN5A; ZSCAN5B; ZSCAN5C; ZSCAN9; ZUFSP; ZXDA; ZXDB; ZXDC; or ZZZ3.

Accordingly, in embodiments, the transcription factor inhibitor (provided by the artificial nucleic acid of the invention) is for reducing or inhibiting the activity of a transcription factor selected from list A in a cell or subject.

In various embodiments, the transcription factor inhibitor (provided by the artificial nucleic acid of the invention) is for reducing or inhibiting the activity of a transcription factor selected from list A in a cell or subject, wherein the cellular transcription factor typically undergoes intracellular trafficking between the nucleus and cytoplasm. An example of such transcription factors are RUNX transcription factors or NF-kappaB.

Accordingly, in preferred embodiments, the target transcription factor is selected from AP1; ATF6; ERG; ETV1; GLI3; HOXA9; MBD2; MEF2A; NF-kappaB; POU3F2 (BRN2); PRDM13; RBPJ; RUNX1; RUNX2; SMAD3; SMAD4; SNAI1; TAZ; TCF21; TWIST1; MAML1; MAML2; EF2A; or YAP.

For example, overactivation or overexpression of ATF6 may be involved in fatty liver disease (Howarth et al., PLOS Genetics, 2014). Overactivation or overexpression of ERG and ETV1 may be involved in prostate cancer (Tomlin et al., Science, 2005). Overactivation or overexpression of GLI3 may be involved in corneal neovascularization (Renault et al., Circulation Research, 2008). Overactivation or overexpression of HOXA9 may be involved in myopia (Liang et al., BMC Ophthalmology, 2019). Overactivation or overexpression of MBD2 may be involved in diabetic retinopathy (Ge et al., Molecular Therapy: Nucleic Acids, 2021). Overactivation or overexpression of MEF2A may be involved in optic neuropathy (Xia et al., PLOS One, 2020). Overactivation or overexpression of Nf-KB may be involved in chronic tendon disease (Abraham et al., Science Translational Medicine, 2019). Overactivation or overexpression of BRN2 may be involved in melanoma, a common eye cancer (Goodall et al., MCB, 2004). Overactivation or overexpression of PRDM13 may be involved in North Carolina macular dystrophy (Small et al., Ophthalmology, 2016; Small et al., Molecular Vision 2021). Over activation or overexpression of RBPJ/Notch may be involved in hematologic cancer (Hurtado et al., Scientific Reports, 2019). Overactivation or overexpression of RUNX2 may be involved in osteoarthritis (Nishimura, J. Bone Metabolism, 2017). Overactivation or overexpression of SMAD3 may be involved in retinal detachment (Saik et al., Laboratory Investigation, 2004). Overactivation or overexpression of SMAD4 may be involved in proliferative vitreoretinopathy (Pao et al., PLOS One, 2021). Overactivation or overexpression of SNAI1 may be involved in ocular neovascularization (Sun et al., Angiogenesis, 2018). Overactivation or overexpression of YAP/TAZ may be involved in atherosclerosis (Wang et al., PNAS, 2016). Overactivation or overexpression of TCF21 may be involved in nephrotic syndrome (Usui, Scientific Reports, 2020). Overactivation or overexpression of Twist1 may be involved in lung cancer (Yochum, Oncogene, 2019). Overactivation or overexpression of YAP/TAZ may be involved atherosclerosis (Wang et al., PNAS, 2016). Overactivation or overexpression of API may be involved in TNFalpha mediated RUNX1 activation (Whitmore, FASEB journal: official publication of the Federation of American Societies for Experimental Biology. 2020). Overactivation or overexpression of RUNX1 may be involved in ocular diseases including proliferative vitreoretinopathy, diabetic retinopathy and in Down syndrome.

Accordingly, in preferred embodiments, the transcription factor inhibitor (provided by the artificial nucleic acid of the invention) is for reducing or inhibiting the activity AP1; ATF6; ERG; ETV1; GLI3; HOXA9; MBD2; MEF2A; NF-kappaB; POU3F2 (BRN2); PRDM13; RBPJ; RUNX1; RUNX2; SMAD3; SMAD4; SNAI1; TAZ; TCF21; TWIST1; MAML1; MAML2; EF2A; or YAP in a cell.

In preferred embodiments, the target transcription factor is selected from GLI3; HOXA9; MBD2; MEF2A; POU3F2 (BRN2); PRDM13; RUNX1; SMAD3; SMAD4; or SNAI1. These transcription factors are particularly suitable in embodiments where the target transcription factor is selected from a transcription factor that is overexpressed or overactive in an ocular disease, disorder, or condition (including aging). A preferred example is RUNX1 that is overexpressed or overactive in various ocular diseases including PVR.

Accordingly, in preferred embodiments, the transcription factor inhibitor (provided by the artificial nucleic acid of the invention) is for reducing or inhibiting the activity of GLI3; HOXA9; MBD2; MEF2A; POU3F2 (BRN2); PRDM13; RUNX1; SMAD3; SMAD4; or SNAI1 in a cell, preferably a cell of an eye.

In particularly preferred embodiments, the target transcription factor is a RUNX transcription factor, for example RUNX1, RUNX2, or RUNX3.

Accordingly, in particularly preferred embodiments, the transcription factor inhibitor (provided by the artificial nucleic acid of the invention) is for reducing or inhibiting the activity of a RUNX transcription factor, for example RUNX1, RUNX2, RUNX3, in a cell.

Runt-related transcription factor 1 (RUNX1), also known as acute myeloid leukaemia 1 protein (AML1) or core-binding factor subunit alpha-2 (CBFA2), is a protein that in humans is encoded by the RUNX1 gene. RUNX proteins (e.g. RUNX1, RUNX2, RUNX3) form a heterodimeric complex with core binding factor b (CBFbeta) which confers increased deoxyribonucleic acid (DNA) binding and stability to the complex. That complex comprising RUNX (CBFalpha) proteins and CBFbeta is often referred to as heterodimeric CBF transcription factor. RUNX1 is a transcription factor that inter alia regulates the differentiation of hematopoietic stem cells into mature blood cells. RUNX1 also plays a role in the development of the neurons that transmit pain. In humans, the RUNX1 gene is 260 kilobases (kb) in length and is located on chromosome 21 (21q22.12). The gene can be transcribed from 2 alternative promoters, promoter 1 (distal) or promoter 2 (proximal). As a result, various isoforms of RUNX1 can be synthesized, facilitated by alternative splicing. The full-length RUNX1 protein is encoded by 12 exons. Among the exons are two defined domains, namely the runt homology domain (RHD) or the runt domain (exons 2, 3 and 4), and the transactivation domain (TAD) (exon 6). These domains are necessary for RUNX1 to mediate DNA binding and protein-protein interactions respectively. The transcription of RUNX1 is regulated by 2 enhancers (regulatory element 1 and regulatory element 2), and these tissue specific enhancers enable the binding of lymphoid or erythroid regulatory proteins, therefore the gene activity of RUNX1 is highly active in the hematopoietic system.

An exemplary isoform of RUNX1 (Q01196-1; SEQ ID NO: 198) has 453 amino acids. As a transcription factor, its DNA binding ability is encoded by the runt domain (residues 50-177 of SEQ ID NO: 198. DNA recognition is achieved by loops of the 12-stranded b-barrel and the C-terminus “tail” (residues 170-177 of SEQ ID NO: 198), which clamp around the sugar phosphate backbone and fits into the major and minor grooves of DNA. Further exemplary landmark sequences and domains include residues 80-84 (DNA binding domain), residues 135-143 (DNA binding domain), residues 168-177 (DNA binding domain), residues 291-371 (interaction with K (lysine) acetyltransferase 6A (KATA6A)), residues 307-400 (interaction with K (lysine) acetyltransferase 6B (KATA6B)), and residues 362-402 (interaction with forkhead box P3 (FOXP3)). The nuclear localization signal (NLS) is present at amino acids 167 to 183 at the end of the Runt domain.

RUNX1 can bind DNA as a monomer, but its DNA binding affinity is enhanced by 10-fold if it heterodimerizes with its co-transcription factor CBFbeta, also via the runt domain.

An amino acid sequence for human RUNX1 is publicly available in the UniProt database under accession number Q01196-1 (or SEQ ID NO: 198). Amino acid sequences of additional isoforms are publicly available in the UniProt database under accession numbers Q01196-2; Q01196-3; Q01196-4; Q01196-5; Q01196-6; Q01196-7; Q01196-8; Q01196-9; Q01196-10; and Q01196-11 (see also SEQ ID NOs: 199-212)

Runt-related transcription factor 2 (RUNX2), also known as core-binding factor subunit alpha-1, is a protein that in humans is encoded by the RUNX2 gene. RUNX2 is a transcription factor that inter alia has been associated with osteoblast differentiation. An amino acid sequence for human RUNX2 is publicly available in the GenBank database under accession number NP_001019801.3 (see also SEQ ID NO: 23 of published patent application WO2019099560). Exemplary landmark sequences and domains include residues 49-71 (polyglutamine repeat), residues 73-89 (polyalanine repeat), residues 109-230 (runt domain), residues 242-258 (domain for interaction with forkhead Box 01 (FOXOI)), residues 336-439 (domain for interaction with K (lysine) acetyltransferase 6A (KATA6A)), residues 374-488 (domain for interaction with K (lysine) acetyltransferase 6B (KATA6B)), and residues 430-521 (RUNX1 inhibition domain). Additional amino acid sequences of human RUNX2 isoforms are public ally available in the GenBank database under accession numbers: NP_001015051.3, Q13950.2, and NP_001265407.1. Amino acid sequences of additional RUNX2 isoforms are publicly available in the GenBank database under accession numbers NP_001139392.1, NP_001139510.1, NP_001258556.1, NP_001258559.1, and NP_001258560.1.

In particularly preferred embodiments, the target transcription factor is RUNX1.

Accordingly, in preferred embodiments, the artificial nucleic acid of the invention comprises at least one coding sequence encoding at least one RUNX1 transcription factor inhibitor (e.g. a RUNX1 trap) for reducing or inhibiting the activity of a RUNX1 in a cell.

### Amino Acid Sequences—Element A

In preferred embodiments, the encoded transcription factor inhibitor comprises at least one amino acid sequence element A that is configured to bind to the cellular target transcription factor or its transcription co-factor.

Accordingly, amino acid sequence element A of the encoded transcription factor inhibitor is configured to bind to the target transcription factor (e.g. RUNX) or its transcription co-factor (e.g. CBFbeta).

In embodiments where amino acid sequence element A is configured to bind to the (cellular) target transcription factor, the produced transcription factor inhibitor (comprising such an element A) binds to its target transcription factor (e.g. RUNX), thereby directly reducing or inhibiting the activity of a target transcription factor in a cell (e.g. RUNX).

In embodiments where amino acid sequence element A is configured to bind to a (cellular) transcription co-factor of the target transcription factor, the produced transcription factor inhibitor (comprising such an element A) binds to the transcription co-factor (e.g. CBFbeta) of the target transcription factor, thereby indirectly reducing or inhibiting the activity of the target transcription factor in a cell (e.g. RUNX).

In various embodiments, the amino acid sequence element A comprises any amino acid sequence that has binding affinity to the (cellular) target transcription factor or its transcription co-factor.

In preferred embodiments, the encoded transcription factor inhibitor comprises at least one amino acid sequence element A that comprises or consists of an amino acid sequence selected or derived from the target transcription factor, an interaction partner of the target transcription factor, a binding partner of the target transcription factor, a transcription co-factor of the target transcription factor, an antibody moiety, an intrabody moiety, a peptide-based aptamer, or a fragment or variant of any of these that preferably binds to the target transcription factor or its transcription co-factor.

In some embodiments, the at least one amino acid sequence element A does not comprise an amino acid sequence selected or derived from an antibody moiety or an intrabody moiety.

In various preferred embodiments, the at least one amino acid sequence element A comprises or consists of an amino acid sequence that lacks a nuclear localization signal (NLS) or that has been modified to lack an NLS. The absence of an NLS in the transcription factor inhibitor of the present invention is particularly important in embodiments where the transcription factor inhibitor is a transcription factor trap as defined herein. Accordingly, the at least one amino acid sequence element A may be selected from a protein that naturally lacks an NLS, e.g. a transcription co-factor of the target transcription factor that lacks an NLS (e.g. CBFbeta). Alternatively, the at least one amino acid sequence element A may be selected from a protein that naturally comprises an NLS, wherein the amino acid sequence has been modified to lack a functional NLS e.g. by removing or mutating the respective amino acid sequence of element A (e.g. a modified RUNX amino acid sequence).

In various preferred embodiments, the at least one amino acid sequence element A comprises or consists of an amino acid sequence that lacks a DNA binding domain or that has been modified to lack a functional DNA binding domain.

Accordingly, the at least one amino acid sequence element A may be selected from a protein that naturally lacks a DNA binding domain, e.g. a transcription co-factor of the target transcription factor that lacks an DNA binding domain (e.g. CBFbeta). Alternatively, the at least one amino acid sequence element A may be selected from a protein that naturally comprises a DNA binding domain, wherein the amino acid sequence has been modified to lack a functional DNA binding domain e.g. by removing or mutating the respective amino acid sequence of element A (e.g. a modified RUNX amino acid sequence).

In preferred embodiments, the at least one amino acid sequence element A is configured to bind to the target transcription factor. Accordingly, in preferred embodiments, the at least one amino acid sequence element A comprises or consists of an amino acid sequence selected or derived from a transcription co-factor of the target transcription factor, or a fragment or variant thereof.

Selecting the amino acid sequence element A from a transcription co-factor of the target transcription factor is particularly suitable in the context of the present invention. Preferably, amino acid sequence element A is selected or derived from a transcription co-factor that binds directly to the target transcription factor. Preferably, amino acid sequence element A is selected or derived from a transcription co-factor that forms a heterodimeric complex with the target transcription factor, preferably in the cytosol.

Accordingly, in preferred embodiments, the at least one amino acid sequence element A comprises or consists of an amino acid sequence selected or derived from a transcription co-factor of the target transcription factor, or a fragment or variant thereof, wherein the transcription co-factor is selected from a co-factor that forms a heterodimeric complex with the transcription factor, preferably in the cytosol.

Suitably, the transcription co-factor is selected from a co-factor of any of the target transcription factors of list A, preferably selected from a transcription co-factor of a target transcription factor selected from AP1; ATF6; ERG; ETV1; GLI3; HOXA9; MBD2; MEF2A; NF-kappaB; POU3F2 (BRN2); PRDM13; RBPJ; RUNX1; RUNX2; SMAD3; SMAD4; SNAI1; TAZ; TCF21; TWIST1; MAML1; MAML2; EF2A; or YAP. More preferably, the transcription co-factor is selected from a co-factor of a target transcription factor selected from GLI3; HOXA9; MBD2; MEF2A; POU3F2 (BRN2); PRDM13; SMAD3; SMAD4; SNAI1; or RUNX, e.g. RUNX1.

The skilled person is able to identify transcription co-factors for respective target transcription factors, to obtain suitable amino acid sequences from those transcription co-factors suitable for binding to the target transcription factor, and to include said suitable amino acid sequence into an amino acid sequence of respective transcription factor inhibitor. For example, suitable transcription co-factors may be selected from PBX3 (co-factor of the target transcription factor HOXA9), HDAC9 or p300 (co-factors of the target transcription factor MEF2), PAX3, SOX10, and OCT1 (co-factors of the target transcription factor BRN2), Smad-binding proteins (co-factors of target transcription factors SMAD3 or SMAD4), LMO4 (co-factors of target transcription factor SNAI1), MAML (co-factor of RBPJ), or CBFbeta (co-factor of RUNX target transcription factors).

In preferred embodiments, the at least one amino acid sequence element A comprises or consists of an amino acid sequence selected or derived from a transcription co-factor of a Runt-related transcription factor (e.g. RUNX1, RUNX2, RUNX3), preferably RUNX1, or a fragment or variant thereof.

Preferably, the transcription co-factor of Runt-related transcription factor (e.g. RUNX1, RUNX2, RUNX3), preferably RUNX1, is selected or derived from a co-factor that forms a heterodimeric complex with a Runt-related transcription factor (e.g. RUNX1, RUNX2, RUNX3), preferably RUNX1 in the cytosol.

In preferred embodiments, the transcription co-factor of RUNX is selected or derived from Core Binding Factor beta (CBFbeta), for example CBFbeta1 or CBFbeta2 isoforms, or a fragment or variant thereof.

Accordingly, the transcription factor inhibitor, preferably the RUNX inhibitor, comprises at least one amino acid sequence element A selected or derived from a transcription co-factor of RUNX, wherein the transcription co-factor of RUNX is selected or derived from CBFbeta, or a fragment or variant thereof.

The transcription co-factor CBFbeta is a subunit of a heterodimeric core-binding transcription factor belonging to the PEBP2/CBF transcription factor family which regulates a host of genes specific to haematopoiesis (e.g., RUNX1) and osteogenesis (e.g., RUNX2). The Core Binding Factor (CBF) regulates transcription via formation of a heterodimeric complex between RUNX, the CBFalpha-DNA-binding subunit, and CBFbeta. CBFbeta is a non-DNA binding regulatory subunit; it allosterically enhances DNA binding by the alpha subunit (of e.g. RUNX) as the complex binds to the core site of various enhancers and promoters. Despite that RUNX can bind DNA as a monomer in vitro, heterodimerization with the non-DNA binding transcription co-factor CBFbeta triggers flexible DNA-recognition loops, thus stabilizing the complex and increasing RUNX binding to DNA. Binding of transcription co-factor CBFbeta enhances DNA binding affinity of RUNX by approximately 10-fold.

There are two different described isoforms of CBFbeta proteins. The CBFbeta isoform 1 (UniProt database entry Q13951-1; SEQ ID NO: 178) has 182 amino acids, and the CBFbeta isoform 2 (UniProt database entry Q13951-2; SEQ ID NO: 181) has 187 amino acids. The amino acid 165 to 166 represent a splice site that leads to the formation of the two different isoforms of CBFbeta with either 17 (CBFbeta2) or 22 (CBFbeta1) distinct amino acid sequences. CBFbeta isoform 1 and CBFbeta isoform 2 are highly similar wherein amino acid 1 to 165 are identical and the C-terminus region varies between the two isoforms. The amino acid sequence elements responsible for heterodimerization with RUNX1 or RUNX2 is located in amino acid sequence 1 to 141 (see SEQ ID NO: 182) in both isoforms. Other isoforms are provided and can be derived from SEQ ID NOs: 179 and 180.

In preferred embodiments, element A comprises an amino acid sequence selected or derived from CBFbeta, preferably wherein the CBFbeta amino acid sequence is an N-terminal fragment of a human CBFbeta. In embodiments, the N-terminal fragment of a human CBFbeta comprises an N-terminal stretch from position 1 to position 100, position 1 to position 110, position 1 to position 115, position 1 to position 120, position 1 to position 125, position 1 to position 130, position 1 to position 135, position 1 to position 140, position 1 to position 145, position 1 to position 150, position 1 to position 165, position 1 to position 170, position 1 to position 175, or position 1 to position 180 of amino acid sequence SEQ ID NO: 178. In embodiments, the N-terminal fragment of a human CBFbeta comprises an N-terminal stretch from position 5 to position 100, position 5 to position 110, position 5 to position 115, position 5 to position 120, position 5 to position 125, position 5 to position 130, position 5 to position 135, position 5 to position 140, position 5 to position 145, position 5 to position 150, position 5 to position 165, position 5 to position 170, position 5 to position 175, or position 5 to position 180 of amino acid sequence SEQ ID NO: 178. In embodiments, the N-terminal fragment of a human CBFbeta comprises an N-terminal stretch from position 10 to position 100, position 10 to position 110, position 10 to position 115, position 10 to position 120, position 10 to position 125, position 10 to position 130, position 10 to position 135, position 10 to position 140, position 10 to position 145, position 10 to position 150, position 10 to position 165, position 10 to position 170, position 10 to position 175, or position 10 to position 180 of amino acid sequence SEQ ID NO: 178. In embodiments, the N-terminal fragment of a human CBFbeta comprises an N-terminal stretch from position 20 to position 100, position 20 to position 110, position 20 to position 115, position 20 to position 120, position 20 to position 125, position 20 to position 130, position 20 to position 135, position 20 to position 140, position 20 to position 145, position 20 to position 150, position 20 to position 165, position 20 to position 170, position 20 to position 175, or position 20 to position 180 of amino acid sequence SEQ ID NO: 178.

In embodiments, the N-terminal fragment of a human CBFbeta comprises an N-terminal stretch comprising at least 100, 110, 120, 130, 140, 150, or 160 amino acid residues of amino acid sequence SEQ ID NO: 178, e.g. 130, 141 or 165 amino acid residues of SEQ ID 178.

In preferred embodiments, element A comprises an amino acid sequence selected or derived from CBFbeta, wherein the CBFbeta amino acid sequence is an N-terminal fragment of a human CBFbeta, preferably comprising up to about 130, preferably comprising up to about 141, more preferably up to about 165 amino acids residues of amino acid sequence SEQ ID NO: 178.

In preferred embodiments, the CBFbeta amino acid sequence is selected or derived from a fragment comprising amino acid 1 to amino acid 130 of CBFbeta of amino acid sequence SEQ ID NO: 178. In other preferred embodiments, the CBFbeta amino acid sequence is selected or derived from a fragment comprising amino acid 1 to amino acid 141 of CBFbeta of amino acid sequence SEQ ID NO: 178, for example CBFbeta (1-141) according to SEQ ID NO: 182. In particularly preferred embodiments, the CBFbeta amino acid sequence is selected or derived from a fragment comprising amino acid 1 to amino acid 165 of CBFbeta of amino acid sequence SEQ ID NO: 178, for example CBFbeta (1-165) according to SEQ ID NO: 183.

In preferred embodiments, the transcription factor inhibitor (that is provided by the artificial nucleic acid of the invention) comprises at least one amino acid sequence element A that comprises or consists of an amino acid sequence selected or derived from CBFbeta being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 178-183, or fragments or variants of any of these. Suitable amino acid sequences are also provided in Table 1.

In particularly preferred embodiments, the transcription factor inhibitor (that is provided by the artificial nucleic acid of the invention) comprises at least one amino acid sequence element A that comprises or consists of an amino acid sequence selected or derived from CBFbeta being identical or at least 90%, 91%, 92%, 93%, 94%, 95%, 96%, 97%, 98%, or 99% identical to any one of SEQ ID NO: 183, or a fragment or variant thereof.

In other preferred embodiments, the at least one amino acid sequence element A is configured to bind to a (cellular) transcription co-factor of the target transcription factor. In preferred embodiments, the transcription co-factor of the target transcription factor is selected from a co-factor protein, a chromatin factor, or a non-coding regulatory nucleic acid.

In particularly preferred embodiments, the at least one amino acid sequence element A is configured to bind to a transcription co-factor protein of the target transcription factor, suitably a transcription co-activator.

In preferred embodiments, the at least one amino acid sequence element A comprises or consists of an amino acid sequence selected or derived from the target transcription factor, or a fragment or variant thereof.

Suitably, the amino acid sequence element A may be selected or derived from any target transcription factors of list A, preferably selected or derived from AP1; ATF6; ERG; ETV1; GLI3; HOXA9; MBD2; MEF2A; NF-kappaB; POU3F2 (BRN2); PRDM13; RBPJ; RUNX1; RUNX2; SMAD3; SMAD4; SNAI1; TAZ; TCF21; TWIST1; or YAP, or fragments or variants of any of these. More preferably, the amino acid sequence element A is selected or derived from GLI3; HOXA9; MBD2; MEF2A; POU3F2 (BRN2); PRDM13; SMAD3; SMAD4; SNAI1; or RUNX1, or fragments or variants of any of these.

In preferred embodiments, the at least one element A comprises or consists of an amino acid sequence selected or derived from RUNX or a fragment or variant thereof, preferably wherein the RUNX amino acid sequence is an N-terminal fragment of a human RUNX1. A preferred N-terminal fragment of a human RUNX comprises the Runt homology domain (RHD).

Accordingly, in preferred embodiments, the N-terminal fragment of RUNX comprises residues 1 to 128 selected or derived from RUNX1, residues 1 to 177 selected or derived from RUNX1, or residues 1 to 241 selected or derived from RUNX1 (positions according to SEQ ID NO: 198). For example, for example the RUNX1 that comprises residues 1 to 128 may comprise an amino acid sequence according to SEQ ID NO: 213. For example, for example the RUNX1 that comprises residues 1 to 177 may comprise an amino acid sequence according to SEQ ID NO: 214. For example, for example the RUNX1 that comprises residues 1 to 241 may comprise an amino acid sequence according to SEQ ID NO: 215.

In preferred embodiments, the amino acid sequence element A selected or derived from the target transcription factor comprises at least one amino acid substitution or deletion that reduces or prevents binding to its target DNA or at least one amino acid substitution or deletion that reduces or prevents nuclear translocation and/or at least one amino acid substitution or deletion that reduces or prevents homodimerization or heterodimerization.

In preferred embodiments, the amino acid sequence is selected or derived from the target transcription factor RUNX, preferably RUNX1, wherein the amino acid sequence comprises at least one amino acid substitution or deletion that reduces or prevents binding to its target DNA or at least one amino acid substitution and/or a deletion that reduces or prevents nuclear translocation.

Suitably, the amino acid substitution or deletion that reduces or prevents binding of the RUNX1 amino acid sequence to its target DNA may be located in any of residues 80-84, 135-143, or 168-177 (positions according to the RUNX1 sequence according to SEQ ID NO: 198). Preferably, a substitution or deletion is introduced at position R80, K83, K83, R135, R139, R142, K167, T169, D171, R174, or R177.

Suitably, the amino acid substitution or deletion that reduces or prevents nuclear translocation of the RUNX1 amino acid sequence may be located in any of residues 167 to 183 (positions according to the RUNX1 sequence according to SEQ ID NO: 198). Preferably, a substitution or deletion is introduced at position K167, T169, D171, R174, or R177.

In preferred embodiments, the at least one amino acid sequence element A comprises or consists of an amino acid sequence selected or derived from RUNX1 or a fragment or variant thereof, wherein the RUNX1 amino acid sequence comprises at least one, two, or more amino acid substitutions or deletions selected from R80A, K83A, K83E, R135A, R139A, R142A, K167A, T169A, D171A, R174A, or R177A, or any functionally equivalent amino acid substitution at position R80, K83, R135, R139, R142, K167, T169, D171, R174, or R177.

In preferred embodiments, at least one amino acid substitution in the RUNX1 amino acid sequence is selected from R174Q and/or K83E, or any functionally equivalent amino acid substitution at position R174 and/or K83. Suitably, the amino acid substitutions are selected from R174Q and K83E, for example wherein the RUNX1 comprises an amino acid sequence according to SEQ ID NO: 197, or a fragment or variant thereof.

In preferred embodiments, the transcription factor inhibitor comprises at least one amino acid sequence element A that comprises or consists of an amino acid sequence selected or derived from RUNX1 being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 197-215, or fragments or variants of any of these. Suitable amino acid sequences are also provided in Table 1.

In particularly preferred embodiments, the transcription factor inhibitor comprises at least one amino acid sequence element A that comprises or consists of an amino acid sequence selected or derived from RUNX1 fragment being identical or at least 90%, 91%, 92%, 93%, 94%, 95%, 96%, 97%, 98%, or 99% identical to any one of SEQ ID NOs: 213 or 214, or fragments or variants of any of these.

In particularly preferred embodiments, the transcription factor inhibitor comprises at least one amino acid sequence element A that comprises or consists of an amino acid sequence selected or derived from RUNX1 (K83E,R174Q) being identical or at least 90%, 91%, 92%, 93%, 94%, 95%, 96%, 97%, 98%, or 99% identical to any one of SEQ ID NO: 197, or fragments or variants of any of these.

### Amino Acid Sequences—Element B

According to preferred embodiments, the transcription factor inhibitor that is encoded by the artificial nucleic acid of the invention comprises at least one amino acid sequence element B.

Notably, amino acid sequence element A and amino acid sequence element B may represent a different amino acid sequence, or amino acid sequence element A and amino acid sequence element B may represent (partially) overlapping amino acid sequences, or amino acid sequence element A and amino acid sequence element B may represent (essentially) the same amino acid sequence.

In preferred embodiments, amino acid sequence element A and amino acid sequence element B of the transcription factor inhibitor represent different amino acid sequences, e.g. element A and element B are located at different positions in the amino acid sequence of the transcription factor inhibitor.

In preferred embodiments, the encoded transcription factor inhibitor comprises at least one amino acid sequence element B that is configured to prevent or reduce the interaction of the cellular target transcription factor with its target DNA. Accordingly, the amino acid sequence element B is selected or derived from a peptide or protein (e.g. a certain protein domain) that prevents or reduces the interaction of the target transcription factor with its target DNA.

In preferred embodiments, the encoded transcription factor inhibitor comprises at least one amino acid sequence element B that is configured to prevent or reduce interaction of the cellular target transcription factor with at least one cellular transcription co-factor of the target transcription factor. Accordingly, the amino acid sequence element B is selected or derived from a peptide or protein (e.g. a certain protein domain) that prevents or reduces interaction of the target transcription factor with at least one co-factor of the target transcription factor.

In preferred embodiments, the encoded transcription factor inhibitor comprises at least one amino acid sequence element B that is configured to prevent or reduce nuclear translocation of the (cellular) target transcription factor. Accordingly, the amino acid sequence element B is selected or derived from a peptide or protein (e.g. a certain protein domain) that prevents or reduces nuclear translocation of the target transcription factor.

In preferred embodiments, the encoded transcription factor inhibitor comprises at least one amino acid sequence element B that is configured to bind the cellular target transcription factor or its transcription co-factor preferably in the cytosol. Accordingly, the amino acid sequence element B is selected or derived from a peptide or protein (e.g. a certain protein domain) that binds the target transcription factor or its transcription co-factor preferably in the cytosol.

In preferred embodiments, the encoded transcription factor inhibitor comprises at least one amino acid sequence element B that is configured to repress the transcription activity of the cellular target transcription factor. Accordingly, the amino acid sequence element B is selected or derived from a peptide or protein (e.g. a certain protein domain) that represses the transcription activity of the target transcription factor.

In particularly preferred embodiments, the encoded transcription factor inhibitor comprises at least one amino acid sequence element B that is configured to prevent or reduce the interaction of the target transcription factor with its target DNA and that is configured to prevent or reduce interaction of the target transcription factor with at least one co-factor of the target transcription factor and is configured to prevent or reduce nuclear translocation of the cellular target transcription factor and is configured to bind the cellular target transcription factor in the cytosol and is optionally configured to repress the transcription activity of the cellular target transcription factor

In particularly preferred embodiments, the transcription factor inhibitor that is encoded by the artificial nucleic acid of the invention comprises at least one amino acid sequence element B that comprises or consists of an amino acid sequence selected or derived from a cytoplasmic protein or a protein that is associated with or binds to a cytoplasmic protein, or a fragment or variant thereof.

A cytoplasmic protein has to be understood as a protein that is typically located in the cytoplasm of a cell. Proteins that undergo nucleocytoplasmic transport (e.g. via NLS signals) are not considered to be a cytoplasmic protein. Proteins that undergo secretion (e.g. via secretory signal peptides) are not considered to be a cytoplasmic protein.

The suitable cytoplasmic protein in the context of the invention can also be a synthetic, engineered, or heterologous protein. A typical cytoplasmic protein is a cytoskeletal protein or a protein that is associated with a cytoskeletal protein (e.g. a peptide or protein that binds to the cytoskeleton of a cell, e.g. an actin-binding protein).

In preferred embodiments, amino acid sequence element B comprises or consists of an amino acid sequence selected or derived from a cytoskeletal protein or a protein that is associated with or binds to a cytoskeletal protein, or a fragment or variant of any of these. Such proteins are particularly suitable as the transcription factor inhibitor of the invention that e.g. binds to a cellular transcription factor (e.g. RUNX via a CBFbeta amino acid sequence element A) such that it can be trapped in the cytosol via an amino acid sequence that optionally interacts with or binds to a cytoskeletal protein (e.g. an actin binding protein) or a protein in the cytosol.

In preferred embodiments in that context, the cytoplasmic protein, preferably the cytoskeletal protein is selected or derived from myofibrillar protein (e.g. actin or myosin), a microtubule protein, an intermediate filament protein, or a fragment or variant of any of these. Particularly suitable in that context is myosin.

In preferred embodiments in that context, the cytoplasmic protein, preferably the cytoskeletal protein is selected or derived from a peptide or protein that is associated with or binds to a myofibrillar protein (e.g. actin), a microtubule protein, an intermediate filament protein, or a fragment or variant of any of these. Particularly suitable in that context are actin-binding peptides or proteins.

Accordingly, it is preferred in the context of the invention that amino acid sequence element B comprises or consists of an amino acid sequence selected or derived from a protein that comprises a myofibrillar binding domain, in particular an actin binding domain, a microtubule binding domain, an intermediate filament binding domain, or a fragment or variant of any of these that comprises a myofibrillar binding domain.

In particularly preferred embodiments, amino acid sequence element B comprises or consists of an amino acid sequence selected or derived from a protein that comprises an actin binding domain, or a fragment or variant of any of these that comprises an actin binding domain.

In various preferred embodiments, the amino acid sequence element B comprises or consists of an amino acid sequence selected or derived from a cytoskeletal protein, preferably a protein that comprises an actin binding domain, wherein the amino acid sequence is selected from any one of the proteins provided in List B: 25 kDa ABP from aorta p185neu; 30 akDA 110 kD dimer ABP; 30 bkDa 110 kD (Drebrin); p53; p58gag; p116rip; a-actinin; Abl; AbLIM Actin-Interacting MAPKKK; ABP120; ABP140; Abp1p; ABP280 (Filamin); ABP50 (EF-1a); Acan 125 (Carmil); ActA; Actibind; Actin; Actinfilin; Actinogelin; Actin-regulating kinases; Actin-Related Proteins; Actobindin; Actolinkin; Actopaxin; Actophorin; Acumentin (=L-plastin); Adducin; ADF/Cofilin; Adseverin (scinderin); Afadin; AFAP-110; Affixin; Aginactin; AIP1; Aldolase; Angiogenin; Anillin; Annexins; Aplyronine; Archvillin; Arginine kinase; Arp2/3 complex; Band 4.1; Band 4.9 (Dematin); b-actinin; b-Cap73; Bifocal; Bistramide A; BPAG1; Brevin (Gelsolin); c-Abl; Calpactin (Annexin); CHO1; Cortactin; CamKinase Il; Calponin; Chondramide; Cortexillin; CAP; Caltropin; CH-ILKBP; CPb3; Cap100; Calvasculin; Ciboulot; Coactosin; CAP23; CARMIL; Acan125; Cingulin; Cytovillin (Ezrin); CapZ/Capping Protein; a-Catenin; Cofilin; CR16; Caldesmon; CCT; Comitin; Calicin; Centuarin; Coronin; DBP40; Drebrin; Dematin (Band 4.9); Dynacortin; Destrin (ADF/cofilin); Dystonins; Diaphanous; Dystroglycan; DNase I; Dystrophin; Doliculide; Dolastatins; EAST; Endossin; EF-1a (ABP50); Eps15; EF-1b; EPLIN; EF-2; Epsin; EGF receptor; ERK; ERZ; ENC-1; ERM proteins (ezrin, radixin, moesin, plus merlin); END3p; Ezrin (the E of ERM protein family); F17R; Fodrin (spectrin); Fascin; Formins; Fessilin; Frabin; FHL3; Fragmin; Fhos; FLNA (filamin A); Fimbrin (plastin); GAP43; Glycogenins; Gas2; G-proteins; Gastrin-Binding Protein; Gelactins I-IV; Gelsolins; Glucokinase; Harmonin b; Hrp36; Hexokinase; Hrp65-2; Hectochlorin; HS1 (actin binding protein); Helicase II; Hsp27; HIP1 (Huntingtin Interacting protein 1); Hsp70; Histactophilin; Hsp90; Histidine rich protein II; Hsp100; Inhibitor of apoptosis (IAP); Insertin; Interaptin; IP3Kinase A (Inositol 1,4,5-trisphosphate 3-kinase A); IQGAP; Integrins; Jaspisamide A; Jasplakinolide; Kabiramide C; Kaptin; Kettin; Kelch protein; keratin (e.g. kreatin-19); 5-Lipoxygenase; Limatin; Lim Kinases; Lim Proteins; L-plastin; Lymphocyte Specific Protein 1 (LSP1); MACF1 [3]; MacMARKS; Mena; Myopodin; MAP1A; Microtubule Associated Protein 7 (MAP7); Merlin (related to the ERM proteins); Myosins (e.g. SMMHC); MAP-1C; Metavinculin; Moesin (the M of ERM proteins); Myosin light chain kinase; MAL; Mip-90; Myosin Light Chain A1; MARKS; MIM; MAYP; Mycalolide (a macroglide drug); Mayven; Myelin basic protein; Naphthylphthalamic acid binding protein (NPA)N-RAP; Nebulin; N-WASP; Neurabin; Nullo; Neurexins; Neurocalcin; Nexillin; OYE2; Palladin; Plastin; p30; PAK (p21-activated Kinase); Plectin; p47PHOX; Parvin (actopaxin); Prefoldin; p53; PASK (Proline, Alanine rich Ste20 related Kinase); Presenilin I; p58; Phalloidin (not a protein; a small cyclic peptide); Profilin; p185neu; Ponticulin; Protein kinase C; Porin; P.IB; Prk1p (actin regulating kinase); Radixin (the R of ERM proteins); Rapsyn; Rhizopodin; RPL45; RTX toxin (Vibrio cholerae); RVS 167; Sac6; Sla1p; Srv2 (CAP); S-adenosyl-L-homocysteine hydrolase, (SAHH); Sla2p; Synaptopodin; Scinderin (adseverin); Synapsins; Scruin; Spectrin; Severin; Spectraplakins; SVSII; Shot (Short stop); Spire; Shroom; Smitin (Smooth Musc.Titin); Supervillin; SipA; Smoothelin; Sucrose synthetase; SipC; Sra-1; Spinophilin; Ssk2p; Swinholide; Talin protein; Toxophilin; Twinfilin; Tau; Trabeculin; Twinstar; TCP-1; Transgelin; Transgelin 2; Transgelin 3; Tensin; Tropomodulin; Thymosin; Tropomyosin; Titin; Troponin; TOR2; Tubulin bIV; Ulapualide; Utrophin; Unc-87; Unc-60 (ADF/cofilins); VASP; Vav; Verprolin; VDAC; Vibrio cholerae RTX toxin; Villin; Vinculin; VIM (Vimentin); Vitamin D-binding protein; WIP; WASp; Y-box proteins; YpkA (YopO); Zipper protein; Zo-1; or Zyxin.

Suitably, the amino acid sequence of element B is selected from or derived from a protein of List B, preferably selected from or derived from an actin binding domain of any of the proteins of List B.

In preferred embodiments, the amino acid sequence element B is selected from a myofibrillar protein. A typical myofibrillar protein may be selected from actin or myosin. Suitably, the myosin is selected from a myosin heavy chain or a myosin light chain.

A particularly preferred cytoplasmic peptide or protein, in particular cytoskeletal protein, is smooth muscle myosin heavy chain (SMMHC), or a fragment or variant of SMMHC that binds to a cytoskeletal protein (e.g., actin).

The cytoskeletal protein smooth muscle myosin heavy chain (SMMHC, Myosin-11, MYH11) is a protein belonging to the myosin heavy chain family. SMMHC is an actin binding protein. SMMHC is a subunit of a hexameric protein that consists of two heavy chain subunits and two pairs of non-identical light chain subunits. There are four different isoforms of SMMHC proteins. The SMMHC isoform 1 (UniProt database entry P35749-1; SEQ ID NO: 184) has a length of 1972 amino acid residues, and three further isoforms (P35749-2, P35749-3, P35749-4; SEQ ID NOs: 185-187).

In the context of the invention, an important amino acid region of the SMMHC protein represent the high affinity binding domain (HABD; position 1539 to 1592 in relation to SEQ ID NO: 184). HABD is a protein domain that may promote a stronger binding to the target transcription factor (e.g. RUNX) which may be important for a dominant negative effect of the encoded transcription factor inhibitor as the HABD may outcompete the interaction of transcriptional co-factors (e.g. CBFbeta). A further important amino acid region of the SMMHC protein represent the Assembly competence domain (ACD; position 1876 to 1903 in relation to SEQ ID NO: 184). ACD is a protein domain that may allows for a self-dimerization of the transcription factor inhibitor (carrying such a sequence). A further important amino acid region of the SMMHC protein represents a transcriptional repression domain (TRD; position 1809 to 1877 in relation to SEQ ID NO: 184). TRD is a protein domain that may further repress the activity of the target transcription factor.

Accordingly, in preferred embodiments, the amino acid sequence selected or derived from SMMHC comprises at least one of a high-affinity binding domain (HABD) and/or an assembly competent domain (ACD) and/or a transcriptional repression domain (TRD), or a fragment or variant of any of these. In the context of RUNX inhibitors, it may be suitable that amino acid sequence element B comprises HABD as that domain may increase the affinity for the target transcription factor RUNX and as that domain may lead to a binding of twice as many cellular RUNX molecules compared to cellular CBFbeta.

In preferred embodiments, the SMMHC amino acid sequence selected for element B is derived or selected from a C-terminal fragment of a human SMMHC (that is, it lacks the N-terminal part).

In preferred embodiments, element B comprises an amino acid sequence selected or derived from SMMHC, preferably wherein the SMMHC amino acid sequence is a C-terminal fragment of a human SMMHC. In embodiments, the C-terminal fragment of a human SMMHC comprises a C-terminal stretch from position 1000 to position 1972, position 1100 to position 1972, position 1200 to position 1972, position 1300 to position 1972, position 1400 to position 1972, position 1500 to position 1972, position 1550 to position 1972, position 1600 to position 1972, position 1650 to position 1972, position 1700 to position 1972, position 1750 to position 1972, position 1800 to position 1972, position 1850 to position 1972, or position 1900 to position 1972 of amino acid sequence SEQ ID NO: 184.

In embodiments, the C-terminal fragment of a human SMMHC comprises a C-terminal stretch of SMMHC comprising at least 900, 800, 700, 600, 500, 300, or 200 amino acid residues of SEQ ID NO: 184.

Suitably, element B may be selected or derived from a C-terminal portion of SMMHC comprising amino acid 1527 to aa1972, or aa1809 to aa1972 (in relation to SEQ ID NO: 184). For example, C-terminal portion of SMMHC comprising amino acid 1527 to aa1972 may comprise an amino acid sequence according to SEQ ID NO: 188). For example, C-terminal portion of SMMHC comprising amino acid 1809 to aa1972 may comprise an amino acid sequence according to SEQ ID NO: 190).

In preferred embodiments, the SMMHC amino acid sequence is additionally C-terminally truncated.

Accordingly, the SMMHC amino acid sequence may additionally comprise a C-terminal deletion of at least about 50 amino acids, e.g. 50, 60, 70, 80, 90, 95, or 100 amino acids of amino acid sequence SEQ ID NO: 184.

Without wishing to be bound to theory, chromosomal rearrangements in humans can lead to a fusion of CBFbeta and SMMHC genes which has been observed inter alia in acute myeloid leukaemia subtype M4Eo (AML with eosinophilia). Interestingly, it has been observed that a deletion of 95 amino acids from the C-terminus of CBFbeta-SMMHC (CBFbeta-SMMHCAC95) blocked leukemogenesis while retaining RUNX1 inhibitory properties in knock in mice. Accordingly, in the context of the invention, a transcription factor inhibitor comprising a deletion in the C-terminus of about 95aa (SMMHCAC95) may be more suitable in certain medical applications. Moreover, a shorter encoded transcription factor inhibitor is beneficial in terms of nucleic acid production.

In preferred embodiments, the SMMHC amino acid sequence comprises a deletion in the C-terminus of about 95aa (SMMHCAC95), for example an SMMHC fragment comprising aa1527 to aa1877 (in relation to SEQ ID NO: 184). Accordingly, element B may be selected or derived from a C-terminal portion of SMMHC comprising aa1527 to aa1877, or aa1809 to aa1877 (in relation to SEQ ID NO: 184). For example, C-terminal portion of SMMHC comprising amino acid 1527 to aa1877 may comprise an amino acid sequence according to SEQ ID NO: 189). For example, C-terminal portion of SMMHC comprising amino acid 1809 to aa1877 may comprise an amino acid sequence according to SEQ ID NO: 191).

SMMHC proteins and fragments thereof as defined herein are particularly preferred in the context of the invention as SMMHC elements as defined herein may confer a negative dominant effect to transcription factor inhibitors of the invention.

Accordingly, amino acid sequence element B of the transcription factor inhibitor can comprise amino acid 1527 to 1972 of human SMMHC (in relation to SEQ ID NO: 184). Another even more preferred SMMHC fragment comprises amino acid 1527 to 1877 (in relation to SEQ ID NO: 184). In some embodiments, the SMMHC fragments comprising amino acid 1809 to 1972 (in relation to SEQ ID NO: 184) or 1809 to 1877 (in relation to SEQ ID NO: 184) may be preferred.

Other preferred cytoplasmic peptide or proteins, in particular cytoskeletal proteins, may be selected or derived from a synthetic peptide that stains filamentous actin (LifeAct®).

LifeAct® Dye is a peptide dye composed of a 17 amino acid recombinant peptide that stains actin (e.g. filamentous actin) structures of cells. In preferred embodiments, the amino acid sequence of element B may be selected or derived from a LifeAct® peptide (e.g. SEQ ID NO: 192 or 193).

In other embodiments, the at least one amino acid sequence element B comprises or consists of an amino acid sequence selected or derived from NFAT5, in particular NFAT5 isoform A, the full sequence being derivable from Uniprot database entry 094916-2 (see also SEQ ID NO: 1521). In preferred embodiments in that context, an N-terminal fragment of the NFAT5 isoform A is selected. For example, an N-terminal fragment comprising amino acid 1 to amino acid 17 of the NFAT5 isoform A according to Uniprot database entry 094916-2 (e.g. MGGACSSFTTSSSPTIY; e.g. SEQ ID NO: 1522). Suitably, introduction of an NFAT5 sequence may keep the target transcription factor (e.g. RUNX1) outside the nucleus via anchoring to plasma membrane e.g. via lipid modification.

In preferred embodiments in that context, the transcription factor inhibitor (provided by the artificial nucleic acid of the invention) comprises at least one amino acid sequence element B that comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 184-193, 1521, 1522, or fragments or variants of any of these. Suitable amino acid sequences are also provided in Table 1.

In particularly preferred embodiments, the transcription factor inhibitor (provided by the artificial nucleic acid of the invention) comprises at least one amino acid sequence element B that comprises or consists of an amino acid sequence selected or derived from a SMMHC fragment being identical or at least 90%, 91%, 92%, 93%, 94%, 95%, 96%, 97%, 98%, or 99% identical to SEQ ID NO: 189, or a fragment or variant thereof.

In preferred embodiments, the at least one amino acid sequence element B comprises or consists of an amino acid sequence selected or derived from a transcriptional repressor of the target transcription factor, or a fragment or variant thereof.

Such proteins are particularly suitable as the transcription factor inhibitor of the invention that e.g. binds to a cellular transcription co-factor (e.g. CBFbeta via a RUNX amino acid sequence element A) may act as a repressor of the activity of the target transcription factor.

In preferred embodiments, the transcriptional repressor of the target transcription factor is selected or derived from any repressor of any of the transcription factors provided in List A.

In preferred embodiments, the encoded transcriptional repressor of the target transcription factor is selected or derived from RUNX1 Partner Transcriptional Co-Repressor 1 (RUNX1T1), or a fragment or variant thereof.

RUNX1T1a (RUNX1T1 or CBFA2T1, AML1T1, CBFA2T1, CDR, ETO, MTG8, ZMYND2, AML1-MTG8, t(8;21)(q22;q22), RUNX1 translocation partner 1, RUNX1 partner transcriptional co-repressor 1) is a protein that in humans is encoded by the RUNX1T1 gene. The protein is a Transcriptional corepressor which facilitates transcriptional repression via its association with DNA-binding transcription factors and recruitment of other corepressors and histone-modifying enzymes. There are several described isoforms of RUNX1T1a including isoform 1 (UniProt database entry Q06455-1; SEQ ID NO: 216) that has a length of 604 amino acid residues, and further different isoforms (UniProt database entry Q06455-2, Q06455-3, Q06455-4, Q06455-5, Q06455-6, Q14244-7; SEQ ID NOs: 217-223).

In preferred embodiments, the RUNX1T1a amino acid sequence selected for element B is derived or selected from an N-terminal fragment of a human RUNX1T1a (that is, it lacks the N-terminal part). The C-terminal region of RUNX1T1a comprises several domains including an NHR1, NHR2, NHR3, NHR4 and an NLS domain. Accordingly, the amino acid sequence of element B may be selected or derived from a N-terminal RUNX1T1a fragment comprising about 574 amino acids of the C-terminus, namely aa31 to aa604 (in relation to SEQ ID NO: 216) or comprising about 583 amino acids of the C-terminus, namely aa22 to aa604 (in relation to SEQ ID NO: 216). For example, the N-terminal RUNX1T1a fragment comprising amino acid 31 to amino acid 604 may comprise an amino acid sequence according to SEQ ID NO: 224).

RUNX1T1b (CBFA2T2; MTGR1; EHT; ZMYND3; MTG8R; CBFA2/RUNX1) is a protein that in humans is encoded by the CBFA2T2 gene. The protein is a Transcriptional corepressor which facilitates transcriptional repression via its association with DNA-binding transcription factors and recruitment of other corepressors and histone-modifying enzymes. There are several described isoforms of RUNX1T1b including isoform 1 (UniProt database entry O43439-1; SEQ ID NO: 225) that has a has a length of 604 amino acid residues, and further different isoforms (UniProt database entry 043439-2, 043439-3, 043439-4, 043439-5; SEQ ID NOs: 226-228).

In preferred embodiments, the RUNX1T1b amino acid sequence selected for element B is derived or selected from an N-terminal fragment of a human RUNX1T1b (that is, it lacks the N-terminal part). The C-terminal region of RUNX1T1b comprises several domains including an NHR2, NHR3 and an NLS domain. Accordingly, the amino acid sequence of element B may be selected or derived from a N-terminal RUNX1T1b fragment comprising about 574 amino acids of the C-terminus, namely aa31 to aa604 (in relation to SEQ ID NO: 225) or comprising about 583 amino acids of the C-terminus, namely aa22 to aa604 (in relation to SEQ ID NO: 225). For example, the N-terminal RUNX1T1b fragment comprising amino acid 22 to amino acid 604 may comprise an amino acid sequence according to SEQ ID NO: 230. Another exemplary RUNX1T1b fragment (having a length of about 16 amino acids) may comprise an amino acid sequence according to SEQ ID NO: 229.

In preferred embodiments, the encoded transcription factor inhibitor comprises at least one amino acid sequence element B that comprises or consists of an amino acid sequence selected or derived from RUNX1T1a or RUNX1T1b being identical or at least 70%, 80%, 85%, 86%, 87%, 88%, 89%, 90%, 91%, 92%, 93%, 94%, 95%, 96%, 97%, 98%, or 99% identical to any one of SEQ ID NOS: 216-230, or fragments or variants of any of these. Suitable amino acid sequences are also provided in Table 1.

In preferred embodiments, the at least one amino acid sequence element B comprises or consists of an amino acid sequence selected or derived from a peptide or protein that promotes degradation of the target transcription factor, or a fragment or variant thereof.

Such proteins are particularly suitable as the transcription factor inhibitor of the invention that e.g. binds to a cellular target transcription factor (e.g. RUNX via a CBFbeta amino acid sequence element A) may act as degradation signal or degradation promoter for the respective cellular target transcription factor (e.g. RUNX).

In preferred embodiments in that context, the encoded peptide or protein that promotes degradation is selected or derived from a protein that binds to E3 ligase. Examples of such proteins comprise HIF1alpha, MDM2, or CRBN, or a fragment or variant of any of these. Particularly preferred in that context is HIF1alpha.

HIF1alpha is a protein that in humans is encoded by the HIF1A gene. There are several described isoforms of HIF1alpha including isoform 1 (UniProt database entry Q16665-1; SEQ ID NO: 194) that has a has a length of 826 amino acid residues, and further different isoforms (UniProt database entry Q16665-2, Q16665-3).

In preferred embodiments, the HIF1alpha amino acid sequence selected for element B is derived or selected from a fragment of a human HIF1alpha. Accordingly, the amino acid sequence of element B may be selected or derived from a HIF1alpha fragment comprising aa549 to aa575 (in relation to SEQ ID NO: 194). For example, the HIF1alpha fragment comprising amino acid 549 to amino acid 575 may comprise an amino acid sequence according to SEQ ID NO: 195.

Transcription factor inhibitors comprising a peptide or protein that promotes degradation (e.g. HIF1alpha) may cause proteasome mediated degradation of the target transcription factor (e.g. RUNX) upon binding (e.g. mediated via CBFbeta). For example, in a cellular context, HIF1alpha modified via Prolyl hydroxylation, ubiquitylated and targeted for proteosomal degradation via binding to E3 ligase like VHL. Other examples of a similar mechanism include peptides with motif binding domains for other E3 ubiquitin ligases like MDM2 and CRBN.

In preferred embodiments, the encoded transcription factor inhibitor comprises at least one amino acid sequence element B that comprises or consists of an amino acid sequence selected or derived from HIF1alpha being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 194 or 195, or fragments or variants of any of these. Suitable amino acid sequences are also provided in Table 1.

Preferred amino acid sequence elements (element A and B) of the encoded transcription factor inhibitors and corresponding nucleic acid sequences are provided in Table 1. Therein, each row corresponds to a suitable amino acid sequence elements that may be comprised in a transcription factor inhibitor. Rows 1 to 8 responds to a suitable amino acid sequence element A as defined herein. Rows 9 to 23 responds to a suitable amino acid sequence element A as defined herein. Column A of Table 1 provides a short description of the respective amino acid element. Column B of Table 1 provides protein (amino acid sequence) SEQ ID NOs of respective amino acid elements. Column C of Table 1 provides SEQ ID NO of the corresponding wild type or reference nucleic acid coding sequences. Column D of Table 1 provides SEQ ID NO of the corresponding G/C optimized nucleic acid coding sequences (opt1). Column E of Table 1 provides SEQ ID NO of corresponding human codon usage adapted nucleic acid coding sequences (opt 3). Column F of Table 1 provides SEQ ID NO of further codon optimized coding sequences (opt4, opt5, opt11). Preferred encoded transcription factor inhibitors that comprise the elements of Table 1 and respective nucleic acid sequences encoding said transcription factor inhibitors are provided in Tables 2, 3, and 4.

TABLE 1
Element A and B of transcription factor inhibitors (amino acid sequences and cds sequences):

| | | | | | | |
|---|---|---|---|---|---|---|
| row | A | B | C | D | E | F |

Element A sequences:

| | | | | | | |
|---|---|---|---|---|---|---|
| 1 | CBFbeta | 178-181 | 240-243, | 364-367, | 488-491, | 612-615, 674- |
| | | | 302-305 | 426-429 | 550-553 | 677, 736-739 |
| 2 | CBFbeta(1-141) | 182 | 244, 306 | 368, 430 | 492, 554 | 616, 678, 740 |
| 3 | CBFbeta(1-165) | 183 | 245, 307 | 369, 431 | 493, 555 | 617, 679, 741 |
| 4 | RUNX1 | 198-212 | 260-274, | 384-398, | 508-522, | 632-646, 694- |
| | | | 322-336 | 446-460 | 570-584 | 708, 756-770 |
| 5 | RUNX1(1-128) | 213 | 275, 337 | 399, 461 | 523, 585 | 647, 709, 771 |
| 6 | RUNX1(1-177) | 214 | 276, 338 | 400, 462 | 524, 586 | 648, 710, 772 |
| 7 | RUNX1(1-241) | 215 | 277, 339 | 401,463 | 525, 587 | 649, 711, 773 |
| 8 | RUNX1(K83E, R174Q) | 197 | 259, 321 | 383, 445 | 507, 569 | 631, 693, 755 |

Element B sequences:

| | | | | | | |
|---|---|---|---|---|---|---|
| 9 | SMMHC | 184-187 | 246-249, | 370-373, | 494-497, | 618-621, 680- |
| | | | 308-311 | 432-435 | 556-559 | 683, 742-745 |
| 10 | SMMHC(1527-1972) | 188 | 250, 312 | 374, 436 | 498, 560 | 622, 684, 746 |
| 11 | SMMHC(1527-1877) | 189 | 251, 313 | 375, 437 | 499, 561 | 623, 685, 747 |
| | (SMMHCΔC95) | | | | | |
| 12 | SMMHC(1809-1972) | 190 | 252, 314 | 376, 438 | 500, 562 | 624, 686, 748 |
| 13 | SMMHC(1809-1877) | 191 | 253, 315 | 377, 439 | 501, 563 | 625, 687, 749 |
| 14 | LifeAct ® | 192, 193 | 254, 255, | 378, 379, | 502, 503, | 626, 627, 688, |
| | | | 316, 317 | 440, 441 | 564, 565 | 689, 750, 751 |
| 15 | RUNX1T1a | 216-223 | 278-285, | 402-409, | 526-533, | 650-657, 712- |
| | | | 340-347 | 464-471 | 588-595 | 719, 774-781 |
| 16 | RUNX1T1a(31-604) | 224 | 286, 348 | 410, 472 | 534, 596 | 658, 720, 782 |
| 17 | RUNX1T1b | 225-228 | 287-290, | 411-414, | 535-538, | 659-662, 721- |
| | | | 349-352 | 473-476 | 597-600 | 724, 783-786 |
| 18 | RUNX1T1b(22-604) | 230 | 292, 354 | 416, 478 | 540, 602 | 664, 726, 788 |
| 19 | RUNX1T1b(short) | 229 | 291, 353 | 415, 477 | 539, 601 | 663, 725, 787 |
| 20 | HIF1alpha | 194 | 256, 318 | 380, 442 | 504, 566 | 628, 690, 752 |
| 21 | HIF1alpha(549-575) | 195 | 257, 319 | 381, 443 | 505, 567 | 629, 691, 753 |
| 22 | NFAT5 | 1521 | 1523, 1525 | 1527, 1529 | 1531, 1533 | 1535, 1537, 1539 |
| 23 | NFAT5(1-17) | 1522 | 1524, 1526 | 1528, 1530 | 1532, 1534 | 1536, 1538, 1540 |

### Amino Acid Sequences Comprising Element A and Element B

In preferred embodiments, the artificial nucleic acid of the invention comprises at least one coding sequence encoding at least one transcription factor inhibitor for reducing or inhibiting the activity of a target transcription factor in a cell, wherein the transcription factor inhibitor comprises or consists of at least one amino acid sequence element A as defined herein (preferably as in Table 1; rows 1 to 8) or at least one amino acid sequence element B as defined herein (preferably as in Table 1; rows 9 to 23).

In particularly preferred embodiments, the artificial nucleic acid of the invention comprises at least one coding sequence encoding at least one transcription factor inhibitor for reducing or inhibiting the activity of a target transcription factor in a cell, wherein the transcription factor inhibitor comprises or consists of at least one amino acid sequence element A as defined herein (preferably as in Table 1; rows 1 to 8) and at least one amino acid sequence element B as defined herein (preferably as in Table 1; rows 9 to 23).

In particularly preferred embodiments, the transcription factor inhibitor (that is provided by the artificial nucleic acid of the invention) is a fusion protein that comprises or consists of at least one amino acid sequence element A as defined herein and at least one amino acid sequence element B as defined herein.

In preferred embodiments, the at least one element A is located at the N-terminus of the transcription inhibitor and the at least one element B is located at the C-terminus of the transcription inhibitor.

In preferred embodiments, the encoded transcription factor inhibitor additionally comprises at least one further amino acid sequence element.

Suitably, the at least one further amino acid sequence element is selected from at least one linker sequence, at least one transmembrane domain, at least one secretion signal, an element that extends protein half-life, or a fragment or variant of any of these.

Suitable multimerization domains may be selected from the list of amino acid sequences according to SEQ ID NOs: 1116-1167 of WO2017081082, or fragments or variants of these sequences. Suitable transmembrane elements may be selected from the list of amino acid sequences according to SEQ ID NOs: 1228-1343 of WO2017081082, or fragments or variants of these sequences. Suitable secretory signal peptides may be selected from the list of amino acid sequences according to SEQ ID NOs: 1-1115 and SEQ ID NO: 1728 of published PCT patent application WO2017081082, or fragments or variants of these sequences.

In particularly preferred embodiments, the encoded transcription factor inhibitor comprises at least one further amino acid sequence element selected from a linker sequence. Suitable peptide linkers may be selected from the list of amino acid sequences according to SEQ ID NOs: 1509-1565 of the patent application WO2017081082, or fragments or variants of these sequences.

A preferred linker in the context of the invention is a flexible linker, preferably a GGS linker, more preferably a GGS linker according to SEQ ID NO: 196, or a variant thereof.

The use of peptide linker sequence may be advantageous in embodiments where the encoded transcription factor inhibitor is a fusion protein comprising at least one element A and the at least one element B. In such embodiments, the peptide linker sequence may be located (in a fusion protein) between element A and element B. As an example, introducing a linker sequence may inter alia improve the binding of the encoded transcription factor inhibitor to the target transcription factor or its co-factor (via element A), or may inter alia improve the binding or capturing of the transcription factor inhibitor to the cytoskeleton of the cell (via element B).

In preferred embodiments, the encoded transcription factor inhibitor comprises at least one element that extends protein half-life. Suitable element that extends protein half-life may be selected from the list of amino acid sequences according to SEQ ID NOs: 1671-1727 of WO2017081082, or fragments or variants of these sequences. The element that extends protein half-life is typically located at the N- or at the C-terminus of the transcription factor inhibitor of the invention. Transcription factor inhibitors comprising element that extends protein half-life are preferred in the context of medical treatments as e.g. the therapeutic effect is prolonged and/or the number of administrations can be reduced. That is particularly preferred in the context of ocular administration.

Accordingly, the encoded transcription factor inhibitor of the invention may comprise the following amino acid sequence elements, preferably selected from Table 1:
element A
element A-linker-element B
element B-linker-element A
element A-element B
element B-element A

Particularly preferred transcription factor inhibitor protein designs are provided in Table 2, column A.

In preferred embodiments, the encoded transcription factor inhibitor is a RUNX inhibitor, for example a RUNX1, RUNX2, and/or RUNX3 inhibitor. In particularly preferred embodiments, the encoded transcription factor inhibitor is a RUNX1 inhibitor.

In preferred embodiments, the encoded transcription factor inhibitor is a RUNX trap, for example a RUNX1, RUNX2, and/or RUNX3 trap. In particularly preferred embodiments, the encoded transcription factor trap is a RUNX1 trap.

Accordingly, in preferred embodiments, the transcription factor inhibitor is a RUNX trap that is configured to bind to a cellular RUNX transcription factor (e.g. via an amino acid sequence that interacts or binds to the target transcription factor, e.g. CBFbeta) and is additionally capable of capturing said RUNX transcription factor in the cytosol (e.g. via an amino acid sequence that blocks nuclear translocation of the target transcription factor, e.g. SMMHC). A “RUNX trap” is particularly suitable in the context of the invention as the target transcription factor RUNX is blocked from entering into the nucleus where RUNX typically acts. Blocking of the RUNX to enter into the nucleus (e.g. RUNX1) may also be associated with a reduced or blocked transport of its cellular transcription co-factors (e.g. CBFbeta), in particular in cases where the transcription co-factors of the target transcription factor is transported via the transcription factor into the nucleus. Of course, in such a case, a capturing of the RUNX in the cytosol (e.g. RUNX1) would, at the same time, prevent or reduce the transport of its transcription co-factors (e.g. CBFbeta) into the nucleus (e.g. CBFbeta needs RUNX for nuclear translocation).

According to various preferred embodiments, the artificial nucleic acid of the invention comprises at least one coding sequence encoding at least one RUNX inhibitor or trap, preferably the RUNX trap, comprising or consisting of
at least one amino acid sequence element A selected or derived from a transcription co-factor of a transcription factor of list A, or a preferred transcription factor as defined herein, and
at least one amino acid sequence element B selected or derived from a cytoplasmic protein as defined herein, preferably selected or derived from a cytoskeletal protein as defined herein, more preferably selected from SMMHC as defined herein.

According to various preferred embodiments, the artificial nucleic acid of the invention comprises at least one coding sequence encoding at least one RUNX inhibitor or trap, preferably the RUNX trap, comprising or consisting of
at least one amino acid sequence element A selected or derived from a transcription co-factor of RUNX as defined herein, more preferably selected or derived from CBFbeta as defined herein, and
at least one amino acid sequence element B selected or derived from a cytoplasmic protein as defined herein, preferably selected or derived from a cytoskeletal protein as defined herein, more preferably selected from SMMHC as defined herein.

In preferred embodiments, the encoded RUNX inhibitor, preferably the RUNX trap, comprises or consists of a fusion protein comprising a CBFbeta amino acid sequence element (element A) as defined herein and an SMMHC amino acid element (element B) as defined herein. Optionally, a linker sequence may be located between element A and element B.

Suitably, the encoded RUNX inhibitor, preferably the RUNX trap, comprises or consists of
at least one amino acid sequence element A selected or derived from CBFbeta, preferably wherein amino acid sequence element A comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOS: 178-183, or fragments or variants of any of these, and
at least one amino acid sequence element B selected or derived from SMMHC, LifeAct®, or NFAT5, preferably wherein amino acid sequence element B comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 184-193, 1521, 1522, or fragments or variants of any of these, and
optionally, at least one linker sequence located between element A and element B, preferably selected from a flexible linker, more preferably a flexible GGS linker according to SEQ ID NO: 196, or a fragment or variant thereof.

In particularly preferred embodiments, the encoded RUNX inhibitor, preferably the RUNX trap, comprises or consists of
at least one amino acid sequence element A selected or derived from CBFbeta, wherein amino acid sequence element A comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 183, or fragment or variant thereof, and
at least one amino acid sequence element B selected or derived from SMMHC, wherein amino acid sequence element B comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 189, or a fragment or variant thereof.

In preferred embodiments, the encoded transcription factor inhibitor, preferably the RUNX trap (CBFbeta-SMMHC), comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 231-233, 1541-1548, or fragments or variants thereof. Suitable amino acid sequences are also provided in Table 2.

In preferred embodiments, the encoded transcription factor inhibitor, preferably the RUNX trap (CBFbeta-SMMHC), comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 231 or 232, or fragments or variants of CBFbeta-SMMHC proteins.

In particularly preferred embodiments, the encoded transcription factor inhibitor, preferably the RUNX trap (CBFbeta-SMMHC), comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 232, or fragments or variants thereof.

In particularly preferred embodiments, upon administration of the artificial nucleic acid to a cell or subject, the produced RUNX inhibitor or trap (e.g. CBFbeta-SMMHC) sequesters cellular RUNX by binding to RUNX in the cytosol and preferably trapping RUNX in the cytosol. Said binding to RUNX in the cytosol leads to the formation of RUNX CBFbeta-SMMHC complexes. Accordingly, the formation of cellular RUNX-CBFbeta heterodimer is reduced or prevented.

In particularly preferred embodiments, upon administration of the artificial nucleic acid to a cell or subject, the produced RUNX inhibitor or trap (e.g. CBFbeta-SMMHC) reduces or prevents the translocation of cellular CBFbeta from the cytosol to the nucleus by reducing or preventing its interaction with cellular RUNX. Accordingly, the formation of cellular RUNX-CBFbeta heterodimer is reduced or prevented.

In preferred embodiments, the RUNX inhibitor or trap (e.g. CBFbeta-SMMHC) is configured to bind more than one cellular RUNX protein, preferably about two cellular RUNX proteins.

In some embodiments, upon administration of the artificial nucleic acid to a cell or subject, the produced RUNX inhibitor or trap (e.g. CBFbeta-SMMHC) binds cellular RUNX transcription factor and a subset of the formed complex (RUNX bound to CBFbeta-SMMHC) may enter the nucleus, wherein a larger subset of RUNX CBFbeta-SMMHC complex is preferably trapped in the cytosol. Advantageously, the subset of RUNX CBFbeta-SMMHC complex that may enter the nucleus drives transcriptional repression of genes that are under control of RUNX.

In other embodiments, the RUNX inhibitor comprises or consists of a fusion protein comprising a CBFbeta amino acid sequence element as defined herein and an HIF1alpha amino acid sequence element as defined herein.

Suitably in that context, the encoded RUNX inhibitor comprises or consists of
at least one amino acid sequence element A selected or derived from CBFbeta, preferably wherein amino acid sequence element A comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 178-183, or fragments or variants of any of these, and
at least one amino acid sequence element B selected or derived from HIF1alpha, preferably wherein amino acid sequence element B comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 194-195, or fragments or variants of any of these, and
optionally, at least one linker sequence located between element A and element B, preferably selected from a flexible linker, more preferably a flexible GGS linker according to SEQ ID NO: 189, or a fragment or variant thereof.

In preferred embodiments, the encoded transcription factor inhibitor, preferably the RUNX inhibitor (CBFbeta-HIF1alpha), comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 234 or 235, or fragments or variants thereof. Suitable amino acid sequences are also provided in Table 2.

Suitably in that context, the RUNX inhibitor (CBFbeta-HIF1alpha) degrades cellular RUNX, preferably cellular RUNX1.

According to other preferred embodiments, the artificial nucleic acid of the invention comprises at least one coding sequence encoding at least one RUNX inhibitor comprising or consisting of
at least one amino acid sequence element A selected or derived from the target transcription factor as defined herein, more preferably selected or derived from RUNX1 as defined herein, and
at least one amino acid sequence element B selected or derived from a transcriptional repressor of the target transcription factor as defined herein, preferably selected or derived from RUNX1T1a or RUNX1T1b.

Accordingly, the encoded RUNX inhibitor comprises or consists of a fusion protein comprising a RUNX1 amino acid sequence element (element A) and a RUNX1T1a or RUNX1T1b amino acid sequence element (element B). Optionally, a linker sequence may be located between element A and element B.

Suitably, the encoded RUNX inhibitor comprises or consists of
at least one amino acid sequence element A selected or derived from RUNX1, preferably wherein amino acid sequence element A comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOS: 197-215, or fragments or variants of any of these, and
at least one amino acid sequence element B selected or derived from RUNX1T1a or RUNX1T1b, preferably wherein amino acid sequence element B comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 216-230, or fragments or variants of any of these, and
optionally, at least one linker sequence located between element A and element B, preferably selected from a flexible linker, more preferably a flexible GGS linker according to SEQ ID NO: 189, or a fragment or variant thereof.

In preferred embodiments, the encoded transcription factor inhibitor, preferably the RUNX inhibitor (RUNX1-RUNX1T1a, RUNX1-RUNX1T1b), comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 236-239, or fragments or variants of any of these. Suitable amino acid sequences are also provided in Table 2.

According to other embodiments, the artificial nucleic acid of the invention comprises at least one coding sequence encoding at least one RUNX inhibitor comprising or consisting of
at least one amino acid sequence element A selected or derived from the target transcription factor that comprises at least one amino acid substitution or deletion that reduces or prevents binding to its target DNA and/or at least one amino acid substitution or deletion that reduces or prevents nuclear translocation as defined herein, more preferably selected or derived from RUNX1 comprising an amino acid substitution selected from R174Q and/or K83E, or any functionally equivalent amino acid substitution at position R174 and/or K83.

In preferred embodiments, the encoded transcription factor inhibitor, preferably the RUNX inhibitor (RUNX1(K83E,R174Q)), comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 197, or a fragment or variant thereof. Suitable amino acid sequences are also provided in Table 2.

In preferred embodiments, upon administration of the artificial nucleic acid to a cell or subject, the produced RUNX inhibitor (e.g. RUNX1-RUNX1T1; RUNX1 (K83E,R174Q)) sequesters cellular CBFbeta by binding to CBFbeta in the cytosol. Accordingly, the formation of cellular RUNX-CBFbeta heterodimer is reduced or prevented.

In preferred embodiments, upon administration of the artificial nucleic acid to a cell or subject, the produced RUNX inhibitor (e.g. RUNX1-RUNX1T1; RUNX1 (K83E,R174Q)) drives transcriptional repression of genes that are under control of RUNX.

In preferred embodiments, upon administration of the artificial nucleic acid to a cell or subject, the produced RUNX inhibitor reduces or prevents the interaction of cellular RUNX with cellular CBFbeta. Accordingly, the formation of a cellular RUNX-CBFbeta heterodimeric complex is inhibited. In particularly preferred embodiments, upon administration of the artificial nucleic acid, the produced RUNX trap (e.g. CBFB-SMMHC) reduces or prevents the interaction of cellular RUNX with cellular CBFbeta. Accordingly, the formation of a cellular RUNX-CBFbeta heterodimeric complex is inhibited.

Accordingly, in preferred embodiments, upon administration of the artificial nucleic acid to a cell or subject, the produced RUNX inhibitor, preferably the RUNX trap, reduces cellular RUNX-CBFbeta complex formation and/or activity, preferably cellular RUNX-CBFbeta complex formation and/or activity. Suitably, as result of reducing cellular RUNX-CBFbeta complex formation and/or activity, the transcription activity of RUNX is reduced in the cell or subject.

Accordingly, in preferred embodiments, upon administration of the artificial nucleic acid to a cell or subject, the produced RUNX inhibitor, preferably the RUNX trap, reduces the cellular expression of RUNX controlled genes or gene products.

In particularly preferred embodiments, upon administration of the artificial nucleic acid to a cell or subject, the produced RUNX inhibitor, preferably the RUNX trap, reduces the cellular expression of TGFbeta2 (TGFβ2), SMAD3, and/or COL1A1.

In preferred embodiments, upon administration of the artificial nucleic acid to a cell or subject, the produced RUNX inhibitor, preferably the RUNX trap, increase the transcription rate of MARVELD2. MARVELD2 is a tight junction associated epithelial marker, as a predictor of the future state of the cell.

In preferred embodiments, upon administration of the artificial nucleic acid to a cell or subject, the produced RUNX inhibitor, preferably the RUNX trap, reduces or prevents pathological epithelial to mesenchymal transition (EMT), pathological cell proliferation, aberrant angiogenesis, aberrant (ocular) neovascularization, degeneration, and/or fibrosis. In embodiments, the administered artificial nucleic acid encoding the RUNX inhibitor or trap reduces or prevents pathological epithelial to mesenchymal transition (EMT), pathological cell proliferation, aberrant angiogenesis, aberrant (ocular) neovascularization, degeneration, and/or fibrosis in a more effective way as a small molecule inhibitor of RUNX (e.g. Ro5-335) or at least comparably effective as a small molecule inhibitor of RUNX (e.g. Ro5-335).

In preferred embodiments, upon administration of the artificial nucleic acid to a cell or subject, the produced RUNX inhibitor, preferably the RUNX trap, reduces or prevents pathological epithelial to mesenchymal transition (EMT).

Accordingly, in preferred embodiments, upon administration of the artificial nucleic acid to a cell or subject, the produced RUNX inhibitor, preferably the RUNX trap, reduces the cellular expression of RUNX, preferably RUNX1.

In cells, the expression of RUNX is regulated by self-regulatory feedback loops. That means that e.g. cellular RUNX proteins can activate their own expression (self-activation). As the RUNX inhibitor of the present invention may reduce or inhibit the activity of the target transcription factor RUNX in a cell (e.g. RUNX1), that can also lead to a reduced expression of RUNX. As exemplified herein for a RUNX1 trap, administration of an artificial nucleic acid encoding CBFB-SMMHC leads to a reduction of the cellular expression of RUNX1 (see Example section).

Preferred transcription factor inhibitors as defined herein are provided in Table 2. Therein, each row corresponds to a suitable transcription factor inhibitor construct. Column A of Table 2 provides a short description of suitable transcription factor inhibitor constructs. Column B of Table 2 provides protein (amino acid sequence) SEQ ID NOs of respective suitable transcription factor inhibitor constructs. Column C of Table 2 provides SEQ ID NO of the corresponding wild type or reference nucleic acid coding sequences. Column D of Table 2 provides SEQ ID NO of the corresponding G/C optimized nucleic acid coding sequences (opt1). Column E of Table 2 provides SEQ ID NO of the corresponding human codon usage adapted nucleic acid coding sequences (opt 3). Column F of Table 2 provides SEQ ID NO of further corresponding codon optimized nucleic acid coding sequences (opt4, opt5, opt11). Notably, the description of the invention explicitly includes the information provided under “feature key”, i.e. “source” (for nucleic acids or proteins) or “misc_feature” (for nucleic acids) or “REGION” (for proteins) of the ST.26 sequence listing of the present application. Preferred RNA constructs comprising coding sequences of Table 2, e.g. mRNA sequences, are provided in Table 3 and Table 4.

TABLE 2
Transcription factor inhibitor constructs (amino acid sequences and coding sequences):

| row | A | B | C | D | E | F |
|---|---|---|---|---|---|---|
| 1 | CBFbeta(1-165)-SMMHC(1527-1877) | 232 | 294, 356 | 418, 480 | 542, 604 | 666, 1557, 728, |
| | (CBFbeta-SMMHCΔC95) | | | | | 790, 1558 |
| 2 | CBFbeta(1-165)-SMMHC(1527-1927) | 231 | 293, 355 | 417, 479 | 541, 603 | 665, 727, 789 |
| 3 | CBFbeta(1-141)-SMMHC(1527-1877) | 1545 | | 1553 | | |
| 4 | CBFbeta(1-130)-Linker-Linker- | 1546 | | 1554 | | |
| | SMMHC(1527-1877) | | | | | |
| 5 | CBFbeta(1-165)-Linker-LifeAct | 233 | 295, 357 | 419, 481 | 543, 605 | 667, 729, 791 |
| 6 | CBFbeta(1-165)-Linker-LifeAct | 1547 | | 1555 | | |
| 7 | CBFbeta(1-165)-Linker-LifeAct | 1548 | | 1556 | | |
| 8 | CBFbeta(1-165)-HIF1alpha(549-575) | 234 | 296, 358 | 420, 482 | 544, 606 | 668, 730, 792 |
| 9 | CBFbeta(1-165)-Linker-HIF1alpha(549- | 235 | 297, 359 | 421, 483 | 545, 607 | 669, 731, 793 |
| | 575) | | | | | |
| 10 | CBFbeta(1-165)-Linker-NFAT5(1-17) | 1541 | | 1549 | | |
| 11 | NFAT5(1-17)-Linker-CBFbeta(1-165) | 1542 | | 1550 | | |
| 12 | CBFbeta(1-165)-NFAT5(1-17) | 1543 | | 1551 | | |
| 13 | NFAT5(1-17)-CBFbeta(1-165) | 1544 | | 1552 | | |
| 14 | RUNX1(1-241)-RUNX1T1b(short) | 237 | 299, 361 | 423, 485 | 547, 609 | 671, 733, 795 |
| 15 | RUNX1(1-241)-RUNX1T1b(22-604) | 238, 239 | 300, 301, | 424, 425, | 548, 549, | 672, 673, 734, |
| | | | 362, 363 | 486, 487 | 610, 611 | 735, 796, 797 |
| 16 | RUNX1(1-177)-RUNX1T1a(31-604) | 236 | 298, 360 | 422, 484 | 546, 608 | 670, 732, 794 |
| 17 | RUNX1(K83E, R174Q) | 197 | 259, 321 | 383, 445 | 507, 569 | 631, 693, 755 |

### Suitable Coding Sequences:

According to preferred embodiments, the artificial nucleic acid of the invention comprises at least one coding sequence encoding at least one transcription factor trap as defined herein. In that context, any coding sequence encoding at least one transcription factor trap as defined herein, or fragments and variants thereof may be understood as suitable coding sequence and may therefore be comprised in the nucleic acid of the invention.

In preferred embodiments, the artificial nucleic acid of the invention comprises or consists of at least one coding sequence encoding at least one transcription factor inhibitor or trap as defined herein, preferably encoding any one of SEQ ID NOS: 178-195, 197-239, 1521, 1522, 1541-1548, or fragments of variants thereof. It has to be understood that, on nucleic acid level, any sequence which encodes an amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 178-195, 197-239, 1521, 1522, 1541-1548, or fragments or variants thereof, may be selected and may accordingly be understood as suitable coding sequence of the invention. Further information regarding said amino acid sequences is also provided in e.g. Tables 1 and 2.

In preferred embodiments, the artificial nucleic acid comprises at least one coding sequence that comprises at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to the sequences according to any one of SEQ ID NOs: 240-797, 1523-1540, 1549-1558, or a fragment or a fragment or variant of any of these sequences. Further information regarding said nucleic acid sequences is also provided in e.g. Tables 1 and 2.

In preferred embodiments, the artificial nucleic acid is a modified and/or stabilized nucleic acid.

According to preferred embodiments, the artificial nucleic acid may thus be provided as a “stabilized nucleic acid” that is to say a nucleic acid showing improved resistance to in vivo degradation and/or a nucleic acid showing improved stability in vivo, and/or a nucleic acid showing improved translatability in vivo. This is particularly important in embodiments where the nucleic acid is an RNA.

Preferably, the artificial nucleic of the present invention may be provided as a “stabilized nucleic acid”, preferably a “stabilized RNA”.

In the following, suitable modifications/adaptations are described that are capable of “stabilizing” the nucleic acid, preferably the RNA.

In particularly preferred embodiments, the artificial nucleic acid comprises at least one codon modified coding sequence.

In preferred embodiments, the at least one coding sequence of the artificial nucleic acid is a codon modified coding sequence. Suitably, the amino acid sequence encoded by the at least one codon modified coding sequence is not being modified compared to the amino acid sequence encoded by the corresponding wild type or reference coding sequence.

The term “codon modified coding sequence” relates to coding sequences that differ in at least one codon (triplets of nucleotides coding for one amino acid) compared to the corresponding wild type or reference coding sequence. Suitably, a codon modified coding sequence in the context of the invention may show improved resistance to in vivo degradation and/or improved stability in vivo, and/or improved translatability in vivo. Codon modifications in the broadest sense make use of the degeneracy of the genetic code wherein multiple codons may encode the same amino acid and may be used interchangeably to optimize/modify the coding sequence for in vivo applications.

In particularly preferred embodiments, the at least one coding sequence of the artificial nucleic acid is a codon modified coding sequence, wherein the codon modified coding sequence is selected from C maximized coding sequence, CAI maximized coding sequence, human codon usage adapted coding sequence, G/C content modified coding sequence, and G/C optimized coding sequence, or any combination thereof.

In preferred embodiments, the at least one codon modified coding sequence is a G/C optimized coding sequence

In preferred embodiments, the at least one coding sequence of the artificial nucleic acid, preferably the RNA, has a G/C content of at least about 50%, 55%, or 60%. In particular embodiments, the at least one coding sequence of the nucleic acid has a G/C content of at least about 50%, 51%, 52%, 53%, 54%, 55%, 56%, 57%, 58%, 59%, 60%, 61%, 62%, 63%, 64%, 65%, 66%, 67%, 68%, 69%, or 70%.

When transfected into mammalian host cells, the nucleic acid comprising the codon modified coding sequence has a stability of between 12-18 hours, or greater than 18 hours, e.g., 24, 36, 48, 60, 72, or greater than 72 hours and is capable of being expressed by the mammalian host cell.

When transfected into mammalian host cells, the artificial nucleic acid comprising the codon modified coding sequence is translated into protein, wherein the amount of protein is at least comparable to, or preferably at least 10% more than, or at least 20% more than, or at least 30% more than, or at least 40% more than, or at least 50% more than, or at least 100% more than, or at least 200% or more than the amount of protein obtained by a naturally occurring or wild type or reference coding sequence transfected into mammalian host cells.

In some embodiments, the artificial nucleic acid may be modified, wherein the C content of the at least one coding sequence may be increased, preferably maximized, compared to the C content of the corresponding wild type or reference coding sequence (herein referred to as “C maximized coding sequence”). The generation of a C maximized nucleic acid sequences may suitably be carried out using a modification method according to WO2015062738. In this context, the disclosure of WO2015062738 is included herewith by reference.

In particularly preferred embodiments, the artificial nucleic acid may be modified, wherein the G/C content of the at least one coding sequence may be optimized compared to the G/C content of the corresponding wild type or reference coding sequence (herein referred to as “G/C optimized coding sequence”). “Optimized” in that context refers to a coding sequence wherein the G/C content is preferably increased to the essentially highest possible G/C content. The generation of a G/C content optimized nucleic acid sequence may be carried out using a method according to WO2002098443. In this context, the disclosure of WO2002098443 is included in its full scope in the present invention. G/C optimized coding sequences are indicated by the abbreviations “opt1” or “opt11”.

In preferred embodiments, the artificial nucleic acid may be modified, wherein the codons in the at least one coding sequence may be adapted to human codon usage (herein referred to as “human codon usage adapted coding sequence”). Codons encoding the same amino acid occur at different frequencies in humans. Accordingly, the coding sequence of the nucleic acid is preferably modified such that the frequency of the codons encoding the same amino acid corresponds to the naturally occurring frequency of that codon according to the human codon usage. For example, in the case of the amino acid Ala, the wild type or reference coding sequence is preferably adapted in a way that the codon “GCC” is used with a frequency of 0.40, the codon “GCT” is used with a frequency of 0.28, the codon “GCA” is used with a frequency of 0.22 and the codon “GCG” is used with a frequency of 0.10 etc. (see e.g. Table 2 of published PCT patent application WO2021156267). Accordingly, such a procedure (as exemplified for Ala) is applied for each amino acid encoded by the coding sequence of the nucleic acid to obtain sequences adapted to human codon usage. Human codon usage adapted coding sequences are indicated by the abbreviation “opt3”.

In preferred embodiments, the artificial nucleic acid may be modified, wherein the G/C content of the at least one coding sequence may be modified compared to the G/C content of the corresponding wild type or reference coding sequence (herein referred to as “G/C modified coding sequence”). In this context, the terms “G/C optimization” or “G/C content modification” relate to a nucleic acid that comprises a modified, preferably an increased number of guanosine and/or cytosine nucleotides as compared to the corresponding wild type or reference coding sequence. Such an increased number may be generated by substitution of codons containing adenosine or thymidine nucleotides by codons containing guanosine or cytosine nucleotides. Advantageously, nucleic acid sequences having an increased G/C content are more stable or show a better expression than sequences having an increased A/U. Preferably, the G/C content of the coding sequence of the nucleic acid is increased by at least 10%, 20%, 30%, preferably by at least 40% compared to the G/C content of the coding sequence of the corresponding wild type or reference nucleic acid sequence (herein referred to as “opt5”).

In other embodiments, the artificial nucleic acid may be modified, wherein the codon adaptation index (CAI) may be increased or preferably maximised in the at least one coding sequence (herein referred to as “CAI maximized coding sequence”). It is preferred that all codons of the wild type or reference nucleic acid sequence that are relatively rare in e.g. a human are exchanged for a respective codon that is frequent in the e.g. a human, wherein the frequent codon encodes the same amino acid as the relatively rare codon. Suitably, the most frequent codons are used for each amino acid of the encoded protein (see Table 2 of published PCT patent application WO2021156267, most frequent human codons are marked with asterisks). Suitably, the RNA comprises at least one coding sequence, wherein the codon adaptation index (CAI) of the at least one coding sequence is at least 0.5, at least 0.8, at least 0.9 or at least 0.95. Most preferably, the codon adaptation index (CAI) of the at least one coding sequence is 1 (CAI=1). For example, in the case of the amino acid Ala, the wild type or reference coding sequence may be adapted in a way that the most frequent human codon “GCC” is always used for said amino acid. Accordingly, such a procedure (as exemplified for Ala) may be applied for each amino acid encoded by the coding sequence of the nucleic acid to obtain CAI maximized coding sequences (herein referred to as “opt4”).

In particularly preferred embodiments, the at least one coding sequence of the nucleic acid of the invention is G/C optimized coding sequence.

In preferred embodiments, at least one coding sequence comprises a nucleic acid sequence encoding an amino acid sequence element A (CBFbeta), wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 240-245, 302-307, 364-369, 426-431, 488-493, 550-555, 612-617, 674-679, 736-741, or a fragment or a variant of any of these.

In preferred embodiments, at least one coding sequence comprises a G/C optimized (opt1, opt5, opt11) nucleic acid sequence encoding an amino acid sequence element A (CBFbeta), wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 364-369, 426-431, 674-679, 736-741, or a fragment or a variant of any of these.

In preferred embodiments, at least one coding sequence comprises a G/C optimized (opt1) nucleic acid sequence encoding an amino acid sequence element A (CBFbeta), wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 364, 369, 431, or 426, or a fragment or a variant of any of these.

In particularly preferred embodiments, at least one coding sequence comprises a G/C optimized (opt1) nucleic acid sequence encoding an amino acid sequence element A (CBFbeta), wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 369 or 431, or a fragment or a variant of any of these.

In preferred embodiments, the at least one coding sequence comprises a nucleic acid sequence encoding an amino acid element B (e.g. SMMHC, LifeAct® or NFAT5), wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 246-255, 308-317, 370-379, 432-441, 494-503, 556-565, 618-627, 680-689, 742-751, 1523-1540, or a fragment or a variant of any of these.

In preferred embodiments, at least one coding sequence comprises a G/C optimized (opt1, opt5, opt11) nucleic acid sequence encoding an amino acid sequence element B (e.g. SMMHC, LifeAct® or NFAT5), wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 370-379, 432-441, 680-689, 742-751, 1527, 1528, 1529, 1530, 1537, 1538, 1539, 1540, or a fragment or a variant of any of these.

In more preferred embodiments, at least one coding sequence comprises a G/C optimized (opt1) nucleic acid sequence encoding an amino acid sequence element B (SMMHC), wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 370, 375, 432, or 437, or a fragment or a variant of any of these.

In particularly preferred embodiments, at least one coding sequence comprises a G/C optimized nucleic acid sequence encoding an amino acid sequence element B (SMMHC), wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 375 or 437, or a fragment or a variant of any of these.

According to various preferred embodiments, the artificial nucleic acid of the invention comprises at least one coding sequence encoding at least one RUNX inhibitor, preferably a RUNX trap, wherein the artificial nucleic acid comprises or consists of
at least one coding sequence encoding an amino acid sequence element A selected or derived from a transcription co-factor of RUNX as defined herein, more preferably selected or derived from CBFbeta as defined herein, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 240-245, 302-307, 364-369, 426-431, 488-493, 550-555, 612-617, 674-679, 736-741, or a fragment or a variant of any of these; and
at least one coding sequence encoding an amino acid sequence element B cytoplasmic protein as defined herein, preferably selected or derived from a cytoskeletal protein as defined herein, more preferably selected from SMMHC, LifeAct® or NFAT5 as defined herein, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 246-255, 308-317, 370-379, 432-441, 494-503, 556-565, 618-627, 680-689, 742-751, 1523-1540, or a fragment or a variant of any of these;

Accordingly, in preferred embodiments, the at least one coding sequence comprises or consists of a nucleic acid sequence encoding an transcription factor inhibitor, preferably a RUNX trap (e.g. CBFbeta-SMMHC, CBFbeta-LifeAct® or CBFbeta-NFAT5), that is identical or at least 70%, 80%, 85%, 86%, 87%, 88%, 89%, 90%, 91%, 92%, 93%, 94%, 95%, 96%, 97%, 98%, or 99% identical to any one of the nucleic acid sequences SEQ ID NOs: 293-295, 355-357, 417-419, 479-481, 541-543, 603-605, 665-667, 727-729, 789-791, 1549-1558, or a fragment or a variant of any of these.

In preferred embodiments, the at least one coding sequence comprises or consists of a G/C optimized (opt1, opt5, opt11) nucleic acid sequence encoding an transcription factor inhibitor, preferably a RUNX trap CBFbeta-SMMHC, that is identical or at least 70%, 80%, 85%, 86%, 87%, 88%, 89%, 90%, 91%, 92%, 93%, 94%, 95%, 96%, 97%, 98%, or 99% identical to any one of the nucleic acid sequences SEQ ID NOs: 418, 480, 728, 790, 1558, or a fragment or a variant of these nucleic acid sequences encoding CBFbeta-SMMHC.

In preferred embodiments, the at least one coding sequence comprises or consists of a G/C optimized (opt1) nucleic acid sequence encoding a RUNX trap CBFbeta-SMMHC, that is identical or at least 70%, 80%, 85%, 86%, 87%, 88%, 89%, 90%, 91%, 92%, 93%, 94%, 95%, 96%, 97%, 98%, or 99% identical to any one of the nucleic acid sequences SEQ ID NOs: 418 or 480, or a fragment or a variant of these nucleic acid sequences encoding CBFbeta-SMMHC.

In particularly preferred embodiments, the at least one coding sequence comprises or consists of a G/C optimized (opt1) nucleic acid sequence encoding a RUNX trap CBFbeta-SMMHC, that is identical or at least 90%, 91%, 92%, 93%, 94%, 95%, 96%, 97%, 98%, or 99% identical to any one of the nucleic acid sequences SEQ ID NO: 418, or a fragment or a variant of that nucleic acid sequence encoding CBFbeta-SMMHC.

In other embodiments, the at least one coding sequence comprises or consists of a G/C optimized (opt1) nucleic acid sequence encoding a RUNX trap (e.g. CBFbeta-Linker-LifeAct), that is identical or at least 90%, 91%, 92%, 93%, 94%, 95%, 96%, 97%, 98%, or 99% identical to any one of the nucleic acid sequences SEQ ID NOs: 419, 481, 1555, 1556, or a fragment or a variant of any of these.

In other embodiments, the at least one coding sequence comprises or consists of a G/C optimized (opt1) nucleic acid sequence encoding a RUNX trap (e.g. CBFbeta-Linker-NFAT5), that is identical or at least 90%, 91%, 92%, 93%, 94%, 95%, 96%, 97%, 98%, or 99% identical to any one of the nucleic acid sequences SEQ ID NOs: 1549-1552, or a fragment or a variant of any of these.

In other preferred embodiments, the at least one coding sequence comprises a nucleic acid sequence encoding an amino acid element B (e.g. HIF1alpha), wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 257, 319, 381, 443, 505, 567, 629, 691, 753, or a fragment or a variant of any of these.

Suitably, the at least one coding sequence comprises or consists of at least one nucleic acid sequence encoding a CBFbeta as defined herein and at least one nucleic acid sequence encoding a HIF1alpha fragment as defined herein.

Accordingly, in preferred embodiments, the at least one coding sequence comprises or consists of a nucleic acid sequence encoding an transcription factor inhibitor, preferably a RUNX inhibitor (e.g. CBFbeta-Linker-HIF1alpha), that is identical or at least 70%, 80%, 85%, 86%, 87%, 88%, 89%, 90%, 91%, 92%, 93%, 94%, 95%, 96%, 97%, 98%, or 99% identical to any one of the nucleic acid sequences SEQ ID NOs: 296-297, 358-359, 420-421, 482-483, 544-545, 606-607, 668-669, 730-731, 792-793, or a fragment or a variant of any of these.

In other preferred embodiments, the at least one coding sequence comprises a nucleic acid sequence encoding an amino acid element A (e.g. RUNX1), wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 259-277, 321-339, 383-401, 445-463, 507-525, 569-587, 631-649, 693-711, 755-773, or a fragment or a variant of any of these.

In preferred embodiments, the at least one coding sequence comprises a nucleic acid sequence encoding an amino acid element B (e.g. RUNX1T1a or RUNX1T1b), wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 278-292, 340-354, 402-416, 464-478, 526-540, 588-602, 650-664, 712-726, 774-788, or a fragment or a variant of any of these.

Suitably, the at least one coding sequence comprises or consists of at least one nucleic acid sequence encoding a RUNX fragment as defined herein and at least one nucleic acid sequence encoding a RUNX1T1a fragment as defined herein or a RUNX1T1b fragment as defined herein.

Accordingly, in preferred embodiments in that context, the at least one coding sequence comprises or consists of a nucleic acid sequence encoding a transcription factor inhibitor, preferably a RUNX inhibitor (RUNX1-RUNX1T1a or RUNX1-RUNX1T1b), that is identical or at least 70%, 80%, 85%, 86%, 87%, 88%, 89%, 90%, 91%, 92%, 93%, 94%, 95%, 96%, 97%, 98%, or 99% identical to any one of the nucleic acid sequences SEQ ID NOs: 298-301, 360-363, 422-425, 484-487, 546-549, 608-611, 670-673, 732-735, 794-797, or a fragment or a variant of any of these.

In other preferred embodiments, the at least one coding sequence comprises a nucleic acid sequence encoding a transcription factor inhibitor, preferably a RUNX inhibitor (RUNX1 (K83E,R174Q), wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 259, 321, 383, 445, 507, 569, 631, 693, 755, or a fragment or a variant of any of these.

In various preferred embodiments, the at least one coding sequence may comprise a nucleic acid sequence encoding at least one linker peptide, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 258, 320, 382, 444, 506, 568, 630, 692, 754, or a fragment or a variant of any of these. For example, the constructs comprising CBFbeta and LifeAct or the constructs comprising CBFbeta and HIF1alpha or the constructs comprising CBFbeta and NFAT5 may comprise a linker or the constructs comprising CBFbeta and SMMHC may comprise a linker.

In preferred embodiments, the at least one coding sequence comprises more than one stop codon to allow sufficient termination of translation. In particularly embodiments, the at least one coding sequence comprises two or three stop codon to allow sufficient termination of translation. These more than one stop codons may optionally be positioned in alternative reading frames.

### UTRs:

In preferred embodiments, the artificial nucleic acid of the invention, preferably the RNA, comprises at least one heterologous untranslated region (UTR). Suitably, the at least one heterologous untranslated region (UTR) can be selected from at least one heterologous 5′-UTR and/or at least one heterologous 3′-UTR.

The term “untranslated region” or “UTR” or “UTR element” will be recognized and understood by the person of ordinary skill in the art, and are e.g. intended to refer to a part of a nucleic acid molecule typically located 5′ or 3′ of a coding sequence. An UTR is not translated into protein. An UTR may be part of the nucleic acid, e.g. an RNA. An UTR may comprise elements for controlling gene expression, also called regulatory elements. Such regulatory elements may be, e.g., ribosomal binding sites, miRNA binding sites, promotor elements etc.

In preferred embodiments, the artificial nucleic acid comprises a protein-coding region (“coding sequence” or “cds”), and a 5′-UTR and/or 3′-UTR. Notably, UTRs may harbour regulatory sequence elements that determine RNA turnover, stability, and localization. Moreover, UTRs may harbour sequence elements that enhance translation. In medical applications, translation of the nucleic acid into at least one peptide or protein is of paramount importance to therapeutic efficacy. Certain combinations of 3′-UTRs and/or 5′-UTRs may enhance the expression of operably linked coding sequences encoding peptides or proteins as defined herein. Nucleic acid molecules harbouring said UTR combinations advantageously enable rapid and transient expression of encoded transcription factor inhibitors after administration to a subject, preferably after ocular administration. Accordingly, the nucleic acid of the invention comprising certain combinations of 3′-UTRs and/or 5′-UTRs is particularly suitable for ocular administration.

Suitably, the artificial nucleic acid comprises at least one heterologous 5′-UTR and/or at least one heterologous 3′-UTR. Said heterologous 5′-UTRs or 3′-UTRs may be derived from naturally occurring genes or may be synthetically engineered. In preferred embodiments, the artificial nucleic acid comprises at least one coding sequence as defined herein operably linked to at least one (heterologous) 3′-UTR and/or at least one (heterologous) 5′-UTR.

In preferred embodiments, the artificial nucleic acid of the invention comprises at least one 3′-UTR.

The term “3′-untranslated region” or “3′-UTR” will be recognized and understood by the person of ordinary skill in the art, and are e.g. intended to refer to a part of an RNA molecule located 3′ (i.e. downstream) of a coding sequence and which is not translated into protein. A 3′-UTR may be part of a nucleic acid located between a coding sequence and an (optional) terminal poly(A) sequence. A 3′-UTR may comprise elements for controlling gene expression, also called regulatory elements. Such regulatory elements may be, e.g., ribosomal binding sites, miRNA binding sites etc.

Preferably, the artificial nucleic acid comprises at least one 3′-UTR, which may be derivable from a gene that relates to an RNA with enhanced half-life (i.e. that provides a stable RNA).

In some embodiments, the 3′-UTR comprises one or more of a polyadenylation signal, a binding site for proteins that affect a nucleic acid stability of location in a cell, or one or more miRNA or binding sites for miRNAs.

MicroRNAs (or miRNA) are 19-25 nucleotide long noncoding RNAs that bind to the 3′-UTR of RNA molecules and down-regulate gene expression either by reducing RNA stability or by inhibiting translation. E.g., microRNAs are known to regulate RNA, and thereby protein expression, e.g. in liver (miR-122), heart (miR-Id, miR-149), endothelial cells (miR-17-92, miR-126), adipose tissue (let-7, miR-30c), kidney (miR-192, miR-194, miR-204), myeloid cells (miR-142-3p, miR-142-5p, miR-16, miR-21, miR-223, miR-24, miR-27), muscle (miR-133, miR-206, miR-208), and lung epithelial cells (let-7, miR-133, miR-126). The RNA may comprise one or more microRNA target sequences, microRNA sequences, or microRNA seeds. Such sequences may correspond to any known microRNA, e.g. to those taught in US20050261218 and US20050059005.

Accordingly, miRNA, or binding sites for miRNAs as defined above may be removed from the 3′-UTR or may be introduced into the 3′-UTR in order to tailor the expression of the nucleic acid to desired cell types or tissues (e.g. eye cells).

In preferred embodiments, the artificial nucleic acid comprises at least one 3′-UTR, wherein the at least one 3′-UTR comprises or consists of a nucleic acid sequence derived or selected from a 3′-UTR of a gene selected from PSMB3, ALB7, alpha-globin, CASP1, COX6B1, GNAS, NDUFA1 and RPS9, or from a homolog, a fragment, or variant of any one of these genes.

In preferred embodiments, the at least one 3′-UTR that is derived or selected from PSMB3, ALB7, alpha-globin, CASP1, COX6B1, GNAS, NDUFA1 or RPS9 comprises or consist of a nucleic acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 67-90, 109-120, or a fragment or a variant of any of these.

In particularly preferred embodiments, the artificial nucleic acid comprises a 3′-UTR derived or selected from a PSMB3 gene.

In other embodiments, the at least one 3′-UTR comprises or consist of a nucleic acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 91-108, or a fragment or a variant of any of these.

In other embodiments, the artificial nucleic acid comprises a 3′-UTR as described in WO2016107877, the disclosure of WO2016107877 relating to 3′-UTR sequences herewith incorporated by reference. Suitable 3′-UTRs are SEQ ID NOs: 1-24 and SEQ ID NOs: 49-318 of WO2016107877, or fragments or variants of these sequences. In other embodiments, the artificial nucleic acid comprises a 3′-UTR as described in WO2017036580, the disclosure of WO2017036580 relating to 3′-UTR sequences herewith incorporated by reference. Suitable 3′-UTRs are SEQ ID NOs: 152-204 of WO2017036580, or fragments or variants of these sequences. In other embodiments, the artificial nucleic acid comprises a 3′-UTR as described in WO2016022914, the disclosure of WO2016022914 relating to 3′-UTR sequences herewith incorporated by reference. Particularly preferred 3′-UTRs are nucleic acid sequences according to SEQ ID NOs: 20-36 of WO2016022914, or fragments or variants of these sequences.

In preferred embodiments, the artificial nucleic acid of the invention comprises at least one 5′-UTR.

The terms “5′-untranslated region” or “5′-UTR” will be recognized and understood by the person of ordinary skill in the art, and are e.g. intended to refer to a part of a nucleic acid located 5′ (i.e. “upstream”) of a coding sequence and which is not translated into protein. A 5′-UTR may be part of a nucleic acid located 5′ of the coding sequence. Typically, a 5′-UTR starts with the transcriptional start site and ends before the start codon of the coding sequence. A 5′-UTR may comprise elements for controlling gene expression, also called regulatory elements. Such regulatory elements may be, e.g., ribosomal binding sites, miRNA binding sites etc. The 5′-UTR may be modified, e.g. by enzymatic or co-transcriptional addition of a 5′-cap structure (e.g. for mRNA as defined below).

Preferably, the artificial nucleic acid comprises at least one 5′-UTR, which may be derivable from a gene that relates to an RNA with enhanced half-life (i.e. that provides a stable RNA).

In some embodiments, the 5′-UTR comprises one or more of a binding site for proteins that affect a nucleic acid stability or nucleic acid location in a cell, or one or more miRNA or binding sites for miRNAs (as defined above).

Accordingly, miRNA or binding sites for miRNAs as defined above may be removed from the 5′-UTR or introduced into the 5′-UTR in order to tailor the expression of the nucleic acid to desired cell types or tissues (e.g. muscle cells).

In preferred embodiments, the artificial nucleic acid comprises at least one 5′-UTR, wherein the at least one 5′-UTR comprises a nucleic acid sequence derived or selected from a 5′-UTR of gene selected from HSD17B4, RPL32, ASAH1, ATP5A1, MP68, NDUFA4, NOSIP, RPL31, SLC7A3, TUBB4B, and UBQLN2, or from a homolog, a fragment or variant of any one of these genes,

In preferred embodiments, the at least one 5′-UTR derived or selected from HSD17B4, RPL32, ASAH1, ATP5A1, MP68, NDUFA4, NOSIP, RPL31, SLC7A3, TUBB4B, and UBQLN2 comprises or consist of a nucleic acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 1-32, 65-66, or a fragment or a variant of any of these.

In particularly preferred embodiments, the nucleic acid comprises a 5′-UTR derived or selected from a HSD17B4 gene.

In other embodiments, the at least one 3′-UTR comprises or consist of a nucleic acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 33-64, or a fragment or a variant of any of these.

In other embodiments, the nucleic acid comprises a 5′-UTR as described in WO2013143700, the disclosure of WO2013143700 relating to 5′-UTR sequences herewith incorporated by reference. Particularly preferred 5′-UTRs are nucleic acid sequences derived from SEQ ID NOs: 1-1363, SEQ ID NO: 1395, SEQ ID NO: 1421 and SEQ ID NO: 1422 of WO2013143700, or fragments or variants of these sequences. In other embodiments, the nucleic acid comprises a 5′-UTR as described in WO2016107877, the disclosure of WO2016107877 relating to 5′-UTR sequences herewith incorporated by reference. Particularly preferred 5′-UTRs are nucleic acid sequences according to SEQ ID NOs: 25-30 and SEQ ID NOs: 319-382 of WO2016107877, or fragments or variants of these sequences. In other embodiments, the nucleic acid comprises a 5′-UTR as described in WO2017036580, the disclosure of WO2017036580 relating to 5′-UTR sequences herewith incorporated by reference. Particularly preferred 5′-UTRs are nucleic acid sequences according to SEQ ID NOs: 1-151 of WO2017036580, or fragments or variants of these sequences. In other embodiments, the nucleic acid comprises a 5′-UTR as described in WO2016022914, the disclosure of WO2016022914 relating to 5′-UTR sequences herewith incorporated by reference. Particularly preferred 5′-UTRs are nucleic acid sequences according to SEQ ID NOs: 3-19 of WO2016022914, or fragments or variants of these sequences.

In preferred embodiments, the artificial nucleic acid, preferably the RNA of the invention comprises at least one coding sequence as specified herein encoding at least one transcription factor inhibitor, operably linked to a 3′-UTR and/or a 5′-UTR selected from the following 5′-UTR/3′-UTR combinations (“also referred to UTR designs”): a-1 (HSD17B4/PSMB3), a-2 (NDUFA4/PSMB3), a-3 (SLC7A3/PSMB3), a-4 (NOSIP/PSMB3), a-5 (MP68/PSMB3), b-1 (UBQLN2/RPS9), b-2 (ASAH1/RPS9), b-3 (HSD17B4/RPS9), b-4 (HSD17B4/CASP1), b-5 (NOSIP/COX6B1), c-1 (NDUFA4/RPS9), c-2 (NOSIP/NDUFA1), c-3 (NDUFA4/COX6B1), c-4 (NDUFA4/NDUFA1), c-5 (ATP5A1/PSMB3), d-1 (Rpl31/PSMB3), d-2 (ATP5A1/CASP1), d-3 (SLC7A3/GNAS), d-4 (HSD17B4/NDUFA1), d-5 (Slc7a3/Ndufa1), e-1 (TUBB4B/RPS9), e-2 (RPL31/RPS9), e-3 (MP68/RPS9), e-4 (NOSIP/RPS9), e-5 (ATP5A1/RPS9), e-6 (ATP5A1/COX6B1), f-1 (ATP5A1/GNAS), f-2 (ATP5A1/NDUFA1), f-3 (HSD17B4/COX6B1), f-4 (HSD17B4/GNAS), f-5 (MP68/COX6B1), g-1 (MP68/NDUFA1), g-2 (NDUFA4/CASP1), g-3 (NDUFA4/GNAS), g-4 (NOSIP/CASP1), g-5 (RPL31/CASP1), h-1 (RPL31/COX6B1), h-2 (RPL31/GNAS), h-3 (RPL31/NDUFA1), h-4 (Slc7a3/CASP1), h-5 (SLC7A3/COX6B1), i-1 (SLC7A3/RPS9), i-2 (RPL32/ALB7), i-2 (RPL32/ALB7), or i-3 (alpha-globin gene).

In preferred embodiments, the at least one heterologous 5′-UTR is selected from HSD17B4 and the at least one heterologous 3′ UTR is selected from PSMB3.

Accordingly, in particularly preferred embodiments, the artificial nucleic acid, preferably the RNA comprises at least one coding sequence as defined herein encoding at least one transcription factor inhibitor as defined herein, wherein said coding sequence is operably linked to a HSD17B4 5′-UTR and a PSMB3 3′-UTR (HSD17B4/PSMB3 (a-1)). It has been shown by the inventors that this embodiment is particularly beneficial for expressing the transcription factor inhibitor in human cells e.g. cells of the eye.

Preferably, the at least one heterologous 3′-UTR, preferably the 3′-UTR derived or selected from PSMB3, comprises or consist of a nucleic acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 67, 68, 109-120, or a fragment or a variant thereof, preferably SEQ ID NO: 68, or a fragment or a variant thereof.

Preferably, the at least one heterologous 5′-UTR, preferably the 5′-UTR derived or selected from HSD17B4, comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 1, 2, 65, 66, or a fragment or a variant thereof, preferably SEQ ID NO: 2, or a fragment or a variant thereof.

In various embodiments, the nucleic acid, e.g. the RNA is monocistronic, bicistronic, or multicistronic.

In preferred embodiments, the nucleic acid, e.g. the RNA of the invention is monocistronic.

The term “monocistronic” will be recognized and understood by the person of ordinary skill in the art, and is e.g. intended to refer to a nucleic acid that comprises only one coding sequence. The terms “bicistronic”, or “multicistronic” as used herein are e.g. intended to refer to an RNA that comprises two (bicistronic) or more (multicistronic) coding sequences.

In preferred embodiments, the A/U (A/T) content in the environment of the ribosome binding site of the nucleic acid is increased compared to the A/U (A/T) content in the environment of the ribosome binding site of its respective wild type or reference nucleic acid. This modification increases the efficiency of ribosome binding to the nucleic acid, which is in turn beneficial for an efficient translation of the nucleic acid into peptides or proteins.

Accordingly, in a particularly preferred embodiment, the artificial nucleic acid comprises a ribosome binding site, also referred to as “Kozak sequence” identical to or at least 80%, 85%, 90%, 95% identical to any one of SEQ ID NOS: 128, 129, or sequences GCCGCCACC (DNA), GCCGCCACC (RNA), GCCACC (DNA), GCCACC (RNA), ACC (DNA) or ACC (RNA), or fragments or variants of any of these. In preferred embodiments, the “Kozak sequence” comprises or consists of RNA sequence ACC.

### Poly(N) Sequences, Histone Stem Loops:

In preferred embodiments, the artificial nucleic acid comprises at least one poly(N) sequence, e.g. at least one poly(A) sequence, at least one poly(U) sequence, at least one poly(C) sequence, or combinations thereof.

In preferred embodiments, the artificial nucleic acid, e.g. the RNA, comprises at least one poly(A) sequence. In some embodiments, the artificial nucleic acid comprises least two, three, or more poly(A) sequences.

The terms “poly(A) sequence”, “poly(A) tail” or “3′-poly(A) tail” as used herein will be recognized and understood by the person of ordinary skill in the art, and are e.g. intended to be a sequence of adenosine nucleotides, typically located at the 3′-end of a linear RNA of up to about 1000 adenosine nucleotides. Preferably, said poly(A) sequence is essentially homopolymeric, e.g. a poly(A) sequence of e.g. 100 adenosine nucleotides has essentially the length of 100 nucleotides. In other embodiments, the poly(A) sequence is interrupted by at least one nucleotide different from an adenosine nucleotide, e.g. a poly(A) sequence of e.g. 100 adenosine nucleotides may have a length of more than 100 nucleotides (comprising 100 adenosine nucleotides and in addition said at least one nucleotide- or a stretch of nucleotides—different from an adenosine nucleotide).

In preferred embodiments, the at least one poly(A) sequence may comprise about 40 to about 500 adenosine nucleotides, about 40 to about 250 adenosine nucleotides, about 60 to about 250 adenosine nucleotides, preferably about 60 to about 150 adenosine nucleotides.

In preferred embodiments, the at least one poly(A) sequence may comprise about 40 to about 500 consecutive adenosine nucleotides, about 40 to about 250 consecutive adenosine nucleotides, about 60 to about 250 consecutive adenosine nucleotides, preferably about 60 to about 150 consecutive adenosine nucleotides.

Suitably, the length of the poly(A) sequence may be at least about or even more than about 10, 50, 64, 75, 100, 200, 300, 400, or 500 adenosine nucleotides, preferably consecutive adenosine nucleotides.

In particularly preferred embodiments, the at least one poly(A) sequence comprises about 100 adenosine nucleotides (A100), preferably about 100 consecutive adenosine nucleotides.

In further embodiments, the artificial nucleic acid comprises at least one interrupted poly(A) sequence comprising about 100 adenosine nucleotides, wherein the poly(A) sequence is interrupted by non-adenosine nucleotides, preferably by about 10 non-adenosine (N10) nucleotides. In that context, a poly(A) sequence A30-N10-A70 is preferred.

The poly(A) sequence as defined herein may be located directly at the 3′ terminus of the artificial nucleic acid, preferably the RNA. In preferred embodiments, the 3′-terminal nucleotide (that is the last 3′-terminal nucleotide in the polynucleotide chain) is the 3′-terminal A nucleotide of the at least one poly(A) sequence. The term “directly located at the 3′ terminus” has to be understood as being located exactly at the 3′ terminus-in other words, the 3′ terminus of the nucleic acid consists of a poly(A) sequence terminating with an A.

Ending on an adenosine nucleotide decreases the induction of interferons, e.g. IFNalpha, by the RNA of the invention if for example administered as a medicament into the eye. This is particularly important as the induction of interferons, e.g. IFNalpha, is thought to be one main factor for induction of side effects.

Accordingly, in particularly preferred embodiments, the artificial nucleic acid of the invention, e.g. the RNA, comprises a poly(A) sequence of about 100 consecutive adenosine nucleotides, wherein said poly(A) sequence is located directly at the 3′ terminus of the RNA, optionally wherein the 3′ terminal nucleotide is an adenosine

In preferred embodiments, the poly(A) sequence of the artificial nucleic acid, e.g. the RNA, is obtained from a DNA template during RNA in vitro transcription. In other embodiments, the poly(A) sequence is obtained in vitro by common methods of chemical synthesis without being necessarily transcribed from a DNA template. In other embodiments, poly(A) sequences are generated by enzymatic polyadenylation of the RNA (after RNA in vitro transcription) using e.g. immobilized poly(A) polymerases according to methods and means as described in WO2016174271.

In some embodiments, the artificial nucleic acid, e.g. the RNA, comprises at least one poly(A) sequence obtained by enzymatic polyadenylation, wherein the majority of RNA molecules comprise about 100 (+/−20) to about 500 (+/−100) adenosine nucleotides, preferably about 100 (+/−20) to about 200 (+/−40) adenosine nucleotides.

In some embodiments, the artificial nucleic acid, e.g. the RNA, comprises at least one poly(A) sequence derived from a template DNA and additionally at least one poly(A) sequence generated by enzymatic polyadenylation, e.g. as described in published PCT patent application WO2016091391.

In some embodiments, the artificial nucleic acid comprises at least one polyadenylation signal.

In some embodiments, the artificial nucleic acid comprises at least one poly(C) sequence. A poly(C) sequence in the context of the invention may be located in an UTR region, preferably in the 3′ UTR.

The term “poly(C) sequence” as used herein is intended to be a sequence of cytosine nucleotides of up to about 200 cytosine nucleotides. In preferred embodiments, the poly(C) sequence comprises about 10 to about 200 cytosine nucleotides, about 10 to about 100 cytosine nucleotides, about 20 to about 70 cytosine nucleotides, about 20 to about 60 cytosine nucleotides, or about 10 to about 40 cytosine nucleotides. In a particularly preferred embodiment, the poly(C) sequence comprises about 30 cytosine nucleotides.

Accordingly, in preferred embodiments, the artificial nucleic acid of the invention comprises at least one poly(C) sequence and/or at least one miRNA binding site and/or histone stem-loop sequence.

In preferred embodiments, the artificial nucleic acid, e.g. the RNA, comprises at least one histone stem-loop (hSL) or histone stem loop structure. A hSL in the context of the invention may be located in an UTR region, preferably in the 3′ UTR.

The term “histone stem-loop” (hSL) is intended to refer to nucleic acid sequences that forms a stem-loop secondary structure predominantly found in histone mRNAs.

Histone stem-loop sequences/structures may suitably be selected from hSL sequences as disclosed in WO2012019780, the disclosure relating to histone stem-loop sequences/histone stem-loop structures incorporated herewith by reference. A hSL sequence that may be used within the present invention may be derived from formulae (I) or (II) of WO2012019780. According to a preferred embodiment, the artificial nucleic acid, e.g. the RNA, comprises at least one hSL sequence derived from at least one of the specific formulae (Ia) or (IIa) of WO2012019780.

In preferred embodiments, the artificial nucleic acid, e.g. the RNA, comprises at least one histone stem-loop sequence, wherein said histone stem-loop sequence comprises or consists of a nucleic acid sequence identical or at least 70%, 80%, 90%, 91%, 92%, 93%, 94%, 95%, 96%, 97%, 98%, or 99% identical to any one of SEQ ID NOs: 136 or 137, or a fragment or variant of any of these. Preferably, the histone stem-loop sequence comprises or consists of a nucleic acid sequence according to SEQ ID NO: 137, or a fragment or thereof.

In other embodiments, the artificial nucleic acid does not comprise a histone stem-loop as defined herein.

In preferred embodiments, the artificial nucleic acid comprises a 3′-terminal sequence element. The 3′-terminal sequence element represents the 3′ terminus of the RNA. A 3′-terminal sequence element may comprise at least one poly(N) sequence as defined herein and, optionally, at least one hSL as defined herein.

In preferred embodiments, the artificial nucleic acid comprises at least one 3′-terminal sequence element comprising or consisting of an RNA sequence being identical or at least 70%, 80%, 90%, 91%, 92%, 93%, 94%, 95%, 96%, 97%, 98%, or 99% identical to any one of SEQ ID NOs: 138-172, or a fragment or variant of these sequences.

In preferred embodiments, the artificial nucleic acid comprises a 3′-terminal sequence element comprising a hSL as defined herein followed by a poly(A) sequence comprising about 100 consecutive adenosines.

In particularly preferred embodiments, the artificial nucleic acid comprises a 3′-terminal sequence element comprising or consisting of a nucleic acid sequence being identical or at least 70%, 80%, 90%, 91%, 92%, 93%, 94%, 95%, 96%, 97%, 98%, or 99% identical to SEQ ID NO: 144, or a fragment or variant thereof.

In some embodiments, the artificial nucleic acid comprises a 5′-terminal sequence element comprising or consisting of a nucleic acid sequence, preferably an RNA sequence, being identical or at least 70%, 80%, 90%, 91%, 92%, 93%, 94%, 95%, 96%, 97%, 98%, or 99% identical to any one of sequences GGGAGA, AGGAGA, GGGAAA, AGAAUA, AGAUUA, GAUGGG or GGGCG, or a fragment or variant of these sequences.

In preferred embodiments, the artificial nucleic acid comprises a 5′-terminal sequence element comprising or consisting of a nucleic acid sequence, preferably an RNA sequence being identical or at least 70%, 80%, 90%, 91%, 92%, 93%, 94%, 95%, 96%, 97%, 98%, or 99% identical to sequence AGGAGA, or a fragment or variant thereof.

Such a 5′-terminal sequence element may comprise e.g. a binding site for T7 RNA polymerase. Further, the first nucleotide of said 5′-terminal start sequence may preferably comprise a 2′O methylation, e.g. 2′O methylated guanosine or a 2′O methylated adenosine.

In particularly preferred embodiments, the artificial nucleic acid of the invention is an isolated nucleic acid. The term “isolated nucleic acid” does not comprise a cell or a subject that comprises said nucleic acid, but relates to the artificial nucleic acid as an isolated molecule or ensemble of isolated molecules. For example, the “isolated nucleic acid” can be an artificial nucleic acid isolated or purified from a cell (e.g. cell culture, bacterial culture), or can be an artificial nucleic acid (e.g. RNA) isolated from an RNA in vitro transcription.

In particularly preferred embodiments, the artificial nucleic acid of the invention is a therapeutic nucleic acid. Accordingly, the artificial nucleic acid is suitably used in a therapeutic context, in particular to provide a therapeutic modality for providing transcription factor inhibitors according to the invention.

In some embodiments, the artificial nucleic acid of the invention is selected from a DNA.

The DNA may be any type of DNA that comprises a coding sequence as defined herein including any type of single stranded DNA, any type of double stranded DNA, any type of linear DNA, and any type of circular DNA.

A suitable DNA in the context of the invention may be selected from bacterial plasmid, an adenovirus, a poxvirus, a parapoxivirus (orf virus), a vaccinia virus, a fowlpox virus, a herpes virus, an adeno-associated virus (AAV), an alphavirus, a lentivirus, a lambda phage, a lymphocytic choriomeningitis virus and a Listeria sp, Salmonella sp.

In preferred embodiments, the DNA a viral DNA, preferably an adeno-associated virus DNA.

In particularly preferred embodiments, the artificial nucleic acid of the invention is an RNA.

The RNA may be any type of RNA that comprises a coding sequence as defined herein including any type of single stranded RNA, any type of double stranded RNA, any type of linear RNA, and any type of circular RNA.

In preferred embodiments, the RNA is selected from mRNA, circular RNA, replicon RNA or self-replicating RNA, or viral RNA, preferably mRNA or a circular RNA.

In embodiments, the RNA is a circular RNA. As used herein, “circular RNA” or “circRNAs” have to be understood as an RNA construct that is connected to form a circle and therefore does not comprise a 3′ or 5′ terminus. In preferred embodiments, said circRNA comprises at least one coding sequence encoding at least one transcription factor inhibitor as defined herein.

In embodiments, the RNA is a replicon RNA. The term “replicon RNA” or “self-replicating RNA” will be recognized and understood by the person of ordinary skill in the art and is preferably intended to be an optimized self-replicating RNA. Such constructs may include replicase elements derived from e.g. alphaviruses (e.g. SFV, SIN, VEE, or RRV) and the substitution of the structural virus proteins with the nucleic acid of interest (that is, the sequence encoding at least one transcription factor inhibitor).

In particularly preferred embodiments, the RNA is selected from an mRNA. Accordingly, the artificial nucleic acid of the invention is an mRNA, suitably an isolated mRNA.

mRNA technology is preferred in the context of the invention to produce transcription factor inhibitors because mRNA allows for regulated dosage, transient expression, complete degradation of the mRNA after protein synthesis, and do not pose the risk of insertional mutations.

Preferably, the artificial nucleic acid, preferably the RNA, comprises about 50 to about 20000 nucleotides, or about 500 to about 10000 nucleotides, or about 1000 to about 10000 nucleotides, or preferably about 1000 to about 5000 nucleotides, or even more preferably about 2000 to about 5000 nucleotides.

### Modified Nucleotides:

According to various embodiments, the artificial nucleic acid, preferably the RNA, is modified, wherein the modification refers to chemical modifications comprising backbone modifications as well as sugar modifications or base modifications.

A modified nucleic acid or RNA may comprise nucleotide analogues/modifications, e.g. backbone modifications, sugar modifications or base modifications. A backbone modification in the context of the invention is a modification in which phosphates of the backbone of the nucleotides of the RNA are chemically modified. A sugar modification in the context of the invention is a chemical modification of the sugar of the nucleotides of the RNA. Furthermore, a base modification in the context of the invention is a chemical modification of the base moiety of the nucleotides of the RNA. In this context, nucleotide analogues or modifications are preferably selected from nucleotide analogues which are applicable for transcription and/or translation.

Accordingly, in preferred embodiments, the nucleic acid, preferably the RNA of the invention comprises at least one modified nucleotide.

In some embodiments, the at least one modified nucleotide is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

Particularly preferred in that context are pseudouridine (ψ) and N1-methylpseudouridine (m1ψ).

In some embodiments, essentially all, e.g. essentially 100% of the uracil in the coding sequence (or the full nucleic acid sequence) have a chemical modification, preferably a chemical modification in the 5-position of the uracil.

In preferred embodiments, 100% of the uracil in the full nucleic acid sequence, preferably the RNA sequence are substituted with N1-methylpseudouridine (m1ψ). Alternatively, 100% of the uracil in the full nucleic acid sequence, preferably the RNA sequence are substituted with pseudouridine (ψ).

Incorporating modified nucleotides such as e.g. pseudouridine (ψ) or N1-methylpseudouridine (m1ψ) into the coding sequence (or the full nucleic acid sequence) may be advantageous as unwanted innate immune responses (upon administration of the RNA) may be adjusted or reduced (if required).

In preferred embodiments, the artificial nucleic acid, preferably the RNA, does not comprise chemically modified nucleotides. Notably, a 5′-cap structure as defined below is typically not considered to be a chemically modified nucleotide. Accordingly, the artificial nucleic acid, preferably the RNA, comprises a sequence that consists only of G, C, A and U nucleotides and therefore does not comprise modified nucleotides, and optionally comprises a 5′-cap structure.

In preferred embodiments, the artificial nucleic acid, preferably the RNA of the invention does not comprise N1-methylpseudouridine (m1ψ) substituted positions or pseudouridine (ψ) substituted positions.

### Cap Structures:

In preferred embodiments, the artificial nucleic acid, preferably the RNA, comprises a 5′-cap structure.

Such a 5′-cap structure suitably stabilizes the nucleic acid and/or enhances expression of the encoded transcription factor inhibitor and/or reduces the stimulation of the innate immune system after administration.

Accordingly, in preferred embodiments, the artificial nucleic acid, preferably the RNA, comprises a 5′-cap structure, preferably m7G, cap0, cap1, cap2, a modified cap0 or a modified cap1 structure.

The term “5′-cap structure” as used herein will be recognized and understood by the person of ordinary skill in the art, and is e.g. intended to refer to a 5′ modified nucleotide, particularly a guanine nucleotide, positioned at the 5′-end of an RNA, e.g. an mRNA. Preferably, the 5′-cap structure is connected via a 5′-5′-triphosphate linkage to the RNA.

5′-cap structures which may be suitable in the context of the present invention are cap0 (methylation of the first nucleobase, e.g. m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse cap analogue), modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

Suitably, a 5′-cap (cap0 or cap1) structure may be formed in chemical RNA synthesis or in RNA in vitro transcription (co-transcriptional capping) using cap analogues.

The term “cap analogue” as used herein will be recognized and understood by the person of ordinary skill in the art, and is e.g. intended to refer to a non-polymerizable di-nucleotide or tri-nucleotide that has cap functionality in that it facilitates translation or localization, and/or prevents degradation of an RNA molecule when incorporated at the 5′-end of the nucleic acid molecule. Non-polymerizable means that the cap analogue will be incorporated only at the 5′-terminus because it does not have a 5′ triphosphate and therefore cannot be extended in the 3′-direction by a template-dependent polymerase, particularly, by template-dependent RNA polymerase. Examples of cap analogues include, but are not limited to, a chemical structure selected from the group consisting of m7GpppG, m7GpppA, m7GpppC; unmethylated cap analogues (e.g. GpppG); dimethylated cap analogue (e.g. m2,7GpppG), trimethylated cap analogue (e.g. m2,2,7GpppG), dimethylated symmetrical cap analogues (e.g. m7Gpppm7G), or anti reverse cap analogues (e.g. ARCA; m7,2′OmeGpppG; m7,2′dGpppG; m7,3′OmeGpppG; m7,3′dGpppG and their tetraphosphate derivatives). Further suitable cap analogues are described in WO2008016473, WO2008157688, WO2009149253, WO2011015347, WO2013059475, WO2017066793, WO2017066781, WO2017066791, WO2017066789, WO2017053297, WO2017066782, WO2018075827 and WO2017066797, the disclosures referring to cap analogues herewith incorporated by reference.

In embodiments, a cap1 structure is generated using tri-nucleotide cap analogue as disclosed in WO2017053297, WO2017066793, WO2017066781, WO2017066791, WO2017066789, WO2017066782, WO2018075827 and WO2017066797. Preferably, cap structures derivable from the structure disclosed in claim 1-5 of WO2017053297 may be suitably used to co-transcriptionally generate a cap1 structure. Further, any cap structures derivable from the structure defined in claim 1 or claim 21 of WO2018075827 may be suitably used to generate a cap1 structure.

In preferred embodiments, the 5′-cap structure may suitably be added co-transcriptionally using tri-nucleotide cap analogue as defined herein, preferably in an RNA in vitro transcription reaction as defined herein.

In particularly preferred embodiments, the artificial nucleic acid, preferably the RNA of the invention comprises a cap1 structure or a modified cap1 structure.

In preferred embodiments, the cap1 structure is formed via co-transcriptional capping using tri-nucleotide cap analogues m7G(5′)ppp(5′)(2′OMeA)pG or m7G(5′)ppp(5′)(2′OMeG)pG. A particularly preferred cap1 analog in that context is m7G(5′)ppp(5′)(2′OMeA)pG.

In other preferred embodiments, the cap1 structure is a modified cap1 structure and is formed using co-transcriptional capping using tri-nucleotide cap analogue 3′OMe-m7G(5′)ppp(5′)(2′OMeA)pG.

In other embodiments, the 5′-cap structure is formed via enzymatic capping using capping enzymes (e.g. vaccinia virus capping enzymes and/or cap-dependent 2′-O methyltransferases) to generate cap0 or cap1 or cap2 structures. In that context, the 5′-cap structure (cap0 or cap1) may be added using immobilized capping enzymes and/or cap-dependent 2′-O methyltransferases using methods and means disclosed in published PCT patent application WO2016193226.

In preferred embodiments, about 70%, 75%, 80%, 85%, 90%, 95% of the RNA (species) comprises a cap structure, preferably a cap1 structure as determined by a capping assay.

For determining the presence or absence of a cap structure, capping assays as described in published PCT application WO2015101416, in particular, as described in claims 27 to 46 of published PCT application WO2015101416 can be used. Other capping assays that may be used to determine the presence or absence of a cap structure of an RNA are described in published PCT application WO2020127959.

### Further RNA Features:

In the context of the invention, the artificial nucleic acid is preferably an RNA that provides at least one coding sequence encoding at least one transcription factor inhibitor as defined herein that is produced after administration to a cell or subject.

Suitable elements the RNA of the invention preferably comprises are for example a 5′ Cap structure as defined herein, a 5′ UTR as defined herein, a 3′ UTR as defined herein, hSL as defined herein, Poly(A) sequence as defined herein, and optional chemical modifications as defined herein.

In preferred embodiments, the RNA is preferably an in vitro transcribed RNA (e.g. an in vitro transcribed mRNA).

In some embodiments, the nucleotide mixture for RNA in vitro transcription comprises modified nucleotides as defined herein. In that context, preferred modified nucleotides may be selected from pseudouridine (ψ) or N1-methylpseudouridine (m1ψ. Suitably, uracil nucleotides in the nucleotide mixture are replaced (either partially or completely) by pseudouridine (ψ) and/or N1-methylpseudouridine (m1ψ) to obtain a modified RNA (e.g. a modified mRNA).

In preferred embodiments, the nucleotide mixture used in RNA in vitro transcription does not comprise modified nucleotides as defined herein. In preferred embodiments, the nucleotide mixture used for RNA in vitro transcription does only comprise G, C, A and U nucleotides, and, optionally, a cap analog as defined herein to obtain a non-modified RNA (e.g. a non-modified mRNA).

In preferred embodiments, the nucleotide mixture (i.e. the fraction of each nucleotide in the mixture) used for RNA in vitro transcription reactions is optimized for the given RNA sequence, preferably as described WO2015188933.

Accordingly, in preferred embodiments, the nucleic acid of the invention is an in vitro transcribed RNA, preferably wherein RNA in vitro transcription has been performed in the presence of a sequence optimized nucleotide mixture.

In a preferred embodiment, the RNA is lyophilized (e.g. according to WO2016165831 or WO2011069586) to yield a temperature stable dried RNA. The RNA may also be dried using spray-drying or spray-freeze drying (e.g. according to WO2016184575 or WO2016184576) to yield a temperature stable RNA (powder).

In the context of the invention (e.g. for RNA-based medicaments), it may be required to provide GMP-grade RNA. GMP-grade RNA is produced using a manufacturing process approved by regulatory authorities. In preferred embodiments, RNA production is performed under current good manufacturing practice (GMP), implementing various quality control steps on DNA and RNA level, preferably quality control steps selected from methods described in WO2016180430. In preferred embodiments, the RNA of the invention is a GMP-grade RNA, particularly a GMP-grade mRNA.

In preferred embodiments, the artificial nucleic acid of the invention is a purified RNA, preferably a purified mRNA. Suitably, the RNA of the invention has been purified by at least one step of purification

The term “purified RNA” or “purified mRNA” as used herein has to be understood as RNA which has a higher purity after certain purification steps (e.g. HPLC, TFF, Oligo d (T) purification, precipitation steps) than the starting material (e.g. in vitro transcribed RNA). Typical impurities that are essentially not present in purified RNA comprise peptides or proteins (e.g. enzymes derived from DNA dependent RNA in vitro transcription, e.g. RNA polymerases, RNases, pyrophosphatase, restriction endonuclease, DNase), spermidine, BSA, abortive RNA sequences, RNA fragments (short double stranded RNA (dsRNA)), free nucleotides (modified nucleotides, conventional NTPs, cap analogue), template DNA fragments, buffer components (HEPES, TRIS, MgCl2) etc. Other potential impurities that may be derived from e.g. fermentation procedures comprise bacterial impurities (bioburden, bacterial DNA) or impurities derived from purification procedures (organic solvents etc.). Accordingly, it is desirable in this regard for the “degree of RNA purity” to be as close as possible to 100%. Accordingly, “purified RNA” as used herein has a degree of purity of more than 75%, 80%, 85%, very particularly 90%, 91%, 92%, 93%, 94%, 95%, 96%, 97%, 98% and most favourably 99% or more. The degree of purity is e.g. determined by an analytical HPLC, wherein the percentages provided above correspond to the ratio between the area of the peak for the target RNA and the total area of all peaks including the peaks representing the by-products. Alternatively, the degree of purity is e.g. determined by an analytical agarose gel electrophoresis or capillary gel electrophoresis.

In preferred embodiments, purification of the nucleic acid, preferably the RNA may comprise at least one step of purification selected from (RP)-HPLC, AEX, size exclusion chromatography (SEC), hydroxyapatite chromatography, tangential flow filtration (TFF), filtration, precipitation, core-bead flow through chromatography, oligo(dT) purification, cellulose-based purification, or any combination thereof.

Preferably, the RNA has been purified using RP-HPLC (preferably as described in WO2008077592) and/or TFF (preferably as described in WO2016193206) and/or oligo d (T) purification (preferably as described in WO2016180430) to e.g. to remove dsRNA, non-capped RNA and/or RNA fragments.

In some embodiments, the RNA has been purified by a step of 5′ dephosphorylation of linear RNA, DNA digestion, protein digestion, and/or dsRNA digestion.

According to preferred embodiments, the purified RNA has a purity level of at least about 70%, 75%, 80%, 85%, 90%, or 95%, preferably more than 95%. Suitably, the degree of purity is determined by an analytical HPLC method.

In embodiments, the nucleic acid, preferably the RNA of the invention has a certain integrity.

The term “integrity” generally describes whether the complete nucleic acid sequence or RNA sequence is present. Low RNA integrity could be due to, amongst others, RNA degradation, RNA cleavage, incorrect or incomplete chemical synthesis of the RNA, incorrect base pairing, integration of modified nucleotides or the modification of already integrated nucleotides, lack of capping or incomplete capping, lack of polyadenylation or incomplete polyadenylation, or incomplete RNA in vitro transcription. RNA is a fragile molecule that can easily degrade, which may be caused e.g. by temperature, ribonucleases, pH or other factors (e.g. nucleophilic attacks, hydrolysis etc.), which may reduce the RNA integrity and, consequently, its functionality.

The skilled person can choose from a variety of different chromatographic or electrophoretic methods for determining integrity of nucleic acid, in particular RNA. Chromatographic and electrophoretic (e.g. capillary gel electrophoresis) methods are well-known in the art. In case chromatography is used (e.g. RP-HPLC), the analysis of the integrity of the RNA may be based on determining the peak area (or “area under the peak”) of the expected full length RNA (the RNA with the correct RNA length) in a corresponding chromatogram.

In embodiments, the nucleic acid of the invention, preferably the RNA has an integrity ranging from about 40% to about 100%. In embodiments, the nucleic acid of the invention, preferably the RNA has an integrity of about 50%, about 60%, about 70%, about 80%, or about 90%. Integrity is suitably determined using analytical HPLC, preferably analytical RP-HPLC.

In preferred embodiments, the nucleic acid of the invention, preferably the RNA has an integrity of at least about 50%, preferably of at least about 60%, more preferably of at least about 70%, most preferably of at least about 80% or about 90% or higher. Integrity is suitably determined using analytical HPLC, preferably analytical RP-HPLC.

In preferred embodiments, the nucleic acid, preferably the RNA, is suitable for use in treatment or prevention of a disease, disorder or condition.

In particularly preferred embodiments, the nucleic acid, preferably the RNA, is suitable for use in treatment or prevention of an ocular disease, disorder or condition.

### Preferred Nucleic Acid Constructs:

In various embodiments, the artificial nucleic acid comprises at least the following elements:
A) a 5′-cap structure, preferably as specified herein;
B) at least one cds encoding at least one transcription factor inhibitor as defined herein;
C) a 5′-UTR and/or a 3′-UTR, preferably as specified herein;
D) at least one poly(A) sequence, preferably as specified herein.

In preferred embodiments, the artificial nucleic acid, preferably the RNA, comprises the following sequence elements preferably in 5′- to 3′-direction:
A) a 5′-cap structure;
B) a 5′-UTR preferably selected or derived from a 5′-UTR of a HSD17B4 gene;
C) a coding sequence encoding a transcription factor inhibitor, preferably a RUNX inhibitor;
D) a 3′-UTR preferably selected or derived from a 3′-UTR of a PSMB3 gene;
E) optionally, a histone stem-loop; and
F) a poly(A) sequence preferably comprising about 100 A nucleotides.

In various embodiments, the artificial nucleic acid, preferably the RNA, comprises the following elements:
A) a 5′-cap structure, preferably as specified herein;
B) a 5′-terminal start element, preferably as specified herein;
C) optionally, a 5′-UTR, preferably as specified herein;
D) a ribosome binding site, preferably as specified herein;
E) at least one cds encoding at least one transcription factor inhibitor as defined herein.
F) a 3′-UTR, preferably as specified herein;
G) optionally, at least one poly(A) sequence, preferably as specified herein;
H) optionally, at least one poly(C) sequence, preferably as specified herein;
I) optionally, histone stem-loop preferably as specified herein;
J) optionally, 3′-terminal sequence element, preferably as specified herein;
K) optionally, chemically modified nucleotides, preferably as specified herein.

In preferred embodiments, the artificial nucleic acid, preferably the RNA comprises the following elements preferably in 5′- to 3′-direction:
A) a cap1 structure preferably as defined herein;
B) a 5′-UTR derived from a HSD17B4 gene as defined herein;
C) at least one cds encoding at least one transcription factor inhibitor as defined herein.
D) a 3′-UTR derived from a 3′-UTR of a PSMB3 gene as defined herein;
E) a poly(A) sequence preferably comprising about 100 A nucleotides;
F) optionally, chemically modified nucleotides, suitably selected from ψ or m1ψ, wherein m1ψ is preferred

In particularly preferred embodiments, the mRNA comprises the following elements in 5′- to 3′-direction:
A) a cap1 structure as defined herein;
B) a 5′-UTR derived from a HSD17B4 gene as defined herein;
C) at least one cds encoding at least one transcription factor inhibitor as defined herein.
D) a 3′-UTR derived from a 3′-UTR of a PSMB3 gene as defined herein;
E) a histone stem-loop as defined herein;
F) a poly(A) sequence comprising about 100 A nucleotides, preferably representing the 3′ terminus;
G) optionally, chemically modified nucleotides, suitably selected from ψ or m1ψ, wherein m1ψ is preferred.

Preferred nucleic acid, preferably RNA sequences of the invention are provided in Table 3. Therein, each row represents a specific suitable RNA construct of the invention (compare with Table 2), wherein the description of the transcription factor inhibitor construct is indicated in column A and the SEQ ID NOs of the amino acid sequence of the respective transcription factor inhibitor construct is provided in column B. The corresponding SEQ ID NOs of the coding sequences encoding the respective transcription factor inhibitor constructs are provided in Table 2. Further information is provided under “feature key”, i.e. “source” (for nucleic acids or proteins) or “misc_feature” (for nucleic acids) or “REGION” (for proteins) of the respective SEQ ID NOs in the ST.26 sequence listing. The corresponding RNA sequences, in particular mRNA sequences comprising preferred coding sequences are provided in columns C-F, wherein column C provides RNA sequences with an UTR combination “HSD17B4/PSMB3” and a 3′ terminal A100 tail, column D provides nucleic acid sequences with an UTR combination “HSD17B4/PSMB3” and 3′ terminal hSL-A100 tail, column E provides RNA sequences with an UTR combination “HSD17B4/PSMB3” and a 3′ terminal A100-N5 tail, column F provides nucleic acid sequences with an UTR combination “HSD17B4/PSMB3” and 3′ terminal hSL-A100-N5 tail.

TABLE 3
RNA sequences encoding transcription factor inhibitors:

| row | A | B | C | D | E | F |
|---|---|---|---|---|---|---|
| 1 | CBFbeta(1-165)-SMMHC(1527- | 232 | 980, 990, | 800, 810, | 1340, 1350, | 1160, 1170, |
| | 1877) | | 1000, 1010, | 820, 830, | 1360, 1370, | 1180, 1190, |
| | (CBFbeta-SMMHCΔC95) | | 1020, 1030, | 840, 850, | 1380, 1390, | 1200, 1210, |
| | | | 1040, 1050, | 860, 870, | 1400, 1410, | 1220, 1230, |
| | | | 1060, 1070, | 880, 890, | 1420, 1430, | 1240, 1250, |
| | | | 1080, 1090, | 900, 910, | 1440, 1450, | 1260, 1270, |
| | | | 1100, 1110, | 920, 930, | 1460, 1470, | 1280, 1290, |
| | | | 1120, 1130, | 940, 950, | 1480, 1490, | 1300, 1310, |
| | | | 1140, 1150 | 960, 970, | 1500, 1510 | 1320, 1330 |
| | | | | 1567, 1568, | | |
| | | | | 1577, 1578- | | |
| | | | | 1582 | | |
| 2 | CBFbeta(1-165)-SMMHC(1527- | 231 | 979, 989, | 799, 809, | 1339, 1349, | 1159, 1169, |
| | 1927) | | 999, 1009, | 819, 829, | 1359, 1369, | 1179, 1189, |
| | | | 1019, 1029, | 839, 849, | 1379, 1389, | 1199, 1209, |
| | | | 1039, 1049, | 859, 869, | 1399, 1409, | 1219, 1229, |
| | | | 1059, 1069, | 879, 889, | 1419, 1429, | 1239, 1249, |
| | | | 1079, 1089, | 899, 909, | 1439, 1449, | 1259, 1269, |
| | | | 1099, 1109, | 919, 929, | 1459, 1469, | 1279, 1289, |
| | | | 1119, 1129, | 939, 949, | 1479, 1489, | 1299, 1309, |
| | | | 1139, 1149 | 959, 969 | 1499, 1509 | 1319, 1329 |
| 3 | CBFbeta(1-141)-SMMHC(1527- | 1545 | | 1563, 1573 | | |
| | 1877) | | | | | |
| 4 | CBFbeta(1-130)-Linker-Linker- | 1546 | | 1564, 1574 | | |
| | SMMHC(1527-1877) | | | | | |
| 5 | CBFbeta(1-165)-Linker-LifeAct | 233 | 981, 991, | 801, 811, | 1341, 1351, | 1161, 1171, |
| | | | 1001, 1011, | 821, 831, | 1361, 1371, | 1181, 1191, |
| | | | 1021, 1031, | 841, 851, | 1381, 1391, | 1201, 1211, |
| | | | 1041, 1051, | 861, 871, | 1401, 1411, | 1221, 1231, |
| | | | 1061, 1071, | 881, 891, | 1421, 1431, | 1241, 1251, |
| | | | 1081, 1091, | 901, 911, | 1441, 1451, | 1261, 1271, |
| | | | 1101, 1111, | 921, 931, | 1461, 1471, | 1281, 1291, |
| | | | 1121, 1131, | 941, 951, | 1481, 1491, | 1301, 1311, |
| | | | 1141, 1151 | 961, 971 | 1501, 1511 | 1321, 1331 |
| 6 | CBFbeta(1-165)-Linker-LifeAct | 1547 | | 1565, 1575 | | |
| 7 | CBFbeta(1-165)-Linker-LifeAct | 1548 | | 1566, 1576 | | |
| 8 | CBFbeta(1-165)-HIF1alpha(549- | 234 | 982, 992, | 802, 812, | 1342, 1352, | 1162, 1172, |
| | 575) | | 1002, 1012, | 822, 832, | 1362, 1372, | 1182, 1192, |
| | | | 1022, 1032, | 842, 852, | 1382, 1392, | 1202, 1212, |
| | | | 1042, 1052, | 862, 872, | 1402, 1412, | 1222, 1232, |
| | | | 1062, 1072, | 882, 892, | 1422, 1432, | 1242, 1252, |
| | | | 1082, 1092, | 902, 912, | 1442, 1452, | 1262, 1272, |
| | | | 1102, 1112, | 922, 932, | 1462, 1472, | 1282, 1292, |
| | | | 1122, 1132, | 942, 952, | 1482, 1492, | 1302, 1312, |
| | | | 1142, 1152 | 962, 972 | 1502, 1512 | 1322, 1332 |
| 9 | CBFbeta(1-165)-Linker- | 235 | 983, 993, | 803, 813, | 1343, 1353, | 1163, 1173, |
| | HIF1alpha(549-575) | | 1003, 1013, | 823, 833, | 1363, 1373, | 1183, 1193, |
| | | | 1023, 1033, | 843, 853, | 1383, 1393, | 1203, 1213, |
| | | | 1043, 1053, | 863, 873, | 1403, 1413, | 1223, 1233, |
| | | | 1063, 1073, | 883, 893, | 1423, 1433, | 1243, 1253, |
| | | | 1083, 1093, | 903, 913, | 1443, 1453, | 1263, 1273, |
| | | | 1103, 1113, | 923, 933, | 1463, 1473, | 1283, 1293, |
| | | | 1123, 1133, | 943, 953, | 1483, 1493, | 1303, 1313, |
| | | | 1143, 1153 | 963, 973 | 1503, 1513 | 1323, 1333 |
| 10 | CBFbeta(1-165)_Linker- | 1541 | | 1559, 1569 | | |
| | NFAT5(1-17) | | | | | |
| 11 | NFAT5(1-17)_Linker-CBFbeta(1- | 1542 | | 1560, 1570 | | |
| | 165) | | | | | |
| 12 | CBFbeta(1-165)-NFAT5(1-17) | 1543 | | 1561, 1571 | | |
| 13 | NFAT5(1-17)-CBFbeta(1-165) | 1544 | | 1562, 1572 | | |
| 14 | RUNX1(1-177)-RUNX1T1a(31- | 236 | 984, 994, | 804, 814, | 1344, 1354, | 1164, 1174, |
| | 604) | | 1004, 1014, | 824, 834, | 1364, 1374, | 1184, 1194, |
| | | | 1024, 1034, | 844, 854, | 1384, 1394, | 1204, 1214, |
| | | | 1044, 1054, | 864, 874, | 1404, 1414, | 1224, 1234, |
| | | | 1064, 1074, | 884, 894, | 1424, 1434, | 1244, 1254, |
| | | | 1084, 1094, | 904, 914, | 1444, 1454, | 1264, 1274, |
| | | | 1104, 1114, | 924, 934, | 1464, 1474, | 1284, 1294, |
| | | | 1124, 1134, | 944, 954, | 1484, 1494, | 1304, 1314, |
| | | | 1144, 1154 | 964, 974 | 1504, 1514 | 1324, 1334 |
| 15 | RUNX1(1-241)- | 237 | 985, 995, | 805, 815, | 1345, 1355, | 1165, 1175, |
| | RUNX1T1b(short) | | 1005, 1015, | 825, 835, | 1365, 1375, | 1185, 1195, |
| | | | 1025, 1035, | 845, 855, | 1385, 1395, | 1205, 1215, |
| | | | 1045, 1055, | 865, 875, | 1405, 1415, | 1225, 1235, |
| | | | 1065, 1075, | 885, 895, | 1425, 1435, | 1245, 1255, |
| | | | 1085, 1095, | 905, 915, | 1445, 1455, | 1265, 1275, |
| | | | 1105, 1115, | 925, 935, | 1465, 1475, | 1285, 1295, |
| | | | 1125, 1135, | 945, 955, | 1485, 1495, | 1305, 1315, |
| | | | 1145, 1155 | 965, 975 | 1505, 1515 | 1325, 1335 |
| 16 | RUNX1(1-241)-RUNX1T1b(22- | 238, 239 | 986, 987, | 806, 807, | 1346, 1347, | 1166, 1167, |
| | 604) | | 996, 997, | 816, 817, | 1356, 1357, | 1176, 1177, |
| | | | 1006, 1007, | 826, 827, | 1366, 1367, | 1186, 1187, |
| | | | 1016, 1017, | 836, 837, | 1376, 1377, | 1196, 1197, |
| | | | 1026, 1027, | 846, 847, | 1386, 1387, | 1206, 1207, |
| | | | 1036, 1037, | 856, 857, | 1396, 1397, | 1216, 1217, |
| | | | 1046, 1047, | 866, 867, | 1406, 1407, | 1226, 1227, |
| | | | 1056, 1057, | 876, 877, | 1416, 1417, | 1236, 1237, |
| | | | 1066, 1067, | 886, 887, | 1426, 1427, | 1246, 1247, |
| | | | 1076, 1077, | 896, 897, | 1436, 1437, | 1256, 1257, |
| | | | 1086, 1087, | 906, 907, | 1446, 1447, | 1266, 1267, |
| | | | 1096, 1097, | 916, 917, | 1456, 1457, | 1276, 1277, |
| | | | 1106, 1107, | 926, 927, | 1466, 1467, | 1286, 1287, |
| | | | 1116, 1117, | 936, 937, | 1476, 1477, | 1296, 1297, |
| | | | 1126, 1127, | 946, 947, | 1486, 1487, | 1306, 1307, |
| | | | 1136, 1137, | 956, 957, | 1496, 1497, | 1316, 1317, |
| | | | 1146, 1147, | 966, 967, | 1506, 1507, | 1326, 1327, |
| | | | 1156, 1157 | 976, 977 | 1516, 1517 | 1336, 1337 |
| 17 | RUNX1(K83E, R174Q) | 197 | 978, 988, | 798, 808, | 1338, 1348, | 1158, 1168, |
| | | | 998, 1008, | 818, 828, | 1358, 1368, | 1178, 1188, |
| | | | 1018, 1028, | 838, 848, | 1378, 1388, | 1198, 1208, |
| | | | 1038, 1048, | 858, 868, | 1398, 1408, | 1218, 1228, |
| | | | 1058, 1068, | 878, 888, | 1418, 1428, | 1238, 1248, |
| | | | 1078, 1088, | 898, 908, | 1438, 1448, | 1258, 1268, |
| | | | 1098, 1108, | 918, 928, | 1458, 1468, | 1278, 1288, |
| | | | 1118, 1128, | 938, 948, | 1478, 1488, | 1298, 1308, |
| | | | 1138, 1148 | 958, 968 | 1498, 1508 | 1318, 1328 |

Particularly preferred nucleic acid of the invention, preferably RNA sequences, are provided in Table 4. Said particularly preferred RNA sequences each encode a RUNX inhibitor, in particular a RUNX trap (CBFbeta-SMMHC) according to the amino acid sequence of SEQ ID NO: 232.

In Table 4, each row represents a specific suitable RNA construct of the invention (compare with Table 3, row 1), wherein the description of the overall RNA design (e.g. UTRs, hSL) is indicated in column A. For example, row 1 relates to RNA constructs with an UTR combination “HSD17B4/PSMB3” and a 3′ terminal hSL-A100 tail, row 2 relates to RNA constructs with an UTR combination “HSD17B4/PSMB3” and a 3′ terminal A100 tail, row 3 relates to RNA constructs with an UTR combination “HSD17B4/PSMB3” and a 3′ terminal hSL-A100-N5 tail, row 4 relates to RNA constructs with an UTR combination “HSD17B4/PSMB3” and a 3′ terminal A100-N5 tail.

The corresponding SEQ ID NOs different RNA constructs comprising different coding sequences (all encoding the RUNX inhibitor “CBFB (1-165)-SMMHC (1527-1877)” according to SEQ ID NO: 232) are provided in Columns, wherein column B relates to RNA sequences comprising wild type or reference coding sequences, column C relates to RNA sequences comprising G/C optimized (opt1) coding sequences, column D relates to RNA sequences comprising human codon usage adapted (opt3) coding sequences, column E relates to RNA sequences comprising CAI maximized (opt4) coding sequences, column F relates to RNA sequences comprising G/C modified (opt5) coding sequences, and column G relates to RNA sequences comprising G/C optimized (opt11) coding sequences.

Further information is provided under “feature key”, i.e. “source” (for nucleic acids or proteins) or “misc_feature” (for nucleic acids) or “REGION” (for proteins) of the respective SEQ ID NOs in the ST.26 sequence listing.

TABLE 4
RNA sequences encoding the preferred RUNX inhibitor according to SEQ ID NO: 232:

| A | B | C | D | E | F | G |
|---|---|---|---|---|---|---|
| HSD17B4/PSMB3 | 800, 810, | 820, 830, | 840, 850, | 860, 950, | 870, 960 | 880, 970, |
| hSL-A100 | 890, 900 | 910, 920, | 930, 940 | 1567 | | 1568 |
| | | 1579-1582 | | | | |
| HSD17B4/PSMB3 | 980, 990, | 1000, 1010, | 1020, 1030, | 1040, | 1050, | 1060, |
| A100 | 1070, 1080 | 1090, 1100 | 1110, 1120 | 1130 | 1140 | 1150 |
| HSD17B4/PSMB3 | 1160, 1170, | 1180, 1190, | 1200, 1210, | 1220, | 1230, | 1240, |
| hSL-A100-N5 | 1250, 1260 | 1270, 1280 | 1290, 1300 | 1310 | 1320 | 1330 |
| HSD17B4/PSMB3 | 1340, 1350, | 1360, 1370, | 1380, 1390, | 1400, | 1410, | 1420, |
| A100-N5 | 1430, 1440 | 1450, 1460 | 1470, 1480 | 1490 | 1500 | 1510 |

In preferred embodiments, the nucleic acid, preferably the RNA, comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence according to any one of SEQ ID NOs: 798-1517, 1559-1582 or a fragment or variant of any of these sequences. Further information regarding respective nucleic acid sequences is provided in Table 3 and Table 4.

In other preferred embodiments, the nucleic acid, preferably the RNA, comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence according to any one of SEQ ID NOs: 798-1517, 1559-1582 or a fragment or variant of any of these sequences, optionally wherein at least one, preferably all uracil nucleotides in said RNA sequences are replaced by pseudouridine (ψ) nucleotides and/or N1-methylpseudouridine (m1ψ) nucleotides.

In preferred embodiments, the nucleic acid, preferably the RNA, comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence according to any one of SEQ ID NOs: 799-801, 809-811, 819-821, 829-831, 839-841, 849-851, 859-861, 869-871, 879-881, 889-891, 899-901, 909-911, 919-921, 929-931, 939-941, 949-951, 959-961, 969-971, 979-981, 989-991, 999-1001, 1009-1011, 1019-1021, 1029-1031, 1039-1041, 1049-1051, 1059-1061, 1069-1071, 1079-1081, 1089-1091, 1099-1101, 1109-1111, 1119-1121, 1129-1131, 1139-1141, 1149-1151, 1159-1161, 1169-1171, 1179-1181, 1189-1191, 1199-1201, 1209-1211, 1219-1221, 1229-1231, 1239-1241, 1249-1251, 1259-1261, 1269-1271, 1279-1281, 1289-1291, 1299-1301, 1309-1311, 1319-1321, 1329-1331, 1339-1341, 1349-1351, 1359-1361, 1369-1371, 1379-1381, 1389-1391, 1399-1401, 1409-1411, 1419-1421, 1429-1431, 1439-1441, 1449-1451, 1459-1461, 1469-1471, 1479-1481, 1489-1491, 1499-1501, 1509-1511, 1559-1582, or a fragment or variant of any of these sequences.

In more preferred embodiments, the nucleic acid, preferably the RNA, comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence according to any one of SEQ ID NOs: 800, 810, 820, 830, 840, 850, 860, 870, 880, 890, 900, 910, 920, 930, 940, 950, 960, 970, 1567, 1568, 1577, 1578-1582, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, or a fragment or variant of any of these sequences encoding CBFbeta-SMMHC (see also Table 4).

In preferred embodiments, the nucleic acid, preferably the RNA, comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence according to SEQ ID NOs: 820, 1579, 1581 or 910 1580, 1582, or a fragment or variant thereof encoding CBFbeta-SMMHC.

In particularly preferred embodiments, the nucleic acid is a N1-methylpseudouridine (m1w) modified RNA, that comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence according to SEQ ID NOs: 1579 or 1580, or a fragment or variant thereof encoding CBFbeta-SMMHC.

In particularly preferred embodiments, the artificial nucleic acid of the invention is a N1-methylpseudouridine (m1w) modified 5′ cap1 mRNA that comprises or consists of an RNA sequence which is identical or at least 90% identical to a nucleic sequence according to SEQ ID NOs: 1579 or 1580, or a fragment or variant of any of these sequences encoding CBFbeta-SMMHC.

In a particularly preferred embodiment, the artificial nucleic acid of the invention is a N1-methylpseudouridine (m1w) modified 5′ cap1 mRNA that comprises or consists of an RNA sequence which is identical or at least 90% identical to a nucleic sequence according to SEQ ID NO: 1580, or a fragment or variant of any of these sequences encoding CBFbeta-SMMHC.

### 2: Composition Comprising at Least One Nucleic Acid Encoding a Transcription Factor Inhibitor:

In a second aspect, the invention provides a pharmaceutical composition comprising at least one nucleic acid encoding at least one transcription factor inhibitor as defined in the first aspect.

Notably, features and embodiments described in the context of the first aspect (the nucleic acid of the invention encoding at least one transcription factor inhibitor) have to be read on and have to be understood as suitable embodiments of the pharmaceutical composition of the second aspect and vice versa.

In the context of the invention, a “composition” refers to any type of composition in which the specified ingredients (e.g. nucleic acid encoding at least one transcription factor inhibitor) may be incorporated, optionally along with any further constituents, usually with at least one pharmaceutically acceptable carrier or excipient. The composition may be a dry composition such as a powder, a granule, or a solid lyophilized form. Alternatively, the composition may be in liquid form, and each constituent may be independently incorporated in dissolved or dispersed (e.g. suspended or emulsified) form.

Preferably, the at least one nucleic acid of the pharmaceutical composition is selected from an RNA as further defined in the first aspect. In particularly preferred embodiments, the at least one nucleic acid of the pharmaceutical composition is selected from an mRNA as further defined in first aspect.

In embodiments, the nucleic acid, preferably the RNA as comprised in the pharmaceutical composition is provided in an amount of about 10 ng to about 500 μg, in an amount of about 1 μg to about 500 μg, in an amount of about 1 μg to about 100 μg, specifically, in an amount of about 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg or 100 μg.

In preferred embodiments, the pharmaceutical composition comprises a plurality or at least more than one nucleic acid species (e.g. RNA species), preferably wherein each nucleic acid species encodes a different transcription factor inhibitor.

Preferably, the pharmaceutical composition as defined herein may comprise 2, 3, 4, 5, 6, 7, 8, 9, or 10 nucleic acid species each as defined in the first aspect, wherein each of the 2, 3, 4, 5, 6, 7, 8, 9, or 10 nucleic acid species encode a different transcription factor inhibitor, wherein the at least one different transcription factor inhibitor differs in at least one amino acid position.

For example, the pharmaceutical composition as defined herein may comprise at least one artificial nucleic acid encoding at least one transcription factor inhibitor comprising a CBFbeta amino acid sequence element and an SMMHC amino acid sequence element (CBFbeta-SMMHC); and at least one selected from
(i) at least one artificial nucleic acid encoding at least one transcription factor inhibitor comprising a RUNX1 amino acid sequence element and an RUNX1T1 amino acid sequence element (RUNX1-RUNX1T1);
(ii) at least one artificial nucleic acid encoding at least one transcription factor inhibitor comprising a mutated RUNX1 amino acid sequence element (RUNX1 (K83E,R174Q));
(iii) at least one artificial nucleic acid encoding at least one transcription factor inhibitor comprising a mutated RUNX1 amino acid sequence element (RUNX1 (K83E,R174Q);
(iv) at least one artificial nucleic acid encoding at least one transcription factor inhibitor comprising a CBFbeta amino acid sequence element and an NFAT5 amino acid sequence element;
(v) at least one artificial nucleic acid encoding at least one transcription factor inhibitor comprising a CBFbeta amino acid sequence element and an HIF1alpha amino acid sequence element.

In various embodiments, the at least one artificial nucleic acid, preferably the at least one RNA of the pharmaceutical composition, is formulated with a pharmaceutically acceptable carrier or excipient.

The term “pharmaceutically acceptable carrier” or “pharmaceutically acceptable excipient” as used herein preferably includes the liquid or non-liquid basis of the composition for administration. If the pharmaceutical composition is provided in liquid form, the carrier may be water, e.g. pyrogen-free water; isotonic saline or buffered (aqueous) solutions, e.g. phosphate, citrate etc. buffered solutions. Water or preferably a buffer, more preferably an aqueous buffer, may be used comprising e.g. a sodium salt, a calcium salt, or a potassium salt. According to preferred embodiments, the sodium, calcium or potassium salts may occur in the form of their halogenides, e.g. chlorides, iodides, or bromides, in the form of their hydroxides, carbonates, hydrogen carbonates, or sulphates, etc. Examples of sodium salts include NaCl, Na₂HPO₄, Na₃PO₄, NaI, NaBr, Na2CO3, NaHCO3, Na2SO4, examples of potassium salts include KCl, Kl, KBr, K2CO3, KHCO3, K2SO4, and examples of calcium salts include CaCl2, CaI2, CaBr2, CaCO3, CaSO4, Ca(OH)2.

Furthermore, organic anions of the aforementioned cations may be in the buffer. Accordingly, in embodiments, the nucleic acid composition may comprise pharmaceutically acceptable carriers or excipients using one or more pharmaceutically acceptable carriers or excipients to e.g. increase stability, increase cell transfection, permit the sustained or delayed, increase the translation of encoded transcription factor inhibitor in vivo, and/or alter the release profile of the encoded transcription factor inhibitor in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles, polymers, lipoplexes, core-shell nanoparticles, peptides, proteins, cells transfected with polynucleotides, hyaluronidase, nanoparticle mimics and combinations thereof. In embodiments, one or more compatible solid or liquid fillers or diluents or encapsulating compounds may be used as well, which are suitable for administration to a subject. The term “compatible” as used herein means that the constituents of the pharmaceutical composition are capable of being mixed with the at least one nucleic acid and, optionally, a plurality of nucleic acids of the composition, in such a manner that no interaction occurs, which would substantially reduce the biological activity or the pharmaceutical effectiveness of the composition under typical use conditions. Pharmaceutically acceptable carriers or excipients must have sufficiently high purity and sufficiently low toxicity to make them suitable for administration to a subject to be treated. Compounds which may be used as pharmaceutically acceptable carriers or excipients may be sugars, such as, for example, lactose, glucose, trehalose, mannose, and sucrose; starches, such as, for example, corn starch or potato starch; dextrose; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; alginic acid.

Pharmaceutical compositions of the present invention are suitably sterile and/or pyrogen-free.

### Formulation/Complexation:

In preferred embodiments, the at least one nucleic acid, preferably the at least one RNA of the pharmaceutical composition is complexed or associated with at least one further compound to obtain a formulated composition. A formulation in that context may have the function of a transfection agent. A formulation in that context may also have the function of protecting the nucleic acid from degradation, e.g. to allow storage, shipment, etc.

In preferred embodiments, the at least one nucleic acid, preferably the at least one RNA of the pharmaceutical composition is formulated with at least one compound, e.g. peptides, proteins, lipids, polysaccharides, and/or polymers.

In preferred embodiments, the at least one artificial nucleic acid, preferably the at least one RNA of the pharmaceutical composition is formulated with at least one cationic (cationic or preferably ionizable) or polycationic compound (cationic or preferably ionizable).

In preferred embodiments, the at least one artificial nucleic acid, preferably the at least one RNA of the pharmaceutical composition is complexed or associated with or at least partially complexed or partially associated with one or more cationic (cationic or preferably ionizable) or polycationic compound.

The term “cationic or polycationic compound” as used herein will be recognized and understood by the person of ordinary skill in the art, and are for example intended to refer to a charged molecule, which is positively charged at a pH value ranging from about 1 to 9, at a pH value ranging from about 3 to 8, at a pH value ranging from about 4 to 8, at a pH value ranging from about 5 to 8, more preferably at a pH value ranging from about 6 to 8, even more preferably at a pH value ranging from about 7 to 8, most preferably at a physiological pH, e.g. ranging from about 7.2 to about 7.5. Accordingly, a cationic component, e.g. a cationic peptide, cationic protein, cationic polymer, cationic polysaccharide, cationic lipid may be any positively charged compound or polymer which is positively charged under physiological conditions. A “cationic or polycationic peptide or protein” may contain at least one positively charged amino acid, or more than one positively charged amino acid, e.g. selected from Arg, His, Lys or Om. Accordingly, “polycationic” components are also within the scope exhibiting more than one positive charge under the given conditions.

In preferred embodiments, the at least one cationic or polycationic compound is selected from a cationic or polycationic polymer, a cationic or polycationic polysaccharide, a cationic or polycationic lipid, a cationic or polycationic protein, a cationic or polycationic peptide, or any combinations thereof.

In preferred embodiments, the at least one cationic or polycationic compound is selected from a cationic or polycationic peptide or protein.

Preferred cationic or polycationic proteins or peptides that may be used for complexation can be derived from formula (Arg)l; (Lys)m; (His)n; (Or)o; (Xaa)x of the patent application WO2009030481 or WO2011026641, the disclosure of WO2009030481 or WO2011026641 relating thereto incorporated herewith by reference.

In preferred embodiments, the at least one artificial nucleic acid, preferably the at least one RNA is complexed, or at least partially complexed, with at least one cationic or polycationic proteins or peptides preferably selected from SEQ ID NOs: 173-177, or any combinations thereof.

In some embodiments, the pharmaceutical composition comprises at least one nucleic acid, preferably the at least one RNA as defined herein, and a polymeric carrier.

The term “polymeric carrier” as used herein will be recognized and understood by the person of ordinary skill in the art, and are e.g. intended to refer to a compound that facilitates transport and/or complexation of another compound. A polymeric carrier is typically a carrier that is formed of a polymer. A polymeric carrier may be associated to its cargo (e.g. RNA) by covalent or non-covalent interaction. A polymer may be based on different subunits, such as a copolymer. Suitable polymeric carriers in that context may include, for example, polyethylenimine (PEI).

A preferred polymeric carrier may be a polymeric carrier formed by disulfide-crosslinked cationic compounds. The disulfide-crosslinked cationic compounds may be the same or different from each other. The polymeric carrier can also contain further components. The polymeric carrier used according to the present invention may comprise mixtures of cationic peptides, proteins or polymers and optionally further components as defined herein, which are cross-linked by disulfide bonds (via —SH groups).

In this context, polymeric carriers according to formula {(Arg)l; (Lys)m; (His)n; (Orn)o; (Xaa)x (Cys)y} and formula Cys, {(Arg)l; (Lys)m; (His)n; (Or)o; (Xaa)x}Cys2 of the patent application WO2012013326 are preferred, the disclosure of WO2012013326 relating thereto incorporated herewith by reference.

In preferred embodiments, the polymeric carrier used to complex the at least one nucleic acid, preferably the at least one RNA may be derived from a polymeric carrier molecule according formula (L-P1-S—[S—P2-S]n-S—P3-L) of the patent application WO2011026641, the disclosure of WO2011026641 relating thereto incorporated herewith by reference.

In preferred embodiments, the polymeric carrier compound is formed by, or comprises or consists of the peptide elements CysArg12Cys (SEQ ID NO: 173) or CysArg12 (SEQ ID NO: 174) or TrpArg12Cys (SEQ ID NO: 175). In particularly preferred embodiments, the polymeric carrier compound consists of a (R12C)-(R12C) dimer, a (WR12C)-(WR12C) dimer, or a (CR12)-(CR12C)-(CR12) trimer, wherein the individual peptide elements in the dimer (e.g. (WR12C)), or the trimer (e.g. (CR12)), are connected via-SH groups.

In preferred embodiment, the at least one artificial nucleic acid, preferably the at least one RNA is complexed or associated with a polyethylene glycol/peptide polymer, preferably comprising HO-PEG5000-S-(S-CHHHHHHRRRRHHHHHHC-S-)7-S-PEG5000-OH (SEQ ID NO: 176 as peptide monomer), HO-PEG5000-S-(S-CHHHHHHRRRRHHHHHHC-S-)4 S-PEG5000-OH (SEQ ID NO: 176 as peptide monomer), HO-PEG5000-S-(S-CGHHHHHRRRRHHHHHGC-S-)7-S-PEG5000-OH (SEQ ID NO: 177 as peptide monomer) and/or a polyethylene glycol/peptide polymer comprising HO-PEG5000-S-(S-CGHHHHHRRRRHHHHHGC-S-) 4-S-PEG5000-OH (SEQ ID NO: 177 as peptide monomer).

In other embodiments, the at least one nucleic acid of the composition, preferably the at least one RNA is complexed or associated with polymeric carriers and, optionally, with at least one lipid component as described in WO2017212008, WO2017212006, WO2017212007, and WO2017212009. In this context, the disclosures of WO2017212008, WO2017212006, WO2017212007, and WO2017212009 are herewith incorporated by reference.

In a particularly preferred embodiment, the polymeric carrier of the pharmaceutical composition is a peptide polymer, preferably a polyethylene glycol/peptide polymer as defined above, and a lipid component, preferably a lipidoid component.

A lipidoid (or lipidoit) is a lipid-like compound, i.e. an amphiphilic compound with lipid-like physical properties. The lipidoid is preferably a compound, which comprises two or more cationic nitrogen atoms and at least two lipophilic tails. In contrast to many conventional cationic lipids, the lipidoid may be free of a hydrolysable linking group, in particular linking groups comprising hydrolysable ester, amide or carbamate groups. The cationic nitrogen atoms of the lipidoid may be cationisable or permanently cationic, or both types of cationic nitrogens may be present in the compound. In the context of the present invention, the term lipid is considered to also encompass lipidoids.

Suitably, the lipidoid is cationic, which means that it is cationisable or permanently cationic. In one embodiment, the lipidoid is cationisable, i.e. it comprises one or more cationisable nitrogen atoms, but no permanently cationic nitrogen atoms. In another embodiment, at least one of the cationic nitrogen atoms of the lipidoid is permanently cationic. Optionally, the lipidoid comprises two permanently cationic nitrogen atoms, three permanently cationic nitrogen atoms, or even four or more permanently cationic nitrogen atoms.

In preferred embodiment at least one artificial nucleic acid, preferably the at least one RNA of the pharmaceutical composition is complexed or associated with a polymeric carrier, preferably with a polyethylene glycol/peptide polymer as defined above, and a lipidoid component,

In preferred embodiments, the lipidoid component is a compound according to formula A

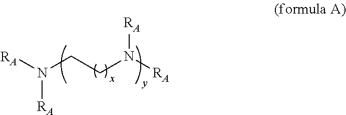

wherein
RA is independently selected for each occurrence an unsubstituted, cyclic or acyclic, branched or unbranched C1-20 aliphatic group; a substituted or unsubstituted, cyclic or acyclic, branched or unbranched C1-20 heteroaliphatic group; a substituted or unsubstituted aryl; a substituted or unsubstituted heteroaryl;

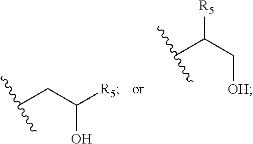

wherein at least one RA is

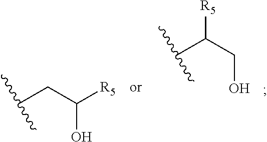

R5 is independently selected for each occurrence of from an unsubstituted, cyclic or acyclic, branched or unbranched C8-16 aliphatic; a substituted or unsubstituted aryl; or a substituted or unsubstituted heteroaryl;
each occurrence of x is an integer from 1 to 10;
each occurrence of y is an integer from 1 to 10;
or a pharmaceutically acceptable salt thereof.

In a preferred embodiment, the lipidoid component may be any one selected from the lipidoids of the lipidoids provided in the table of page 50-54 of published PCT patent application WO2017212009, the specific lipidoids provided in said table, and the specific disclosure relating thereto herewith incorporated by reference.

In preferred embodiments, the lipidoid component may be any one selected from 3-C12-OH, 3-C12-OH-cat, 3-C12-amide, 3-C12-amide monomethyl, 3-C12-amide dimethyl, RevPEG(10)-3-C12-OH, RevPEG(10)-DLin-pAbenzoic, 3C12amide-TMA cat., 3C12amide-DMA, 3C12amide-NH2, 3C12amide-OH, 3C12Ester-OH, 3C12 Ester-amin, 3C12Ester-DMA, 2C12Amid-DMA, 3C12-lin-amid-DMA, 2C12-sperm-amid-DMA, or 3C12-sperm-amid-DMA (see table of published PCT patent application WO2017212009 (pages 50-54)).

Particularly preferred lipidoid components in the context of the invention are 3-C12-OH, 3-C12-OH-cat, 3-C12-C3-OH.

In preferred embodiments, the polyethylene glycol/peptide polymer comprising a lipidoid as specified above (e.g. 3-C12-OH or 3-C12-OH-cat), is used to complex the at least one nucleic acid to form complexes having an N/P ratio from about 0.1 to about 20, or from about 0.2 to about 15, or from about 2 to about 15, or from about 2 to about 12, wherein the N/P ratio is defined as the mole ratio of the nitrogen atoms of the basic groups of the cationic peptide or polymer to the phosphate groups of the nucleic acid. In that context, the disclosure of published PCT patent application WO2017212009, in particular claims 1 to 10 of WO2017212009, and the specific disclosure relating thereto is herewith incorporated by reference.

Further suitable lipidoids may be derived from published PCT patent application WO2010053572. In particular, lipidoids derivable from claims 1 to 297 of published PCT patent application WO2010053572 may be used in the context of the invention, e.g. incorporated into the peptide polymer as described herein, or e.g. incorporated into the lipid nanoparticle (as described below). Accordingly, claims 1 to 297 of published PCT patent application WO2010053572, and the specific disclosure relating thereto, is herewith incorporated by reference.

In particularly preferred embodiments, the pharmaceutical composition comprises at least one nucleic acid, preferably RNA that comprises or consists of a nucleic acid sequence encoding at least one transcription factor trap, preferably a RUNX inhibitor as defined herein, wherein the nucleic acid is formulated in a polyethylene glycol/peptide polymer as defined herein comprising a lipidoid as defined herein.

Preferably, said formulations are particularly suitable for ocular administration.

### Formulation in Lipid-Based Carriers:

In particularly preferred embodiments, the at least one artificial nucleic acid, preferably the at least one RNA of the pharmaceutical composition is formulated in lipid-based carriers.

In the context of the invention, the term “lipid-based carriers” encompass lipid-based delivery systems for nucleic acid (e.g. RNA) that comprise a lipid component. A lipid-based carrier may additionally comprise other components suitable for encapsulating/incorporating/complexing a nucleic acid (e.g. RNA) including a cationic or polycationic polymer, a cationic or polycationic polysaccharide, a cationic or polycationic protein, a cationic or polycationic peptide, or any combinations thereof.

In the context of the invention, a typical “lipid-based carrier” is selected from liposomes, lipid nanoparticles (LNPs), lipoplexes, solid lipid nanoparticles, and/or nanoliposomes. The nucleic acid, preferably the RNA of the pharmaceutical composition may completely or partially incorporated or encapsulated in a lipid-based carrier, wherein the nucleic acid (e.g. RNA) may be located in the interior space of the lipid-based carrier, within the lipid layer/membrane of the lipid-based carrier, or associated with the exterior surface of the lipid-based carrier. The incorporation of nucleic acid, preferably the RNA into lipid-based carriers may be referred to as “encapsulation”. A “lipid-based carrier” is not restricted to any particular morphology, and include any morphology generated when e.g. an aggregation reducing lipid and at least one further lipid are combined, e.g. in an aqueous environment in the presence of nucleic acid (e.g. RNA). For example, an LNP, a liposome, a lipid complex, a lipoplex and the like are within the scope of the term “lipid-based carrier”. Lipid-based carriers can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 nm and 500 nm in diameter. Liposomes, a specific type of lipid-based carrier, are characterized as microscopic vesicles having an interior aqua space sequestered from an outer medium by a membrane of one or more bilayers. In a liposome, the at least one nucleic acid (e.g. RNA) is typically located in the interior aqueous space enveloped by some or the entire lipid portion of the liposome. Bilayer membranes of liposomes are typically formed by amphiphilic molecules, such as lipids of synthetic or natural origin that comprise spatially separated hydrophilic and hydrophobic domains. Lipid nanoparticles (LNPs), a specific type of lipid-based carrier, are characterized as microscopic lipid particles having a solid core or partially solid core. Typically, an LNP does not comprise an interior aqua space sequestered from an outer medium by a bilayer. In an LNP, the at least one nucleic acid (e.g. RNA) may be encapsulated or incorporated in the lipid portion of the LNP enveloped by some or the entire lipid portion of the LNP. An LNP may comprise any lipid capable of forming a particle to which the nucleic acid (e.g. RNA) may be attached, or in which the nucleic acid may be encapsulated. Preferably, said lipid-based carriers are particularly suitable for ocular administration.

In preferred embodiments, the lipid-based carriers of the pharmaceutical composition are selected from liposomes, lipid nanoparticles, lipoplexes, solid lipid nanoparticles, lipo-polylexes, and/or nanoliposomes.

In preferred embodiments, the lipid-based carriers of the pharmaceutical composition are lipid nanoparticles (LNPs). In particularly preferred embodiments, the lipid nanoparticles of the pharmaceutical composition encapsulate the at least one nucleic acid, preferably the at least one RNA of the invention.

The term “encapsulated”, e.g. incorporated, complexed, encapsulated, partially encapsulated, associated, partially associated, refers to the essentially stable combination of nucleic acid, preferably RNA with one or more lipids into lipid-based carriers (e.g. larger complexes or assemblies) preferably without covalent binding of the nucleic acid. The lipid-based carriers-encapsulated nucleic acid (e.g. RNA) may be completely or partially located in the interior of the lipid-based carrier (e.g. the lipid portion and/or an interior space) and/or within the lipid layer/membrane of the lipid-based carriers. The encapsulation of a nucleic acid (e.g. RNA) into lipid-based carriers is also referred to herein as “incorporation” as the nucleic acid (e.g. RNA) is preferably contained within the interior of the lipid-based carriers. Without wishing to be bound to theory, the purpose of incorporating or encapsulating nucleic acid into lipid-based carriers may be to protect the nucleic acid from an environment which may contain enzymes, chemicals, or conditions that degrade the nucleic acid (e.g. RNA). Moreover, incorporating nucleic acid into lipid-based carriers may promote the uptake of the nucleic acid and their release from the endosomal compartment, and hence, may enhance the therapeutic effect of the nucleic acid (e.g. RNA) when administered to a cell or a subject.

In preferred embodiments, the lipid-based carriers of the pharmaceutical composition comprise at least one or more lipids selected from at least one aggregation-reducing lipid, at least one cationic lipid, at least one neutral lipid or phospholipid, or at least one steroid or steroid analog, or any combinations thereof.

In preferred embodiments, the lipid-based carriers of the pharmaceutical composition comprise (i) an aggregation-reducing lipid, (ii) a cationic lipid or ionizable lipid, and (iii) a neutral lipid/phospholipid or a steroid/steroid analog.

In particularly preferred embodiments, the lipid-based carriers of the pharmaceutical composition comprise an (i) aggregation-reducing lipid, (ii) a cationic lipid or ionizable lipid, (iii) a neutral lipid or phospholipid, (iv) and a steroid or steroid analog.

### Cationic Lipids:

In preferred embodiments, the lipid-based carriers comprise at least one cationic or ionizable lipid.

The cationic or ionizable lipid of the lipid-based carriers may be cationisable or ionizable, i.e. it becomes protonated as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

In preferred embodiments, the lipid-based carriers comprise a cationic or ionizable lipid that preferably carries a net positive charge at physiological pH, more preferably the cationic or ionizable lipid comprises a tertiary nitrogen group or quaternary nitrogen group. Accordingly, in preferred embodiments, the lipid-based carriers comprise a cationic or ionizable lipid selected from an amino lipid.

In further embodiments, the lipid formulation comprises cationic or ionizable lipids as defined in Formula I of paragraph [00251] of WO2021222801 or a lipid selected from the disclosure of paragraphs [00260] or [00261] of WO2021222801. In other embodiments, the lipid formulation comprises cationic or ionizable lipids selected from the group consisting of ATX-001 to ATX-132 as disclosed in claim 90 of WO2021183563, preferably ATX-0126. The disclosure of WO2021222801 and WO2021183563, especially aforementioned lipids, are incorporated herewith by reference.

Accordingly, the at least one cationic or ionizable lipid can be a lipid selected or derived from formula (III-1)

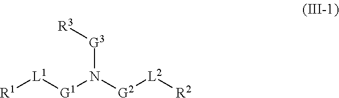

preferably, wherein one of L1 or L2 is —O(C═O)—, —(C═O)O—, —C(═O)—, —O—, —S(O)x-, —S—S—, —C(═O)S—, SC(═O)—, —NRaC(═O)—, —C(═O)NRa—, —NRaC(═O)NRa—, —OC(═O)NRa— or —NRaC(═O)O—, and the other of L1 or L2 is —O(C═O)—, —(C═O)O—, —C(═O)—, —O—, —S(O)x—, —S S—, —C(═O)S—, SC(═O)—, —NRaC(═O)—, —C(═O)NRa—, —NRaC(═O)NRa—, —OC(═O)NRa— or —NRaC(═O)O— or a direct bond; G1 and G2 are each independently unsubstituted C1-C12 alkylene or C1-C12 alkenylene; G3 is C1-C24 alkylene, C1-C24 alkenylene, C3-C8 cycloalkylene, C3-C8 cycloalkenylene; Ra is H or C1-C12 alkyl; R1 and R2 are each independently C6-C24 alkyl or C6-C24 alkenyl; R3 is H, OR5, CN, C(═O)OR4, OC(═O)R4 or —NR5C(═O)R4; R4 is C1-C12 alkyl; R5 is H or C1-C6 alkyl; and x is 0, 1 or 2.

In some embodiments, cationic or ionizable lipids may be selected from the lipids disclosed in WO2018078053 (i.e. lipids derived from formula I, II, and Ill of WO2018078053, or lipids as specified in claims 1 to 12 of WO2018078053), the disclosure of WO2018078053 hereby incorporated by reference in its entirety. In that context, lipids disclosed in Table 7 of WO2018078053 (e.g. lipids derived from formula I-1 to 1-41) and lipids disclosed in Table 8 of WO2018078053 (e.g. lipids derived from formula II-1 to II-36) may be suitably used in the context of the invention. Accordingly, formula I-1 to formula I-41 and formula II-1 to formula II-36 of WO2018078053A, and the specific disclosure relating thereto, are herewith incorporated by reference.

In preferred embodiments, the lipid-based carriers of the pharmaceutical composition comprise a cationic lipid selected or derived from structures III-1 to III-36 of Table 9 of published PCT patent application WO2018078053. Accordingly, formula III-1 to III-36 of WO2018078053, and the specific disclosure relating thereto, are herewith incorporated by reference.

In preferred embodiments, the lipid-based carriers comprise a cationic lipid selected or derived from formula III-3:

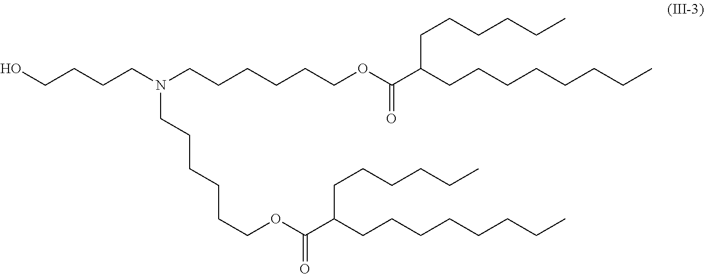 [SMILES: CCCCCCCCC(CCCCCC)C(=O)OCCCCCCN(CCCCO)CCCCCCOC(=O)C(CCCCCC)CCCCCCCC]

The lipid of formula III-3 as suitably used herein has the chemical term ((4-hydroxybutyl)azanediyl)bis(hexane-6,1-diyl)bis(2-hexyldecanoate), also referred to as ALC-0315 i.e. CAS Number 2036272-55-4.

Further suitable cationic lipids may be selected or derived from cationic lipids according to PCT claims 1 to 14 of published patent application WO2021123332, or table 1 of WO2021123332, the disclosure relating to claims 1 to 14 or table 1 of WO2021123332 herewith incorporated by reference.

Accordingly, suitable cationic lipids may be selected or derived from cationic lipids according Compound 1 to Compound 27 (C1-C27) of Table 1 of WO2021123332.

In other preferred embodiments, the lipid-based carriers of the pharmaceutical composition comprise a cationic lipid selected or derived from (COATSOMERSS-EC) SS-33/4PE-15 (see C23 in Table 1 of WO2021123332).

In other preferred embodiments, the lipid-based carriers of the pharmaceutical composition comprise a cationic lipid selected or derived from HEXA-C5DE-PipSS (see C2 in Table 1 of WO2021123332). In most preferred embodiments, the lipid-based carriers of the pharmaceutical composition comprise a cationic lipid selected or derived from compound C26 as disclosed in Table 1 of WO2021123332:

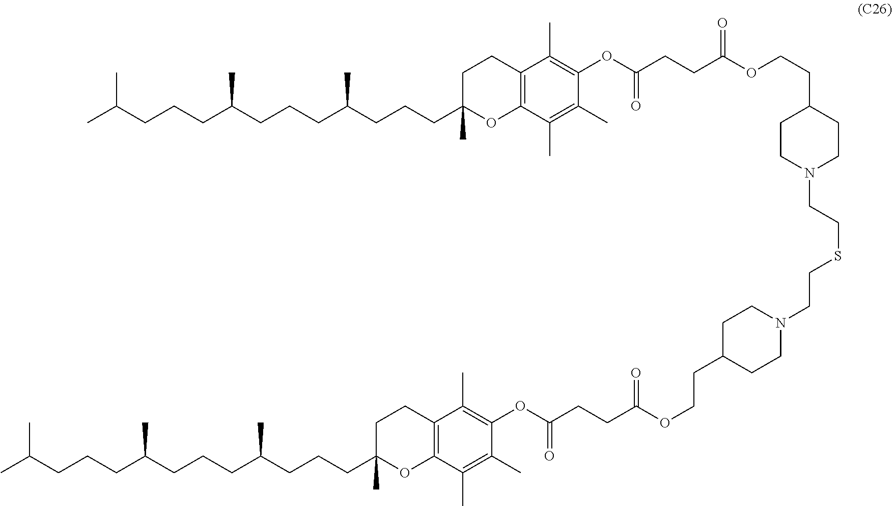 [SMILES: Cc1c(C)c2c(c(C)c1OC(=O)CCC(=O)OCCC1CCN(CCSCCN3CCC(CCOC(=O)CCC(=O)Oc4c(C)c(C)c5c(c4C)CC[C@@](C)(CCC[C@H](C)CCC[C@H](C)CCCC(C)C)O5)CC3)CC1)CC[C@@](C)(CCC[C@H](C)CCC[C@H](C)CCCC(C)C)O2]

In other embodiments, the lipid-based carriers of the pharmaceutical composition comprise a cationic lipid selected or derived from 9-Heptadecanyl 8-{(2-hydroxyethyl)[6-oxo-6-(undecyloxy)hexyl]amino}octanoate, also referred to as SM-102. Other preferred lipid-based carriers (e.g. LNPs) of the pharmaceutical composition comprise a squaramide ionizable amino lipid, more preferably a cationic lipid selected from the group consisting of formulas (M1) and (M2):

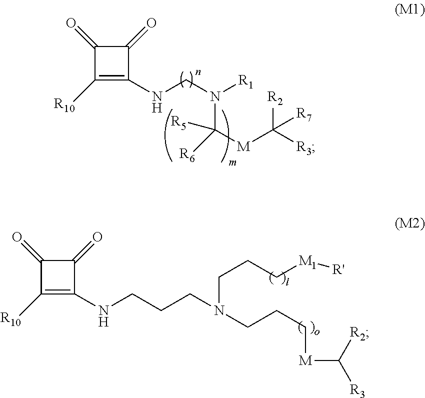

wherein the substituents (e.g. R₁, R₂, R₃, R₅, R₆, R₇, R₁₀, M, M₁, m, n, o, l) are defined in claims 1 to 13 of U.S. Pat. Nos. 10,392,341B2; 10,392,341B2 being incorporated herein in its entirety.

Accordingly, in preferred embodiments, the lipid-based carriers of the pharmaceutical composition comprise a cationic lipid selected or derived from above mentioned ALC-0315, SM-102, SS-33/4PE-15, HEXA-C5DE-PipSS, or compound C26 (see C26 in Table 1 of WO2021123332).

In preferred embodiments, the lipid-based carriers of the pharmaceutical composition comprise a cationic lipid ALC-0315.

In preferred embodiments the lipid-based carriers of the pharmaceutical composition comprising a cationic lipid ALC-0315 are used for treating eye disease.

In preferred embodiments the lipid-based carriers of the pharmaceutical composition comprising a cationic lipid ALC-0315 are used for treating EMT-associated diseases include pathologic ocular fibrosis and proliferation, for example PVR, conjunctival fibrosis (e.g. ocular cicatricial pemphigoid), corneal scarring, corneal epithelial down growth, and/or aberrant fibrosis, diseases in the anterior segment of the eye (e.g., comeal opacification and glaucoma), corneal dystrophies, herpetic keratitis, inflammation (e.g., pterygium), macula edema, retinal and vitreous hemorrhage, fibrovascular scarring, neovascular glaucoma, age-related macular degeneration (ARMD), geographic atrophy, diabetic retinopathy (DR), retinopathy of prematurity (ROP), subretinal fibrosis, epiretinal fibrosis, and gliosis. Other conditions associated with EMT including cancer, e.g., mesothelioma, ocular chronic graft-versus-host disease, corneal scarring, commeal epithelial downgrowth, conjunctival scarring, eye tumors such as melanoma and metastatic tumors, or fibrosis.

In preferred embodiments the lipid-based carriers of the pharmaceutical composition comprising a cationic lipid ALC-0315 are used for treating PVR.

In some embodiments, the lipid-based carriers of the invention comprise two or more (different) cationic lipids as defined herein.

In certain embodiments, the cationic lipid as defined herein, more preferably cationic lipid ALC-0315, is present in the lipid-based carriers in an amount from about 30 mol % to about 95 mol %, relative to the total lipid content of the lipid-based carriers. If more than one cationic lipid is incorporated within the lipid-based carriers, such percentages apply to the combined cationic lipids.

In certain embodiments, the cationic lipid as defined herein is present in the lipid-based carriers in an amount from about 30 to about 95 mole percent, relative to the total lipid content of the lipid-based carriers. If more than one cationic lipid is incorporated within the lipid-based carriers, such percentages apply to the combined cationic lipids.

In embodiments, the cationic lipid is present in the lipid-based carriers in an amount from about 30 to about 70 mole percent. In one embodiment, the cationic lipid is present in the lipid-based carriers in an amount from about 40 to about 60 mole percent, such as about 40, 41, 42, 43, 44, 45, 46, 47, 48, 49, 50, 51, 52, 53, 54, 55, 56, 57, 58, 59 or 60 mole percent, respectively. In embodiments, the cationic lipid is present in the lipid-based carriers in an amount from about 47 to about 48 mole percent, such as about 47.0, 47.1, 47.2, 47.3, 47.4, 47.5, 47.6, 47.7, 47.8, 47.9, 50.0 mole percent, respectively, wherein 47.4 mole percent are particularly preferred. In other preferred embodiments, the cationic lipid is present in the lipid-based carriers in an amount from about 55 to about 65 mole percent, such as about 55, 56, 57, 58, 59, 60, 61, 62, 63, 64 or 65 mole percent, respectively, wherein 59 mole percent are particularly preferred.

In some embodiments, the cationic lipid is present in a ratio of from about 20 mol % to about 70 or 75 mol % or from about 45 to about 65 mol % or about 20, 25, 30, 35, 40, 45, 50, 55, 60, 65, or about 70 mol % of the total lipid present in the lipid-based carriers. In further embodiments, the LNPs comprise from about 25% to about 75% on a molar basis of cationic lipid, e.g., from about 20 to about 70%, from about 35 to about 65%, from about 45 to about 65%, about 60%, about 57.5%, about 57.1%, about 50% or about 40% on a molar basis (based upon 100% total moles of lipid in the lipid nanoparticle). In a most preferred embodiment, the lipid-based carrier includes from about 59% on a molar basis of cationic or ionizable lipid.

In some embodiments, the ratio of cationic lipid to nucleic acid, preferably to RNA is from about 3 to about 15, such as from about 5 to about 13 or from about 7 to about 11.

### Neutral Lipids:

In preferred embodiments, the lipid-based carriers (e.g. LNPs) comprise at least one neutral lipid or phospholipid.

The term “neutral lipid” refers to any one of a number of lipid species that exist in either an uncharged or neutral zwitterionic form at physiological pH. Suitable neutral lipids include diacylphosphatidylcholines, diacylphosphatidylethanolamines, ceramides, sphingomyelins, dihydrosphingomyelins, cephalins, and cerebrosides. The selection of neutral lipids for use in the particles described herein is generally guided by consideration of, e.g., lipid particle size and stability of the lipid particle in the bloodstream. Preferably, the neutral lipid is a lipid having two acyl groups (e.g., diacylphosphatidylcholine and diacylphosphatidylethanolamine). In one embodiment, the neutral lipids contain saturated fatty acids with carbon chain lengths in the range of C10 to C20. In another embodiment, neutral lipids with mono or diunsaturated fatty acids with carbon chain lengths in the range of C10 to C20 are used. Additionally, neutral lipids having mixtures of saturated and unsaturated fatty acid chains can be used.

In some embodiments, the lipid-based carriers comprises one or more neutral lipids, wherein the neutral lipid is selected from the group comprising distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE), 1,2-diphytanoyl-sn-glycero-3-phosphoethanolamine (DPhyPE), or mixtures thereof.

In preferred embodiments, the neutral lipid of the lipid-based carriers of the pharmaceutical composition is selected or derived from 1,2-diheptanoyl-sn-glycero-3-phosphocholine (DHPC).

In other preferred embodiments, the neutral lipid of the lipid-based carriers of the pharmaceutical composition is selected or derived from 1,2-diphytanoyl-sn-glycero-3-phosphoethanolamine (DPhyPE).

Accordingly, in preferred embodiments, the lipid-based carriers of the pharmaceutical composition comprise a neutral lipid selected or derived from DSPC, DHPC, or DPhyPE. Therefore, in another embodiment, the invention is related to the use of a lipid with high fusogenicity in a lipid-based carrier or nucleic acid-lipid particle, preferably DPhyPE, as depicted here:

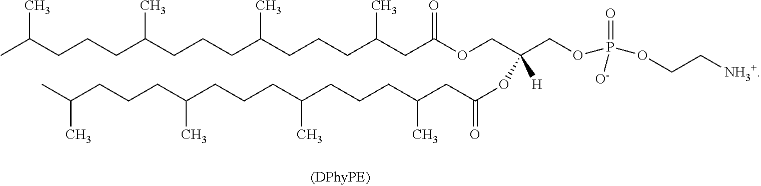

In preferred embodiments, the lipid-based carriers, preferably the LNPs of the pharmaceutical composition comprise a neutral lipid selected or derived from 1,2-distearoyl-sn-glycero-3-phosphocholine (DSPC).

In various embodiments, the molar ratio of the cationic lipid to the neutral lipid in the lipid-based carriers ranges from about 2:1 to about 8:1.

The neutral lipid is preferably from about 5 mol % to about 90 mol %, about 5 mol % to about 10 mol %, about 5, 10, 15, 20, 25, 30, 35, 40, 45, 50, 55, 60, 65, 70, 75, 80, 85, or about 90 mol % of the total lipid present in the lipid-based carrier. In one embodiment, the lipid-based carrier includes from about 0% to about 15% or 45% on a molar basis of neutral lipid, e.g., from about 3% to about 12% or from about 5% to about 10%. For instance, the lipid-based carrier may include about 15%, about 10%, about 7.5%, or about 7.1% of neutral lipid on a molar basis (based upon 100% total moles of lipid in the lipid-based carrier).

### Steroids, Steroid Analogs or Sterols:

In preferred embodiments, the lipid-based carriers of the pharmaceutical composition comprise a steroid, steroid analog or sterol.

Suitably, the steroid, steroid analog or sterol may be derived or selected from cholesterol, cholesteryl hemisuccinate (CHEMS) and a derivate thereof. In other embodiments, the lipid-based carriers of the pharmaceutical composition comprise a steroid, steroid analog or sterol derived from a phytosterol (e.g., a sitosterol, such as beta-sitosterol), preferably from a compound having the structure of Formula I as disclosed in claim 1 of WO2020061332; the disclosure of WO2020061332, especially the disclosure of Formula I and phytosterols being incorporated herewith by reference. In a further embodiment, the steroid is an imidazole cholesterol ester or “ICE” as disclosed in paragraphs [0320] and [0339]-[0340] of WO2019226925A1; WO2019226925A1 being incorporated herein by reference in its entirety.

In particularly preferred embodiments, the lipid-based carriers of the pharmaceutical composition comprise cholesterol.

The molar ratio of the cationic lipid to cholesterol in the lipid-based carriers may be in the range from about 2:1 to about 1:1. In some embodiments, the cholesterol may be PEGylated.

In some embodiments, the lipid-based carrier comprises about 10 mol % to about 60 mol % or about 25 mol % to about 40 mol % sterol (based on 100% total moles of lipids in the lipid-based carrier). In one embodiment, the sterol is about 10, 15, 20, 25, 30, 35, 40, 45, 50, 55, or about 60 mol % of the total lipid present in the lipid-based carrier. In another embodiment, the lipid-based carriers include from about 5% to about 50% on a molar basis of the sterol, e.g., about 15% to about 45%, about 20% to about 40%, about 48%, about 40%, about 38.5%, about 35%, about 34.4%, about 31.5% or about 30% on a molar basis (based upon 100% total moles of lipid in the lipid-based carrier). In preferred embodiments, the lipid-based carrier comprises about 28%, about 29% or about 30% sterol (based on 100% total moles of lipids in the lipid-based carrier). In most preferred embodiments, the lipid-based carrier comprises about 40.9% sterol (based on 100% total moles of lipids in the lipid-based carrier). In another most preferred embodiment, the lipid-based carrier includes from about 28.5% on a molar basis of sterol, preferably cholesterol.

### Aggregation Reducing Lipids/Polymer Conjugated Lipids:

In preferred embodiments, the lipid-based carriers comprise at least one aggregation reducing lipid or moiety.

The term “aggregation reducing moiety” refers to a molecule comprising a moiety suitable of reducing or preventing aggregation of the lipid-based carriers.

The term “aggregation reducing lipid” refers to a molecule comprising both a lipid portion and a moiety suitable of reducing or preventing aggregation of the lipid-based carriers. Under storage conditions or during formulation, the lipid-based carriers may undergo charge-induced aggregation, a condition which can be undesirable for the stability of the lipid-based carriers. Therefore, it can be desirable to include a compound or moiety which can reduce aggregation, for example by sterically stabilizing the lipid-based carriers. Such a steric stabilization may occur when a compound having a sterically bulky but uncharged moiety that shields or screens the charged portions of a lipid-based carriers from close approach to other lipid-based carriers in the composition. In the context of the invention, stabilization of the lipid-based carriers is achieved by including lipids which may comprise a lipid bearing a sterically bulky group which, after formation of the lipid-based carrier, is preferably located on the exterior of the lipid-based carrier. Suitable aggregation reducing groups include hydrophilic groups, e.g. monosialoganglioside GM1, polyamide oligomers (PAO), or certain polymers, such as poly(oxyalkylenes), e.g., poly(ethylene glycol) or poly(propylene glycol).

Lipids comprising a polymer as aggregation reducing group are herein referred to as “polymer conjugated lipid”.

The term “polymer conjugated lipid” refers to a molecule comprising both a lipid portion and a polymer portion, wherein the polymer is suitable of reducing or preventing aggregation of lipid-based carriers comprising the RNA. A polymer has to be understood as a substance or material consisting of very large molecules, or macromolecules, composed of many repeating subunits. A suitable polymer in the context of the invention may be a hydrophilic polymer. An example of a polymer conjugated lipid is a PEGylated or PEG-conjugated lipid.

In preferred embodiments, the lipid-based carriers of the pharmaceutical composition comprise an aggregation reducing lipid selected from a polymer conjugated lipid.

In preferred embodiments, the polymer conjugated lipid is a PEG-conjugated lipid (or PEGylated lipid, PEG lipid).

The average molecular weight of the PEG moiety in the PEG-conjugated lipid preferably ranges from about 500 to about 8,000 Daltons (e.g., from about 1,000 to about 4,000 Daltons). In one preferred embodiment, the average molecular weight of the PEG moiety is about 2,000 Daltons.

In some embodiments, the PEG-conjugated lipid is selected from PEG-modified phosphatidylethanolamine, PEG-modified phosphatidic acid, PEG-modified ceramides (e.g. PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol) 2000) carbamyl]-1,2-dimyristyloxlpropyl-3-amine (PEG-c-DMA). In a preferred embodiment, the polyethylene glycol-lipid is PEG-2000-DMG. In one embodiment, the polyethylene glycol-lipid is PEG-c-DOMG). In other embodiments, the LNPs comprise a PEGylated diacylglycerol (PEG-DAG) such as 1-(monomethoxy-polyethyleneglycol)-2,3-dimyristoylglycerol (PEG-DMG), a PEGylated phosphatidylethanoloamine (PEG-PE), a PEG succinate diacylglycerol (PEG-S-DAG) such as 4-O-(2′,3′-di (tetradecanoyloxy) propyl-1-O-(w-methoxy (polyethoxy) ethyl) butanedioate (PEG-S-DMG), a PEGylated ceramide (PEG-cer), or a PEG dialkoxypropylcarbamate such as ω-methoxy(polyethoxy)ethyl-N-(2,3di(tetradecanoxy)propyl)carbamate or 2,3-di(tetradecanoxy)propyl-N-(ω-methoxy (polyethoxy)ethyl)carbamate.

In preferred embodiments, the polymer conjugated lipid, e.g. the PEG-conjugated lipid, is selected or derived from 1,2-dimyristoyl-rac-glycero-3-methoxypolyethylene glycol-2000 (PEG2000 DMG or DMG-PEG 2000). As used in the art, “DMG-PEG 2000” is typically considered a mixture of 1,2-DMG PEG2000 and 1,3-DMG PEG2000 in ˜97:3 ratio.

In other embodiments, the polymer conjugated lipid, e.g. the PEG-conjugated lipid, is selected or derived from C10-PEG2K, or Cer8-PEG2K.

In preferred embodiments, the polymer conjugated lipid, e.g. the PEG-conjugated lipid is selected or derived from formula (IVa):

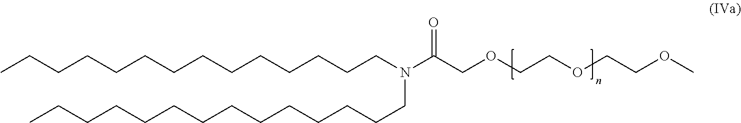

preferably wherein n has a mean value ranging from 30 to 60, such as about 30±2, 32±2, 34±2, 36±2, 38±2, 40±2, 42±2, 44±2, 46±2, 48±2, 50±2, 52±2, 54±2, 56±2, 58±2, or 60±2. In a most preferred embodiment n is about 49. In another very preferred embodiment n is 45. In further preferred embodiments said PEG lipid is of formula (IVa), wherein n is an integer selected such that the average molecular weight of the PEG lipid is about 2000 g/mol to about 3000 g/mol or about 2300 g/mol to about 2700 g/mol. In another preferred embodiment said PEG lipid is of formula (IVa), wherein n is an integer selected such that the average molecular weight of the PEG lipid is about 2000 g/mol.

The PEG-conjugated lipid of formula IVa as suitably used herein has the chemical 2[(polyethylene glycol)-2000]-N,N-ditetradecylacetamide, also referred to as ALC-0159.

Accordingly, in preferred embodiments, the aggregation reducing lipid is a PEG-conjugated lipid preferably selected or derived from DMG-PEG 2000, C10-PEG2K, Cer8-PEG2K, or ALC-0159.

In preferred embodiments, the lipid-based carriers of the pharmaceutical composition comprise a PEG-conjugated lipid ALC-0159.

In other preferred embodiments, the lipid-based carriers of the pharmaceutical composition comprise an aggregation reducing lipid, wherein the aggregation reducing lipid is not a PEG-conjugated lipid. Accordingly, the aggregation reducing lipid may suitably be selected from a PEG-less lipid, e.g. a PEG-less polymer conjugated lipid.

In some embodiments, lipid-based carriers include less than about 3, 2, or 1 mole percent of aggregation reducing lipid, based on the total moles of lipid in the lipid-based carrier. In further embodiments, lipid-based carriers comprise from about 0.1% to about 10% of the aggregation reducing lipid on a molar basis, e.g., about 0.5 to about 10%, about 0.5 to about 5%, about 10%, about 5%, about 4%, about 3%, about 2%, about 1.5%, about 1%, about 0.5%, or about 0.3% on a molar basis (based on 100% total moles of lipids in the lipid-based carrier). In other preferred embodiments, lipid-based carriers comprise from about 1.0% to about 2.0% of the aggregation reducing lipid on a molar basis, e.g., about 1.2 to about 1.9%, about 1.2 to about 1.8%, about 1.3 to about 1.8%, about 1.4 to about 1.8%, about 1.5 to about 1.8%, about 1.6 to about 1.8%, in particular about 1.4%, about 1.5%, about 1.6%, about 1.7%, about 1.8%, about 1.9%, most preferably 1.7% (based on 100% total moles of lipids in the lipid-based carrier). In other preferred embodiments, lipid-based carriers comprise about 2.5%, 3%, 3.5%, 4%, 4.5% or 5%, preferably 2.5% of the aggregation reducing lipid on a molar basis (based on 100% total moles of lipids in the lipid-based carrier). In various embodiments, the molar ratio of the cationic lipid to the aggregation reducing lipid ranges from about 100:1 to about 25:1.

In preferred embodiments, the lipid-based carriers of the pharmaceutical composition comprise an aggregation reducing lipid, wherein the aggregation reducing lipid is not a PEG-conjugated lipid. In preferred embodiments, the aggregation reducing lipid (or polymer conjugated lipid) is a PEG-free lipid that comprises a polymer different from PEG.

A PEG-free lipid in the context of the invention may be selected or derived from a POZ-lipid. In preferred embodiments, the POZ lipids or respectively preferred polymer conjugated lipids are described in PCT/EP2022/074439, the full disclosure herewith incorporated by reference. In particular, the disclosure relating to polymer conjugated lipids as shown in any one of claims 1 to 8 the disclosure relating to polymer conjugated lipids as shown in any one of claims 9 to 46 of PCT/EP2022/074439 are incorporated by reference.

In preferred embodiments, the lipid-based carriers of the pharmaceutical composition do not comprise a PEG-conjugated lipid. Accordingly, in further preferred embodiments, the polymer conjugated lipid is a POZ-lipid, which is defined as a compound according to formula (POZ):[H]-[linker]-[M]
wherein
[H] is a homopolymer moiety comprising at least one polyoxazoline (POZ) monomer unit

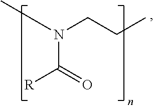

wherein R is C1-9 alkyl or C2-9 alkenyl and n has a mean value ranging from 2 to 200, preferably from 20 to 100, more preferably from 24 to 26 or 45 to 50
[linker] is an optional linker group, and
[M] is a lipid moiety.

In an embodiment in that context, [H] is a heteropolymer moiety or homopolymer moiety comprising multiple monomer units selected from the group consisting of
poly(2-methyl-2-oxazoline) (PMOZ)

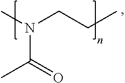

poly(2-ethyl-2-oxazoline) (PEOZ)

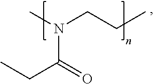

poly(2-propyl-2-oxazoline) (PPOZ)

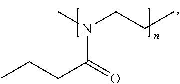

poly(2-butyl-2-oxazoline) (PBOZ)

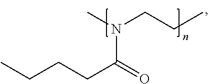

poly(2-isopropyl-2-oxazoline) (PIPOZ)

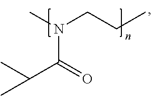

poly(2-methoxymethyl-2-oxazoline) (PMeOMeOx), and
poly(2-dimethylamino-2-oxazoline) (PDMAOx),
preferably wherein [H] is a homopolymer moiety comprising multiple PMOZ or PEOZ monomer units, more
preferably wherein [H] comprises or preferably consists of multiple PMOZ monomer units,
wherein
(i) n has a mean value ranging from 2 to 200, preferably from 20 to 100, more preferably from 24 to 26 or 45 to 50 or wherein
(ii) n is selected such that the [H] moiety has an average molecular weight of 1.5 to 22 kDa, more preferably of 2 to 19 kDa, even more preferably of about 7.5 kDa or of about 15 kDa, preferably from 1 to 15 kDa, more preferably of 2 to 12.5 kDa, even more preferably of about 5 kDa or of about 10 kDa.

In another embodiment in that context, [H] is a heteropolymer moiety or homopolymer moiety comprising multiple monomer units selected from the group consisting of

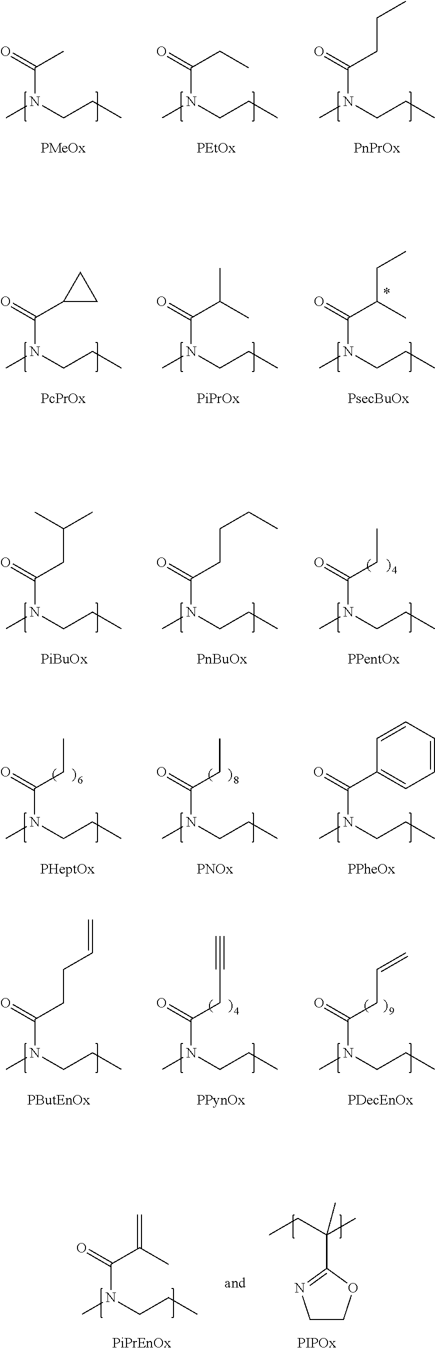

In yet another embodiment, the [H] from the polymer conjugated lipid according to formula (POZ) is selected from the group consisting of poly(2-methoxymethyl-2-oxazoline) (PMeOMeOx) and poly(2-dimethylamino-2-oxazoline) (PDMAOx).

In one embodiment, the lipid moiety [M] as shown in formula (POZ) comprises at least one straight or branched, saturated or unsaturated alkyl chain containing from 6 to 30 carbon atoms, preferably wherein the lipid moiety [M] comprises at least one straight or branched saturated alkyl chain, wherein the alkyl chain is optionally interrupted by one or more biodegradable group(s) and/or optionally comprises one terminal biodegradable group, wherein the biodegradable group is selected from the group consisting of but not limited to a pH-sensitive moiety, a zwitterionic linker, non-ester containing linker moieties and ester-containing linker moieties (—C(O)O— or —OC(O)—), amido (—C(O)NH—), disulfide (—S—S—), carbonyl (—C(O)—), ether (—O—), thioether (—S—), oxime (e.g., —C(H)═N—O— or —O—N═C(H)—), carbamate (—NHC(O)O—), urea (—NHC(O)NH—), succinyl (—(O)CCH2CH2C(O)—), succinamidyl (—NHC(O)CH2CH2C(O)NH—), —C(R5)=N—, —N═C(R5)-, —C(R5)=N—O—, —O—N═C(R5)-, —O—C(O)O—, —C(O)N(R5), —N(R5)C(O)—, —C(S)(NR5)-, (NR5)C(S)—, —N(R5)C(O)N(R5)-, —C(O)S—, —SC(O)—, —C(S)O—, —OC(S)—, —OSi(R5)2O—, —C(O)(CR3R4)C(O)O—, or —OC(O)(CR3R4)C(O)—, carbonate (—OC(O)O—), succinoyl, phosphate esters (—O—(O)POH—O—), cyclic compound, heterocyclic compound, piperidine, pyrazine, pyridine, piperazine, and sulfonate esters, as well as combinations thereof, wherein R3, R4 and R5 are, independently H or alkyl (e.g. C1-C4 alkyl).

In another embodiment, the the lipid moiety [M] comprises at least one straight or branched, saturated or unsaturated alkyl chain comprising 6, 7, 8, 9, 10, 11, 12, 13, 14, 15, 16, 17, 18, 19, 20, 21, 22, 23, 24, 25, 26, 27, 28, 29, or 30 carbon atoms, preferably in the range of 10 to 20 carbon atoms, more preferably in the range of 12 to 18 carbon atoms, even more preferably 14, 16 or 18 carbon atoms, even more preferably 16 or 18 carbon atoms, most preferably 14 carbon atoms, wherein all selections are independent of one another.

In one embodiment, the linker group [linker] as shown in formula (POZ) is selected from the group consisting of but not limited to a pH-sensitive moiety, a zwitterionic linker, non-ester containing linker moieties and ester-containing linker moieties (—C(O)O— or —OC(O)—), amido (—C(O)NH—), disulfide (—S—S—), carbonyl (—C(O)—), ether (—O—), thioether (—S—), oxime (e.g., —C(H)═N—O— or —O—N═C(H)—), carbamate (—NHC(O)O—), urea (—NHC(O)NH—), succinyl (—(O)CCH2CH2C(O)—), succinamidyl (—NHC(O)CH2CH2C(O)NH—), —C(R5)=N—, —N═C(R5)-, —C(R5)=N—O—, —O—N═C(R5)-, —O—C(O)O—, —C(O)N(R5), —N(R5)C(O)—, —OSi(R5)2O—, —C(O)(CR3R4)C(O)O—, or —OC(O)(CR3R4)C(O)—, carbonate (—OC(O)O—), succinoyl, phosphate esters (—O—(O)POH—O—), and sulfonate esters, as well as combinations thereof, wherein R3, R4 and R5 are, independently H or alkyl (e.g. C1-C4 alkyl).

In a very preferred embodiment, the polymer conjugated lipid has the structure of

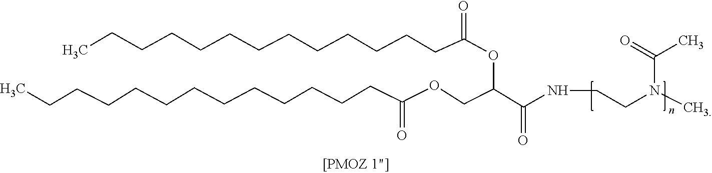

In another very preferred embodiment, the polymer conjugated lipid has the structure of

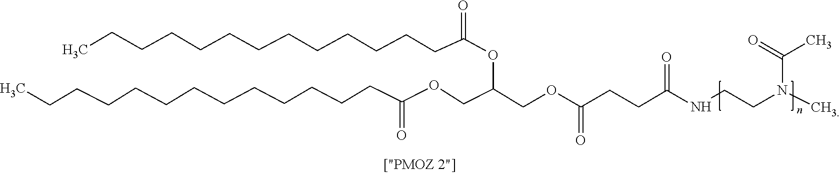

In another very preferred embodiment, the polymer conjugated lipid has the structure of “PMOZ 2” with n=50 i.e. having 50 monomer repeats.

In an even further preferred embodiment, the polymer conjugated lipid has the structure of

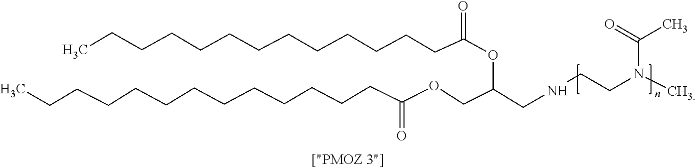

In another preferred embodiment, the polymer conjugated lipid has the structure of

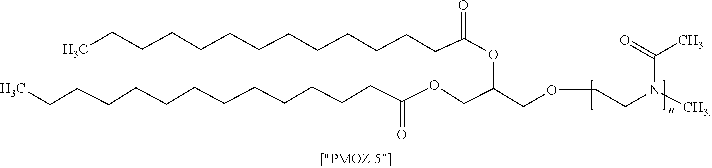

In an even further very preferred embodiment, the polymer conjugated lipid has the structure of

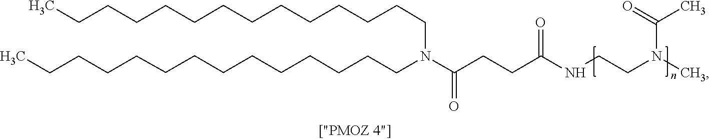

preferably with n=50 i.e. having 50 monomer repeats, i.e.

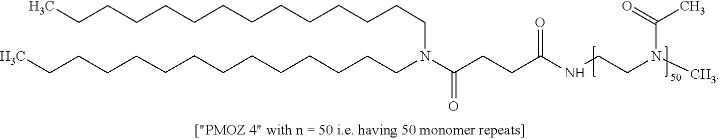

For “PMOZ 1” to “PMOZ 5”, preferably n has a mean value ranging from 2 to 200, preferably from 20 to 100, more preferably from 24 to 26, even more preferably about 100, or further even more preferably from 45 to 50, most preferably 50 or wherein n is selected such that the [P] moiety has an average molecular weight of about 4.2 kDa to about 4.4 kDa, or most preferably about 4.3 kDa.

In another very preferred embodiment, the linker group [linker] comprises preferably an amide linker moiety.

In a further very preferred embodiment, the linker group [linker] comprises preferably an ester linker moiety.

In a further very preferred embodiment, the linker group [linker] comprises preferably a succinate linker moiety.

In another very preferred embodiment, the linker group [linker] comprises both an ester linker and an amid linker moiety. In another preferred embodiment, the linker group [linker] comprises both an ester linker, an amine linker and an amid linker moiety.

It is noted herein, that all chemical compounds mentioned throughout the whole specification may be produced via processes known to a skilled worker; starting materials and/or reagents used in the processes are obtainable through routine knowledge of a skilled worker on the basis of common general knowledge (e.g. from text books or from e.g. patent applications WO2022173667, WO2009043027, WO2013067199, WO2010006282, WO2009089542, WO2016019340, WO2008106186, WO2020264505, and WO2020023947, the complete disclosure of said patent applications is incorporated by reference herein).

In yet a further embodiment, the lipid nanoparticle does not comprise a polyethylene glycol-(PEG)-lipid conjugate or a conjugate of PEG and a lipid-like material, and preferably do not comprise PEG and/or (ii) the polymer conjugated lipid of the invention does not comprise a sulphur group (—S—), a terminating nucleophile, and/or is covalently coupled to a biologically active ingredient is a nucleic acid compound selected from the group consisting of RNA, an artificial mRNA, chemically modified or unmodified messenger RNA (mRNA) comprising at least one coding sequence, self-replicating RNA, circular RNA, viral RNA, and replicon RNA; or any combination thereof, preferably wherein the biologically active ingredient is chemically modified mRNA or chemically unmodified mRNA, more preferably wherein the biologically active ingredient is chemically unmodified mRNA.

In another very preferred embodiment, the polymer conjugated lipid of the invention does not comprise sulphur(S) or a sulphur group (—S—).

In further preferred embodiments, lipid nanoparticles and/or polymer conjugated lipids may be selected from the lipid nanoparticles and/or lipids as disclosed in PCT/EP2022/074439 (i.e. lipids derived from formula I, II, and Ill of PCT/EP2022/074439, or lipid nanoparticles and/or lipids as specified in claims 1 to 46 of PCT/EP2022/074439), the disclosure of PCT/EP2022/074439 hereby incorporated by reference in its entirety.

In preferred embodiments, the at least one aggregation-reducing lipid, preferably the PEG-conjugated lipid, is selected or derived from ALC-0159, DMG-PEG 2000, C10-PEG2K, Cer8-PEG2K. In particularly preferred embodiments, the aggregation-reducing lipid is ALC-0159.

Alternatively, the aggregation reducing lipid is selected or derived a POZ-lipid, which is defined as a compound according to formula (POZ) as defined herein.

In some embodiments, lipid-based carriers include less than about 3 mol %, 2 mol %, or 1 mol % of aggregation reducing lipid, based on the total moles of lipid in the lipid-based carrier. In further embodiments, lipid-based carriers comprise from about 0.1% to about 10% of the aggregation reducing lipid on a molar basis, e.g. about 0.5% to about 10%, about 0.5% to about 5%, about 10%, about 5%, about 4%, about 3%, about 2%, about 1.5%, about 1%, about 0.5%, or about 0.3% on a molar basis (based on 100% total moles of lipids in the lipid-based carrier). In other preferred embodiments, lipid-based carriers comprise from about 1.0% to about 2.0% of the aggregation reducing lipid on a molar basis, e.g. about 1.2% to about 1.9%, about 1.2% to about 1.8%, about 1.3% to about 1.8%, about 1.4% to about 1.8%, about 1.5% to about 1.8%, about 1.6% to about 1.8%, in particular about 1.4%, about 1.5%, about 1.6%, about 1.7%, about 1.8%, about 1.9%, most preferably 1.7% (based on 100% total moles of lipids in the lipid-based carrier). In other preferred embodiments, lipid-based carriers comprise about 2.5%, 3%, 3.5%, 4%, 4.5% or 5%, preferably 2.5% of the aggregation reducing lipid on a molar basis (based on 100% total moles of lipids in the lipid-based carrier). In various embodiments, the molar ratio of the cationic lipid to the aggregation reducing lipid ranges from about 100:1 to about 25:1.

Accordingly, in a very preferred embodiment, the lipid-based carriers of the pharmaceutical composition preferably comprises 59 mol % of cationic lipid “C26” (described herein above and/or below), 10 mol % DPhyPE, 28.5 mol % cholesterol and 2.5 mol % PMOZ 4 (described herein above and/or below).

### Other Suitable Cationic or Ionizable, Neutral, Steroid/Sterol or Aggregation Reducing Lipids:

Other suitable cationic or ionizable, neutral, steroid/sterol or aggregation reducing lipids are disclosed in WO2010053572, WO2011068810, WO2012170889, WO2012170930, WO2013052523, WO2013090648, WO2013149140, WO2013149141, WO2013151663, WO2013151664, WO2013151665, WO2013151666, WO2013151667, WO2013151668, WO2013151669, WO2013151670, WO2013151671, WO2013151672, WO2013151736, WO2013185069, WO2014081507, WO2014089486, WO2014093924, WO2014144196, WO2014152211, WO2014152774, WO2014152940, WO2014159813, WO2014164253, WO2015061461, WO2015061467, WO2015061500, WO2015074085, WO2015105926, WO2015148247, WO2015164674, WO2015184256, WO2015199952, WO2015200465, WO2016004318, WO2016022914, WO2016036902, WO2016081029, WO2016118724, WO2016118725, WO2016176330, WO2017004143, WO2017019935, WO2017023817, WO2017031232, WO2017049074, WO2017049245, WO2017070601, WO2017070613, WO2017070616, WO2017070618, WO2017070620, WO2017070622, WO2017070623, WO2017070624, WO2017070626, WO2017075038, WO2017075531, WO2017099823, WO2017106799, WO2017112865, WO2017117528, WO2017117530, WO2017180917, WO2017201325, WO2017201340, WO2017201350, WO2017201352, WO2017218704, WO2017223135, WO2018013525, WO2018081480, WO2018081638, WO2018089540, WO2018089790, WO2018089801, WO2018089851, WO2018107026, WO2018118102, WO2018119163, WO2018157009, WO2018165257, WO2018170245, WO2018170306, WO2018170322, WO2018170336, WO2018183901, WO2018187590, WO2018191657, WO2018191719, WO2018200943, WO2018231709, WO2018231990, WO2018232120, WO2018232357, WO2019036000, WO2019036008, WO2019036028, WO2019036030, WO2019040590, WO2019089818, WO2019089828, WO2019140102, WO2019152557, WO2019152802, WO2019191780, WO2019222277, WO2019222424, WO2019226650, WO2019226925, WO2019232095, WO2019232097, WO2019232103, WO2019232208, WO2020061284, WO2020061295, WO2020061332, WO2020061367, WO2020081938, WO2020097376, WO2020097379, WO2020097384, WO2020102172, WO2020106903, WO2020146805, WO2020214946, WO2020219427, WO2020227085, WO2020232276, WO2020243540, WO2020257611, WO2020257716, WO2021007278, WO2021016430, WO2021022173, WO2021026358, WO2021030701, WO2021046260, WO2021050986, WO2021055833, WO2021055835, WO2021055849, WO2021127394, WO2021127641, WO2021202694, WO2021231697, WO2021231901, WO2008103276, WO2009086558, WO2009127060, WO2010048536, WO2010054406, WO2010080724, WO2010088537, WO2010129709, WO201021865, WO2011022460, WO2011043913, WO2011090965, WO2011149733, WO2011153120, WO2011153493, WO2012040184, WO2012044638, WO2012054365, WO2012061259, WO2013063468, WO2013086354, WO2013086373, U.S. Pat. Nos. 7,893,302B2, 7,404,969B2, 8,158,601B2, 8,283,333B2, 8,466,122B2, 8,569,256B2, US20100036115, US20110256175, US20120202871, US20120027803, US20120128760, US20130064894, US20130129785, US20130150625, US20130178541, US20130225836, and US20140039032; the disclosures specifically relating to cationic or ionizable, neutral, sterol or aggregation reducing lipids suitable for lipid-based carriers of the foregoing publications are incorporated herewith by reference.

For example, suitable cationic lipids or cationisable or ionizable lipids include, but are not limited to, DSDMA, N,N-dioleyl-N,N-dimethylammonium chloride (DODAC), N,N-distearyl-N,N-dimethylammonium bromide (DDAB), 1,2-dioleoyltrimethyl ammonium propane chloride (DOTAP) (also known as N-(2,3-dioleoyloxy) propyl)-N,N,N-trimethylammonium chloride and 1,2-Dioleyloxy-3-trimethylaminopropane chloride salt), N-(1-(2,3-dioleyloxy) propyl)-N,N,N-trimethylammonium chloride (DOTMA), N,N-dimethyl-2,3-dioleyloxy) propylamine (DODMA), ckk-E12 (WO2015200465), 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (γ-DLenDMA), 98N12-5, 1,2-Dilinoleylcarbamoyloxy-3-dimethylaminopropane (DLin-C-DAP), ICE (Imidazol-based), HGT5000, HGT5001, DMDMA, CLinDMA, CpLinDMA, DMOBA, DOcarbDAP, DLincarbDAP, DLinCDAP, KLin-K-DMA, DLin-K-XTC2-DMA, XTC (2,2-Dilinoleyl-4-dimethylaminoethyl-[1,3]-dioxolane) HGT4003, 1,2-Dilinoleoyl-3-trimethylaminopropane chloride salt (DLin-TAP.Cl), 1,2-Dilinoleyloxo-3-(2-N,N-dimethylamino) ethoxypropane (DLin-EG-DM A), 2,2-Dilinoleyl-4-dimethylaminomethyl-[1,3]-dioxolane (DLin-K-DMA), (6Z,9Z,28Z,31Z)-heptatriaconta-6,9,28,31-tetraen-19-yl-4-(dimethylamino) butanoate (MC3, US20100324120), ALNY-100 ((3aR,5s,6aS)-N,N-dimethyl-2,2-di((9Z,12Z)-octadeca-9,12-dienyl) tetrahydro-3aH-cyclopenta[d][1,3]dioxol-5-amine)), NC98-5 (4,7,13-tris(3-oxo-3-(undecylamino) propyl)-N,N 16-diundecyl-4,7,10,13-tetraazahexadecane-1,16-diamide), (6Z,9Z,28Z,31Z)-heptatriaconta-6,9,28,31-tetraen-19-yl 4-(dimethylamino) butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31Z)-heptatriaconta-6,9,28,31-tetraen-19-yloxy)-N,N-dimethylbutan-1-amine (MC4 Ether), LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy) propyl)-N-(2-(sperminecarboxamido) ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.) or any combination of any of the foregoing. Further suitable cationic or ionizable lipids include those described in international patent publications WO2010053572 (and particularly, 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200) described at paragraph [00225] of WO2010053572) and WO2012170930, both of which are incorporated herein by reference, HGT4003, HGT5000, HGTS001, HGT5001, HGT5002 (see US2015140070), 1,2-dilinoleyoxy-3-(dimethylamino) acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.Cl), 1,2-dilinoleyloxy-3-(N-methylpiperazino) propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino) ethoxypropane (DLin-EG-DMA), 2,2-dilinoleyl-4-(2-dimethylaminoethyl)-[1,3]-dioxolane (DLin-KC2-DMA, WO2010042877); dilinoleyl-methyl-4-dimethylaminobutyrate (DLin-MC3-DMA).

### Lipid-Based Carrier Compositions:

In preferred embodiments, the lipid-based carriers of the pharmaceutical composition comprise at least one nucleic acid, preferably at least one RNA encoding a transcription factor inhibitor as defined herein, a cationic lipid as defined herein, an aggregation reducing lipid as defined herein, optionally, a neutral lipid as defined herein, and, optionally, a steroid or steroid analog as defined herein.

In embodiments, the lipid-based carriers comprising the at least one nucleic acid, preferably the at least one RNA comprise
(i) at least one cationic lipid or ionizable lipid, preferably as defined herein;
(ii) at least one neutral lipid or phospholipid, preferably as defined herein;
(iii) at least one steroid or steroid analogue, preferably as defined herein; and
(iv) at least one aggregation reducing lipid, preferably as defined herein.

In other embodiments, the lipid-based carriers comprising the at least one nucleic acid, preferably the at least one RNA comprise
(i) at least one cationic lipid or ionizable lipid, preferably as defined herein;
(ii) at least one neutral lipid or phospholipid, preferably as defined herein or at least one steroid or steroid analogue, preferably as defined herein; and
(iii) at least one aggregation reducing lipid, preferably as defined herein.

In preferred embodiments, the lipid-based carriers comprising the at least one nucleic acid, preferably the at least one RNA comprise
(i) at least one cationic lipid selected or derived from ALC-0315, SM-102, SS-33/4PE-15, HEXA-C5DE-PipSS or compound C26 (see C26 in Table 1 of WO2021123332);
(ii) at least one neutral lipid selected or derived from DSPC, DHPC, or DPhyPE;
(iii) at least one steroid or steroid analog selected or derived from cholesterol; and
(iv) at least one aggregation reducing lipid selected or derived from DMG-PEG 2000, C10-PEG2K, Cer8-PEG2K, or ALC-0159 or “PMOZ 4”; and wherein the lipid-based carriers encapsulate the nucleic acid (e.g. the RNA).

In a preferred embodiment, the lipid-based carriers comprising the at least one nucleic acid, preferably the at least one RNA comprise
(i) at least one cationic lipid selected or derived from ALC-0315;
(ii) at least one neutral lipid selected or derived from DSPC;
(iii) at least one steroid or steroid analog selected or derived from cholesterol; and
(iv) at least one aggregation reducing lipid selected or derived from ALC-0159; and wherein the lipid-based carriers encapsulate the nucleic acid (e.g. the RNA).

In preferred embodiments, the cationic lipids (as defined herein), neutral lipid (as defined herein), steroid or steroid analog (as defined herein), and/or aggregation reducing lipid (as defined herein) may be combined at various relative ratios.

In preferred embodiments, the lipid-based carriers comprise (i) to (iv) in a molar ratio of about 20-60% cationic lipid or ionizable lipid, about 5-25% neutral lipid, about 25-55% steroid or steroid analogue, and about 0.5-15% aggregation reducing lipid e.g. polymer conjugated lipid, preferably wherein the lipid-based carriers encapsulate the nucleic acid (e.g. the RNA).

For example, the ratio of cationic lipid or ionizable lipid to neutral lipid to steroid or steroid analogue to aggregation reducing lipid may be between about 30-60:20-35:20-30:1-15, or at a ratio of about 40:30:25:5, 50:25:20:5, 50:20:25:5, 50:27:20:3, 40:30:20:10, 40:32:20:8, 40:32:25:3 or 40:33:25:2, respectively.

In preferred embodiments, the lipid-based carriers, preferably the LNPs comprising at least one RNA of the first aspect comprise
at least one cationic lipid selected from SM-102;
at least one neutral lipid selected from DSPC;
at least one steroid or steroid analogue selected from cholesterol; and
at least one aggregation reducing lipid selected from DMG-PEG 2000 or “PMOZ 4”;
and
wherein the lipid-based carriers encapsulate the RNA, preferably wherein i) to (iv) are n a weight ratio of about 50% cationic lipid, about 10% neutral lipid, about 38.5% steroid or steroid analogue, and about 1.5% aggregation reducing lipid,
preferably wherein the lipid-based carriers encapsulate the RNA. Such LNPs are herein referred to as SM-102-LNPs.

In preferred embodiments, the lipid-based carriers, preferably the LNPs comprising at least one RNA of the first aspect comprise
at least one cationic lipid selected from SM-102;
at least one neutral lipid selected from DSPC;
at least one steroid or steroid analogue selected from cholesterol; and
at least one aggregation reducing lipid selected from DMG-PEG 2000 or “PMOZ 4”;
and
wherein the lipid-based carriers encapsulate the RNA, preferably wherein i) to (iv) are n a weight ratio of about 48.5% cationic lipid, about 11.1% neutral lipid, about 38.9% steroid or steroid analogue, and about 1.5% aggregation reducing lipid,
preferably wherein the lipid-based carriers encapsulate the RNA. A preferred N/P ratio for this formulation is about 4.85 (lipid to RNA mol ratio). Such LNPs are herein referred to as SM-102-LNPs.

In preferred embodiments, the lipid-based carriers, preferably the LNPs comprising the at least one nucleic acid, preferably the at least one RNA comprise
(i) at least one cationic lipid selected from SS-33/4PE-15, HEXA-C5DE-PipSS or compound C26 (see C26 in Table 1 of WO2021123332);
(ii) at least one neutral lipid selected from DPhyPE;
(iii) at least one steroid or steroid analog selected from cholesterol; and
(iv) at least one aggregation reducing lipid selected from DMG-PEG 2000 or most preferably “PMOZ 4”
and wherein the lipid-based carriers encapsulate the RNA, preferably wherein the lipid-based carriers encapsulate the nucleic acid (e.g. the RNA). Such LNPs are herein referred to as GN-LNPs.

In another preferred embodiment, lipid-based carriers, preferably the LNPs comprising the nucleic acid (e.g. the RNA) comprise 59 mol % HEXA-C5DE-PipSS lipid (see compound C2 in Table 1 of WO2021123332) as cationic lipid or preferably 59 mol % compound C26 (see C26 in Table 1 of WO2021123332), 10 mol % DPhyPE as neutral lipid, 29.3 mol % cholesterol as steroid and 1.7 mol % DMG-PEG 2000 as aggregation reducing lipid, or further preferably 59 mol % compound C26 (see C26 in Table 1 of WO2021123332), 10 mol % DPhyPE as neutral lipid, 28.5 mol % cholesterol as steroid and 2.5 mol % aggregation reducing lipid, preferably DMG-PEG 2000 or most preferably “PMOZ 4”.

In particularly preferred embodiments, the lipid-based carriers, preferably the LNPs comprising the at least one nucleic acid, preferably the at least one RNA, comprise
(i) at least one cationic lipid selected from ALC-0315;
(ii) at least one neutral lipid selected from DSPC;
(iii) at least one steroid or steroid analog selected from cholesterol; and
(iv) at least one aggregation reducing lipid selected from ALC-0159 and wherein the lipid-based carriers encapsulate the nucleic acid (e.g. the RNA), preferably wherein i) to (iv) are in a molar ratio of about 47.4% cationic lipid, about 10% neutral lipid, about 40.9% steroid or steroid analogue, and about 1.7% aggregation reducing lipid, preferably wherein the lipid-based carriers encapsulate the nucleic acid (e.g. the RNA). Such LNPs are herein referred to as 315-LNPs.

In particularly preferred embodiments, the lipid-based carriers, preferably the LNPs comprising the at least one nucleic acid, preferably the at least one RNA, comprise
(i) at least one cationic lipid selected from ALC-0315;
(ii) at least one neutral lipid selected from DSPC;
(iii) at least one steroid or steroid analog selected from cholesterol; and
(iv) at least one aggregation reducing lipid selected from ALC-0159 and wherein the lipid-based carriers encapsulate the nucleic acid (e.g. the RNA), preferably wherein i) to (iv) are in a molar ratio of about 47.4% cationic lipid, about 10% neutral lipid, about 40.9% steroid or steroid analogue, and about 1.7% aggregation reducing lipid, preferably wherein the lipid-based carriers encapsulate the nucleic acid (e.g. the RNA). Such LNPs are herein referred to as 315-LNPs. In such preferred embodiments, the RNA is preferably an mRNA that comprises a cap1 structure and an RNA sequence that is not chemically modified (e.g. consisting of non-modified ribonucleotides).

In particularly preferred embodiments, the lipid-based carriers, preferably the LNPs comprising the at least one nucleic acid, preferably the at least one RNA, comprise
(i) at least one cationic lipid selected from ALC-0315;
(ii) at least one neutral lipid selected from DSPC;
(iii) at least one steroid or steroid analog selected from cholesterol; and
(iv) at least one aggregation reducing lipid selected from ALC-0159 and wherein the lipid-based carriers encapsulate the nucleic acid (e.g. the RNA), preferably wherein i) to (iv) are in a molar ratio of about 47.4% cationic lipid, about 10% neutral lipid, about 40.9% steroid or steroid analogue, and about 1.7% aggregation reducing lipid, preferably wherein the lipid-based carriers encapsulate the nucleic acid (e.g. the RNA). Such LNPs are herein referred to as 315-LNPs. In such preferred embodiments, the RNA is preferably an mRNA that comprises a cap1 structure and an RNA sequence wherein all uracils are substituted by pseudouridine (ψ) or N (1)-methylpseudouridine (m1ψ).

In particular preferred embodiments, 315 LNPs and 315-like LNPs are used herein for treating eye disease

In particular preferred embodiments, the pharmaceutical composition comprises lipid nanoparticles (LNPs) which have a molar ratio of approximately 50:10:38.5:1.5, preferably 47.5:10:40.8:1.7 or more preferably 47.4:10:40.9:1.7 (i.e. proportion (mol %) of cationic lipid (preferably above mentioned lipid III-3 (ALC-0315)), DSPC, cholesterol and PEG-lipid (preferably above mentioned PEG-lipid of formula (IVa) with n=49, even more preferably above mentioned PEG-lipid of formula (IVa) with n=45 (ALC-0159)); solubilized in ethanol).

In preferred embodiments, the wt/wt ratio of lipid to nucleic acid (e.g. RNA) in the lipid-based carrier is from about 10:1 to about 60:1, e.g. about 40:1. In particularly preferred embodiments, the wt/wt ratio of lipid to nucleic acid is from about 20:1 to about 30:1, e.g. about 25:1. In other preferred embodiments, the wt/wt ratio of lipid to nucleic acid is in the range of 20 to 60, preferably from about 3 to about 15, about 5 to about 13, about 4 to about 8 or from about 7 to about 11.

The amount of lipid comprised in the lipid-based carriers may be selected taking the amount of the nucleic acid cargo into account. In one embodiment, these amounts are selected such as to result in an N/P ratio of the lipid-based carriers encapsulating the nucleic acid in the range of about 0.1 to about 20. The N/P ratio is defined as the mole ratio of the nitrogen atoms (“N”) of the basic nitrogen-containing groups of the lipid to the phosphate groups (“P”) of the nucleic acid which is used as cargo. The N/P ratio may be calculated on the basis that, for example, 1 μg nucleic acid typically contains about 3 nmol phosphate residues, provided that the nucleic acid exhibits a statistical distribution of bases. The “N”-value of the lipid or lipidoid may be calculated on the basis of its molecular weight and the relative content of permanently cationic and—if present—cationisable groups.

In embodiments, the N/P ratio can be in the range of about 1 to about 50. In other embodiments, the range is about 1 to about 20, and preferably about 1 to about 15. For “GN-LNPs”, a suitable N/P (lipid to nucleic acid mol ratio) is about 14 or about 17. For “315 LNPs”, a suitable N/P (lipid to nucleic acid mol ratio) is about 6. Another preferred N/P ratio is about 4.85 or 5 (lipid to RNA mol ratio).

In particularly preferred embodiments, the pharmaceutical composition comprises at least one RNA encoding at least one transcription factor inhibitor, wherein the transcription factor inhibitor, preferably the RUNX inhibitor, comprises or consists of an amino acid sequence being identical or at least 90%, 91%, 92%, 93%, 94%, 95%, 96%, 97%, 98%, or 99% identical to any one of SEQ ID NO: 232, or fragments or variants thereof, wherein the nucleic acid is encapsulated in lipid based carriers, preferably in GN-LNPs as defined herein or 315 LNPs as defined herein, wherein 315 LNPs are particularly preferred Alternatively in that context, the nucleic acid is encapsulated in SM-102 LNPs as defined herein.

In particularly preferred embodiments, the pharmaceutical composition comprises at least one RNA that comprises or consists of a nucleic acid sequence which is identical or at least 90%, 91%, 92%, 93%, 94%, 95%, 96%, 97%, 98%, or 99% identical to a nucleic acid sequence selected from SEQ ID NOs: 1579 or 1580, or a fragment or variant of that sequence, wherein the nucleic acid is encapsulated in lipid based carriers, preferably in GN-LNPs as defined herein or 315 LNPs as defined herein, wherein 315 LNPs are particularly preferred Alternatively in that context, the nucleic acid is encapsulated in SM-102 LNPs as defined herein.

In particularly preferred embodiments, the pharmaceutical composition comprises at least one RNA that comprises or consists of a nucleic acid sequence which is identical or at least 90%, 91%, 92%, 93%, 94%, 95%, 96%, 97%, 98%, or 99% identical to a nucleic acid sequence selected from SEQ ID NO: 1580, or a fragment or variant of that sequence, wherein the nucleic acid is encapsulated in lipid based carriers, preferably in GN-LNPs as defined herein or 315 LNPs as defined herein, wherein 315 LNPs are particularly preferred. Alternatively in that context, the nucleic acid is encapsulated in SM-102 LNPs as defined herein.

In various embodiments, the pharmaceutical composition comprises lipid-based carriers (encapsulating nucleic acid, preferably RNA as defined herein) that have a defined size (particle size, homogeneous size distribution).

The size of the lipid-based carriers of the pharmaceutical composition is typically described herein as Z-average size. The terms “average diameter”, “mean diameter”, “diameter” or “size” for particles (e.g. lipid-based carrier) are used synonymously with the value of the Z-average. The term “Z-average size” refers to the mean diameter of particles as measured by dynamic light scattering (DLS) with data analysis using the so-called cumulant algorithm, which provides as results the so-called Z-average with the dimension of a length, and the polydispersity index (PI), which is dimensionless (Koppel, D., J. Chem. Phys. 57, 1972, pp 4814-4820, ISO 13321).

The term “dynamic light scattering” or “DLS” refers to a method for analyzing particles in a liquid, wherein the liquid is typically illuminated with a monochromatic light source and wherein the light scattered by particles in the liquid is detected. DLS can thus be used to measure particle sizes in a liquid. Suitable DLS protocols are known in the art. DLS instruments are commercially available (such as the Zetasizer Nano Series, Malvern Instruments, Worcestershire, UK). DLS instruments employ either a detector at 90° (e.g., DynaPro® NanoStar® from Wyatt Technology or Zetasizer Nano S90® from Malvern Instruments) or a backscatter detection system at 173° (e.g., Zetasizer Nano S® from Malvern Instruments) and at 158° (DynaPro Plate Reader® from Malvern Instruments) close to the incident light of 180°. Typically, DLS measurements are performed at a temperature of about 25° C. DLS is also used in the context of the present invention to determine the polydispersity index (PDI) and/or the main peak diameter of the lipid-based carriers incorporating nucleic acid (e.g. RNA).

In various embodiments, the lipid-based carriers of the pharmaceutical composition encapsulating the nucleic acid (e.g. RNA) have a Z-average size ranging from about 50 nm to about 200 nm, from about 50 nm to about 190 nm, from about 50 nm to about 180 nm, from about 50 nm to about 170 nm, from about 50 nm to about 160 nm, 50 nm to about 150 nm, 50 nm to about 140 nm, 50 nm to about 130 nm, 50 nm to about 120 nm, 50 nm to about 110 nm, 50 nm to about 100 nm, 50 nm to about 90 nm, 50 nm to about 80 nm, 50 nm to about 70 nm, 50 nm to about 60 nm, 60 nm to about 200 nm, from about 60 nm to about 190 nm, from about 60 nm to about 180 nm, from about 60 nm to about 170 nm, from about 60 nm to about 160 nm, 60 nm to about 150 nm, 60 nm to about 140 nm, 60 nm to about 130 nm, 60 nm to about 120 nm, 60 nm to about 110 nm, 60 nm to about 100 nm, 60 nm to about 90 nm, 60 nm to about 80 nm, or 60 nm to about 70 nm, for example about 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 160 nm, 170 nm, 180 nm, 190 nm, or 200 nm.

In preferred embodiments, the lipid-based carriers of the pharmaceutical composition encapsulating the nucleic acid (e.g. RNA) have a Z-average size ranging from about 50 nm to about 200 nm, preferably in a range from about 50 nm to about 150 nm, more preferably from about 50 nm to about 120 nm.

Preferably, the pharmaceutical composition comprises less than about 10%, 9%, 8%, 7%, 6%, 5%, 4%, 3%, 2%, 1% lipid-based carriers that have a particle size exceeding about 500 nm.

Preferably, the pharmaceutical composition comprises less than about 10%, 9%, 8%, 7%, 6%, 5%, 4%, 3%, 2%, 1% LNPs that have a particle size smaller than about 20 nm.

Preferably, at least about 80%, 85%, 90%, 95% of lipid-based carriers have a spherical morphology.

In preferred embodiments, the polydispersity index (PDI) of the lipid-based carriers is typically in the range of 0.1 to 0.5. In a particular embodiment, a PDI is below 0.2. Typically, the PDI is determined by dynamic light scattering.

In preferred embodiments, 80% of the nucleic acid (e.g. RNA) comprised in the pharmaceutical composition is encapsulated in lipid-based carriers, preferably 85% of the nucleic acid (e.g. RNA) comprised in the pharmaceutical composition is encapsulated in lipid-based carriers, more preferably 90% of the nucleic acid (e.g. RNA) comprised in the pharmaceutical composition is encapsulated in lipid-based carriers, most preferably 95% of the nucleic acid (e.g. RNA) comprised in the pharmaceutical composition is encapsulated in lipid-based carriers. The percentage of encapsulation may be determined by a RiboGreen assay as known in the art.

The lipid-based carriers of the pharmaceutical composition have been prepared using according to the general procedures described in PCT Pub. Nos. WO2015199952, WO2017004143 and WO2017075531, the full disclosures of which are incorporated herein by reference.

According to a preferred embodiments the lipid-based carriers, preferably the LNPs encapsulating or comprising the nucleic acid (e.g. RNA), are purified by at least one purification step, preferably by at least one step of TFF and/or at least one step of clarification and/or at least one step of filtration.

Accordingly, in preferred embodiments, the pharmaceutical composition comprises purified lipid-based carriers encapsulating an mRNA encoding the transcription factor inhibitor as defined herein.

### Antagonists of RNA Sensing Pattern Recognition Receptors:

In preferred embodiments, in particular in embodiments where the nucleic acid of the composition is an RNA, the pharmaceutical composition may comprise at least one antagonist of at least one RNA sensing pattern recognition receptor.

In preferred embodiments in that context, the pharmaceutical composition comprises at least one antagonist of at least one RNA sensing pattern recognition receptor selected from a Toll-like receptor, preferably a TLR7 antagonist and/or a TLR8 antagonist.

Suitable antagonist of at least one RNA sensing pattern recognition receptor are disclosed in published PCT patent application WO2021028439, the full disclosure herewith incorporated by reference. In particular, the disclosure relating to suitable antagonist of at least one RNA sensing pattern recognition receptors as defined in any one of the claims 1 to 94 of WO2021028439 are incorporated by reference.

In preferred embodiments, the at least one antagonist of at least one RNA sensing pattern recognition receptor is a single stranded oligonucleotide that comprises or consists of a nucleic acid sequence being identical or at least 90%, 91%, 92%, 93%, 94%, 95%, 96%, 97%, 98%, or 99% identical to a nucleic acid sequence selected from the group consisting of SEQ ID NOs: 85-212 of WO2021028439, or fragments of any of these sequences. A particularly preferred antagonist in that context is 5′-GAG CGmG CCA-3′ (SEQ ID NO: 85 of WO2021028439), or a fragment or variant thereof.

In preferred embodiments, the molar ratio of the at least one antagonist of at least one RNA sensing pattern recognition receptor to the at least one RNA suitably ranges from about 20:1 to about 80:1.

In preferred embodiments, the weight to weight ratio of the at least one antagonist of at least one RNA sensing pattern recognition receptor to the at least one RNA suitably ranges from about 1:2 to about 1:10.

In embodiments, the at least one antagonist of at least one RNA sensing pattern recognition receptor and the at least one RNA encoding are separately formulated in the lipid-based carriers as defined herein or co-formulated in the lipid-based carriers as defined herein.

### Other Inhibitors:

In embodiments, the pharmaceutical composition additionally comprises at least one small molecule inhibitor or an inhibitory nucleic acid (siRNA) of the target transcription factor, preferably a small molecule inhibitor or an inhibitory nucleic acid (siRNA) of RUNX.

For example, any of the inhibitors of RUNX may be used that are provided in WO2019099560, WO2018093797, WO2019099595, and WO2021216378, the full disclosure herewith incorporated by reference. A suitable small molecule is ro5-3335 (see e.g. WO2018093797). The CAS Registry Number for Ro5-3335 is 30195-30-3.

### Presentation of the Pharmaceutical Composition:

In preferred embodiments, the pharmaceutical composition is a liquid composition or a dried composition.

In preferred embodiments, the pharmaceutical composition is a lyophilized, a spray-dried or a spray-freeze dried composition. The pharmaceutical composition is lyophilized (e.g. according to WO2016165831 or WO2011069586) to yield a temperature stable and dried composition. The pharmaceutical composition may also be dried using spray-drying or spray-freeze drying (e.g. according to WO2016184575 or WO2016184576) to yield a temperature stable dried composition.

Lyoprotectants for lyophilization and or spray drying may be selected from trehalose, sucrose, mannose, dextran and inulin. A preferred lyoprotectant is sucrose, optionally comprising a further lyoprotectant. A further preferred lyoprotectant is trehalose, optionally comprising a further lyoprotectant. Accordingly, the pharmaceutical composition may comprise at least one lyoprotectant.

In preferred embodiments, the pharmaceutical composition is a liquid composition or a lyophilized/spray-dried composition reconstituted in a liquid carrier.

In preferred embodiments, the pharmaceutical composition is a liquid composition.

In preferred embodiments, the pharmaceutical composition (or the liquid carrier) comprises at least one sugar preferably in a concentration of about 50 mM to about 300 mM, and/or a at least one salt preferably in a concentration of about 10 mM to about 200 mM, and/or at least one buffering agent. In preferred embodiments, the pharmaceutical composition (or the liquid carrier) has a pH in a range of about pH 7.0 to about pH 8.0.

### Administration of the Pharmaceutical Composition or the Nucleic Acid

Suitably, upon administration of the pharmaceutical composition or nucleic acid to a cell, tissue, or subject, the transcription factor inhibitor, e.g. the RUNX inhibitor, is produced in an amount sufficient for reducing and/or inhibiting the activity of the target transcription factor in said cell, tissue, or subject.

In preferred embodiments, upon intramuscular, intranasal, transdermal, intradermal, intralesional, intracranial, subcutaneous, intracardial, intratumoral, intravenous, intrapulmonal, intraarticular, sublingual, pulmonary, intrathecal, or ocular administration of the pharmaceutical composition or nucleic acid to a cell, tissue, or subject, the transcription factor inhibitor is produced, preferably in an amount sufficient for reducing and/or inhibiting the activity of the target transcription factor in said cell, tissue, or subject.

In preferred embodiments, upon local administration of the pharmaceutical composition or nucleic acid to a cell, tissue, or subject, the transcription factor inhibitor is produced, preferably in an amount sufficient for reducing and/or inhibiting the activity of the target transcription factor in said cell, tissue, or subject.

In preferred embodiments, the administration of the pharmaceutical composition or nucleic acid is an ocular administration, preferably in an amount sufficient for reducing and/or inhibiting the activity of the target transcription factor in said cell, tissue, or subject.

In preferred embodiments, the ocular administration is selected from topical, intravitreal, intracameral, subconjunctival, subretinal, subtenon, retrobulbar, orbital, topical, suprachoroidal, posterior juxtascleral, or intraoperative administration (e.g. during an ocular surgery), preferably intravitreal or intraoperative administration.

In another embodiment, the ocular administration may be via a device, for example a device for intravitreal delivery.

Suitably, the device is configured to be a depot for the pharmaceutical composition. Such a device allows controlled administration to the eye (e.g. in regular intervals, e.g. one a day) e.g. via a port.

Suitably, ocular administration of the pharmaceutical composition or nucleic acid (e.g. an RNA encoding a RUNX inhibitor) leads to a production of the encoded target transcription factor inhibitor in an amount sufficient for reducing and/or inhibiting the activity of the target transcription factor (e.g. RUNX) in cells and/or tissues of the eye.

In preferred embodiments in that context, the ocular administration is intravitreal administration.

Intravitreal administration e.g. via injection is one of the most common ways of administering a medicament into an eye. Accordingly, administration of the pharmaceutical composition or nucleic acid (e.g. an RNA encoding a RUNX inhibitor) via intravitreal administration is preferred in medical applications where a transcription factor is to be inhibited in the eye, for example RUNX.

In the context of intravitreal administration, a preferred injection volume of the pharmaceutical composition is ranging from about 25 μl to about 150 μl, preferably from about 25 μl to about 100 μl, more preferably from about 50 μl to about 100 μl. In a particularly preferred embodiment, the injection volume is about 50 μl.

In preferred embodiments in that context, the ocular administration is intraoperative administration. Some disease, disorders or conditions in the eye occur after an ocular surgery or operation (e.g. PVR). Accordingly, administration of the pharmaceutical composition or nucleic acid (e.g. an RNA encoding a RUNX inhibitor) via intraoperative administration is preferred in medical applications where a transcription factor is to be inhibited in the eye, for example RUNX, that is associated with a disease, disorders or condition that occurs after an ocular surgery or operation.

In preferred embodiments in that context, ocular administration of the pharmaceutical composition or the nucleic acid leads to a production of the transcription factor inhibitor (e.g. RUNX inhibitor) in cells and/or tissues of the eye, preferably in cells and/or tissues selected from cornea, lens, ciliary body, vitreous, sclera, choroid, retina, optic nerve, macula, scleral cells, choroid plexus epithelial cells, retinal cells, inflammatory cells, retinal pigment epithelium, Bruch's membrane, and retinal or choridial blood vessels.

In particularly preferred embodiments, ocular administration of the pharmaceutical composition or the nucleic acid (e.g. an RNA encoding a RUNX inhibitor) leads to a production of the transcription factor inhibitor (e.g. RUNX inhibitor) in retinal pigment epithelium (RPE) cells.

The retinal pigment epithelium (RPE) is the pigmented cell layer just outside the neurosensory retina that nourishes retinal visual cells and is firmly attached to the underlying choroid and overlying retinal visual cells. The RPE forms a monolayer of cells beneath the sensory retina that is normally mitotically inactive except when it is participating in retinal wound repair, where it plays a central role. When wound repair is complete, the RPE usually stops proliferating; failure to do so can result in blinding disorders such as e.g. proliferative vitreoretinopathy (PVR) and disciform scarring. For instance, after detachment of the sensory retina, the RPE changes in morphology and begins to proliferate. Multilayered colonies of dedifferentiated and transdifferentiated RPE cells are formed. In some instances, cells migrate onto the surface of the retina and form epiretinal membranes. These events have been implicated in the pathogenesis of proliferative vitreoretinopathy, severe scarring occurring in association with exudative macular degeneration, and poor or delayed recovery of vision after retinal reattachment.

Accordingly for some disease, disorders or conditions (e.g. PVR) it is preferred that the transcription factor inhibitor (e.g. RUNX inhibitor) is produced in RPE cells upon ocular administration to e.g. inhibit an overactive and/or overexpressed transcription factor (e.g. RUNX).

In particularly preferred embodiments in that context, ocular administration of the pharmaceutical composition or the nucleic acid (e.g. an RNA encoding a RUNX inhibitor) leads to a production of the transcription factor inhibitor (e.g. RUNX inhibitor) in retinal cells selected from photoreceptor cells, bipolar cells, ganglion cells, horizontal cells, Muller cells, mural cells, microglia, and amacrine cells. Particularly preferred are Muller cells, and microglia.

In some embodiments, the ocular administration of the pharmaceutical composition or the nucleic acid (e.g. an RNA encoding a RUNX inhibitor) is performed into a tamponade agent-filled human eye.

In the context of ocular surgery (e.g. to treat retinal detachment) tamponade agents are used to provide surface tension across retinal breaks, which prevents further fluid flow into the subretinal space until the retinopexy (photocoagulation or cryopexy) provides a permanent seal (Vaziri et al 2016, Clin. Ophtamol.)

Accordingly, tamponade agents prevent fluid flow through the retinal break into the subretinal space by filling up the vitreous space. Commonly used tamponade agents include various gases and silicone oils. Different tamponade agents have unique benefits and risks, and choice of the agent should be individualized according to the characteristics of the patient as well as perioperative and postoperative factors.

Accordingly, tamponade agents which are slowly injected into the eye will make room for following ocular administration of cells (e.g PVR cells), the pharmaceutical composition or the nucleic acid (e.g. an RNA encoding a RUNX inhibitor).

In preferred embodiments, the ocular administration of the pharmaceutical composition or the nucleic acid (e.g. an RNA encoding a RUNX inhibitor) is performed into a silicone-filled human eye.

The final step in vitreous surgery is to decide whether it is necessary to fill the vitreous space by using a tamponade agent. Silicone oil (SO, polydimethylsiloxane) has proven itself to be an effective (long-term) tamponade agent as vitreous fluid substitute, especially in the management of complex retinal detachments associated with proliferative vitreoretinopathy. This clear viscous liquid, which is immiscible with water, replaces the vitreous. Its surface tension and mild buoyant force mechanically hold the retina against the choroid (Foster et al 2008, Expert. Rev Optalmol).

In preferred embodiments, the ocular administration of the pharmaceutical composition or the nucleic acid (e.g. an RNA encoding a RUNX inhibitor) is performed into a gas-filled human eye.

Gas is a vitreous substitute that serves to keep the retinal surface dry until it heals properly. There are two main types of gases utilized for vitreoretinal surgery: Sulfur hexafluoride (SF6) or Octa (per) fluoro (n-) propane (C3F8).

Furthermore, hexafluoroethane (C2F6) or (n-) perfluoropropane (C3F8) may be used as well. They can also be mixed with sterile air and the gas bubble is then gradually absorbed over a period of weeks and the eye returns to a fluid-filled state (Lim et al 2014, Case Rep Emerg Med).

In some embodiments, the ocular administration of the pharmaceutical composition or the nucleic acid (e.g. an RNA encoding a RUNX inhibitor) is performed simultaneously or after gas or silicone extraction.

In some preferred embodiments, the ocular administration of the pharmaceutical composition or the nucleic acid (e.g. an RNA encoding a RUNX inhibitor) is performed into a C3F8-perfluoropropane-filled human eye or a human eye which was filled prior ocular administration with C3F8-perfluoropropane.

### 3: A Kit or Kit of Parts:

In a third aspect, the invention provides a kit or kit of parts comprising at least one nucleic acid of the first aspect or at least one pharmaceutical composition of the second aspect, e.g., for use in a method described herein.

Notably, embodiments relating to the artificial nucleic acid of the first aspect may likewise be read on and be understood as suitable embodiments of the kit or kit of parts of the third aspect. Also, embodiments relating to the pharmaceutical composition of the second aspect may likewise be read on and be understood as suitable embodiments of the kit or kit of parts of the third aspect.

In preferred embodiments, the kit or kit of parts comprises at least one nucleic acid of the first aspect, preferably at least one RNA, and/or at least one pharmaceutical composition of the second aspect.

In addition, the kit or kit of parts may comprise a liquid vehicle for solubilising, and/or technical instructions providing information on administration and dosage of the components.

The kit may further comprise additional components as described in the context of the pharmaceutical composition of the second aspect, and/or the vaccine of the third aspect.

The technical instructions of said kit may contain information about administration and dosage and patient groups. Such kits, preferably kits of parts, may be applied e.g. for any of the applications or uses mentioned herein, preferably for the use of the nucleic acid of the first aspect or the pharmaceutical composition of the second aspect for the treatment or prophylaxis of diseases, disorder, or condition.

In some embodiments, the nucleic acid or the pharmaceutical composition is lyophilised or spray (freeze) dried.

In embodiments where the nucleic acid or the pharmaceutical composition is provided as a lyophilized or spray-freeze dried or spray dried composition, the kit or kit of parts may suitably comprise a buffer for re-constitution of lyophilized or spray-freeze dried or spray dried nucleic acid or composition.

Accordingly, the kit or kit of parts may additionally comprise a buffer for re-constitution and/or dilution of the nucleic acid or the pharmaceutical composition.

In preferred embodiments, the buffer for re-constitution and/or dilution is a sterile buffer. In preferred embodiments, the buffer comprises a salt, preferably NaCl, optionally in a concentration of about 0.9%. Such a buffer may optionally comprise an antimicrobial preservative.

In preferred embodiments, the kit or kit of parts as defined herein comprises at least one syringe or application device. Suitably, a syringe or application device for ocular delivery (e.g. intravitreal delivery).

### 4: Medical Uses:

In a further aspect, the present invention relates to the medical use of the nucleic acid encoding at least one transcription factor inhibitor as defined herein, the pharmaceutical composition comprising at least one artificial nucleic acid as defined herein, or the kit or kit of parts as defined herein.

Notably, embodiments relating to the previous aspects may likewise be read on and be understood as suitable embodiments of medical uses of the invention. In addition, embodiments relating to medical uses as described herein of course also relate to methods of treatments.

Accordingly, the invention provides an artificial nucleic acid of the first aspect, or a pharmaceutical composition of the second aspect, or a kit or kit of the third aspect, for use as a medicament in treating or preventing a disease, disorder, or condition in a subject.

Suitably, the provided at least one transcription factor inhibitor as defined herein, preferably at least one RUNX inhibitor as defined herein, more preferably at least one RUNX trap (CBFbeta-SMMHC) as defined herein.

Accordingly, in particularly preferred embodiments, the invention provides an artificial nucleic acid encoding a RUNX inhibitor or a pharmaceutical composition comprising an artificial nucleic acid encoding a RUNX inhibitor, wherein the RUNX inhibitor comprises at least one amino acid sequence element A selected or derived from CBFbeta and at least one amino acid sequence element B selected or derived from SMMHC, for use in treating or preventing a disease, disorder, or condition in a subject. Preferably, the RUNX inhibitor comprises or consists of an amino acid sequence being identical or at least 90%, 91%, 92%, 93%, 94%, 95%, 96%, 97%, 98%, or 99% identical to SEQ ID NO: 232, or a fragment or variant thereof. More preferably, the at least one coding sequence encoding the RUNX inhibitor comprises or consists of a nucleic acid sequence that is identical or at least 90%, 91%, 92%, 93%, 94%, 95%, 96%, 97%, 98%, or 99% identical to any one of the nucleic acid sequences SEQ ID NOs: 418 or 480, or a fragment or a variant of any of these. Even more preferably, the nucleic acid, preferably the mRNA encoding the RUNX inhibitor comprises or consists of a nucleic acid sequence which is identical or at least 90%, 91%, 92%, 93%, 94%, 95%, 96%, 97%, 98%, or 99% identical to a nucleic acid sequence SEQ ID NOs: 1579 or 1580, or a fragment or variant of that sequence. Suitably, the artificial nucleic of the pharmaceutical composition is an mRNA encapsulated in a lipid-based carrier as defined herein, preferably LNPs.

In preferred embodiments, the use may be for human medical purposes and also for veterinary medical purposes, preferably for human medical purposes.

In other preferred embodiments, the use may be for human medical purposes, in particular for young infants, newborns, immunocompromised recipients, pregnant and breast-feeding women, and elderly people.

In various embodiments, the nucleic acid, the pharmaceutical composition, or the kit or kit of parts is administered by intramuscular, intranasal, transdermal, intradermal, intralesional, intracranial, subcutaneous, intracardial, intratumoral, intravenous, intrapulmonal, intraarticular, sublingual, pulmonary, intrathecal, or ocular administration.

In a further aspect, the present invention provides an artificial nucleic acid of the first aspect, or a pharmaceutical composition of the second aspect, or a kit or kit of parts of the third aspect, for use as a medicament in treating or preventing an ocular disease, disorder, or condition in a subject.

In preferred embodiments, the disease, disorder, or condition is associated with or caused by an overexpressed and/or an overactive target transcription factor (including aging).

Suitable target transcription factors may be selected from list A, more preferably from AP1; ATF6; ERG; ETV1; GLI3; HOXA9; MBD2; MEF2A; NF-kappaB; POU3F2 (BRN2); PRDM13; RBPJ; RUNX1; RUNX2; SMAD3; SMAD4; SNAI1; TAZ; TCF21; TWIST1; MAML1; MAML2; EF2A; or YAP, even more preferably from GLI3; HOXA9; MBD2; MEF2A; POU3F2 (BRN2); PRDM13; RUNX1; SMAD3; SMAD4; or SNAI1.

Particularly Preferred is RUNX, e.g. RUNX1.

In preferred embodiments, the disease, disorder, or condition is associated with or caused by pathological epithelial to mesenchymal transition (EMT), pathological cell proliferation, aberrant angiogenesis, aberrant neovascularization, degeneration, solid tumors, and/or fibrosis.

As shown in the example section for a RUNX inhibitor, administration of an artificial nucleic acid encoding CBFB-SMMHC leads to a reduction of the cellular expression of EMT-associated genes including TGFbeta2, SMAD3, and/or COL1A1. As exemplified herein for a RUNX inhibitor, administration of an artificial nucleic acid encoding CBFB-SMMHC leads to a reduction of the EMT markers and pathological cell proliferation. Additionally, treatment with artificial nucleic acid encoding the RUNX inhibitor also reversed EMT. This was illustrated by an increase of the epithelial cell clusters at the expense of mesenchymal cell clusters and by an increase in the transcription rate of MARVELD2, a tight junction associated epithelial marker, as a predictor of the future state of the cell.

Accordingly, in preferred embodiments, the present invention provides an artificial nucleic acid of the first aspect, or a pharmaceutical composition of the second aspect, or a kit or kit of parts of the third aspect, for use as a medicament in treating or preventing a disease, disorder, or condition is associated with or caused by pathological epithelial to mesenchymal transition (EMT).

Examples of EMT-associated diseases include pathologic ocular fibrosis and proliferation, for example PVR, conjunctival fibrosis (e.g. ocular cicatricial pemphigoid), corneal scarring, corneal epithelial down growth, and/or aberrant fibrosis, diseases in the anterior segment of the eye (e.g., corneal opacification and glaucoma), corneal dystrophies, herpetic keratitis, inflammation (e.g., pterygium), macula edema, retinal and vitreous hemorrhage, fibrovascular scarring, neovascular glaucoma, age-related macular degeneration (ARMD), geographic atrophy, diabetic retinopathy (DR), retinopathy of prematurity (ROP), subretinal fibrosis, epiretinal fibrosis, and gliosis. Other conditions associated with EMT including cancer, e.g., mesothelioma, ocular chronic graft-versus-host disease, corneal scarring, corneal epithelial downgrowth, conjunctival scarring, eye tumors such as melanoma and metastatic tumors, or fibrosis.

In other preferred embodiments, the present invention provides an artificial nucleic acid of the first aspect, or a pharmaceutical composition of the second aspect, or a kit or kit of parts of the third aspect, for use as a medicament in treating or preventing a disease, disorder, or condition is associated with or caused by aberrant angiogenesis.

Aberrant angiogenesis is observed in numerous diseases, such as proliferative diabetic retinopathy, ROP, DR,

AMD, retinal vein occlusions, ocular ischemic syndrome, neovascular glaucoma, retinal hemangiomas, and cancer (especially in solid tumors) and cerebral small vessel disease. It is also observed in genetic diseases such as Coats disease, Nome's Disease, FEVR and Von Hippel Lindau. Aberrant angiogenesis includes any angiogenesis that is not a normal (nonpathological) part of an organism's development, growth, or healing. Ocular neovascularization includes retinal neovascularization as well as neovascularization in the anterior segment of the eye.

In some instances, aberrant angiogenesis may manifest itself as anterior ocular neovascularization, e.g., aberrant angiogenesis that occurs as a part of corneal graft rejection. Corneal angiogenesis is involved in corneal graft rejection.

Any disease, disorder, or condition associated with or caused by pathological epithelial to mesenchymal transition (EMT), pathological cell proliferation, aberrant angiogenesis, aberrant neovascularization, and/or fibrosis (e.g. lung fibrosis, and fibrosis in virus infections, e.g. COVID-19) may be inhibited, treated, or prevented using methods and compositions disclosed herein, e.g. using the artificial nucleic acid or composition encoding a transcription factor inhibitor (e.g. a RUNX inhibitor as defined herein).

Metabolic conditions that trigger RUNX1 hyperactivation such as diabetes (e.g. high blood sugar) or genetic conditions leading to RUNX1 overexpression such as Down syndrome may be inhibited, treated or prevented using methods and compositions disclosed herein, e.g. using the artificial nucleic acid or composition encoding a transcription (e.g. a RUNX inhibitor as defined herein). RUNX1 is located on chromosome 21.

In preferred embodiments, the ocular disease, disorder, or condition is associated with or caused by aberrant proliferation or migration of retinal pigment epithelial (RPE) cells in a subject.

For example, upon retinal detachment or trauma, RPE cells may be misplaced from their anatomical location and induced to undergo EMT under the stimuli of growth factors, inflammatory cytokines, and exposure to vitreous, a collagenous gel that fills the space between the lens and the retina. For example, EMT of retinal pigment epithelial (RPE) cells plays a critical role in the pathobiology of PVR.

In preferred embodiments, the ocular disease, disorder, or condition is selected from proliferative diabetic retinopathy (PDR), macular edema, non-proliferative diabetic retinopathy, age-related macular degeneration, geographic atrophy, ocular neovascularization, retinopathy of prematurity (ROP), a retinal vein occlusion, ocular ischemic syndrome, neovascular glaucoma, a retinal hemangioma, Coats' disease, FEVR, or Norrie disease, or persistent hyperplastic primary vitreous (PHPV), or epiretinal membrane, small vessel disease, thyroid eye disease, or proliferative vitreoretinopathy (PVR).

In preferred embodiments, the ocular disease, disorder, or condition is selected from age-related macular degeneration (AMD). Age-related macular degeneration (AMD) is an eye disease that is a leading cause of vision loss in older people in developed countries. The vision loss usually becomes noticeable in a person's sixties or seventies and tends to worsen over time. Age-related macular degeneration mainly affects central vision. The vision loss in this condition results from a gradual deterioration of light-sensing cells in the tissue at the back of the eye that detects light and colour (the retina). Specifically, age-related macular degeneration affects a small area near the center of the retina, called the macula, which is responsible for central vision. Side (peripheral) vision and night vision are generally not affected. There are two major types of age-related macular degeneration, known as the dry form and the wet form. The dry form is much more common, accounting for 85 to 90 percent of all cases of AMD. It is characterized by a build-up of yellowish deposits called drusen beneath the retina and slowly progressive vision loss. The condition typically affects vision in both eyes, although vision loss often occurs in one eye before the other. The wet form of age-related macular degeneration is associated with severe vision loss that can worsen rapidly. This form of the condition is characterized by the growth of abnormal, fragile blood vessels underneath the macula. These vessels leak blood and fluid, which damages the macula and makes central vision appear blurry and distorted. Any symptom, type, or stage of AMD may be inhibited, treated, or prevented using methods and compositions disclosed herein, e.g. using the nucleic acid or composition encoding a transcription factor inhibitor (e.g. a RUNX inhibitor as defined herein).

In preferred embodiments, the ocular disease, disorder, or condition is selected from diabetic retinopathy. Diabetic retinopathy is a condition that occurs in people who have diabetes. It causes progressive damage to the retina, which is the light-sensitive lining at the back of the eye. Over time, diabetes damages the blood vessels in the retina. Diabetic retinopathy occurs when these tiny blood vessels leak blood and other fluids. This causes the retinal tissue to swell, resulting in cloudy or blurred vision. The condition usually affects both eyes. The longer a person has diabetes, without being properly treated, the more likely they will develop diabetic retinopathy. If left untreated, diabetic retinopathy can cause blindness. Symptoms of diabetic retinopathy include (i) seeing spots or floaters; (ii) blurred vision; (iii) having a dark or empty spot in the center of vision; and (iv) difficulty seeing well at night. Often the early stages of diabetic retinopathy have no visual symptoms. Early detection and treatment can limit the potential for significant vision loss from diabetic retinopathy. PDR is a more advanced form of the disease. At this stage, new fragile blood vessels can begin to grow in the retina and into the vitreous. The new blood vessels may leak blood into the vitreous, clouding vision. Without wishing to be bound by any scientific theory, diabetic retinopathy results from the damage diabetes causes to the small blood vessels located in the retina. These damaged blood vessels can cause vision loss. For example, fluid can leak into the macula, the area of the retina responsible for clear central vision. Although small, the macula is the part of the retina that allows us to see colours and fine detail. The fluid causes the macula to swell, resulting in blurred vision. In an attempt to improve blood circulation in the retina, new blood vessels may form on its surface. These fragile, abnormal blood vessels can leak blood into the back of the eye and block vision. Diabetic retinopathy is classified into two types: (1) Non-proliferative diabetic retinopathy (PDR) is the early stage of the disease in which symptoms will be mild or non-existent. In NPDR, the blood vessels in the retina are weakened. Tiny bulges in the blood vessels, called microaneurysms, may leak fluid into the retina. This leakage may lead to swelling of the macula. (2) PDR is the more advanced form of the disease. At this stage, circulation problems deprive the retina of oxygen. As a result new, fragile blood vessels can begin to grow in the retina and into the vitreous, the gel-like fluid that fills the eye. The new blood vessels may leak blood into the vitreous, clouding vision. Both NPDR and PDR may also result in macular edema. Other complications of PDR include detachment of the retina due to scar tissue formation and the development of neovascular glaucoma. Glaucoma is an eye disease in which there is progressive damage to the optic nerve. In PDR, new blood vessels grow into the area of the eye that drains fluid from the eye. This greatly raises the eye pressure, which damages the optic nerve. If left untreated, PDR can cause severe vision loss and even blindness. Any symptom, type, or stage of diabetic retinopathy may be inhibited, treated, or prevented using methods and compositions disclosed herein, e.g. using the nucleic acid or composition encoding a transcription factor inhibitor (e.g. a RUNX inhibitor).

In preferred embodiments, the ocular disease, disorder, or condition is selected from retinopathy of prematurity. Retinopathy of prematurity (ROP) is a potentially blinding eye disorder that primarily affects premature infants. The smaller a baby is at birth, the more likely that baby is to develop ROP. This disorder, which usually develops in both eyes, is one of the most common causes of visual loss in childhood and can lead to lifelong vision impairment and blindness. These infants are at a much higher risk for ROP. About 90 percent of all infants with ROP are in the milder category and do not need treatment. However, infants with more severe disease can develop impaired vision or even blindness. About 1, 100-1,500 infants annually develop ROP that is severe enough to require medical treatment. About 400-600 infants each year in the US become legally blind from ROP. ROP occurs when abnormal blood vessels grow and spread throughout the retina, the tissue that lines the back of the eye. These abnormal blood vessels are fragile and can leak, scarring the retina and pulling it out of position. This causes a retinal detachment. Retinal detachment is the main cause of visual impairment and blindness in ROP. Without wishing to be bound by any scientific theory, several complex factors may be responsible for the development of ROP. The eye starts to develop at about 16 weeks of pregnancy, when the blood vessels of the retina begin to form at the optic nerve in the back of the eye. The blood vessels grow gradually toward the edges of the developing retina, supplying oxygen and nutrients. During the last 12 weeks of a pregnancy, the eye develops rapidly. When a baby is born full-term, the retinal blood vessel growth is mostly complete (the retina usually finishes growing a few weeks to a month after birth). If a baby is born prematurely, before these blood vessels have reached the edges of the retina, normal vessel growth may stop. The edges of the retina (the periphery) may not get enough oxygen and nutrients. The periphery of the retina may then send out signals to other areas of the retina for nourishment. As a result, new abnormal vessels begin to grow. These new blood vessels are fragile and weak and can bleed, leading to retinal scarring. When these scars shrink, they pull on the retina, causing it to detach from the back of the eye. Aspects of the present invention relate to inhibiting, preventing, or treating the onset of or the progression of a ROP in a premature infant using a nucleic acid or composition of the invention. Any symptom or stage of ROP may be inhibited, treated, or prevented using methods and compositions disclosed herein, e.g. using the nucleic acid or composition encoding a transcription factor inhibitor (e.g. a RUNX inhibitor).

In preferred embodiments, the ocular disease, disorder, or condition is selected from retinal vein occlusion. Retinal vein occlusion (RVO) is a blockage of the small veins that carry blood away from the retina. Retinal vein occlusion is most often caused by hardening of the arteries (atherosclerosis) and the formation of a blood clot. Blockage of smaller veins (branch veins or BRVO) in the retina often occurs in places where retinal arteries that have been thickened or hardened by atherosclerosis cross over and place pressure on a retinal vein. Risk factors for retinal vein occlusion include: (i) atherosclerosis; (ii) diabetes; (iii) high blood pressure (hypertension; e.g., a systolic pressure of at least 140 mmHg or a diastolic pressure of at least 90 mmHg); and (iv) other eye conditions, such as glaucoma, macular edema, or vitreous hemorrhage. The risk of these disorders increases with age, therefore retinal vein occlusion most often affects older people. Blockage of retinal veins may cause other eye problems, including: (i) glaucoma (high pressure in the eye), caused by new, abnormal blood vessels growing in the front part of the eye; (ii) neovascularization. RVO can cause the retina to develop new, abnormal blood vessels, a condition called neovascularization. These new vessels may leak blood or fluid into the vitreous, the jelly-like substance that fills the inside of the eye. Small spots or clouds, called floaters, may appear in the field of vision. With severe neovascularization, the retina may detach from the back of the eye.); (iii) macular edema, caused by the leakage of fluid in the retina; and (iv) neovascular glaucoma (New blood vessels in certain parts of the eye can cause pain and a dangerous increase in pressure inside the eye.). Any symptom, type, or stage of retinal vein occlusion may be inhibited, treated, or prevented using methods and compositions disclosed herein, e.g. using the nucleic acid or composition encoding a transcription factor inhibitor (e.g. a RUNX inhibitor as defined herein).

In preferred embodiments, the ocular disease, disorder, or condition is selected from ocular ischemic syndrome. Ocular ischemic syndrome (OIS) encompasses the ocular signs and symptoms that result from chronic vascular insufficiency. Common anterior segment findings include advanced cataract, anterior segment inflammation, and iris neovascularization. Posterior segment signs include narrowed retinal arteries, dilated but no tortuous retinal veins, midperipheral dot-and-blot retinal haemorrhages, cotton-wool spots, and optic nerve/retinal neovascularization. The presenting symptoms include ocular pain and abrupt or gradual visual loss. Without wishing to be bound by any scientific theory, the most common etiology of OIS is severe unilateral or bilateral atherosclerotic disease of the internal carotid artery or marked stenosis at the bifurcation of the common carotid artery. OIS may also be caused by giant cell arteritis. The decreased vascular perfusion results in tissue hypoxia and increased ocular ischemia, leading to neovascularization. Any symptom, type, or stage of ocular ischemic syndrome may be inhibited, treated, or prevented using methods and compositions disclosed herein, e.g. using the nucleic acid or composition encoding a transcription factor inhibitor (e.g. a RUNX inhibitor as defined herein).

In preferred embodiments, the ocular disease, disorder, or condition is selected from neovascular glaucoma. Neovascular glaucoma (NVG) is classified as a secondary glaucoma. Numerous secondary ocular and systemic diseases that share one common element, retinal ischemia/hypoxia and subsequent release of an angiogenesis factor, cause NVG. This angiogenesis factor causes new blood vessel growth from pre-existing vascular structure. Depending on the progression of NVG, it can cause glaucoma either through secondary open-angle or secondary closed-angle mechanisms. This is accomplished through the growth of a fibrovascular membrane over the trabecular meshwork in the anterior chamber angle, resulting in obstruction of the meshwork and/or associated peripheral anterior synechiae. NVG is a potentially devastating glaucoma, where delayed diagnosis or poor management can result in complete loss of vision or, quite possibly, loss of the globe itself. In managing NVG, it is essential to treat both the elevated intraocular pressure (IOP) and the underlying cause of the disease. Retinal ischemia is the most common and important mechanism in most, if not all, cases that result in the anterior segment changes causing NVG. Various predisposing conditions cause retinal hypoxia and, consequently, production of an angiogenesis factor. Once released, the angiogenic factor(s) diffuses into the aqueous and the anterior segment and interacts with vascular structures in areas where the greatest aqueous-tissue contact occurs. The resultant growth of new vessels at the pupillary border and iris surface [neovascularization of the iris (NVI)] and over the iris angle [neovascularization of the angle (NVA)] ultimately leads to formation of fibrovascular membranes. The fibrovascular membranes, which may be invisible on gonioscopy, accompany NVA and progressively obstruct the trabecular meshwork. This causes secondary open-angle glaucoma. As the disease process continues, the fibrovascular membranes along the NVA tend to mature and contract, thereby tenting the iris toward the trabecular meshwork and resulting in peripheral anterior synechiae and progressive synechial angle closure. Elevated IOP is a direct result of this secondary angle-closure glaucoma. Any symptom, type, or stage of neovascular glaucoma may be inhibited, treated, or prevented using methods and compositions disclosed herein, e.g. using the nucleic acid or composition encoding a transcription factor inhibitor (e.g. a RUNX inhibitor as defined herein).

In preferred embodiments, the ocular disease, disorder, or condition is selected from retinal hemangiomas. Retinal hemangiomas, also known as retinal capillary hemangiomas (RCHs) and retinal hemangioblastomas, occur most frequently in conjunction with von Hippel-Lindau (VHL) syndrome. These lesions are characterized by plump, but otherwise normal, retinal capillary endothelial cells with normal pericytes and basement membrane. Astrocytes with lipid vacuoles are found in the tumor interstitia. Isolated RCH outside of VHL do occur, although they are more likely to be single, unilateral, and present later. Von Hippel-Lindau syndrome has an autosomal dominant inheritance pattern, with an incidence of 1 in 36,000 live births. These lesions can occur either singly, or more often, multiply and bilaterally, with a greater than 80% predilection for peripheral location. Vision loss can occur from exudation, strabismus, hemorrhage, and retinal detachment, as well secondary causes such as macular edema, lipid maculopathy, and epiretinal membrane. Early lesions often present as indistinct areas of redness in the retina, which appear to be retinal hemorrhages. Patients may be relatively asymptomatic until the lesions achieve larger size, and it is imperative to perform life-long surveillance of even asymptomatic individuals with VHL because smaller lesions are more easily eradicated than larger lesions. Any symptom, type, or stage of retinal hemangioma may be inhibited, treated, or prevented using methods and compositions disclosed herein. In some embodiments, a subject at risk of developing a retinal hemangioma, such as a subject with VHL, is treated to delay or prevent the onset of a retinal hemangioma, e.g. using the nucleic acid or composition encoding a transcription factor inhibitor (e.g. a RUNX inhibitor as defined herein).

In preferred embodiments, the ocular disease, disorder, or condition is selected from Coats' disease. Coats' disease, (also known as exudative retinitis or retinal telangiectasis, sometimes spelled Coates' disease), is a rare congenital, nonhereditary eye disorder, causing full or partial blindness, characterized by abnormal development of blood vessels behind the retina. Coats' disease results in a gradual loss of vision. Blood leaks from the abnormal vessels into the back of the eye, leaving behind cholesterol deposits and damaging the retina. Coats' disease normally progresses slowly. At advanced stages, retinal detachment is likely to occur. Glaucoma, atrophy, and cataracts can also develop secondary to Coats' disease. In some cases, removal of the eye may be necessary (enucleation). The most common sign at presentation is leukocoria (abnormal white reflection of the retina). Symptoms typically begin as blurred vision, usually pronounced when one eye is closed (due to the unilateral nature of the disease). Often the unaffected eye will compensate for the loss of vision in the other eye; however, this results in some loss of depth perception and parallax. Deterioration of sight may begin in either the central or peripheral vision. Deterioration is likely to begin in the upper part of the vision field as this corresponds with the bottom of the eye where blood usually pools. Flashes of light, known as photopsia, and floaters are common symptoms. Persistent color patterns may also be perceived in the affected eye. Initially, these may be mistaken for psychological hallucinations, but are actually the result of both retinal detachment and foreign fluids mechanically interacting with the photoreceptors located on the retina. One early warning sign of Coats' disease is yellow-eye in flash photography. An eye affected by Coats' will glow yellow in photographs as light reflects off cholesterol deposits. Coats' disease is thought to result from breakdown of the blood-retinal barrier in the endothelial cell, resulting in leakage of blood products containing cholesterol crystals and lipid-laden macrophages into the retina and subretinal space. Over time, the accumulation of this proteinaceous exudate thickens the retina, leading to massive, exudative retinal detachment. On funduscopic eye examination, the retinal vessels in early Coats' disease appear tortuous and dilated, mainly confined to the peripheral and temporal portions of retina. In moderate to severe Coats' disease, massive retinal detachment and hemorrhage from the abnormal vessels may be seen. Any symptom, type, or stage of Coats' disease may be inhibited, treated, or prevented using methods and compositions disclosed herein. In some embodiments, a subject at risk of developing Coats' disease is treated to delay or prevent the onset of Coats' disease, e.g. using the nucleic acid or composition encoding a transcription factor inhibitor (e.g. a RUNX inhibitor as defined herein).

In preferred embodiments, the ocular disease, disorder, or condition is selected from Norrie disease. Norrie disease is an inherited eye disorder that leads to blindness in male infants at birth or soon after birth. It causes abnormal development of the retina, the layer of sensory cells that detect light and colour, with masses of immature retinal cells accumulating at the back of the eye. As a result, the pupils appear white when light is shone on them, a sign called leukocoria. The irises (colored portions of the eyes) or the entire eyeballs may shrink and deteriorate during the first months of life, and cataracts (cloudiness in the lens of the eye) may eventually develop. About one third of individuals with Norrie disease develop progressive hearing loss, and more than half experience developmental delays in motor skills such as sitting up and walking. Other problems may include mild to moderate intellectual disability, often with psychosis, and abnormalities that can affect circulation, breathing, digestion, excretion, or reproduction. Mutations in the norrin cystine knot growth factor (NDP) gene cause Norrie disease. The NDP gene provides instructions for making a protein called norrin. Mutations in the Norrie gene are often unique to a family and have been described throughout the extent of the Norrie gene. Although Norrie disease itself does not seem to shorten lifespan, individuals with blindness, deafness and/or mental disability may have a reduced lifespan as a result of these conditions. Any symptom, type, or stage of Norrie disease may be inhibited, treated, or prevented using methods and compositions disclosed herein, e.g. using the nucleic acid or composition encoding a transcription factor inhibitor (e.g. a RUNX inhibitor as defined herein).

In preferred embodiments, the ocular disease, disorder, or condition is selected from familial exudative vitreoretinopathy (FEVR). FEVR is a rare hereditary ocular disorder characterized by a failure of peripheral retinal vascularization which may be abnormal or incomplete. FEVR is a condition with fundus changes similar to those in retinopathy of prematurity but appearing in children who had been born full-term with normal birthweight. With respect to genetics, about 50% of cases can be linked to 4 causative genes (DP, LRP5, FZD4, and TSPAN12), all of which form part of the Wnt signalling pathway, which is vital for normal retinal vascular development. Any symptom, type, or stage of FEVR may be inhibited, treated, or prevented using methods and compositions disclosed herein. In some embodiments, a subject at risk of developing FEVR is treated to delay or prevent the onset or progression of FEVR. In some embodiments, a subject at risk of developing FEVR is treated to delay or prevent the onset or progression of aberrant angiogenesis due to FEVR, e.g. using the nucleic acid or composition encoding a transcription factor inhibitor (e.g. a RUNX inhibitor as defined herein).

In particularly preferred embodiments, the ocular disease, disorder, or condition is selected from proliferative vitreoretinopathy (PVR). Proliferative vitreoretinopathy (PVR) is a clinical syndrome that develops as a complication of rhegmatogenous retinal detachment and is also commonly associated with eye trauma. PVR is the most common cause of failure in retinal detachment surgery, however, it can also occur with untreated eyes with retinal detachment. In particular, PVR can occur with vitreous hemorrhage, after cryotherapy, after laser retinopexy, pneumatic retinopexy, scleral buckling, or vitrectomy, and after a variety of surgical complications. PVR is also common after eye traumas (e.g., penetrating injuries) and other conditions associated with prolonged inflammation. PVR occurs in about 8-10% of patients undergoing primary retinal detachment surgery and prevents the successful surgical repair of rhegmatogenous retinal detachment. PVR can be treated with surgery to reattach the retina, however, the visual outcome of the surgery is very poor. If PVR is progressive, then despite complex surgery, low vision in the eye results. PVR is characterized by proliferation or migration of cells derived from retinal pigment epithelium (RPE), glia, or inflammatory recruitment on the retinal surface and within the vitreous. These cells transdifferentiate and take on contractile properties. The process of PVR can start when there is an interruption to the surface lining (e.g., through posterior vitreous detachment and local preretinal membrane formation or retinal tears in the periphery). The PVR process is self-propagating and is often considered an inappropriate excess wound-healing response. The cellular proliferation can increase the influx of inflammatory cytokines and inflammatory cells. In embodiments, as described herein proliferation or migration of RPE cells describes their transdifferentiation to assume contractile properties through internal cellular contractile proteins and by laying down extracellular collagen. The cells can multiply and grow along any available scaffolding (e.g., the retinal surfaces or elements of the residual vitreous). The mass contraction can lead to retinal wrinkles, folds, tears, and traction retinal detachment. During rhegmatogenous retinal detachment, fluid from the vitreous humor enters a retinal hole. The accumulation of fluid in the subretinal space and the tractional force of the vitreous on the retina result in rhegmatogenous retinal detachment. During this process the retinal cell layers come in contact with vitreous cytokines. These cytokines trigger the ability of the retinal pigmented epithelium (RPE) to proliferate and migrate. The process involved resembles fibrotic wound healing by the RPE cells. The RPE cells undergo epithelial-mesenchymal transition (EMT) and develop the ability to migrate out into the vitreous. During this process the RPE cell layer-neural retinal adhesion and RPE-ECM (extracellular matrix) adhesions are lost. The RPE cells lay down fibrotic membranes while they migrate and these membranes contract and pull at the retina. Thus, this leads to secondary retinal detachment after primary retinal detachment surgery. During RPE disruption, inflammation may play an important role in the development of PVR. Cytokines IL-6, IL-I, TNFalpha have been identified in high concentrations in the vitreous in the early, proliferative stages of PVR, but they decrease to normal levels in the scarring phase. Other molecules involved in PVR include TGF and IL-6. The transcription factor RUNX1 is involved in the development and progression of PVR.

Risk factors and clinical signs: As described above, the most common development of PVR is after a retinal detachment surgery and/or repair, although patients can develop PVR spontaneously with retinal detachment prior to surgery or with longstanding primary detachments. Multiple factors have been associated with the formation of PVR. In general, processes that increase vascular permeability are more likely to increase the probability of PVR formation. Specific risk factors that have been identified include: uveitis; large, giant, or multiple tears; vitreous hemorrhage, preoperative or postoperative choroidal detachments; aphakia; multiple previous surgeries; and large detachments involving greater than 2 quadrants of the eye. Early signs of PVR are often subtle and can include cellular dispersion in the vitreous and on the retinal surface, which can appear as a white opacification of the retinal surface and small wrinkles or folds. More developed PVR is characteristic with fixed folds and retinal detachment. Diagnosis is typically done by indirect ophthalmoscopy and slit-lamp biomicroscopy. Additionally, an ultrasound can help visualize immobile retinal folds of detachment and prominent vitreous membranes. Also, wide-field fundus photography can be used to visualize retinal detachments. However, the clinical history and exam is often enough to make the diagnosis of a retinal detachment.

Development Stages: Ocular wound healing typically occurs in 3 stages: (1) an inflammatory stage, (2) a proliferative stage, and (3) a modulatory stage. PVR can be viewed in a similar fashion, with the wound being the retinal detachment. This healing response often takes place over many weeks. Early on, preretinal PVR adopts an immature appearance and consistency. During this phase, the retina may still remain compliant, and the PVR membrane may be difficult to remove due to its amorphous form. By 6 to 8 weeks, however, the PVR membrane becomes more mature, taking on a white, fibrotic appearance. In this stage, the PVR is more easily identifiable, causes rigidity of the retina, and can be more identifiably removed.

Classification: The extent of PVR in patients is often classified (or graded) depending on the severity. The most commonly used classification system was published by the Retina Society Terminology Committee. It classifies the appearance of PVR based on clinical signs and its geographic location (Grade A, B, C, or D). Grade A is characterized by the appearance of vitreous haze and RPE cells in the vitreous, or by pigment clumping. Grade B is characterized by wrinkling of the edges of the retinal tear or the inner retinal surface. Grade C is characterized by posterior or anterior full thickness retinal folds with the presence of epi/subretinal membranes/bands. Grade D is characterized by fixed retinal folds in all four quadrants. Diagnosis via clinical examination and imaging for PVR is known in the art, e.g., as described in the classification of retinal detachment with proliferative vitreoretinopathy. Ophthalmology, 1983; 90 (2): p. 121-5. Clinical examination and classification schemes are further described in Di Lauro et ak, J Ophthalmol. 2016; Volume 2016, Article ID 7807596, 6 pages 2016:7807596. (PMCID: PMC4939352); hereby incorporated by reference. PVR is a condition distinct from proliferative diabetic retinopathy (PDR). PVR is a condition distinct from a small blood vessel disease. The fundamental process involved in PDR is aberrant angiogenesis, and therefore impacting vascular endothelial cells. To the contrary, in PVR, the fundamental processes is the aberrant epithelial to mesenchymal transition (EMT) of retinal pigment epithelial derived cells, and other cells within the eye.

Any symptom, type, or stage of PVR may be inhibited, treated, or prevented using methods and compositions disclosed herein, e.g. using the nucleic acid or composition encoding a transcription factor inhibitor (e.g. a RUNX inhibitor as defined herein).

In preferred embodiments, the ocular disease, disorder, or condition is epiretinal membrane, a very common disease that occurs after retinal detachment surgery and can be considered a very mild form of PVR, that can cause visual change, due to a very thin membrane forming on top of the retina.

Accordingly, in particularly preferred embodiments, the ocular disease, disorder, or condition is PVR, preferably the prevention of PVR.

As shown in the example section, treatment of C-PVR cells (human primary cell cultures obtained from surgically removed PVR membranes) with nucleic acid encoding a RUNX inhibitor (CBFbeta-SMMHC) led to a significant reduction in RUNX1 expression and significant reduction in EMT-associated genes. In addition, treatment of C-PVR cells with nucleic acid encoding a RUNX inhibitor (CBFbeta-SMMHC) strongly reduced proliferation and ocular pathology triggered by injection of human PVR cells in a rabbit eye. Further shown in the Example section, administration of the artificial nucleic acid or composition led to a prevention of EMT in PVR.

In preferred embodiments in that context, cell proliferation and/or cell growth is reduced in eyes with PVR.

In preferred embodiments, the invention provides an artificial nucleic acid, or a pharmaceutical composition, or a kit or kit of parts, for use as a medicament in treating or preventing a disease, disorder, or condition in a subject, wherein the subject has suffered a trauma to the eye, comprises a retinal hole, a retinal tear, a retinal detachment disorder, or has undergone an ocular surgery.

Retinal detachment disorder is a disorder of the eye in which the neurosensory retina separates from the retinal pigment epithelial layer underneath. The mechanism most commonly involves a break in the retina that then allows the fluid in the eye to get behind the retina. A break in the retina can occur from a posterior vitreous detachment, injury to the eye, or inflammation of the eye. Other risk factors include being short sighted and previous cataract surgery. Typically, diagnosis is accomplished by either looking at the back of the eye with an ophthalmoscope or by ultrasound. Symptoms include an increase in the number of floaters, flashes of light, and worsening of the outer part of the visual field, which may be described as a curtain over part of the field of vision. In about 7% of cases both eyes are affected. Without treatment permanent loss of vision may occur. Retinal detachments affect between 0.6 and 1.8 people per 10,000 per year. About 0.3% of people are affected at some point in their life. It is most common in people who are in their 60s or 70s, and males are more often affected than females. The long term outcomes depend on the duration of the detachment and whether the macula was detached. If treated before the macula detaches outcomes are generally good. Optionally, the subject has not been diagnosed or characterized with some other ocular disorder comprising age-related macular degeneration or an ocular angiogenesis disease or disorder. When the retina is pulled away from the back of the eye, it is a retinal detachment. Typically, the vitreous moves away from the retina without causing problems. But sometimes the vitreous pulls hard enough to tear the retina in one or more places, and thus causing a retinal tear. Fluid may pass through a retinal tear, lifting the retina off the back of the eye.

The symptoms of vitreous separation, retinal tear, and retinal detachment are similar and sometimes can overlap. On occasion, the patient may notice the floaters and flashing lights (photopsia) more commonly associated with isolated vitreous separation. An ophthalmologist, optometrist, or primary care physician may be suspicious about a more serious problem if symptoms are of very recent or sudden onset and are accompanied by a shower of spots or “cobwebs”. Of even greater concern is the loss of peripheral vision, which may present as a shadow moving toward the center of one's field of vision. Additionally, in retinal detachment, a retinal hole may develop. Because the vitreous is attached to the retina with tiny strands of collagen, it can pull on the retina as it shrinks. Sometimes, this shrinkage can tear off a small piece of the retina in the periphery, causing a hole or tear of the periphery retina. If this missing piece of retina is in the macula, it is called a macular hole. Additionally, another direct cause of macular holes due to vitreous shrinkage is when the collagen strands stay attached to the retina forming an epiretinal membrane. These membranes can contract around the macula, causing the macula to develop a hole from the traction. Retinal detachments commonly occur secondary to peripheral retinal tears/holes, and rarely form macular holes. A minority of retinal detachments result from trauma, including blunt blows to the orbit, penetrating trauma, and concussions to the head. There are three types of retinal detachment: (1) rhegmatogenous retinal detachment-a rhegmatogenous retinal detachment occurs due to a break in the retina (e.g., a retinal tear) that allows fluid to pass from the vitreous space into the subretinal space between the sensory retina and the retinal pigment epithelium. Retinal breaks are divided into three types-holes, tears and dialyses. Holes form due to retinal atrophy especially within an area of lattice degeneration. Tears are due to vitreoretinal traction. Dialyses are very peripheral and circumferential and may be either tractional or atrophic. The atrophic form most often occurs as idiopathic dialysis of the young. (2) Exudative, serous, or secondary retinal detachment—an exudative retinal detachment occurs due to inflammation, injury or vascular abnormalities that results in fluid accumulating underneath the retina without the presence of a hole, tear, or break. In evaluation of retinal detachment, it is critical to exclude exudative detachment as surgery will make the situation worse, not better. Although rare, exudative detachment can be caused by the growth of a tumor on the layers of tissue beneath the retina, namely the choroid. This cancer is called a choroidal melanoma. (3) Tractional retinal detachment-a tractional retinal detachment occurs when fibrous (from PVR membrane) or fibrovascular (from neovascular disorders such as proliferative diabetic retinopathy) tissue, caused by an injury, inflammation or neovascularization, pulls the sensory retina from the retinal pigment epithelium

Accordingly, in preferred embodiments, the retinal detachment disorder is selected from rhegmatogenous retinal detachment, exudative retinal detachment, or tractional retinal detachment.

In preferred embodiments, the nucleic acid, the pharmaceutical composition, or the kit or kit of parts is administered by local administration, preferably by ocular administration.

In some embodiments, the artificial nucleic acid or composition of the invention may be administered using an ocular delivery device. The ocular delivery device may be designed for the controlled release of the artificial nucleic acid or the pharmaceutical composition with multiple defined release rates and sustained dose kinetics and permeability.

In preferred embodiments, the ocular administration is selected from intravitreal administration, by administration prior to an ocular surgery, during an ocular surgery, or after an ocular surgery.

In particularly preferred embodiments, the ocular administration is selected from intravitreal administration.

In particularly preferred embodiments in that context, at least one ocular administration is prior to an ocular surgery, during an ocular surgery, and/or after an ocular surgery. Some disease, disorders or conditions in the eye occur after an ocular surgery or operation as described herein (e.g. PVR). Accordingly, administration of the pharmaceutical composition or nucleic acid (e.g. an RNA encoding a RUNX inhibitor) via intraoperative administration is preferred in medical applications where a transcription factor is to be inhibited in the eye, for example RUNX, that is associated with a disease, disorders or condition that occurs after an ocular surgery or operation.

In some embodiments, a first dose of the nucleic acid or composition is administered during an ocular surgery, and second and further doses are administered via intravitreal administration. For example, a nucleic acid encoding a RUNX inhibitor of the invention may be administered at the time of diagnosis of a retinal detachment, or during an ocular surgery (e.g. to prevent the development of a disease, e.g. PVR) and a second and optional further doses are administered via intravitreal administration (e.g. to prevent the development of a disease, e.g. PVR).

In particularly preferred embodiments, the disease, disorder, or condition is associated with or caused by overexpressed and/or overactive RUNX transcription factor. Preferred in that context is RUNX1. Suitably, the disease, disorder, or condition is associated with or caused by overexpressed and/or overactive RUNX transcription factor is an ocular disease.

RUNX1 has non-detectable basal expression in the healthy retina, whereas in pathologies such as proliferative diabetic retinopathy and choroidal neovascularization, aberrant RUNX1 signalling occurs and is believed to drive the angiogenic process. These data indicate that use of the nucleic acid encoding a RUNX inhibitor may be applicable in a very broad range of pathologies characterized by increased RUNX1 transcription factor activity. RUNX1 functions as a transcriptional switch allowing organisms to control for delicate cell fate decisions in multiple critical developmental processes including hematopoiesis. In addition, our group uncovered multiple instances where RUNX1 expression, which is normally silent, is strongly induced to drive control pathological processes associated with aberrant angiogenesis, EMT, and fibrosis in the eye and elsewhere. These processes are fundamental to prevalent conditions including cancer, proliferative diabetic retinopathy, exudative age-related macular degeneration, proliferative vitreoretinopathy, lung fibrosis, and virus-caused lung fibrosis e.g. COVID-19.

For example, essentially all ocular diseases characterized by aberrant angiogenesis or pathologic epithelial-mesenchymal transition (EMT) are associated with RUNX1 overexpression. Examples of diseases that involve aberrant angiogenesis that are associated with RUNX1 overexpression include non-proliferative diabetic retinopathy, diabetic macular edema, exudative age-related macular degeneration, retinal neovascularization, iris neovascularization, neovascular glaucoma, central retinal vein occlusion, branch retinal vein occlusion, Coats' disease, familial exudative vitreoretinopathy (FEVR), Von Hippel Lindau disease, retinal hemangioma, Leber's military aneurysms, macula telangiectasia, polypoidal choroidal vasculopathy, myopic choroidal neovascularization, idiopathic choroidal neovascularization, corneal neovascularization, thyroid eye disease, small vessel disease. Examples of diseases that involve aberrant EMT that are associated with RUNX1 overexpression include Proliferative vitreoretinopathy (PVR), open angle glaucoma, exudative age-related macular degeneration (fibrosis of CNV lesions), uveal metastatic cancers, geographic atrophy. Further, examples of diseases that may be associated with RUNX1 overexpression include primary ocular tumors including uveal melanoma, retinoblastoma, astrocytomas. Additional diseases that may be associated with RUNX1 overexpression include corneal scarring after trauma, infection, chemical injury, cataracts, epiretinal membranes, corneal neovascularization, corneal scarring, which has implications for corneal transplants and corneal chemical injury/trauma, corneal viral infections, ocular cancers, uveal melanomas.

In various embodiments of the invention, the artificial nucleic acid or composition of the invention may be administered only once or multiple times. For example, a nucleic acid encoded RUNX inhibitor may be administered using a method disclosed herein at least about once, twice, three times, four times, five times, six times, or seven times per day, week, month, or year. In some embodiments, a nucleic acid encoded RUNX inhibitor is administered once per month. In certain embodiments, a nucleic acid encoded RUNX inhibitor is administered once per week, once every two weeks, once a month via intravitreal injection. In various embodiments, such as embodiments involving eye drops, a composition is self-administered.

In particularly preferred embodiment, the invention provides an artificial nucleic acid encoding a RUNX inhibitor or a pharmaceutical composition comprising an artificial nucleic acid encoding a RUNX inhibitor, wherein the RUNX inhibitor comprises at least one amino acid sequence element A selected or derived from CBFbeta and at least one amino acid sequence element B selected or derived from SMMHC, for use in treating or preventing an ocular disease, disorder, or condition associated with or caused by overexpressed and/or overactive RUNX transcription factor, preferably proliferative vitreoretinopathy (PVR), preferably wherein the artificial nucleic acid or the pharmaceutical composition is to be administered via ocular administration. Preferably, the RUNX inhibitor comprises or consists of an amino acid sequence being identical or at least 90%, 91%, 92%, 93%, 94%, 95%, 96%, 97%, 98%, or 99% identical to SEQ ID NO: 232, or a fragment or variant thereof. More preferably, the at least one coding sequence encoding the RUNX inhibitor comprises or consists of a nucleic acid sequence that is identical or at least 90%, 91%, 92%, 93%, 94%, 95%, 96%, 97%, 98%, or 99% identical to any one of the nucleic acid sequences SEQ ID NOs: 418 or 480, or a fragment or a variant of any of these. Even more preferably, the nucleic acid, preferably the mRNA encoding the RUNX inhibitor comprises or consists of a nucleic acid sequence which is identical or at least 90%, 91%, 92%, 93%, 94%, 95%, 96%, 97%, 98%, or 99% identical to a nucleic acid sequence SEQ ID NOs: 1579 or 1580, or a fragment or variant of that sequence. Suitably, the artificial nucleic of the pharmaceutical composition is an mRNA encapsulated in lipid-based carriers, preferably LNPs.

A further aspect of the invention relates to a pharmaceutical composition comprising at least one RNA encoding a therapeutic protein formulated in a lipid nanoparticle (LNP), wherein the LNP comprise an aggregation reducing lipid, a cationic lipid selected or derived from formula (III-1), a neutral lipid or phospholipid and a steroid or steroid analog, for use in treatment or prevention of an ophthalmic disease, disorder or condition, wherein said composition is administered via intravitreal administration to a subject in need thereof.

Surprisingly in that context, LNPs having cationic lipids as defined herein, in particular cationic lipids selected or derived from formula (III-1) are suitable for ocular delivery.

Accordingly, the at least one cationic or ionizable lipid is a lipid selected or derived from formula (III-1)

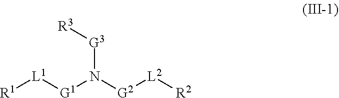

preferably, wherein one of L1 or L2 is —O(C═O)—, —(C═O)O—, —C(═O)—, —O—, —S(O)x-, —S—S—, —C(═O)S—, SC(═O)—, —NRaC(═O)—, —C(═O)NRa—, —NRaC(═O)NRa—, —OC(═O)NRa— or —NRaC(═O)O—, and the other of L1 or L2 is —O(C═O)—, —(C═O)O—, —C(═O)—, —O—, —S(O)x-, —S S—, —C(═O)S—, SC(═O)—, —NRaC(═O)—, —C(═O)NRa—, —NRaC(═O)NRa—, —OC(═O)NRa— or —NRaC(═O)O— or a direct bond; G1 and G2 are each independently unsubstituted C1-C12 alkylene or C1-C12 alkenylene; G3 is C1-C24 alkylene, C1-C24 alkenylene, C3-C8 cycloalkylene, C3-C8 cycloalkenylene; Ra is H or C1-C12 alkyl; R1 and R2 are each independently C6-C24 alkyl or C6-C24 alkenyl; R3 is H, OR5, CN, C(═O)OR4, OC(═O)R4 or —NR5C(═O)R4; R4 is C1-C12 alkyl; R5 is H or C1-C6 alkyl; and x is 0, 1 or 2.

Preferably, the at least one cationic or ionizable lipid is selected or derived from a lipid according to formula III-3:

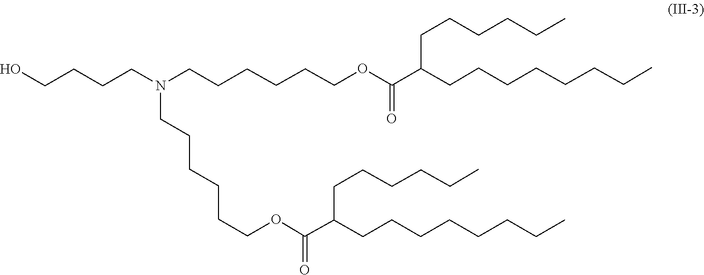 [SMILES: CCCCCCCCC(CCCCCC)C(=O)OCCCCCCN(CCCCO)CCCCCCOC(=O)C(CCCCCC)CCCCCCCC]

Most preferably, the cationic lipid is selected or derived from ALC-0315, SM-102.

Suitably, the LNPs comprise at least one aggregation-reducing lipid, at least one cationic lipid or ionizable lipid, at least one neutral lipid or phospholipid, and at least one steroid or steroid analog.

The aggregation reducing lipid is selected from a polymer conjugated lipid, for example a PEG-conjugated lipid or a PEG-free lipid.

The polymer conjugated lipid is selected or derived from DMG-PEG 2000, C10-PEG2K, Cer8-PEG2K, POZ-lipid, or ALC-0159. Alternatively, the polymer conjugated lipid is a POZ-lipid as defined herein.

Suitably, the neutral lipid is selected or derived from DSPC, DHPC, or DphyPE.

Suitably, the steroid or steroid analog selected or derived from cholesterol, cholesteryl hemisuccinate (CHEMS), preferably cholesterol.

Accordingly, in that context, the LNPs encapsulating the mRNA comprise
(i) at least one cationic lipid selected from ALC-0315;
(ii) at least one neutral lipid selected from DSPC;
(iii) at least one steroid or steroid analogue selected from cholesterol; and
(iv) at least one aggregation reducing lipid selected from ALC-0159. Suitably, the LNPs in that context are 315 LNPs as defined herein.

Alternatively, in that context, the LNPs encapsulating the mRNA comprise
(i) at least one cationic lipid selected from SM-102;
(ii) at least one neutral lipid selected from DSPC;
(iii) at least one steroid or steroid analogue selected from cholesterol; and
(iv) at least one aggregation reducing lipid selected from DMG-PEG 2000. Suitably, the LNPs in that context are SM-102 LNPs as defined herein.

In preferred embodiments, the LNPs comprise about 20-60% cationic lipid, about 5-25% neutral lipid, about 25-55% steroid or steroid analogue, and about 0.5-15% aggregation reducing lipid.

In preferred embodiments, the wt/wt ratio of lipid to RNA in the LNPs is from about 10:1 to about 60:1, preferably from about 20:1 to about 30:1. Suitably, the N/P ratio of the lipid-based carriers encapsulating the mRNA is in a range from about 1 to about 10, preferably in a range from about 5 to about 7.

Preferably, the LNPs have a Z-average size in a range of about 50 nm to about 120 nm.

In a preferred embodiment, intravitreal administration to a subject in need thereof is performed into a tamponade agent-filled human eye (e.g. gas agent or a silicone agent)

In a preferred embodiment, the ophthalmic disease, disorder or condition may be selected from PVR, neovascularization, retinal degenerative disease, diabetic eye disease, retinal detachment, optic nerve disease, endocrine disorders, cancer disease, infectious disease, parasitic disease, in particular, pigmentary uveitis (PU), branch retinal vein occlusion (BRVO), central retinal vein occlusion (CRVO), macular edema, cystoid macular edema (CME), uveitic macular edema (UME), cytomegalovirus retinitis, endophthalmitis, scleritis, choriotetinitis, dry eye syndrome, Norris disease, Coat's disease, persistent hyperplastic primary vitreous, familial exudative vitreoretinopathy, Leber congenital amaurosis, X-linked retinoschisis, Leber's hereditary optic neurophathy, uveitis, refraction and accommodation disorders, keratoconus, amblyopia, conjunctivitis, comeal ulcers, dacryocystitis, Duane retraction syndrome, optic neuritis, ocular inflammation, glaucoma, macular degeneration, and uveitis, or any disease, disorder or condition related or associated thereto.

In a preferred embodiment, the mRNA provides a coding sequence encoding a therapeutic protein. Suitably, the therapeutic protein is selected from an antibody, an antibody fragment, an intrabody, a receptor, a binding protein, a CRISPR-associated endonuclease, a transcription factor, an enzyme, a growth factor, a structural protein, cytoplasmic or cytoskeletal proteins, or fragments, variants, or combinations of any of these.

### 5: Methods of Treatment:

In a further aspect, the present invention relates to a method of treating or preventing a disease, disorder or condition.

Notably, embodiments relating to the previous aspects may likewise be read on and be understood as suitable embodiments of method of treatments of the invention. In particular, specific features and embodiments relating to method of treatments as provided herein may also apply for medical uses of the invention and vice versa.

Preventing (inhibiting) or treating a disease relates to inhibiting the full development of a disease or condition, for example, in a subject who is at risk for a disease such as an infection. “Treatment” refers to a therapeutic intervention that ameliorates a sign or symptom of a disease or pathological condition after it has begun to develop. The term “ameliorating”, with reference to a disease or pathological condition, refers to any observable beneficial effect of the treatment. Inhibiting a disease can include preventing or reducing the risk of the disease. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters that are specific to the particular disease. A “prophylactic” treatment is a treatment administered to a subject who does not exhibit signs of a disease or exhibits only early signs for the purpose of decreasing the risk of developing pathology.

In preferred embodiments, the present invention relates to a method of treating or preventing a disease, disorder or condition, wherein the method comprises applying or administering to a subject in need thereof an effective amount of the artificial nucleic acid of the first aspect, or the pharmaceutical composition of the second aspect, or the kit or kit of parts of the third aspect.

As used herein, “effective” when referring to an amount of a therapeutic compound refers to the quantity of the compound that is sufficient to yield a desired therapeutic response without undue adverse side effects (such as toxicity, irritation, or allergic response) commensurate with a reasonable benefit/risk ratio when used in the manner of this disclosure.

Accordingly, in particularly preferred embodiments, the method of treating or preventing a disease, disorder or condition provides an artificial nucleic acid encoding a RUNX inhibitor or a pharmaceutical composition comprising an artificial nucleic acid encoding a RUNX inhibitor, wherein the RUNX inhibitor comprises at least one amino acid sequence element A selected or derived from CBFbeta and at least one amino acid sequence element B selected or derived from SMMHC, for use in treating or preventing a disease, disorder, or condition in a subject. Preferably, the RUNX inhibitor comprises or consists of an amino acid sequence being identical or at least 90%, 91%, 92%, 93%, 94%, 95%, 96%, 97%, 98%, or 99% identical to SEQ ID NO: 232, or a fragment or variant thereof. More preferably, the at least one coding sequence encoding the RUNX inhibitor comprises or consists of a nucleic acid sequence that is identical or at least 90%, 91%, 92%, 93%, 94%, 95%, 96%, 97%, 98%, or 99% identical to any one of the nucleic acid sequences SEQ ID NOs: 418 or 480, or a fragment or a variant of any of these. Even more preferably, the nucleic acid, preferably the mRNA encoding the RUNX inhibitor comprises or consists of a nucleic acid sequence which is identical or at least 90%, 91%, 92%, 93%, 94%, 95%, 96%, 97%, 98%, or 99% identical to a nucleic acid sequence SEQ ID NOs: 1579 or 1580, or a fragment or variant of that sequence. Suitably, the artificial nucleic of the pharmaceutical composition is an mRNA encapsulated in lipid-based carriers, preferably LNPs.

In preferred embodiments, the method is for human medical purposes and also for veterinary medical purposes, preferably for human medical purposes.

In other preferred embodiments, method is for human medical purposes, in particular for young infants, newborns, immunocompromised recipients, pregnant and breast-feeding women, and elderly people.

In various embodiments of the method, the nucleic acid, the pharmaceutical composition, or the kit or kit of parts is administered by intramuscular, intranasal, transdermal, intradermal, intralesional, intracranial, subcutaneous, intracardial, intratumoral, intravenous, intrapulmonal, intraarticular, sublingual, pulmonary, intrathecal, or ocular administration.

In preferred embodiments, the disease, disorder, or condition is an ocular disease, disorder, or condition.

In preferred embodiments, the disease, disorder, or condition is associated with or caused by an overexpressed and/or an overactive transcription factor (including e.g. aging).

In preferred embodiments, the disease, disorder, or condition associated with or caused by pathological epithelial to mesenchymal transition (EMT), pathological cell proliferation, aberrant angiogenesis, aberrant neovascularization, degeneration, solid tumors, and/or fibrosis.

In preferred embodiments, the ocular disease, disorder, or condition is associated with or caused by aberrant proliferation or migration of retinal pigment epithelial (RPE) cells in a subject.

In preferred embodiments, the ocular disease, disorder, or condition is selected from proliferative diabetic retinopathy (PDR), macular edema, non-proliferative diabetic retinopathy, age-related macular degeneration, geographic atrophy, ocular neovascularization, retinopathy of prematurity (ROP), a retinal vein occlusion, ocular ischemic syndrome, neovascular glaucoma, a retinal hemangioma, Coates' disease, FEVR, or Norrie disease, persistent hyperplastic primary vitreous, epiretinal membrane, small vessel disease, thyroid eye disease, or proliferative vitreoretinopathy (PVR).

In preferred embodiments, the ocular disease, disorder, or condition is PVR, preferably the prevention of PVR.

In preferred embodiments, cell proliferation and/or cell growth is reduced in eyes with PVR.

In preferred embodiments, the subject has suffered a trauma to the eye, comprises a retinal hole, a retinal tear, a retinal detachment disorder, or has undergone an ocular surgery.

In preferred embodiments, the retinal detachment disorder is selected from rhegmatogenous retinal detachment, exudative retinal detachment, or tractional retinal detachment.

In preferred embodiments, the applying or administering is performed more than once, for example two times, three times, or four times, for example periodically.

In preferred embodiments, the applying or administering is performed by local administration, preferably by ocular administration.

In preferred embodiments, the applying or administering is performed by intravitreal administration, by administration prior to an ocular surgery, during an ocular surgery, and/or after an ocular surgery.

In preferred embodiments, the disease, disorder, or condition is associated with or caused by overexpressed RUNX and/or overactive RUNX, preferably RUNX1.

In a preferred aspect, the present invention relates to a method of treating or preventing or preventing an ocular disease in a subject comprising
a) administering to the subject an effective amount of a composition comprising an artificial nucleic acid comprising at least one coding sequence encoding the RUNX inhibitor, the RUNX inhibitor comprising:
   at least one amino acid element A selected or derived from CBFbeta configured to bind to the target transcription factor RUNX in the cytosol; and.
   at least one amino acid element B selected or derived from SMMHC configured to trap the target transcription factor RUNX in the cytosol.

Suitably, the RUNX inhibitor comprises or consists of an amino acid sequence being identical or at least 90%, 91%, 92%, 93%, 94%, 95%, 96%, 97%, 98%, or 99% identical to SEQ ID NO: 232, or a fragment or variant thereof.

In preferred embodiments in that context, the composition is administered by ocular administration.

In preferred embodiments in that context, the ocular administration is selected from intravitreal or intraoperative administration.

In preferred embodiments in that context, the artificial nucleic acid is an RNA, preferably an mRNA.

In alternative embodiments in that context, the artificial nucleic acid is comprised in a viral vector, preferably an AAV vector.

Suitably, the at least one coding sequence of the artificial nucleic acid encoding the RUNX inhibitor comprises or consists of a nucleic acid sequence that is identical or at least 90%, 91%, 92%, 93%, 94%, 95%, 96%, 97%, 98%, or 99% identical to any one of the nucleic acid sequences SEQ ID NOs: 418 or 480, or a fragment or a variant of any of these.

In preferred embodiments, the nucleic acid, preferably the mRNA encoding the RUNX inhibitor comprises or consists of a nucleic acid sequence which is identical or at least 90%, 91%, 92%, 93%, 94%, 95%, 96%, 97%, 98%, or 99% identical to a nucleic acid sequence SEQ ID NOs: 1579 or 1580, or a fragment or variant of that sequence.

In preferred embodiments, the nucleic acid, preferably the mRNA encoding the RUNX inhibitor comprises or consists of a nucleic acid sequence which is identical or at least 90%, 91%, 92%, 93%, 94%, 95%, 96%, 97%, 98%, or 99% identical to a nucleic acid sequence SEQ ID NO: 1580, or a fragment or variant of that sequence.

In preferred embodiments in that context, the artificial nucleic acid of the composition is formulated in lipid-based carriers, preferably LNPs as defined in the context of the second aspect.

In preferred embodiments in that context, the ocular disease is associated with or caused by overexpressed RUNX and/or overactive RUNX, preferably RUNX1.

In preferred embodiments in that context, the ocular disease is associated with or caused by overexpressed RUNX and/or overactive RUNX, preferably, wherein the ocular disease is PVR.

### 6: A Method of Reducing the Activity of a Transcription Factor in a Cell or a Subject:

In a further aspect, the present invention relates to a method of reducing the activity of a transcription factor in a cell or a subject.

Notably, embodiments relating to the previous aspects (e.g. artificial nucleic acid, pharmaceutical composition) may likewise be read on and be understood as suitable embodiments of the method of reducing the activity of a transcription factor in a cell or a subject of the present aspect.

In preferred embodiments, the method of reducing the activity of a transcription factor in a cell or a subject comprises
a) applying or administering an artificial nucleic acid comprising at least one coding sequence encoding at least one transcription factor inhibitor as defined in the first aspect; or
b) applying or administering a pharmaceutical composition comprising the artificial nucleic acid comprising at least one coding sequence encoding at least one transcription factor inhibitor as defined in the second aspect;
to a cell, tissue, or subject, wherein the transcription factor inhibitor is produced in the cell, tissue, or subject after administration or application to said cell, tissue, or subject.

In preferred embodiments in that context, the transcription factor inhibitor is a transcription factor trap.

In various embodiments, the target transcription factor is selected from list A.

In preferred embodiments, the transcription factor inhibitor is produced in the cell, tissue, or subject after administration or application to said cell, tissue, or subject, wherein
the produced transcription factor inhibitor is a dominant negative inhibitor of the target transcription factor; and/or
the produced transcription factor inhibitor binds to the target transcription factor or binds to at least one transcription co-factor of the target transcription factor; and/or
the produced transcription factor inhibitor reduces or prevents interaction of a target transcription factor with its target DNA; and/or
the produced transcription factor inhibitor reduces or prevents interaction of a target transcription factor with at least one of its transcription co-factors; and/or
the produced transcription factor inhibitor reduces or prevents nuclear translocation of a target transcription factor; and/or
the produced transcription factor inhibitor or transcription factor trap reduces the activity of a target transcription factor; and/or
the produced transcription factor inhibitor or transcription factor trap reduces the cellular expression of a target transcription factor; and/or
the produced transcription factor inhibitor or transcription factor trap reduces the cellular expression of proteins that are controlled or regulated by a target transcription factor, and/or
the produced transcription factor inhibitor or transcription factor trap triggers plasma membrane anchoring of transcription factors.

In preferred embodiments, the produced transcription factor inhibitor is a RUNX inhibitor or a RUNX trap and the target transcription factor is a RUNX transcription factor, preferably RUNX1.

In preferred embodiments, the produced transcription factor inhibitor is a RUNX inhibitor or a RUNX trap, wherein the produced RUNX inhibitor or RUNX trap
sequesters cellular RUNX by binding to RUNX in the cytosol or sequesters cellular CBFbeta by binding to CBFbeta in the nucleus
reduces or prevents the interaction of cellular RUNX with cellular CBFbeta; and/or
reduces cellular RUNX-CBFbeta complex formation and/or activity; and/or
reduces the cellular expression of RUNX controlled or regulated gene products; and/or
reduces the cellular expression of TGFbeta2, SMAD3, and/or COL1A1; and/or
increase the transcription rate of MARVELD2; and/or
reduces the cellular expression of RUNX; and/or
reduces or prevents pathological epithelial to mesenchymal transition (EMT), pathological cell proliferation, aberrant angiogenesis, aberrant neovascularization, degeneration, and/or fibrosis; and/or
reduces or prevents cell proliferation and/or cell growth in eyes with PVR.

In embodiments, the applying or administering is selected from intramuscular, intranasal, transdermal, intradermal, intralesional, intracranial, subcutaneous, intracardial, intratumoral, intravenous, intrapulmonal, intraarticular, sublingual, pulmonary, intrathecal, local administration, or ocular administration.

In preferred embodiments, the ocular administration is selected from topical, intravitreal, intracameral, subconjunctival, subretinal, subtenon, retrobulbar, topical, orbital, suprachoroidal, posterior juxtascleral, or intraoperative administration, preferably intravitreal or intraoperative administration.

In another embodiment, the ocular administration may be via a device, for example a device for intravitreal delivery. Suitably, the device is configured to be a depot for the pharmaceutical composition. Such a device allows controlled administration to the eye (e.g. in regular intervals, e.g. one a day) e.g. via a port.

In preferred embodiments, an ocular administration leads to a production of the transcription factor inhibitor in cornea, lens, ciliary body, vitreous, sclera, choroid, retina, optic nerve, macula, scleral cells, choroid plexus epithelial cells, retinal cells, inflammatory cells, retinal pigment epithelium, Bruch's membrane, and retinal blood vessels or choroidal blood vessels.

In particularly preferred embodiments, an ocular administration leads to a production of the transcription factor inhibitor in cells and/or tissues of the eye, preferably in retinal pigment epithelial (RPE) cells or cells derived from RPE cells.

In preferred embodiments, an ocular administration leads to a production of the transcription factor inhibitor in retinal cells, preferably selected from photoreceptor cells, bipolar cells, ganglion cells, horizontal cells, Muller cells, mural cells, microglia, and amacrine cells. Particularly preferred are Muller cells, and microglia.

In particularly preferred embodiments, the reduction of the activity of a transcription factor is a transient reduction of the activity of a transcription factor. This is particularly important in the context of the whole invention, as a permanent reduction of the activity of a transcription factor would potentially be associated with side effects. Using a transient molecule such as mRNA is particularly suitable in that context as mRNA is typically degraded, and the encoded transcription factor trap protein has also a limited half-life (depending on the tissue, the protein sequence etc.).

In particularly preferred embodiments, the transcription factor inhibitor is a dominant negative inhibitor of the target transcription factor.

### Item List

Preferred embodiments of the present invention are provided in the following item list:
1. An artificial nucleic acid comprising at least one coding sequence encoding at least one transcription factor inhibitor for reducing or inhibiting the activity of a target transcription factor in a cell, wherein the nucleic acid comprises at least one heterologous untranslated region (UTR).
2. The artificial nucleic acid of item 1, wherein the transcription factor inhibitor is produced in the cytosol upon administration of the artificial nucleic acid to a cell, tissue, or subject.
3. The artificial nucleic acid of item 1 or 2, wherein the transcription factor inhibitor is a dominant negative inhibitor of the target transcription factor and/or its transcription co-factor.
4. The artificial nucleic acid of item 1 to 3, wherein the transcription factor inhibitor binds to the target transcription factor.
5. The artificial nucleic acid of item 1 to 4, wherein the transcription factor inhibitor binds to at least one transcription co-factor of the target transcription factor, preferably a co-activator of the target transcription factor.
6. The artificial nucleic acid of item 1 or 5, wherein the transcription factor inhibitor reduces or prevents interaction of the target transcription factor with its target DNA.
7. The artificial nucleic acid of item 1 to 6, wherein the transcription factor inhibitor reduces or prevents interaction of the target transcription factor with at least one of its transcription co-factors.
8. The artificial nucleic acid of item 1 to 7, wherein the transcription factor inhibitor reduces or prevents nuclear translocation of the target transcription factor and/or its transcription co-factor.
9. The artificial nucleic acid of item 1 to 8, wherein the transcription factor inhibitor reduces the activity of the target transcription factor.
10. The artificial nucleic acid of item 1 to 9, wherein the transcription factor inhibitor reduces the cellular expression of the target transcription factor.
11. The artificial nucleic acid of item 1 to 10, wherein the transcription factor inhibitor reduces the cellular expression of proteins that are controlled or regulated by the target transcription factor.
12. The artificial nucleic acid of any one of the preceding items, wherein the transcription factor inhibitor is a transcription factor trap preferably configured to bind and trap the target transcription factor in the cytosol.
13. The artificial nucleic acid of any one of the preceding items, wherein the target transcription factor is selected from a transcription factor that has an aberrant or pathologic transcription factor activity.
14. The artificial nucleic acid of any one of the preceding items, wherein the target transcription factor is selected from a transcription factor that has aberrant or pathologic transcription factor activity associated with epithelial to mesenchymal transition (EMT), pathological cell proliferation, aberrant angiogenesis, aberrant neovascularization, degeneration, and/or fibrosis.
15. The artificial nucleic acid of any one of the preceding items, wherein the target transcription factor is selected from a transcription factor that is overexpressed and/or overactive in a disease, disorder, or condition, including e.g. aging.
16. The artificial nucleic acid of any one of the preceding items, wherein the target transcription factor is selected from a transcription factor that is overexpressed and/or overactive in an ocular disease, disorder, or condition.
17. The artificial nucleic acid of any one of the preceding items, wherein the target transcription factor is selected from AC008770.3; AC023509.3; AC092835.1; AC138696.1; ADNP; ADNP2; AEBP1; AEBP2; AHCTF1; AHDC1; AHR; AHRR; AIRE; AKAP8; AKAP8L; AKNA; ALX1; ALX3; ALX4; ANHX; ANKZF1; AP1; AR; ARGFX; ARHGAP35; ARID2; ARID3A; ARID3B; ARID3C; ARID5A; ARID5B; ARNT; ARNT2; ARNTL; ARNTL2; ARX; ASCL1; ASCL2; ASCL3; ASCL4; ASCL5; ASH1L; ATF1; ATF2; ATF3; ATF4; ATF5; ATF6; ATF6B; ATF7; ATMIN; ATOH1; ATOH7; ATOH8; BACH1; BACH2; BARHL1; BARHL2; BARX1; BARX2; BATF; BATF2; BATF3; BAZ2A; BAZ2B; BBX; BCL11A; BCL11B; BCL6; BCL6B; BHLHA15; BHLHA9; BHLHE22; BHLHE23; BHLHE40; BHLHE41; BNC1; BNC2; BORCS8-MEF2B; BPTF; BRF2; BSX; C11orf95; CAMTA1; CAMTA2; CARF; CASZ1; CBX2; CC2D1A; CCDC169-SOHLH2; CCDC17; CDC5L; CDX1; CDX2; CDX4; CEBPA; CEBPB; CEBPD; CEBPE; CEBPG; CEBPZ; CENPA; CENPB; CENPBD1; CENPS; CENPT; CENPX; CGGBP1; CHAMP1; CHCHD3; CIC; CLOCK; CPEB1; CPXCR1; CREB1; CREB3; CREB3L1; CREB3L2; CREB3L3; CREB3L4; CREB5; CREBL2; CREBZF; CREM; CRX; CSRNP1; CSRNP2; CSRNP3; CTCF; CTCFL; CUX1; CUX2; CXXC1; CXXC4; CXXC5; DACH1; DACH2; DBP; DBX1; DBX2; DDIT3; DEAF1; DLX1; DLX2; DLX3; DLX4; DLX5; DLX6; DMBX1; DMRT1; DMRT2; DMRT3; DMRTA1; DMRTA2; DMRTB1; DMRTC2; DMTF1; DNMT1; DNTTIP1; DOT1L; DPF1; DPF3; DPRX; DR1; DRAP1; DRGX; DUX1; DUX3; DUX4; DUXA; DZIP1; E2F1; E2F2; E2F3; E2F4; E2F5; E2F6; E2F7; E2F8; E4F1; EBF1; EBF2; EBF3; EBF4; EEA1; EGR1; EGR2; EGR3; EGR4; EHF; ELF1; ELF2; ELF3; ELF4; ELF5; ELK1; ELK3; ELK4; EMX1; EMX2; EN1; EN2; EOMES; EPAS1; ERF; ERG; ESR1; ESR2; ESRRA; ESRRB; ESRRG; ESX1; ETS1; ETS2; ETV1; ETV2; ETV3; ETV3L; ETV4; ETV5; ETV6; ETV7; EVX1; EVX2; FAM170A; FAM200B; FBXL19; FERD3L; FEV; FEZF1; FEZF2; FIGLA; FIZ1; FLI1; FLYWCH1; FOS; FOSB; FOSL1; FOSL2; FOXA1; FOXA2; FOXA3; FOXB1; FOXB2; FOXC1; FOXC2; FOXD1; FOXD2; FOXD3; FOXD4; FOXD4L1; FOXD4L3; FOXD4L4; FOXD4L5; FOXD4L6; FOXE1; FOXE3; FOXF1; FOXF2; FOXG1; FOXH1; FOXI1; FOXI2; FOXI3; FOXJ1; FOXJ2; FOXJ3; FOXK1; FOXK2; FOXL1; FOXL2; FOXM1; FOXN1; FOXN2; FOXN3; FOXN4; FOXO1; FOXO3; FOXO4; FOXO6; FOXP1; FOXP2; FOXP3; FOXP4; FOXQ1; FOXR1; FOXR2; FOXS1; GABPA; GATA1; GATA2; GATA3; GATA4; GATA5; GATA6; GATAD2A; GATAD2B; GBX1; GBX2; GCM1; GCM2; GFI1; GFI1B; GLI1; GLI2; GLI3; GLI4; GLIS1; GLIS2; GLIS3; GLMP; GLYR1; GMEB1; GMEB2; GPBP1; GPBP1L1; GRHL1; GRHL2; GRHL3; GSC; GSC2; GSX1; GSX2; GTF2B; GTF21; GTF2IRD1; GTF2IRD2; GTF2IRD2B; GTF3A; GZF1; HAND1; HAND2; HBP1; HDX; HELT; HES1; HES2; HES3; HES4; HES5; HES6; HES7; HESX1; HEY1; HEY2; HEYL; HHEX; HIC1; HIC2; HIF1A; HIF3A; HINFP; HIVEP1; HIVEP2; HIVEP3; HKR1; HLF; HLX; HMBOX1; HMG20A; HMG20B; HMGA1; HMGA2; HMGN3; HMX1; HMX2; HMX3; HNF1A; HNF1B; HNF4A; HNF4G; HOMEZ; HOXA1; HOXA10; HOXA11; HOXA13; HOXA2; HOXA3; HOXA4; HOXA5; HOXA6; HOXA7; HOXA9; HOXB1; HOXB13; HOXB2; HOXB3; HOXB4; HOXB5; HOXB6; HOXB7; HOXB8; HOXB9; HOXC10; HOXC11; HOXC12; HOXC13; HOXC4; HOXC5; HOXC6; HOXC8; HOXC9; HOXD1; HOXD10; HOXD11; HOXD12; HOXD13; HOXD3; HOXD4; HOXD8; HOXD9; HSF1; HSF2; HSF4; HSF5; HSFX1; HSFX2; HSFY1; HSFY2; IKZF1; IKZF2; IKZF3; IKZF4; IKZF5; INSM1; INSM2; IRF1; IRF2; IRF3; IRF4; IRF5; IRF6; IRF7; IRF8; IRF9; IRX1; IRX2; IRX3; IRX4; IRX5; IRX6; ISL1; ISL2; ISX; JAZF1; JDP2; JRK; JRKL; JUN; JUNB; JUND; KAT7; KCMF1; KCNIP3; KDM2A; KDM2B; KDM5B; KIN; KLF1; KLF10; KLF11; KLF12; KLF13; KLF14; KLF15; KLF16; KLF17; KLF2; KLF3; KLF4; KLF5; KLF6; KLF7; KLF8; KLF9; KMT2A; KMT2B; L3MBTL1; L3MBTL3; L3MBTL4; LBX1; LBX2; LCOR; LCORL; LEF1; LEUTX; LHX1; LHX2; LHX3; LHX4; LHX5; LHX6; LHX8; LHX9; LIN28A; LIN28B; LIN54; LMX1A; LMX1B; LTF; LYL1; MAF; MAFA; MAFB; MAFF; MAFG; MAFK; MAX; MAML1; MAML2; MAZ; MBD1; MBD2; MBD3; MBD4; MBD6; MBNL2; MECOM; MECP2; MEF2A; MEF2B; MEF2C; MEF2D; MEIS1; MEIS2; MEIS3; MEOX1; MEOX2; MESP1; MESP2; MGA; MITF; MIXL1; MKX; MLX; MLXIP; MLXIPL; MNT; MNX1; MSANTD1; MSANTD3; MSANTD4; MSC; MSGN1; MSX1; MSX2; MTERF1; MTERF2; MTERF3; MTERF4; MTF1; MTF2; MXD1; MXD3; MXD4; MXI1; MYB; MYBL1; MYBL2; MYC; MYCL; MYCN; MYF5; MYF6; MYNN; MYOD1; MYOG; MYPOP; MYRF; MYRFL; MYSM1; MYT1; MYT1L; MZF1; NACC2; NAIF1; NANOG; NANOGNB; NANOGP8; NCOA1; NCOA2; NCOA3; NEUROD1; NEUROD2; NEUROD4; NEUROD6; NEUROG1; NEUROG2; NEUROG3; NF kappa B; NFAT5; NFAT5A; NFATC1; NFATC2; NFATC3; NFATC4; NFE2; NFE2L1; NFE2L2; NFE2L3; NFE4; NFIA; NFIB; NFIC; NFIL3; NFIX; NFKB1; NFKB2; NFX1; NFXL1; NFYA; NFYB; NFYC; NHLH1; NHLH2; NKRF; NKX1-1; NKX1-2; NKX2-1; NKX2-2; NKX2-3; NKX2-4; NKX2-5; NKX2-6; NKX2-8; NKX3-1; NKX3-2; NKX6-1; NKX6-2; NKX6-3; NME2; NOBOX; NOTO; NOTCH1; NOTCH2; NOTCH3; NOTCH4; NPAS1; NPAS2; NPAS3; NPAS4; NROB1; NR1D1; NR1D2; NR1H2; NR1H3; NR1H4; NR112; NR113; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRF1; NRL; OLIG1; OLIG2; OLIG3; ONECUT1; ONECUT2; ONECUT3; OSR1; OSR2; OTP; OTX1; OTX2; OVOL1; OVOL2; OVOL3; PA2G4; PATZ1; PAX1; PAX2; PAX3; PAX4; PAX5; PAX6; PAX7; PAX8; PAX9; PBX1; PBX2; PBX3; PBX4; PCGF2; PCGF6; PDX1; PEG3; PGR; PHF1; PHF19; PHF20; PHF21A; PHOX2A; PHOX2B; PIN1; PITX1; PITX2; PITX3; PKNOX1; PKNOX2; PLAG1; PLAGL1; PLAGL2; PLSCR1; POGK; POU1F1; POU2AF1; POU2F1; POU2F2; POU2F3; POU3F1; POU3F2 (BRN2); POU3F3; POU3F4; POU4F1; POU4F2; POU4F3; POU5F1; POU5F1B; POU5F2; POU6F1; POU6F2; PPARA; PPARD; PPARG; PRDM1; PRDM10; PRDM12; PRDM13; PRDM14; PRDM15; PRDM16; PRDM2; PRDM4; PRDM5; PRDM6; PRDM8; PRDM9; PREB; PRMT3; PROP1; PROX1; PROX2; PRR12; PRRX1; PRRX2; PTF1A; PURA; PURB; PURG; RAG1; RARA; RARB; RARG; RAX; RAX2; RBAK; RBCK1; RBPJ; RBPJL; RBSN; REL; RELA; RELB; REPIN1; REST; REXO4; RFX1; RFX2; RFX3; RFX4; RFX5; RFX6; RFX7; RFX8; RHOXF1; RHOXF2; RHOXF2B; RLF; RORA; RORB; RORC; RREB1; RUNX1; RUNX2; RUNX3; RXRA; RXRB; RXRG; SAFB; SAFB2; SALL1; SALL2; SALL3; SALL4; SATB1; SATB2; SCMH1; SCML4; SCRT1; SCRT2; SCX; SEBOX; SETBP1; SETDB1; SETDB2; SGSM2; SHOX; SHOX2; SIM1; SIM2; SIX1; SIX2; SIX3; SIX4; SIX5; SIX6; SKI; SKIL; SKOR1; SKOR2; SLC2A4RG; SMAD1; SMAD3; SMAD4; SMAD5; SMAD9; SMYD3; SNAI1; SNAI2; SNAI3; SNAPC2; SNAPC4; SNAPC5; SOHLH1; SOHLH2; SON; SOX1; SOX10; SOX11; SOX12; SOX13; SOX14; SOX15; SOX17; SOX18; SOX2; SOX21; SOX3; SOX30; SOX4; SOX5; SOX6; SOX7; SOX8; SOX9; SP1; SP100; SP110; SP140; SP140L; SP2; SP3; SP4; SP5; SP6; SP7; SP8; SP9; SPDEF; SPEN; SPI1; SPIB; SPIC; SPZ1; SRCAP; SREBF1; SREBF2; SRF; SRY; ST18; STAT1; STAT2; STAT3; STAT4; STAT5A; STAT5B; STAT6; T; TAZ; TAL1; TAL2; TBP; TBPL1; TBPL2; TBR1; TBX1; TBX10; TBX15; TBX18; TBX19; TBX2; TBX20; TBX21; TBX22; TBX3; TBX4; TBX5; TBX6; TCF12; TCF15; TCF20; TCF21; TCF23; TCF24; TCF3; TCF4; TCF7; TCF7L1; TCF7L2; TCFL5; TEAD1; TEAD2; TEAD3; TEAD4; TEF; TERB1; TERF1; TERF2; TET1; TET2; TET3; TFAP2A; TFAP2B; TFAP2C; TFAP2D; TFAP2E; TFAP4; TFCP2; TFCP2L1; TFDP1; TFDP2; TFDP3; TFE3; TFEB; TFEC; TGIF1; TGIF2; TGIF2LX; TGIF2LY; THAP1; THAP10; THAP11; THAP12; THAP2; THAP3; THAP4; THAP5; THAP6; THAP7; THAP8; THAP9; THRA; THRB; THYN1; TIGD1; TIGD2; TIGD3; TIGD4; TIGD5; TIGD6; TIGD7; TLX1; TLX2; TLX3; TMF1; TOPORS; TP53; TP63; TP73; TPRX1; TRAFD1; TRERF1; TRPS1; TSC22D1; TSHZ1; TSHZ2; TSHZ3; TTF1; TWIST1; TWIST2; UBP1; UNCX; USF1; USF2; USF3; VAX1; VAX2; VDR; VENTX; VEZF1; VSX1; VSX2; WIZ; WT1; XBP1; XPA; YBX1; YAP; YBX2; YBX3; YY1; YY2; ZBED1; ZBED2; ZBED3; ZBED4; ZBED5; ZBED6; ZBED9; ZBTB1; ZBTB10; ZBTB11; ZBTB12; ZBTB14; ZBTB16; ZBTB17; ZBTB18; ZBTB2; ZBTB20; ZBTB21; ZBTB22; ZBTB24; ZBTB25; ZBTB26; ZBTB3; ZBTB32; ZBTB33; ZBTB34; ZBTB37; ZBTB38; ZBTB39; ZBTB4; ZBTB40; ZBTB41; ZBTB42; ZBTB43; ZBTB44; ZBTB45; ZBTB46; ZBTB47; ZBTB48; ZBTB49; ZBTB5; ZBTB6; ZBTB7A; ZBTB7B; ZBTB7C; ZBTB8A; ZBTB8B; ZBTB9; ZC3H8; ZEB1; ZEB2; ZFAT; ZFHX2; ZFHX3; ZFHX4; ZFP1; ZFP14; ZFP2; ZFP28; ZFP3; ZFP30; ZFP37; ZFP41; ZFP42; ZFP57; ZFP62; ZFP64; ZFP69; ZFP69B; ZFP82; ZFP90; ZFP91; ZFP92; ZFPM1; ZFPM2; ZFX; ZFY; ZGLP1; ZGPAT; ZHX1; ZHX2; ZHX3; ZIC1; ZIC2; ZIC3; ZIC4; ZIC5; ZIK1; ZIM2; ZIM3; ZKSCAN1; ZKSCAN2; ZKSCAN3; ZKSCAN4; ZKSCAN5; ZKSCAN7; ZKSCAN8; ZMAT1; ZMAT4; ZNF10; ZNF100; ZNF101; ZNF107; ZNF112; ZNF114; ZNF117; ZNF12; ZNF121; ZNF124; ZNF131; ZNF132; ZNF133; ZNF134; ZNF135; ZNF136; ZNF138; ZNF14; ZNF140; ZNF141; ZNF142; ZNF143; ZNF146; ZNF148; ZNF154; ZNF155; ZNF157; ZNF16; ZNF160; ZNF165; ZNF169; ZNF17; ZNF174; ZNF175; ZNF177; ZNF18; ZNF180; ZNF181; ZNF182; ZNF184; ZNF189; ZNF19; ZNF195; ZNF197; ZNF2; ZNF20; ZNF200; ZNF202; ZNF205; ZNF207; ZNF208; ZNF211; ZNF212; ZNF213; ZNF214; ZNF215; ZNF217; ZNF219; ZNF22; ZNF221; ZNF222; ZNF223; ZNF224; ZNF225; ZNF226; ZNF227; ZNF229; ZNF23; ZNF230; ZNF232; ZNF233; ZNF234; ZNF235; ZNF236; ZNF239; ZNF24; ZNF248; ZNF25; ZNF250; ZNF251; ZNF253; ZNF254; ZNF256; ZNF257; ZNF26; ZNF260; ZNF263; ZNF264; ZNF266; ZNF267; ZNF268; ZNF273; ZNF274; ZNF275; ZNF276; ZNF277; ZNF28; ZNF280A; ZNF280B; ZNF280C; ZNF280D; ZNF281; ZNF282; ZNF283; ZNF284; ZNF285; ZNF286A; ZNF286B; ZNF287; ZNF292; ZNF296; ZNF3; ZNF30; ZNF300; ZNF302; ZNF304; ZNF311; ZNF316; ZNF317; ZNF318; ZNF319; ZNF32; ZNF320; ZNF322; ZNF324; ZNF324B; ZNF326; ZNF329; ZNF331; ZNF333; ZNF334; ZNF335; ZNF337; ZNF33A; ZNF33B; ZNF34; ZNF341; ZNF343; ZNF345; ZNF346; ZNF347; ZNF35; ZNF350; ZNF354A; ZNF354B; ZNF354C; ZNF358; ZNF362; ZNF365; ZNF366; ZNF367; ZNF37A; ZNF382; ZNF383; ZNF384; ZNF385A; ZNF385B; ZNF385C; ZNF385D; ZNF391; ZNF394; ZNF395; ZNF396; ZNF397; ZNF398; ZNF404; ZNF407; ZNF408; ZNF41; ZNF410; ZNF414; ZNF415; ZNF416; ZNF417; ZNF418; ZNF419; ZNF420; ZNF423; ZNF425; ZNF426; ZNF428; ZNF429; ZNF43; ZNF430; ZNF431; ZNF432; ZNF433; ZNF436; ZNF438; ZNF439; ZNF44; ZNF440; ZNF441; ZNF442; ZNF443; ZNF444; ZNF445; ZNF446; ZNF449; ZNF45; ZNF451; ZNF454; ZNF460; ZNF461; ZNF462; ZNF467; ZNF468; ZNF469; ZNF470; ZNF471; ZNF473; ZNF474; ZNF479; ZNF48; ZNF480; ZNF483; ZNF484; ZNF485; ZNF486; ZNF487; ZNF488; ZNF490; ZNF491; ZNF492; ZNF493; ZNF496; ZNF497; ZNF500; ZNF501; ZNF502; ZNF503; ZNF506; ZNF507; ZNF510; ZNF511; ZNF512; ZNF512B; ZNF513; ZNF514; ZNF516; ZNF517; ZNF518A; ZNF518B; ZNF519; ZNF521; ZNF524; ZNF525; ZNF526; ZNF527; ZNF528; ZNF529; ZNF530; ZNF532; ZNF534; ZNF536; ZNF540; ZNF541; ZNF543; ZNF544; ZNF546; ZNF547; ZNF548; ZNF549; ZNF550; ZNF551; ZNF552; ZNF554; ZNF555; ZNF556; ZNF557; ZNF558; ZNF559; ZNF560; ZNF561; ZNF562; ZNF563; ZNF564; ZNF565; ZNF566; ZNF567; ZNF568; ZNF569; ZNF57; ZNF570; ZNF571; ZNF572; ZNF573; ZNF574; ZNF575; ZNF576; ZNF577; ZNF578; ZNF579; ZNF580; ZNF581; ZNF582; ZNF583; ZNF584; ZNF585A; ZNF585B; ZNF586; ZNF587; ZNF587B; ZNF589; ZNF592; ZNF594; ZNF595; ZNF596; ZNF597; ZNF598; ZNF599; ZNF600; ZNF605; ZNF606; ZNF607; ZNF608; ZNF609; ZNF610; ZNF611; ZNF613; ZNF614; ZNF615; ZNF616; ZNF618; ZNF619; ZNF620; ZNF621; ZNF623; ZNF624; ZNF625; ZNF626; ZNF627; ZNF628; ZNF629; ZNF630; ZNF639; ZNF641; ZNF644; ZNF645; ZNF646; ZNF648; ZNF649; ZNF652; ZNF653; ZNF654; ZNF655; ZNF658; ZNF66; ZNF660; ZNF662; ZNF664; ZNF665; ZNF667; ZNF668; ZNF669; ZNF670; ZNF671; ZNF672; ZNF674; ZNF675; ZNF676; ZNF677; ZNF678; ZNF679; ZNF680; ZNF681; ZNF682; ZNF683; ZNF684; ZNF687; ZNF688; ZNF689; ZNF69; ZNF691; ZNF692; ZNF695; ZNF696; ZNF697; ZNF699; ZNF7; ZNF70; ZNF700; ZNF701; ZNF703; ZNF704; ZNF705A; ZNF705B; ZNF705D; ZNF705E; ZNF705G; ZNF706; ZNF707; ZNF708; ZNF709; ZNF71; ZNF710; ZNF711; ZNF713; ZNF714; ZNF716; ZNF717; ZNF718; ZNF721; ZNF724; ZNF726; ZNF727; ZNF728; ZNF729; ZNF730; ZNF732; ZNF735; ZNF736; ZNF737; ZNF74; ZNF740; ZNF746; ZNF747; ZNF749; ZNF750; ZNF75A; ZNF75D; ZNF76; ZNF761; ZNF763; ZNF764; ZNF765; ZNF766; ZNF768; ZNF77; ZNF770; ZNF771; ZNF772; ZNF773; ZNF774; ZNF775; ZNF776; ZNF777; ZNF778; ZNF780A; ZNF780B; ZNF781; ZNF782; ZNF783; ZNF784; ZNF785; ZNF786; ZNF787; ZNF788; ZNF789; ZNF79; ZNF790; ZNF791; ZNF792; ZNF793; ZNF799; ZNF8; ZNF80; ZNF800; ZNF804A; ZNF804B; ZNF805; ZNF808; ZNF81; ZNF813; ZNF814; ZNF816; ZNF821; ZNF823; ZNF827; ZNF829; ZNF83; ZNF830; ZNF831; ZNF835; ZNF836; ZNF837; ZNF84; ZNF841; ZNF843; ZNF844; ZNF845; ZNF846; ZNF85; ZNF850; ZNF852; ZNF853; ZNF860; ZNF865; ZNF878; ZNF879; ZNF880; ZNF883; ZNF888; ZNF891; ZNF90; ZNF91; ZNF92; ZNF93; ZNF98; ZNF99; ZSCAN1; ZSCAN10; ZSCAN12; ZSCAN16; ZSCAN18; ZSCAN2; ZSCAN20; ZSCAN21; ZSCAN22; ZSCAN23; ZSCAN25; ZSCAN26; ZSCAN29; ZSCAN30; ZSCAN31; ZSCAN32; ZSCAN4; ZSCAN5A; ZSCAN5B; ZSCAN5C; ZSCAN9; ZUFSP; ZXDA; ZXDB; ZXDC; or ZZZ3.
18. The artificial nucleic acid of any one of the preceding items, wherein the target transcription factor is selected from AP1; ATF6; ERG; ETV1; GLI3; HOXA9; MBD2; MEF2A; NF kappa B; POU3F2 (BRN2); PRDM13; RBPJ; RUNX1; RUNX2; SMAD3; SMAD4; SNAI1; TAZ; TCF21; TWIST1; MAML1; MAML2; EF2A; or YAP.
19. The artificial nucleic acid of any one of the preceding items, wherein the target transcription factor is selected from GLI3; HOXA9; MBD2; MEF2A; POU3F2 (BRN2); PRDM13; RUNX1; SMAD3; SMAD4; or SNAI1.
20. The artificial nucleic acid of any one of the preceding items, wherein the target transcription factor is a Runt-related transcription factor (RUNX), for example RUNX1, RUNX2, RUNX3.
21. The artificial nucleic acid of any one of the preceding items, wherein the target transcription factor is RUNX1.
22. The artificial nucleic acid of any one of the preceding items, wherein the transcription factor inhibitor comprises at least one amino acid sequence element A that is configured to bind to the target transcription factor or its transcription co-factor.
23. The artificial nucleic acid of any one of the preceding items, wherein the transcription factor inhibitor comprises at least one amino acid sequence element A that comprises or consists of an amino acid sequence selected or derived from the target transcription factor, an interaction partner of the target transcription factor, a binding partner of the target transcription factor, a transcription co-factor of the target transcription factor, an antibody moiety, an intrabody moiety, a peptide-based aptamer, or a fragment or variant of any of these.
24. The artificial nucleic acid of items 22 or 23, wherein the at least one amino acid sequence element A comprises or consists of an amino acid sequence that lacks a nuclear localization signal (NLS) or that has been modified to lack a functional NLS.
25. The artificial nucleic acid of items 22 to 24, wherein the at least one amino acid sequence element A comprises or consists of an amino acid sequence selected or derived from a transcription co-factor of the target transcription factor, or a fragment or variant thereof.
26. The artificial nucleic acid of items 25, wherein the transcription co-factor is selected from a transcription co-factor that forms a heterodimeric complex with the target transcription factor, preferably in the cytosol.
27. The artificial nucleic acid of items 22 to 26, wherein the at least one amino acid sequence element A comprises or consists of an amino acid sequence selected or derived from a transcription co-factor of RUNX, or a fragment or variant thereof.
28. The artificial nucleic acid of item 27, wherein the transcription co-factor of RUNX is selected or derived from CBFbeta, for example CBFbeta1 or CBFbeta2, or a fragment or variant thereof.
29. The artificial nucleic acid of item 28, wherein the CBFbeta amino acid sequence is an N-terminal fragment of a human CBFbeta, preferably a fragment comprising amino acid 1 to amino acid 141 of CBFbeta, more preferably a fragment comprising amino acid 1 to amino acid 162 of CBFbeta.
30. The artificial nucleic acid of any one of the preceding items, wherein the transcription factor inhibitor comprises at least one amino acid sequence element A that comprises or consists of an amino acid sequence selected or derived from CBFbeta being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 178-183, or fragments or variants of any of these.
31. The artificial nucleic acid of item 22 to 24, wherein the at least one amino acid sequence element A comprises or consists of an amino acid sequence selected or derived from the target transcription factor, or a fragment or variant thereof.
32. The artificial nucleic acid of item 31, wherein the amino acid sequence selected or derived from the target transcription factor comprises at least one amino acid substitution or deletion that reduces or prevents binding to its target DNA and/or at least one amino acid substitution or deletion that reduces or prevents nuclear translocation and/or at least one amino acid substitution or deletion that reduces or prevents homodimerization or heterodimerization.
33. The artificial nucleic acid of item 31 or 32, wherein the at least one amino acid sequence element A comprises or consists of an amino acid sequence selected or derived from RUNX or a fragment or variant thereof, preferably wherein the RUNX amino acid sequence is an N-terminal fragment of a human RUNX1, e.g. a fragment comprising the Runt homology domain (RHD).
34. The artificial nucleic acid of item 33, wherein at least one amino acid substitution in the RUNX1 amino acid sequence is selected from R80A, K83A, K83E, R135A, R139A, R142A, K167A, T169A, D171A, R174A, or R177A, or any functionally equivalent amino acid substitution at position R80, K83, R135, R139, R142, K167, T169, D171, R174, or R177.
35. The artificial nucleic acid of item 33 or 34, wherein at least one amino acid substitution in the RUNX1 amino acid sequence is selected from R174Q and/or K83E, or any functionally equivalent amino acid substitution at position R174 and/or K83.
36. The artificial nucleic acid of items 31 to 35, wherein the transcription factor inhibitor comprises at least one amino acid sequence element A that comprises or consists of an amino acid sequence selected or derived from RUNX1 being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 197-215, or fragments or variants of any of these.
37. The artificial nucleic acid of any one of the preceding items, wherein the transcription factor inhibitor comprises at least one amino acid sequence element B that is configured to prevent or reduce the interaction of the target transcription factor with its target DNA.
38. The artificial nucleic acid of any one of the preceding items, wherein the transcription factor inhibitor comprises at least one amino acid sequence element B that is configured to prevent or reduce interaction of the target transcription factor with at least one transcription co-factor of the target transcription factor.
39. The artificial nucleic acid of any one of the preceding items, wherein the transcription factor inhibitor comprises at least one amino acid sequence element B that is configured to prevent or reduce nuclear translocation of the target transcription factor or its transcription co-factor.
40. The artificial nucleic acid of any one of the preceding items, wherein the transcription factor inhibitor comprises at least one amino acid sequence element B that is configured to bind the target transcription factor or its transcription co-factor preferably in the cytosol.
41. The artificial nucleic acid of any one of the preceding items, wherein the transcription factor inhibitor comprises at least one amino acid sequence element B that is configured to repress the transcription activity of the (cellular) target transcription factor.
42. The artificial nucleic acid of any one of the preceding items, wherein the transcription factor inhibitor comprises at least one amino acid sequence element B that comprises or consists of an amino acid sequence selected or derived from a cytoplasmic protein, a protein that is associated with or binds to a cytoplasmic protein, or a fragment or variant thereof.
43. The artificial nucleic acid of items 37 to 42, wherein the amino acid sequence element B is selected or derived from a cytoskeletal protein or a protein that is associated with or binds to a cytoskeletal protein, or a fragment or variant of any of these.
44. The artificial nucleic acid of items 37 to 43, wherein the amino acid sequence element B is selected or derived from a myofibrillar protein, a microtubule protein, an intermediate filament protein, or a fragment or variant of any of these.
45. The artificial nucleic acid of items 37 to 44, wherein the amino acid sequence element B is selected or derived from a peptide or protein that is associated with or binds to a myofibrillar protein, a microtubule protein, an intermediate filament protein, or a fragment or variant of any of these.
46. The artificial nucleic acid of items 37 to 45, wherein the amino acid sequence element B is selected or derived from a protein that comprises an myofibrillar binding domain (e.g. actin), a microtubule binding domain, an intermediate filament binding domain.
47. The artificial nucleic acid of items 37 to 46, wherein the amino acid sequence element B is selected or derived from a protein that comprises an actin binding domain.
48. The artificial nucleic acid of items 37 to 47, wherein the amino acid sequence element B is selected from smooth muscle myosin heavy chain (SMMHC), or a fragment or variant thereof.
49. The artificial nucleic acid of item 48, wherein the amino acid sequence selected or derived from SMMHC comprises at least one of a high-affinity binding domain (HABD) and/or an assembly competent domain (ACD) and/or a transcriptional repression domain (TRD), or fragments or variants of any of these.
50. The artificial nucleic acid of item 48 or 49, wherein the SMMHC amino acid sequence is a C-terminal fragment of a human SMMHC, for example an SMMHC fragment comprising amino acid 1527 to amino acid 1972.
51. The artificial nucleic acid of item 48 to 50, wherein the SMMHC amino acid sequence comprises a deletion in the C-terminus of about 95aa (SMMHCAC95), for example an SMMHC fragment comprising amino acid 1527 to amino acid 1877.
52. The artificial nucleic acid of any one of the preceding items, wherein the transcription factor inhibitor comprises at least one amino acid sequence element B that comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 184-193, 1521, 1522, or fragments or variants of any of these.
53. The artificial nucleic acid of items 37 to 41, wherein the at least one element B comprises or consists of an amino acid sequence selected or derived from a transcriptional repressor of the target transcription factor, or a fragment or variant thereof.
54. The artificial nucleic acid of items 53, wherein the transcriptional repressor of the target transcription factor is selected or derived from RUNX1T1a, RUNX1T1b, or a fragment or variant of any of these.
55. The artificial nucleic acid of item 53 or 54, wherein the transcription factor inhibitor comprises at least one amino acid sequence element B that comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 216-230, or fragments or variants of any of these.
56. The artificial nucleic acid of items 37 to 41, wherein the at least one element B comprises or consists of an amino acid sequence selected or derived from a peptide or protein that promotes degradation of the target transcription factor, or a fragment or variant thereof.
57. The artificial nucleic acid of items 56, wherein the peptide or protein that promotes degradation may be selected from a protein that binds to E3 ligase, preferably a protein selected from HIF1alpha, MDM2, or CRBN, or a fragment or variant of any of these.
58. The artificial nucleic acid of items 56 or 57, wherein the peptide or protein that promotes degradation is selected or derived from HIF1alpha, preferably a HIF1alpha fragment comprising amino acid 549 to amino acid 575.
59. The artificial nucleic acid of items 56 to 58, wherein the transcription factor inhibitor comprises at least one amino acid sequence element B that comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 194 or 195, or fragments or variants of any of these.
60. The artificial nucleic acid of any one of the preceding items, wherein the transcription factor inhibitor is a fusion protein that comprises or consists of at least one amino acid sequence element A and at least one amino acid sequence element B, preferably wherein the at least one element A is located at the N-terminus of the transcription inhibitor and the at least one element B is located at the C-terminus of the transcription inhibitor.
61. The artificial nucleic acid of any one of the preceding items, wherein the transcription factor inhibitor comprises at least one further amino acid sequence element, preferably at least one further amino acid sequence element selected from a linker sequence, a transmembrane domain, a secretion signal, an element that extends protein half-life, or a fragment or variant of any of these.
62. The artificial nucleic acid of item 61, wherein the at least one further amino acid sequence element is selected from a linker sequence, preferably a flexible linker, more preferably a flexible GGS linker according to SEQ ID NO: 196, or a variant thereof.
63. The artificial nucleic acid of any one of the preceding items, wherein the transcription factor inhibitor is a RUNX inhibitor, preferably a RUNX1 inhibitor.
64. The artificial nucleic acid of any one of the preceding items, wherein the transcription factor inhibitor is a RUNX trap, preferably a RUNX1 trap.
65. The artificial nucleic acid of item 63 or 64, wherein the RUNX inhibitor, preferably the RUNX trap, comprises or consists of a fusion protein comprising a CBFbeta amino acid sequence element and an SMMHC amino acid sequence element.
66. The artificial nucleic acid of any one of the preceding items, wherein the transcription factor inhibitor, preferably the RUNX trap, comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 231-233, 1541-1548, or fragments or variants of any of these.
67. The artificial nucleic acid of any one of the preceding items, wherein the transcription factor inhibitor, preferably the RUNX trap, comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 232, or a fragment or variant thereof.
68 The artificial nucleic acid of items 63 to 67, wherein the RUNX inhibitor, preferably the RUNX trap, sequesters cellular RUNX by binding to RUNX in the cytosol and preferably trapping RUNX1 in the cytosol.
69. The artificial nucleic acid of item 63, wherein the RUNX inhibitor comprises or consists of a fusion protein comprising a CBFbeta amino acid sequence element and an HIF1alpha amino acid sequence element.
70. The artificial nucleic acid of item 69, wherein the RUNX inhibitor comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 234 or 235, or fragments or variants thereof.
71. The artificial nucleic acid of 69 or 70, wherein the RUNX inhibitor degrades cellular RUNX.
72. The artificial nucleic acid of items 63 to 71, wherein the RUNX inhibitor, preferably the RUNX trap, reduces or prevents the translocation of cellular CBFbeta from the cytosol to the nucleus by reducing or preventing its interaction with cellular RUNX.
73. The artificial nucleic acid of item 63, wherein the RUNX inhibitor comprises or consists of a fusion protein comprising a RUNX1 amino acid sequence element and a RUNX1T1a or RUNX1T1b amino acid sequence element.
74. The artificial nucleic acid of item 73, wherein the RUNX inhibitor comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 236-239, or fragments or variants thereof.
75. The artificial nucleic acid of item 63, wherein the RUNX inhibitor comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 197, or a fragment or a variant thereof.
76. The artificial nucleic acid of items 73 to 75, wherein the RUNX inhibitor sequesters cellular CBFbeta by binding to CBFbeta in the cytosol.
77. The artificial nucleic acid of items 73 to 76, wherein the RUNX inhibitor drives transcriptional repression of genes that are under control of RUNX.
78. The artificial nucleic acid of items 63 to 77, wherein the RUNX inhibitor, preferably the RUNX trap, reduces or prevents the interaction of cellular RUNX with cellular CBFbeta.
79. The artificial nucleic acid of items 63 to 78, wherein the RUNX inhibitor, preferably the RUNX trap, reduces cellular RUNX-CBFbeta complex formation and/or activity.
80. The artificial nucleic acid of items 63 to 79, wherein the RUNX inhibitor, preferably the RUNX trap, reduces the cellular expression of RUNX controlled or regulated gene products.
81. The artificial nucleic acid of items 63 to 80, wherein the RUNX inhibitor, preferably the RUNX trap, reduces the cellular expression of TGFbeta2, SMAD3, and/or COL1A1.
82. The artificial nucleic acid of items 63 to 81, wherein the RUNX inhibitor, preferably the RUNX trap, reduces the cellular expression of RUNX.
83. The artificial nucleic acid of items 63 to 82, wherein the RUNX inhibitor, preferably the RUNX trap, reduces or prevents pathological epithelial to mesenchymal transition (EMT), pathological cell proliferation, aberrant angiogenesis, aberrant neovascularization, degeneration, and/or fibrosis.
84. The artificial nucleic acid of any one of the preceding items, wherein the at least one coding sequence is a codon modified coding sequence, preferably wherein codon modified coding sequence is selected from a C maximized coding sequence, a CAI maximized coding sequence, human codon usage adapted coding sequence, a G/C content modified coding sequence, and a G/C optimized coding sequence, or any combination thereof, preferably wherein the at least one codon modified coding sequence is a G/C optimized coding sequence
85. The artificial nucleic acid of any one of the preceding items, wherein the at least one coding sequence comprises a nucleic acid sequence encoding an amino acid sequence element A, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 240-245, 302-307, 364-369, 426-431, 488-493, 550-555, 612-617, 674-679, 736-741, or a fragment or a variant of any of these.
86. The artificial nucleic acid of any one of the preceding items, wherein the at least one coding sequence comprises a nucleic acid sequence encoding an amino acid element B, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 246-255, 308-317, 370-379, 432-441, 494-503, 556-565, 618-627, 680-689, 742-751, 1523-1540, or a fragment or a variant of any of these.
87. The artificial nucleic acid of any one of the preceding items, wherein the at least one coding sequence comprises or consists of a nucleic acid sequence encoding a transcription factor inhibitor, preferably a RUNX trap, that is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 293-295, 355-357, 417-419, 479-481, 541-543, 603-605, 665-667, 727-729, 789-791, 1549-1558, or a fragment or a variant of any of these.
88. The artificial nucleic acid of any one of the preceding items, wherein the at least one coding sequence comprises or consists of a nucleic acid sequence encoding an transcription factor inhibitor, preferably a RUNX trap CBFbeta-SMMHC, that is identical or at least 70%, 80%, 90%, 91%, 92%, 93%, 94%, 95%, 96%, 97%, 98%, or 99% identical to SEQ ID NO: 418, or a fragment or a variant thereof.
89. The artificial nucleic acid of items 1 to 85, wherein the at least one coding sequence comprises a nucleic acid sequence encoding an amino acid sequence element B, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 257, 319, 381, 443, 505, 567, 629, 691, 753, or a fragment or a variant of any of these.
90. The artificial nucleic acid of items 89, wherein the at least one coding sequence comprises or consists of a nucleic acid sequence encoding a transcription factor inhibitor, preferably a RUNX inhibitor CBFbeta-HIF1alpha, that is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 296-297, 358-359, 420-421, 482-483, 544-545, 606-607, 668-669, 730-731, 792-793, or a fragment or a variant of any of these.
91. The artificial nucleic acid of items 1 to 84, wherein the at least one coding sequence comprises a nucleic acid sequence encoding an amino acid element A, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 259-277, 321-339, 383-401, 445-463, 507-525, 569-587, 631-649, 693-711, 755-773, or a fragment or a variant of any of these.
92. The artificial nucleic acid of item 91, wherein the at least one coding sequence comprises a nucleic acid sequence encoding an amino acid element B, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 278-292, 340-354, 402-416, 464-478, 526-540, 588-602, 650-664, 712-726, 774-788, or a fragment or a variant of any of these.
93. The artificial nucleic acid of items 91 to 92, wherein the at least one coding sequence comprises or consists of a nucleic acid sequence encoding a transcription factor inhibitor, preferably a RUNX inhibitor RUNX1-RUNX1T1, that is identical or at least 70%, 80%, 85%, 86%, 87%, 88%, 89%, 90%, 91%, 92%, 93%, 94%, 95%, 96%, 97%, 98%, or 99% identical to any one of the nucleic acid sequences SEQ ID NOs: 298-301, 360-363, 422-425, 484-487, 546-549, 608-611, 670-673, 732-735, 794-797, or a fragment or a variant of any of these.
94. The artificial nucleic acid of item 1 to 84, wherein the at least one coding sequence comprises or consists of a nucleic acid sequence encoding a transcription factor inhibitor, preferably a RUNX inhibitor RUNX1 (K83E,R174Q), that is identical or at least 70%, 80%, 85%, 86%, 87%, 88%, 89%, 90%, 91%, 92%, 93%, 94%, 95%, 96%, 97%, 98%, or 99% identical to any one of the nucleic acid sequences SEQ ID NOS: 259, 321, 383, 445, 507, 569, 631, 693, 755, or a fragment or a variant of any of these.
95. The artificial nucleic acid of any one of the preceding items, wherein the at least one heterologous untranslated region (UTR) is selected from at least one heterologous 5′-UTR and/or at least one heterologous 3′-UTR.
96. The artificial nucleic acid of item 95, wherein the at least one heterologous 3′-UTR comprises or consists of a nucleic acid sequence derived from a 3′-UTR of a gene selected from PSMB3, ALB7, alpha-globin, CASP1, COX6B1, GNAS, NDUFA1 and RPS9, or from a homolog, a fragment or a variant of any one of these genes, preferably wherein the at least one heterologous 3′-UTR comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 67-90, 109-120, or a fragment or a variant of any of these.
97. The artificial nucleic acid of item 95 or 96, wherein the at least one heterologous 5′-UTR comprises or consists of a nucleic acid sequence derived from a 5′-UTR of a gene selected from HSD17B4, RPL32, ASAH1, ATP5A1, MP68, NDUFA4, NOSIP, RPL31, SLC7A3, TUBB4B and UBQLN2, or from a homolog, a fragment or variant of any one of these genes, preferably selected from HSD17B4, preferably wherein the at least one heterologous 3′-UTR comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 1-32, 65-66, or a fragment or a variant of any of these.
98. The artificial nucleic acid of items 95 to 97, wherein the at least one heterologous 5′-UTR is selected from HSD17B4 and the at least one heterologous 3′ UTR is selected from PSMB3.
99. The artificial nucleic acid of items 95 to 98, wherein the at least one heterologous 3′-UTR comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 68, or a fragment or a variant thereof.
100. The artificial nucleic acid of items 95 to 99, wherein the at least one heterologous 5′-UTR comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 2, or a fragment or a variant thereof.
101. The artificial nucleic acid of any one of the preceding items, additionally comprising at least one poly(A) sequence, preferably wherein the at least one poly(A) sequence comprises about 40 to about 500 adenosine nucleotides, preferably about 60 to about 250 adenosine nucleotides, more preferably about 60 to about 150 adenosine nucleotides.
102. The artificial nucleic acid of item 101, wherein the at least one poly(A) sequence comprises about 100 adenosine nucleotides.
103. The artificial nucleic acid of item 101 or 102, wherein the at least one poly(A) sequence is located at the 3′ terminus, optionally, wherein the 3′ terminal nucleotide is an adenosine.
104. The artificial nucleic acid of any one of the preceding items, additionally comprising at least one poly(C) sequence and/or at least one miRNA binding site and/or histone stem-loop sequence.
105. The artificial nucleic acid of item 104, wherein the histone stem-loop sequence comprises or consists of a nucleic acid sequence identical or at least 70%, 80%, 90%, 91%, 92%, 93%, 94%, 95%, 96%, 97%, 98%, or 99% identical to any one of SEQ ID NO: 137, or a fragment or a variant thereof.
106. The artificial nucleic acid of any one of the preceding items, wherein the nucleic acid is an isolated nucleic acid.
107. The artificial nucleic acid of any one of the preceding items, wherein the artificial nucleic acid is a DNA, preferably a viral DNA, more preferably an Adeno-associated virus DNA.
108. The artificial nucleic acid of items 1 to 106, wherein the artificial nucleic acid is an RNA.
109. The artificial nucleic acid of item 108, wherein the RNA is selected from mRNA, circular RNA, replicon RNA, or viral RNA.
110. The artificial nucleic acid of item 108 or 109, wherein the RNA is an mRNA.
111a. The artificial nucleic acid of any one of the preceding items, wherein the nucleic acid, preferably the RNA, comprises at least one modified nucleotide, preferably selected from pseudouridine (ψ) or N1-methylpseudouridine (m1ψ).
111b. The artificial nucleic acid of item 111a, wherein the nucleic acid, preferably the RNA, comprises N1-methylpseudouridine (m1ψ).
111c. The artificial nucleic acid of item 111a or 111b, wherein the nucleic acid is a modified RNA wherein each Uracil is substituted by N1-methylpseudouridine (m1ψ).
112. The artificial nucleic acid of any one of the preceding items, wherein the nucleic acid, preferably the RNA, comprises a 5′-cap structure.
113. The artificial nucleic acid of item 112, wherein the 5′-cap structure is selected from a cap1 structure or a modified cap1 structure.
114. The artificial nucleic acid of any one of the preceding items, wherein the nucleic acid is an in vitro transcribed RNA, preferably wherein RNA in vitro transcription has been performed in the presence of a sequence optimized nucleotide mixture.
115. The artificial nucleic acid of any one of the preceding items, wherein the nucleic acid is a purified RNA, preferably wherein the RNA has been purified by RP-HPLC, AEX, SEC, hydroxyapatite chromatography, TFF, filtration, precipitation, core-bead flow through chromatography, oligo(dT) purification, cellulose-based purification, or any combination thereof.
116. The artificial nucleic acid of item 115, wherein the at least one step of purification is selected from RP-HPLC and/or TFF.
117. The artificial nucleic acid of any one of the preceding items, wherein the nucleic acid, preferably the RNA, has an integrity of at least about 50%, preferably of at least about 60%, more preferably of at least about 70%, most preferably of at least about 80%
118. The artificial nucleic acid of any one of the preceding items, wherein the nucleic acid, preferably the RNA, is suitable for use in treatment or prevention of a disease, disorder or condition, preferably an ocular disease, disorder or condition
119. The artificial nucleic acid of any one of the preceding items, wherein the nucleic acid, preferably the RNA, comprises the following sequence elements preferably in 5′- to 3′-direction:
   A) a 5′-cap structure;
   B) a 5′-UTR preferably selected or derived from a 5′-UTR of a HSD17B4 gene;
   C) a coding sequence encoding a transcription factor inhibitor, preferably a RUNX inhibitor;
   D) a 3′-UTR preferably selected or derived from a 3′-UTR of a PSMB3 gene;
   E) optionally, a histone stem-loop; and
   F) a poly(A) sequence preferably comprising about 100 A nucleotides.
120. The artificial nucleic acid of any one of the preceding items, wherein the nucleic acid, preferably the RNA, comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 798-1517, 1559-1582, or a fragment or variant of any of these sequences.
121. The artificial nucleic acid of any one of the preceding items, wherein the nucleic acid, preferably the RNA, comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 799-801, 809-811, 819-821, 829-831, 839-841, 849-851, 859-861, 869-871, 879-881, 889-891, 899-901, 909-911, 919-921, 929-931, 939-941, 949-951, 959-961, 969-971, 979-981, 989-991, 999-1001, 1009-1011, 1019-1021, 1029-1031, 1039-1041, 1049-1051, 1059-1061, 1069-1071, 1079-1081, 1089-1091, 1099-1101, 1109-1111, 1119-1121, 1129-1131, 1139-1141, 1149-1151, 1159-1161, 1169-1171, 1179-1181, 1189-1191, 1199-1201, 1209-1211, 1219-1221, 1229-1231, 1239-1241, 1249-1251, 1259-1261, 1269-1271, 1279-1281, 1289-1291, 1299-1301, 1309-1311, 1319-1321, 1329-1331, 1339-1341, 1349-1351, 1359-1361, 1369-1371, 1379-1381, 1389-1391, 1399-1401, 1409-1411, 1419-1421, 1429-1431, 1439-1441, 1449-1451, 1459-1461, 1469-1471, 1479-1481, 1489-1491, 1499-1501, 1509-1511, 1559-1582, or a fragment or variant of any of these sequences.
122. The artificial nucleic acid of any one of the preceding items, wherein the nucleic acid, preferably the RNA, comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 800, 810, 820, 830, 840, 850, 860, 870, 880, 890, 900, 910, 920, 930, 940, 950, 960, 970, 1567, 1568, 1577, 1578-1582, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, or a fragment or variant of any of these sequences.
123a. The artificial nucleic acid of any one of the preceding items, wherein the nucleic acid, preferably the RNA, comprises or consists of a nucleic acid sequence which is identical or at least 70%, 80%, 90%, 91%, 92%, 93%, 94%, 95%, 96%, 97%, 98%, or 99% identical to a nucleic acid sequence selected from SEQ ID NOS: 820, 1579, 1581, 910, 1580, 1582, or a fragment or variant of that sequence.
123b. The artificial nucleic acid of any one of the preceding items, wherein the nucleic acid, preferably the RNA, comprises or consists of a nucleic acid sequence which is identical or at least 90% identical to a nucleic acid sequence selected from SEQ ID NOs: 1579 or 1580, or a fragment or variant of that sequence.
123c. The artificial nucleic acid of item 123a or 123b, wherein the nucleic acid, preferably the RNA, comprises or consists of a nucleic acid sequence which is identical to the nucleic acid sequence SEQ ID NO: 1580.
124. A pharmaceutical composition comprising at least one artificial nucleic acid comprising at least one coding sequence encoding at least one RUNX transcription factor inhibitor as defined in any one of the items 1 to 123c.
125. The pharmaceutical composition of item 124, comprising at least one pharmaceutically acceptable carrier or excipient.
126. The pharmaceutical composition of item 124 or 125, wherein the at least one artificial nucleic acid is formulated in at least one cationic or polycationic compound.
127. The pharmaceutical composition of item 126, wherein the at least one cationic or polycationic compound is selected from a cationic or polycationic polymer, a cationic or polycationic polysaccharide, a cationic or polycationic lipid, a cationic or polycationic protein, a cationic or polycationic peptide, or any combinations thereof.
128. The pharmaceutical composition of item 127, wherein the one or more cationic or polycationic peptides are selected from SEQ ID NOs: 173-177, or any combinations thereof.
129. The pharmaceutical composition of item 127, wherein the cationic or polycationic polymer is selected from a polyethylene glycol/peptide polymer, preferably comprising HO-PEG5000-S-(S-CHHHHHHRRRRHHHHHHC-S-)7-S-PEG5000-OH (SEQ ID NO: 176 as peptide monomer), HO-PEG5000-S-(S-CHHHHHHRRRRHHHHHHC-S-) 4-S-PEG5000-OH (SEQ ID NO: 176 as peptide monomer), HO-PEG5000-S-(S-CGHHHHHRRRRHHHHHGC-S-) 7-S-PEG5000-OH (SEQ ID NO: 177 as peptide monomer), or HO-PEG5000-S-(S-CGHHHHHRRRRHHHHHGC-S-) 4-S-PEG5000-OH (SEQ ID NO: 177 as peptide monomer).
130. The pharmaceutical composition of item 127 or 129, wherein the cationic or polycationic polymer additionally comprises at least one lipidoid component.
131. The pharmaceutical composition of item 130, wherein the at least one lipidoid component is selected from 3-C12-OH, 3-C12-OH-cat, or 3-C12-C3-OH.
132. The pharmaceutical composition of items 124 to 127, wherein the at least one artificial nucleic acid is formulated in lipid-based carriers.
133. The pharmaceutical composition of item 132, wherein the lipid-based carriers are selected from liposomes, lipid nanoparticles, lipoplexes, solid lipid nanoparticles, lipo-polylexes, and/or nanoliposomes.
134. The pharmaceutical composition of item 132 or 133, wherein the lipid-based carriers are lipid nanoparticles, preferably wherein the lipid nanoparticles encapsulate the artificial nucleic acid.
135. The pharmaceutical composition of items 132 to 134, wherein the lipid-based carriers (e.g. LNPs) of the pharmaceutical composition comprise a cationic lipid selected or derived from 9-Heptadecanyl 8-{(2-hydroxyethyl)[6-oxo-6-(undecyloxy)hexyl]amino}octanoate, also referred to as SM-102.
136. The pharmaceutical composition of items 132 to 135, wherein the lipid-based carriers comprise at least one lipid selected from an aggregation-reducing lipid, a cationic lipid or ionizable lipid, a neutral lipid or phospholipid, or a steroid or steroid analog, or any combinations thereof.
137a. The pharmaceutical composition of item 136, wherein the I aggregation reducing lipid selected from a polymer conjugated lipid.
137b. The pharmaceutical composition of item 137a, wherein the polymer conjugated lipid is selected from a PEG-conjugated lipid or a PEG-free lipid.
138. The pharmaceutical composition of item 137a or 137b, wherein the polymer conjugated lipid is selected or derived from DMG-PEG 2000, C10-PEG2K, Cer8-PEG2K, a POZ-lipid, or ALC-0159.
139. The pharmaceutical composition of item 137 or 138, wherein the polymer conjugated lipid is not a PEG-conjugated lipid.
140a. The pharmaceutical composition of item 136 to 139, wherein the cationic lipid or ionizable lipid is selected from an amino lipid, preferably wherein the amino lipid comprises a tertiary amine group
140b. The pharmaceutical composition of item 136 or 140a, wherein one cationic or ionizable lipid is a lipid selected or derived from formula (III-1)

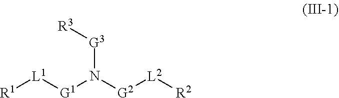

preferably, wherein one of L1 or L2 is —O(C═O)—, —(C═O)O—, —C(═O)—, —O—, —S(O)x-, —S—S—, —C(═O)S—, SC(═O)—, —NRaC(═O)—, —C(═O)NRa—, —NRaC(═O)NRa—, —OC(═O)NRa— or —NRaC(═O)O—, and the other of L1 or L2 is —O(C═O)—, —(C═O)O—, —C(═O)—, —O—, —S(O)x-, —S S—, —C(═O)S—, SC(═O)—, —NRaC(═O)—, —C(═O)NRa—, —NRaC(═O)NRa—, —OC(═O)NRa— or —NRaC(═O)O— or a direct bond; G1 and G2 are each independently unsubstituted C1-C12 alkylene or C1-C12 alkenylene; G3 is C1-C24 alkylene, C1-C24 alkenylene, C3-C8 cycloalkylene, C3-C8 cycloalkenylene; Ra is H or C1-C12 alkyl; R1 and R2 are each independently C6-C24 alkyl or C6-C24 alkenyl; R3 is H, OR5, CN, C(═O) OR4, OC(═O) R4 or —NR5C(═O) R4; R4 is C1-C12 alkyl; R5 is H or C1-C6 alkyl; and x is 0, 1 or 2.
140c. The pharmaceutical composition of item 136 to 140b, wherein one cationic or ionizable lipid is selected or derived from a lipid according to formula III-3:

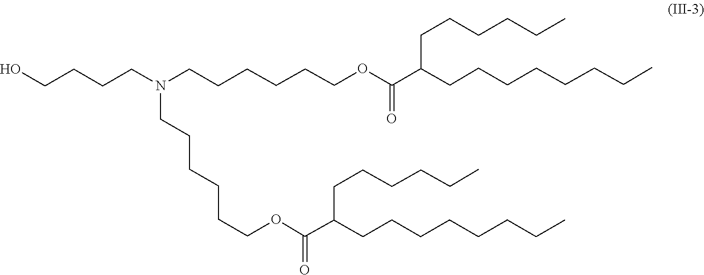 [SMILES: CCCCCCCCC(CCCCCC)C(=O)OCCCCCCN(CCCCO)CCCCCCOC(=O)C(CCCCCC)CCCCCCCC]

140d. The pharmaceutical composition of items 132 to 140c, wherein the lipid-based carriers comprise a cationic lipid selected or derived from ALC-0315, SM-102, SS-33/4PE-15, HEXA-C5DE-PipSS, or compound C26.
141. The pharmaceutical composition of items 132 to 140d, wherein the lipid-based carriers comprise a neutral lipid selected or derived from DSPC, DHPC, or DphyPE.
142. The pharmaceutical composition of items 132 to 141, wherein the lipid-based carriers comprise a steroid or steroid analog selected or derived from cholesterol, cholesteryl hemisuccinate (CHEMS), preferably cholesterol.
143. The pharmaceutical composition of items 132 to 142, wherein the lipid-based carriers comprise
   (i) at least one cationic lipid, preferably as defined in item 140;
   (ii) at least one neutral lipid, preferably as defined in item 141;
   (iii) at least one steroid or steroid analogue, preferably as defined in item 142; and
   (iv) at least one aggregation reducing lipid, preferably as defined in items 137 to 139.
144a. The pharmaceutical composition of items 132 to 143, wherein the lipid-based carriers comprise
   (i) at least one cationic lipid selected from ALC-0315;
   (ii) at least one neutral lipid selected from DSPC;
   (iii) at least one steroid or steroid analogue selected from cholesterol; and
   (iv) at least one aggregation reducing lipid selected from ALC-0159.
144b. The pharmaceutical composition of items 132 to 144a, wherein the lipid-based carriers comprise
   (i) at least one cationic lipid selected from lipid SM-102
   (ii) at least one neutral lipid selected from DSPC
   at least one steroid or steroid analogue selected from cholesterol
   (iv) at least one aggregation reducing lipid selected from-PEG 2000.
145. The pharmaceutical composition of items 132 to 144b, wherein the lipid-based carriers comprise about 20-60% cationic lipid, about 5-25% neutral lipid, about 25-55% steroid or steroid analogue, and about 0.5-15% aggregation reducing lipid.
146. The pharmaceutical composition of items 132 to 145, wherein the wt/wt ratio of lipid to nucleic acid in the lipid-based carrier is from about 10:1 to about 60:1, preferably from about 20:1 to about 30:1.
147. The pharmaceutical composition of items 132 to 146, wherein the N/P ratio of the lipid-based carriers encapsulating the nucleic acid is in a range from about 1 to about 10, preferably in a range from about 5 to about 7.
148. The pharmaceutical composition of items 132 to 147, wherein the lipid-based carriers have a Z-average size in a range of about 50 nm to about 120 nm.
149. The pharmaceutical composition of items 124 to 148, additionally comprising at least one antagonist of at least one RNA sensing pattern recognition receptor selected from a Toll-like receptor, preferably a TLR7 antagonist and/or a TLR8 antagonist.
150. The pharmaceutical composition of items 124 to 149, additionally comprising at least one small molecule inhibitor an inhibitory nucleic acid (siRNA) of the target transcription factor, preferably a small molecule inhibitor or an inhibitory nucleic acid (siRNA) of RUNX.
151. The pharmaceutical composition of items 124 to 150, wherein the composition is a liquid composition or a dried composition.
152. The pharmaceutical composition or the artificial nucleic acid of any one of the preceding items, wherein upon intramuscular, intranasal, transdermal, intradermal, intralesional, intracranial, subcutaneous, intracardial, intratumoral, intravenous, intrapulmonal, intraarticular, sublingual, pulmonary, intrathecal, or ocular administration of the composition or nucleic acid to a cell, tissue, or subject, the transcription factor inhibitor is produced.
153. The pharmaceutical composition or the artificial nucleic acid of any one of the preceding items, wherein upon local administration of the composition or nucleic acid to a cell, tissue, or subject, the transcription factor inhibitor is produced.
154. The pharmaceutical composition or the artificial nucleic acid of item 152 or 153, wherein the administration is an ocular administration.
155. The pharmaceutical composition or the artificial nucleic acid of item 154, wherein the ocular administration is selected from topical, intravitreal, intracameral, subconjunctival, subretinal, subtenon, retrobulbar, topical, orbital, suprachoroidal, posterior juxtascleral, or intraoperative administration, preferably intravitreal or intraoperative administration.
156. The pharmaceutical composition or the artificial nucleic acid of any one of the preceding items, wherein the administration is performed into a tamponade agent-filled human eye.
157. The pharmaceutical composition or the artificial nucleic acid of item 156 wherein the tamponade agent is a gas agent or a silicone agent.
158. The pharmaceutical composition or the artificial nucleic acid of any of the preceding items, wherein the ocular administration is performed using lipid-based carriers, preferably lipid nanoparticles (LNPs) as defined in item 144a or 144b.
159. The pharmaceutical composition or the artificial nucleic acid of item 158, wherein the lipid-based carriers are LNPs composed of lipids as defined in items 144a or 144b
160. The pharmaceutical composition or the artificial nucleic acid of any one of the preceding items, wherein ocular administration of the composition or the nucleic acid leads to a production of the transcription factor inhibitor in cells and/or tissues of the eye, preferably in cells and/or tissues selected from cornea, lens, ciliary body, vitreous, sclera, choroid, retina, optic nerve, macula, scleral cells, choroid plexus epithelial cells, retinal cells, inflammatory cells, retinal pigment epithelium (RPE), Bruch's membrane, and retinal or choroidal blood vessels.
161. The pharmaceutical composition or the artificial nucleic acid of any one of the preceding items, wherein ocular administration of the composition or the nucleic acid leads to a production of the transcription factor inhibitor in retinal pigment epithelial (RPE) cells or cells derived from them.
162. The pharmaceutical composition or the artificial nucleic acid of any one of the preceding items, wherein ocular administration of the composition or the nucleic acid leads to a production of the transcription factor inhibitor in retinal cells selected from photoreceptor cells, bipolar cells, ganglion cells, horizontal cells, Muller cells, mural cells, microglia, and amacrine cells.
163a. A pharmaceutical composition comprising at least one mRNA encoding a therapeutic protein, formulated in a lipid nanoparticle (LNP), wherein the LNP comprise an aggregation reducing lipid, a cationic lipid selected or derived from formula (III), a neutral lipid or phospholipid and a steroid or steroid analog for use in treatment or prevention of an ophthalmic disease, disorder or condition, wherein said composition is administered via intravitreal administration to a subject in need thereof.
163b. The pharmaceutical composition comprising at least one mRNA encoding a therapeutic protein, formulated in a lipid nanoparticle (LNP) for use in treatment or prevention of an ophthalmic disease of item 163a, wherein the LNP is further characterized by items 140 to 148.
164. A Kit or kit of parts comprising at least one artificial nucleic acid of any one of items 1 to 123, and/or at least one pharmaceutical composition of any one of items 124 to 163, optionally comprising a liquid vehicle for solubilising, and optionally comprising technical instructions providing information on administration and dosage of the components.
165. An artificial nucleic acid of any one of items 1 to 123, or a pharmaceutical composition of any one of items 124 to 163, or a kit or kit of parts of item 164, for use as a medicament in treating or preventing a disease, disorder, or condition in a subject.
166. An artificial nucleic acid of any one of items 1 to 123, or a pharmaceutical composition of any one of items 124 to 163, or a kit or kit of parts of item 164, for use as a medicament in treating or preventing an ocular disease, disorder, or condition in a subject.
167. The artificial nucleic acid of any one of items 1 to 123, or the pharmaceutical composition of any one of items 124 to 163, or the kit or kit of parts of item 164, for use as a medicament of item 165 or 166, wherein the disease, disorder, or condition is associated with or caused by an overexpressed and/or an overactive transcription factor including aging.
168. The artificial nucleic acid of any one of items 1 to 123, or the pharmaceutical composition of any one of items 124 to 163, or the kit or kit of parts of item 164, for use as a medicament of item 165 to 166, wherein the disease, disorder, or condition is associated with or caused by pathological epithelial to mesenchymal transition (EMT), pathological cell proliferation, aberrant angiogenesis, aberrant neovascularization, degeneration, fibrosis and/or solid tumors.
169. The artificial nucleic acid of any one of items 1 to 123, or the pharmaceutical composition of any one of items 124 to 163, or the kit or kit of parts of item 164 for use as a medicament of item 165 to 166, wherein the ocular disease, disorder, or condition is associated with or caused by aberrant proliferation or migration of retinal pigment epithelial (RPE) cells in a subject.
170. The artificial nucleic acid of any one of items 1 to 123, or the pharmaceutical composition of any one of items 124 to 163, or the kit or kit of parts of item 164 for use as a medicament of item 165 to 166, wherein the ocular disease, disorder, or condition is selected from proliferative diabetic retinopathy (PDR), macular edema, non-proliferative diabetic retinopathy, age-related macular degeneration, geographic atrophy, ocular neovascularization, retinopathy of prematurity (ROP), a retinal vein occlusion, ocular ischemic syndrome, neovascular glaucoma, a retinal hemangioma, Coats' disease, FEVR, or Norrie disease, persistent hyperplastic primary vitreous (PHPV), thyroid eye disease, epiretinal membrane, small vessel disease or proliferative vitreoretinopathy (PVR).
171. The artificial nucleic acid of any one of items 1 to 123, or the pharmaceutical composition of any one of items 124 to 163, or the kit or kit of parts of item 164 for use as a medicament of item 165 to 166, wherein the ocular disease, disorder, or condition is PVR, preferably the prevention of PVR.
172. The artificial nucleic acid of any one of items 1 to 123, or the pharmaceutical composition of any one of items 124 to 163, or the kit or kit of parts of item 164 for use as a medicament of item 171, wherein cell proliferation and/or cell growth is reduced in eyes with PVR.
173. The artificial nucleic acid of any one of items 1 to 123, or the pharmaceutical composition of any one of items 124 to 163, or the kit or kit of parts of item 164 for use as a medicament of item 165 to 172, wherein the subject has suffered a trauma to the eye, comprises a retinal hole, a retinal tear, a retinal detachment disorder, or has undergone an ocular surgery.
174. The artificial nucleic acid of any one of items 1 to 123, or the pharmaceutical composition of any one of items 124 to 163, or the kit or kit of parts of item 164 for use as a medicament of item 173, wherein the retinal detachment disorder is selected from rhegmatogenous retinal detachment, exudative retinal detachment, or tractional retinal detachment.
175. The artificial nucleic acid of any one of items 1 to 123, or the pharmaceutical composition of any one of items 124 to 163, or the kit or kit of parts of item 164 for use as a medicament of item 165 to 174, wherein the use comprises administration of the artificial nucleic acid, the pharmaceutical composition, or the kit or kit of parts by local administration, preferably by ocular administration.
176. The artificial nucleic acid of any one of items 1 to 123, or the pharmaceutical composition of any one of items 124 to 163, or the kit or kit of parts of item 164 for use as a medicament of item 165 to 175, wherein the use comprises administration of the artificial nucleic acid, the pharmaceutical composition, or the kit or kit of parts by intravitreal administration, by administration prior to an ocular surgery, during an ocular surgery, or after an ocular surgery.
177. The artificial nucleic acid of any one of items 1 to 123, or the pharmaceutical composition of any one of items 124 to 163, or the kit or kit of parts of item 164 for use as a medicament of item 165 to 176, wherein the disease, disorder, or condition is associated with or caused by overexpressed and/or overactive RUNX, preferably RUNX1.
178. A method of treating or preventing a disease, disorder or condition, wherein the method comprises applying or administering to a subject in need thereof an effective amount of the artificial nucleic acid of any one of items 1 to 123, or the pharmaceutical composition of any one of items 124 to 163, or the kit or kit of parts of item 164.
179. The method of treating or preventing a disease, disorder or condition of item 178, wherein the disease, disorder, or condition is an ocular disease, disorder, or condition.
180. The method of treating or preventing a disease, disorder or condition of item 178 or 179, wherein the disease, disorder, or condition is associated with or caused by an overexpressed and/or an overactive transcription factor.
181. The method of treating or preventing a disease, disorder or condition of item 177 to 179, wherein the disease, disorder, or condition associated with or caused by pathological epithelial to mesenchymal transition (EMT), pathological cell proliferation, aberrant angiogenesis, aberrant neovascularization, degeneration, solid tumors, and/or fibrosis.
182. The method of treating or preventing a disease, disorder or condition of items 178 to 181, wherein the ocular disease, disorder, or condition is associated with or caused by aberrant proliferation or migration of retinal pigment epithelial (RPE) cells in a subject.
183. The method of treating or preventing a disease, disorder or condition of items 178 to 182, wherein the ocular disease, disorder, or condition is selected from proliferative diabetic retinopathy (PDR), macular edema, non-proliferative diabetic retinopathy, age-related macular degeneration, geographic atrophy, ocular neovascularization, retinopathy of prematurity (ROP), a retinal vein occlusion, ocular ischemic syndrome, neovascular glaucoma, a retinal hemangioma, Coats' disease, FEVR, or Norrie disease, persistent hyperplastic primary vitreous (PHPV), thyroid eye disease, epiretinal membrane, small vessel disease, or proliferative vitreoretinopathy (PVR).
184. The method of treating or preventing a disease, disorder or condition of items 178 to 183, wherein the ocular disease, disorder, or condition is PVR, preferably the prevention of PVR.
185. The method of treating or preventing a disease, disorder or condition of items 184, wherein cell proliferation and/or cell growth is reduced in eyes with PVR.
186. The method of treating or preventing a disease, disorder or condition of items 178 to 185, wherein the subject has suffered a trauma to the eye, comprises a retinal hole, a retinal tear, a retinal detachment disorder, or has undergone an ocular surgery.
187. The method of treating or preventing a disease, disorder or condition of items 186, wherein the retinal detachment disorder is selected from rhegmatogenous retinal detachment, exudative retinal detachment, or tractional retinal detachment.
188. The method of treating or preventing a disease, disorder or condition of items 178 to 187, wherein the applying or administering is performed more than once, for example two times, three times, or four times, for example periodically.
189. The method of treating or preventing a disease, disorder or condition of items 178 to 188, wherein the applying or administering is performed by local administration, preferably by ocular administration.
190. The method of treating or preventing a disease, disorder or condition of items 178 to 189, wherein the applying or administering is performed by intravitreal administration, by administration prior to an ocular surgery, during an ocular surgery, and/or after an ocular surgery.
191. The method of treating or preventing a disease, disorder or condition of items 178 to 190, wherein the disease, disorder, or condition is associated with or caused by overexpressed and/or overactive RUNX, preferably RUNX1.
192. A method of treating or preventing or preventing an ocular disease in a subject comprising, administering to the subject an effective amount of a composition comprising an artificial nucleic acid comprising at least one coding sequence encoding the RUNX inhibitor, the RUNX inhibitor comprising:
   at least one amino acid element A selected or derived from CBFbeta configured to bind to the target transcription factor RUNX in the cytosol, preferably wherein element A is further characterized by any of items 22 to 30; and
   at least one amino acid element B selected or derived from SMMHC configured to trap the target transcription factor RUNX in the cytosol, preferably wherein element B is further characterized by any of items 37 to 52.
193. The method of item 192, wherein the composition is administered by ocular administration.
194. The method of item 193, wherein the ocular administration is selected from intravitreal or intraoperative administration.
195. The method of items 192 to 194, wherein the artificial nucleic acid is an RNA, preferably an mRNA as defined by any of items 110 to 123.
196. The method of items 192 to 194, wherein the artificial nucleic acid is comprised in a viral vector, preferably an AAV vector.
197. The method of items 192 to 196, wherein the artificial nucleic acid is formulated in lipid-based carriers, preferably as defined by any of items 132 to 146.
198. The method of items 192 to 197, wherein the ocular disease is associated with or caused by overexpressed RUNX and/or overactive RUNX, preferably, wherein the ocular disease is PVR.
199. A method of reducing the activity of a transcription factor in a cell or a subject, wherein the method comprises
   a) applying or administering an artificial nucleic acid comprising at least one coding sequence encoding at least one transcription factor inhibitor of items 1 to 123; or
   b) applying or administering a pharmaceutical composition comprising the artificial nucleic acid comprising at least one coding sequence encoding at least one transcription factor inhibitor of items 124 to 157;
   to a cell, tissue, or subject, wherein the transcription factor inhibitor is produced in the cell, tissue, or subject after administration or application to said cell, tissue, or subject.
200. The method of item 199, wherein the transcription factor inhibitor is a transcription factor trap.
201. The method of item 199 or 200, wherein
   the produced transcription factor inhibitor is a dominant negative inhibitor of the target transcription factor;
   and/or
   the produced transcription factor inhibitor binds to the target transcription factor or binds to at least one transcription co-factor of the target transcription factor; and/or
   the produced transcription factor inhibitor reduces or prevents interaction of a target transcription factor with its target DNA; and/or
   the produced transcription factor inhibitor reduces or prevents interaction of a target transcription factor with at least one of its transcription co-factors; and/or
   the produced transcription factor inhibitor reduces or prevents nuclear translocation of a target transcription factor; and/or
   the produced transcription factor inhibitor or transcription factor trap reduces the activity of a target transcription factor; and/or
   the produced transcription factor inhibitor or transcription factor trap reduces the cellular expression of a target transcription factor; and/or
   the produced transcription factor inhibitor or transcription factor trap reduces the cellular expression of proteins that are controlled or regulated by a target transcription factor; and/or
   the produced transcription factor inhibitor or transcription factor trap triggers plasma membrane anchoring of transcription factors.
202. The method of items 199 to 201, wherein the produced transcription factor inhibitor is a RUNX inhibitor or a RUNX trap and the target transcription factor is RUNX.
203. The method of items 202, wherein the produced RUNX inhibitor or RUNX trap
   sequesters cellular RUNX by binding to RUNX in the cytosol or sequesters cellular CBFbeta by binding to CBFbeta in the nucleus
   reduces or prevents the interaction of cellular RUNX with cellular CBFbeta; and/or
   reduces cellular RUNX-CBFbeta complex formation and/or activity; and/or
   reduces the cellular expression of RUNX controlled or regulated gene products; and/or
   reduces the cellular expression of TGFbeta2, SMAD3, and/or COL1A1; and/or
   reduces the cellular expression of RUNX; and/or
   reduces or prevents pathological epithelial to mesenchymal transition (EMT), pathological cell proliferation, aberrant angiogenesis, aberrant neovascularization, degeneration, and/or fibrosis; and/or
   reduces or prevents cell proliferation and/or cell growth in eyes with PVR.
204. The method of item 200 to 203, wherein an ocular administration leads to a production of the transcription factor inhibitor in cells and/or tissues of the eye, preferably in retinal pigment epithelial (RPE) cells.
205. The method of items 200 to 203, wherein the reduction of the activity of a transcription factor is a transient reduction of the activity of a transcription factor.
206. The method of items 200 to 205, wherein the transcription factor inhibitor is a dominant negative inhibitor of the target transcription factor.

### Preferred Item List

Particularly preferred embodiments of the present invention are provided in the following item list:
1: An artificial RNA comprising at least one coding sequence encoding at least one RUNX inhibitor comprising
   (i) at least one amino acid sequence element A that is configured to bind a RUNX target transcription factor; and
   (ii) at least one amino acid sequence element B that is configured to prevent or reduce the interaction of the target transcription factor with its target DNA, wherein the RNA comprises at least one heterologous untranslated region (UTR).
2: The artificial RNA of item 1, wherein the at least one amino acid sequence element A comprises or consists of an amino acid sequence selected or derived from a transcription co-factor of RUNX, or a fragment or variant thereof.
3: The artificial RNA of item 2, wherein the transcription co-factor of RUNX is selected or derived from CBFbeta, for example CBFbeta1 or CBFbeta2, or a fragment or variant thereof.
4: The artificial RNA of item 3, wherein the CBFbeta amino acid sequence is an N-terminal fragment of a human CBFbeta, preferably a fragment comprising amino acid 1 to amino acid 141 of CBFbeta, more preferably a fragment comprising amino acid 1 to amino acid 162 of CBFbeta.
5: The artificial RNA of any one of the preceding items, wherein the RUNX inhibitor comprises at least one amino acid sequence element A that comprises or consists of an amino acid sequence selected or derived from CBFbeta being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 178-183, or fragments or variants of any of these.
6: The artificial RNA of any one of the preceding items, wherein the amino acid sequence element B is configured to bind or trap the RUNX target transcription factor in the cytosol.
7: The artificial RNA of any one of the preceding items, wherein the at least one amino acid sequence element B comprises or consists of an amino acid sequence selected or derived from a cytoplasmic protein, a protein that is associated with or binds to a cytoplasmic protein, or a fragment or variant thereof.
8: The artificial RNA of any one of the preceding items, wherein the amino acid sequence element B is selected or derived from a protein that comprises a myofibrillar binding domain (e.g. actin), a microtubule binding domain, or an intermediate filament binding domain.
9: The artificial RNA of any one of the preceding items, wherein the amino acid sequence element B is selected or derived from a protein that comprises an actin binding domain
10: The artificial RNA of any one of the preceding items, wherein the amino acid sequence element B is selected or derived from a smooth muscle myosin heavy chain (SMMHC), or a fragment or variant thereof.
11: The artificial RNA of any item 10, wherein the SMMHC amino acid sequence is a C-terminal fragment of a human SMMHC, for example an SMMHC fragment comprising amino acid 1527 to amino acid 1972.
12: The artificial RNA of item 10 or 11, wherein the SMMHC amino acid sequence comprises a deletion in the C-terminus of about 95aa (SMMHCAC95), for example an SMMHC fragment comprising amino acid 1527 to amino acid 1877.
13: The artificial RNA of any one of the preceding items, wherein RUNX inhibitor comprises at least one amino acid sequence element B that comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 184-193, 1521, 1522, or fragments or variants of any of these.
14: The artificial RNA of any one of the preceding items, wherein the RUNX inhibitor comprises or consists of a fusion protein comprising a CBFbeta amino acid sequence element and an SMMHC amino acid sequence element.
15: The artificial RNA of any one of the preceding items, wherein the RUNX inhibitor comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 231-233, 1541-1548, or fragments or variants of any of these.
16: The artificial RNA of any one of the preceding items, wherein the RUNX inhibitor comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 232, or a fragment or variant thereof.
17: The artificial RNA of any one of the preceding items, wherein the RUNX inhibitor is a RUNX trap, preferably wherein the RUNX trap sequesters cellular RUNX by binding to RUNX in the cytosol and/or trapping RUNX in the cytosol
18: The artificial RNA of any one of the preceding items, wherein the RUNX inhibitor, preferably the RUNX trap, reduces cellular RUNX-CBFbeta complex formation and/or activity.
19: The artificial RNA of any one of the preceding items, wherein the at least one coding sequence is a G/C optimized coding sequence
20: The artificial RNA of any one of the preceding items, wherein the at least one coding sequence comprises or consists of a nucleic acid sequence encoding a RUNX inhibitor, preferably a RUNX trap, that is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 293-295, 355-357, 417-419, 479-481, 541-543, 603-605, 665-667, 727-729, 789-791, 1549-1558, or a fragment or a variant of any of these.
21: The artificial RNA of item 20, wherein the at least one coding sequence comprises or consists of a nucleic acid sequence identical or at least 70%, 80%, 90%, 91%, 92%, 93%, 94%, 95%, 96%, 97%, 98%, or 99% identical to SEQ ID NO: 418, or a fragment or a variant thereof.
22: The artificial RNA of any one of the preceding items, wherein the at least one heterologous untranslated region (UTR) is selected from at least one heterologous 5′-UTR and/or at least one heterologous 3′-UTR.
23: The artificial RNA of item 22, wherein the at least one heterologous 5′-UTR is selected from HSD17B4 and the at least one heterologous 3′ UTR is selected from PSMB3.
24: The artificial RNA of any one of the preceding items, additionally comprising at least one poly(A) sequence, preferably wherein the at least one poly(A) sequence comprises about 60 to about 150 adenosine nucleotides, preferably about 100 adenosine nucleotides.
25: The artificial RNA of any one of the preceding items, wherein the RNA is an isolated RNA.
26: The artificial RNA of any one of the preceding items, wherein the RNA is selected from mRNA, circular RNA, replicon RNA, or viral RNA, preferably an mRNA
27: The artificial RNA of any one of the preceding items, wherein the RNA comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 799-801, 809-811, 819-821, 829-831, 839-841, 849-851, 859-861, 869-871, 879-881, 889-891, 899-901, 909-911, 919-921, 929-931, 939-941, 949-951, 959-961, 969-971, 979-981, 989-991, 999-1001, 1009-1011, 1019-1021, 1029-1031, 1039-1041, 1049-1051, 1059-1061, 1069-1071, 1079-1081, 1089-1091, 1099-1101, 1109-1111, 1119-1121, 1129-1131, 1139-1141, 1149-1151, 1159-1161, 1169-1171, 1179-1181, 1189-1191, 1199-1201, 1209-1211, 1219-1221, 1229-1231, 1239-1241, 1249-1251, 1259-1261, 1269-1271, 1279-1281, 1289-1291, 1299-1301, 1309-1311, 1319-1321, 1329-1331, 1339-1341, 1349-1351, 1359-1361, 1369-1371, 1379-1381, 1389-1391, 1399-1401, 1409-1411, 1419-1421, 1429-1431, 1439-1441, 1449-1451, 1459-1461, 1469-1471, 1479-1481, 1489-1491, 1499-1501, 1509-1511, 1559-1582, or a fragment or variant of any of these sequences.
28: The artificial RNA of any one of the preceding items, wherein the RNA comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 800, 810, 820, 830, 840, 850, 860, 870, 880, 890, 900, 910, 920, 930, 940, 950, 960, 970, 1567, 1568, 1577, 1578-1582, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, or a fragment or variant of any of these sequences.
29: The artificial RNA of any one of the preceding items, wherein the nucleic acid, preferably the RNA, comprises or consists of a nucleic acid sequence which is identical or at least 70%, 80%, 90%, 91%, 92%, 93%, 94%, 95%, 96%, 97%, 98%, or 99% identical to a nucleic acid sequence selected from SEQ ID NOs: 820, 1579, 1581, 910, 1580, 1582, or a fragment or variant of that sequence.
30: The artificial RNA of any one of the preceding items, wherein the RNA comprises at least one modified nucleotide, preferably selected from pseudouridine (w) or N1-methylpseudouridine (m1w).
31: The artificial RNA of any one of the preceding items, wherein the RNA comprises or consists of an N1-methylpseudouridine (m1w) or pseudouridine (w) modified nucleic acid sequence which is identical or at least 70%, 80%, 90%, 91%, 92%, 93%, 94%, 95%, 96%, 97%, 98%, or 99% identical to a nucleic acid sequence selected from SEQ ID NOs: 1579-1582, or a fragment or variant of that sequence.
32: The artificial RNA of item 31, wherein the RNA comprises or consists of an N1-methylpseudouridine (m1w) modified nucleic acid sequence which is identical or at least 70%, 80%, 90%, 91%, 92%, 93%, 94%, 95%, 96%, 97%, 98%, or 99% identical to a nucleic acid sequence selected from SEQ ID NOs: 1580 or 1579, or a fragment or variant of that sequence.
33: The artificial RNA of any one of the preceding items, wherein the RNA is an mRNA that comprises a 5′-cap structure, preferably a 5′-cap1 structure.
34: A pharmaceutical composition comprising at least one artificial RNA comprising at least one coding sequence encoding at least one RUNX inhibitor as defined in any one of the items 1 to 33.
35: The pharmaceutical composition of item 34, wherein the at least one artificial RNA is formulated in at least one cationic or polycationic compound preferably selected from a cationic or polycationic polymer, a cationic or polycationic polysaccharide, a cationic or polycationic lipid, a cationic or polycationic protein, a cationic or polycationic peptide, or any combinations thereof.
36: The pharmaceutical composition of items 34 or 35, wherein the at least one artificial RNA is formulated in lipid-based carriers preferably selected from liposomes, lipid nanoparticles, lipoplexes, solid lipid nanoparticles, lipo-polylexes, and/or nanoliposomes.
37: The pharmaceutical composition of item 36, wherein the lipid-based carriers are lipid nanoparticles, preferably wherein the lipid nanoparticles encapsulate the artificial RNA.
38: The pharmaceutical composition of items 36 or 37, wherein the lipid-based carriers comprise at least one lipid selected from an aggregation-reducing lipid, at least one lipid selected from a cationic lipid or ionizable lipid, at least one lipid selected from a neutral lipid or phospholipid, and at least one lipid selected from or a steroid or steroid analog.
39: The pharmaceutical composition of item 38, wherein the lipid-based carriers comprise an aggregation reducing lipid selected from a polymer conjugated lipid.
40: The pharmaceutical composition of item 38 or 39, wherein the aggregation reducing lipid is a polymer conjugated lipid selected or derived from DMG-PEG 2000, C10-PEG2K, Cer8-PEG2K, a POZ-lipid, or ALC-0159
41: The pharmaceutical composition of items 38 to 40, wherein the at least one cationic lipid or ionizable lipid is selected from an amino lipid, preferably wherein the amino lipid comprises a tertiary amine group.
42: The pharmaceutical composition of items 38 to 41, wherein the at least one cationic or Ionizable lipid is a lipid selected or derived from formula (III-1).
43: The pharmaceutical composition of items 38 to 42, wherein the at least one cationic or ionizable lipid is selected or derived from a lipid according to formula III-3.
44: The pharmaceutical composition of items 38 to 43, wherein the lipid-based carriers comprise a cationic lipid selected or derived from ALC-0315, SM-102, SS-33/4PE-15, HEXA-C5DE-PipSS, or compound C26.
45: The pharmaceutical composition of items 38 to 44, wherein the lipid-based carriers comprise a neutral lipid selected or derived from DSPC, DHPC, or DphyPE.
46: The pharmaceutical composition of items 38 to 45, wherein the lipid-based carriers comprise a steroid or steroid analog selected or derived from cholesterol, cholesteryl hemisuccinate (CHEMS), preferably cholesterol.
47: The pharmaceutical composition of items 38 to 46, wherein the lipid-based carriers comprise
   (i) at least one cationic lipid, preferably as defined in item 41 to 44;
   (ii) at least one neutral lipid, preferably as defined in item 45;
   (iii) at least one steroid or steroid analogue, preferably as defined in item 46; and
   (iv) at least one aggregation reducing lipid, preferably as defined in item 39 or 40.
48: The pharmaceutical composition of items 38 to 47, wherein the lipid-based carriers comprise
   (i) at least one cationic lipid selected from ALC-0315;
   (ii) at least one neutral lipid selected from DSPC;
   (iii) at least one steroid or steroid analogue selected from cholesterol; and
   (iv) at least one aggregation reducing lipid selected from ALC-0159.
49: The pharmaceutical composition of items 38 to 48, wherein the lipid-based carriers comprise about 20-60% cationic lipid, about 5-25% neutral lipid, about 25-55% steroid or steroid analogue, and about 0.5-15% aggregation reducing lipid.
50: The pharmaceutical composition of items 38 to 49, wherein the wt/wt ratio of lipid to nucleic acid in the lipid-based carrier is from about 10:1 to about 60:1, preferably from about 20:1 to about 30:1.
51: The pharmaceutical composition of items 38 to 50, wherein the N/P ratio of the lipid-based carriers encapsulating the nucleic acid is in a range from about 1 to about 10, preferably in a range from about 5 to about 7.
52: The pharmaceutical composition of items 38 to 51, wherein the lipid-based carriers have a Z-average size in a range of about 50 nm to about 120 nm.
53: The pharmaceutical composition or the artificial RNA of any one of the preceding items, wherein administration of the pharmaceutical composition or artificial RNA reduces or prevents pathological epithelial to mesenchymal transition (EMT), pathological cell proliferation, aberrant angiogenesis, aberrant neovascularization, degeneration, and/or ocular fibrosis.
54: The pharmaceutical composition or the artificial RNA of item 53, wherein the administration is an ocular administration preferably selected from topical, intravitreal, intracameral, intranasal, subconjunctival, subretinal, subtenon, orbital, retrobulbar, topical, suprachoroidal, posterior juxtascleral, or intraoperative administration, preferably intravitreal or intraoperative administration.
55: The pharmaceutical composition or the artificial RNA of item 55, wherein the ocular administration is performed into a tamponade agent-filled human eye, optionally wherein the tamponade agent is a gas agent or a silicone agent.
56: A Kit or kit of parts comprising at least one artificial RNA of any one of items 1 to 33, and/or at least one pharmaceutical composition of any one of items 34 to 55, optionally comprising a liquid vehicle for solubilising, and optionally comprising technical instructions providing information on administration and dosage of the components.
57: An artificial RNA of any one of items 1 to 33, or a pharmaceutical composition of any one of items 34 to 55, or a kit or kit of parts of item 56, for use as a medicament in treating or preventing a disease, disorder, or condition in a subject.
58: An artificial RNA of any one of items 1 to 33, or a pharmaceutical composition of any one of items 34 to 55, or a kit or kit of parts of item 56, for use as a medicament in treating or preventing an ocular disease, disorder, or condition in a subject.
59: An artificial RNA of any one of items 1 to 33, or a pharmaceutical composition of any one of items 34 to 55, or the kit or kit of parts of item 56, for use as a medicament of item 57 or 58, wherein the disease, disorder, or condition is associated with or caused by pathological epithelial to mesenchymal transition (EMT), pathological cell proliferation, aberrant angiogenesis, aging, aberrant neovascularization, degeneration, solid tumors, and/or fibrosis.
60: An artificial RNA of any one of items 1 to 33, or a pharmaceutical composition of any one of items 34 to 55, or the kit or kit of parts of item 56 for use as a medicament of item 58 or 59, wherein the ocular disease, disorder, or condition is selected from proliferative diabetic retinopathy (PDR), macular edema, non-proliferative diabetic retinopathy, age-related macular degeneration, geographic atrophy, ocular neovascularization, retinopathy of prematurity (ROP), a retinal vein occlusion, ocular ischemic syndrome, neovascular glaucoma, a retinal hemangioma, Coats' disease, FEVR, or Norrie disease, persistent hyperplastic primary vitreous (PHPV), thyroid eye disease, epiretinal membrane, small vessel disease, or proliferative vitreoretinopathy (PVR), preferably wherein the ocular disease, disorder, or condition is PVR.
61: An artificial RNA of any one of items 1 to 33, or the pharmaceutical composition of any one of items 34 to 55, or the kit or kit of parts of item 56 for use as a medicament in treating or preventing proliferative vitreoretinopathy (PVR).
62: An artificial RNA of any one of items 1 to 33, or the pharmaceutical composition of any one of items 34 to 55, or the kit or kit of parts of item 56 for use as a medicament of items 58 to 61, wherein the subject has suffered a trauma to the eye, comprises a retinal hole, a retinal tear, a retinal detachment disorder, or has undergone an ocular surgery, preferably wherein the retinal detachment disorder is selected from rhegmatogenous retinal detachment, exudative retinal detachment, or tractional retinal detachment.
63: An artificial RNA of any one of items 1 to 33, or the pharmaceutical composition of any one of items 34 to 55, or the kit or kit of parts of item 56 for use as a medicament of items 58 to 62, wherein the use comprises administration of the artificial RNA, the pharmaceutical composition, or the kit or kit of parts by intravitreal administration, by administration prior to an ocular surgery, during an ocular surgery, or after an ocular surgery.
64: A method of treating or preventing a disease, disorder or condition, wherein the method comprises applying or administering to a subject in need thereof an effective amount of the artificial RNA of any one of items 1 to 33, or the pharmaceutical composition of any one of items 34 to 55, or the kit or kit of parts of item 56, preferably further characterized by any of the features of items 57 to 63.

### Brief Description of Tables:

Table 1: Element A and B of the transcription factor inhibitors (amino acid sequences and cds)
Table 2: Transcription factor inhibitor fusion constructs (amino acid sequences and cds)
Table 3: RNA sequences encoding transcription factor inhibitors
Table 4: RNA sequences encoding the preferred RUNX inhibitor according to SEQ ID NO: 232
5 Table 5: RNA constructs used in the examples
Table 6: Summary of primary and secondary antibody dilutions used in examples
Table 7: RUNX trap variant constructs used in Example 7.
Table 8: LNP formulations and RUNX trap constructs used in Example 8
Table 9: Different opt1-optimized mRNAs encoding RUNX trap used in Example 9
10 Table 10: RUNX trap formulations used in Example 10
Table 11: Study groups used in Example 12
List A: Suitable target transcription factors
List B: Suitable cytoskeletal proteins that may be used for element B

### BRIEF DESCRIPTION OF THE FIGURES

FIG. 1: Merely as an illustration, the proposed mechanism of a nucleic acid encoded transcription factor inhibitor, in that example, a RUNX trap is shown. Nucleic acid delivery in a delivery vehicle (e.g. lipid-based carrier) and the mechanism of action of the produced RUNX trap is shown. The produced RUNX trap prevents entry of the cellular target transcription factor into the nucleus by binding to RUNX, thereby, inter alia suppressing the activity of RUNX.

FIG. 2: Primary cultures from human PVR contain cells representative of the EMT continuum. 2A: Cell clustering analysis was done according to the cell cycle phase. The UMAP identified 3 distinguishable clusters. 2B and 2C: Genes classified under epithelial marker category were enriched in Cluster 2 whereas genes classified under mesenchymal marker category were enriched in Cluster 0. Further information is provided in the Example section, Example 3.

FIG. 3: shows that mRNA-encoded RUNX trap sequestered RUNX1 in the cytoplasm and reduced proliferation of C-PVRs in vitro. 3A: Fluorescence microscopy shows expression of GFP in C-PVR cells with CVCM1 and CVCM2 or negative control (NC). 3B: Expression of the RUNX trap in C-PVR cells by Western blot. 3C: Immunocytochemistry showing expression and localization of the RUNX trap (white), N-Cadherin, RUNX1 and DAPI in C-PVR (Scale bar—50 μm). 3D: Western blot and immunocytochemistry. 3E: shows the effects of nucleic acid encoded RUNX trap treatment on TGFbeta2-mediated induction of N-Cadherin. 3F and 3G: In vitro functional assays show the effect of treatment with RUNX trap in proliferation and cell viability measured by LDH (3H). 3I: Dot plot diagram comparing the expression of SMMHC as the marker for RUNX trap and RUNX1 expression in control vs RUNX1 treatment group. Further information is provided in the Example section, Example 4.

FIG. 4: shows that mRNA-encoded RUNX trap reduced PVR membrane growth and eye pathology in a rabbit model and reversed EMT in C-PVR. 4A: Significant expression of a mRNA encoded Luciferase reporter at 24 hours within the vitreous of rabbit eyes injected with C-PVR cells. 4B: Timeline of preclinical efficacy of the RUNX trap in a rabbit model of PVR. 4C: Representative H&E images from rabbit eyes treated with RUNX-Trap or control after 2 weeks (Scale Bar—200 μm). 4D: OCT images at 2-weeks comparing eyes treated with the RUNX trap compared to vehicle injected eyes. 4E: PVR severity score and Fastenberg score. 4F: Percentage number of the cells per cluster based on the treatment in the scRNAseq analysis of C-PVR primary culture. 4G: The figure plot showing expression pattern according to the ratio unspliced/spliced molecule counts transcribed from the gene MARVELD2, which is a marker of epithelial cells, and COL4A1, a mesenchymal marker. 4H: Effect of the RUNX1 trap on EMT related genes in C-PVR cells visualized by Dotplot. 4I: Proposed mechanism of RUNX1 regulating EMT in C-PVR primary culture (left) and proposed RUNX trap mechanism. Further information is provided in the Example section, Examples 5 and 6.

FIG. 5: shows that mRNA-encoded RUNX trap variants 1˜4 (5A-5D) and variant 6 (5E) reduce proliferation of C-PVRs in vitro after 24 h after transfection. NC=Negative control. Further information is provided in the Example section, Example 7.

FIG. 6: shows that mRNA-encoded RUNX trap variants 1-7 (6A-6G) reduce proliferation of C-PVRs in vitro after 48 h after transfection. NC=Negative control. Further information is provided in the Example section, Example 7.

FIG. 7: shows the comparison of CBFbeta expression of SM-102 LNP formulated modified (N1-methylpseudouridine (m1ψ) or unmodified mRNA encoding RUNX trap after transfection of ARPE-19 cells. Further information is provided in the Example section, Example 8.

FIG. 8: shows that ARPE-19 cells transfected with LNP-formulated mRNA encoding RUNX traps display an efficient level of CBFbeta. Further information is provided in the Example section, Example 9.

FIG. 9: shows that 315 LNP-formulated mRNA encoding RUNX trap reduce proliferation of C-PVRs (9A). 9B shows low levels of IFNalpha for the formulations F5-7. The transfection also does not have an effect cell viability as shown for all formulations in 9C. Levels of cyclin D, a proliferation marker, have been decreased in cells transfected with LNPs carrying mRNAs encoding RUNX traps (9D). Further information is provided in the Example section, Example 10.

FIG. 10: shows the effect on leakage of LNP-formulated RUNX trap mRNA in a laser-induced choroidal neovascularization (CNV) mice model after 7 days of injection (10A). 10B shows that doses of 25 ng are more effective in reducing lesion size compared to Eylea after 7 days post injection. Further information is provided in the Example section, Example 11.

### EXAMPLES

In the following, examples illustrating various embodiments and aspects of the invention are presented. However, the present invention shall not to be limited in scope by the specific embodiments presented herein and should rather be understood as being applicable to other compositions or uses as for example defined in the specification. Accordingly, the following preparations and examples are given to enable those skilled in the art to more clearly understand and to practice the present invention. Indeed, various modifications of the invention in addition to those described herein will become readily apparent to those skilled in the art from the foregoing description, accompanying figures and the examples below.

### Example 1: Preparation of Nucleic Acid Formulations Encoding Transcription Factor Inhibitors

The present example provides methods of obtaining the RNA of the invention as well as methods of generating composition of the invention comprising nucleic acid, in particular RNA formulated in polyethylene glycol/peptide polymers or RNA formulated in lipid-based carriers.

### 1.1. Preparation of DNA Templates for RNA In Vitro Transcription:

DNA sequences encoding transcription factor inhibitors of the invention were prepared and used for subsequent RNA in vitro transcription reactions. Some DNA sequences were prepared by modifying the wild type or reference encoding DNA sequences by introducing a G/C optimized coding sequence for stabilization and expression optimization. Sequences were introduced into a pUC derived DNA vector to comprise a stabilizing heterologous UTR sequences and a stretch of adenosines and optionally a histone stem-loop (hSL). The obtained plasmid DNA templates were transformed and propagated in bacteria using common protocols known in the art. Eventually, the plasmid DNA templates were extracted, purified, and linearized using a restriction enzyme. The herein used RNA constructs are provided in Table 5.

TABLE 5
RNA constructs used in the Examples

| | | SEQ | SEQ | SEQ |
|---|---|---|---|---|
| | | ID NO: | ID NO: | ID NO: |
| RNA ID | Name | Protein | CDS | RNA |
| R9714 | CBFB(1-165)-SMMHC(1527-1927) | 231 | 355 | 809 |
| R9715 | CBFB(1-165)-SMMHC(1527-1927) | 231 | 417 | 810 |
| R9716 | CBFB(1-165)-SMMHC(1527-1877) | 232 | 356 | 819 |
| | (CBFbeta-SMMHCΔC95) | | | |
| R9717 | CBFB(1-165)-SMMHC(1527-1877) | 232 | 418 | 820 |
| | (CBFbeta-SMMHCΔC95) | | | |
| R11302 | CBFbeta(1-165)-SMMHC(1527-1877) | 232 | 418 | 820 |
| R11315 | CBFB(1-165)-SMMHC(1527-1877) | 232 | 418 | 910 |
| R11316 | CBFbeta(1-165)-SMMHC(1527-1877) | 232 | 418 | 1580 |
| R11350 | CBFB(1-165)-SMMHC(1527-1877) | 232 | 480 | 830 |
| R11352 | CBFB(1-165)-SMMHC(1527-1877) | 232 | 604 | 850 |
| R11353 | CBFbeta(1-165)-SMMHC(1527-1877) | 232 | 418 | 1582 |
| R9744 | CBFbeta(1-165)-Linker-LifeAct | 1548 | 1556 | 1566 |
| R11345 | CBFbeta(1-165)-HIF1alpha(549-575) | 234 | 420 | 822 |
| R11346 | RUNX1(1-177)-RUNX1T1a(31-604) | 236 | 422 | 824 |
| R11349 | RUNX1(K83E,R174Q) | 197 | 383 | 818 |
| R11344 | CBFbeta(1-141)-SMMHC(1527-1877) | 1545 | 1553 | 1563 |
| R7386 | luciferase | | | 1518 |
| R8720 | eGFP | | | 1519 |
| R8730 | luciferase | | | 1520 |

1.2. RNA In Vitro Transcription from Plasmid DNA Templates:

Linearized DNA templates were used for DNA dependent RNA in vitro transcription (IVT) using T7 RNA polymerase in the presence of a sequence optimized nucleotide mixture (ATP/GTP/CTP/UTP) and cap analog (for Cap1: m7G(5′)ppp(5′)(2′OMeA)pG; TriLink) under suitable buffer conditions. After RNA in vitro transcription, the obtained RNA IVT reaction was subjected to purification steps comprising RP-HPLC.

1.3. Preparation of Polymer-Lipidoid Complexes Carrying mRNA (CVCMs);

20 mg of peptide (CGH₅R₄H₅GC-NH₂) was dissolved in N-methylpyrrolidone. After addition of 2 ml of borate buffer (pH 8.5), the solution was stirred at RT for >18 h. Subsequently, 12.6 mg of PEG-SH 5000, dissolved in N-methylpyrrolidone, was added to the peptide solution and the vial filled up to 3 ml with borate buffer (pH 8.5). After 18 h, the reaction mixture was purified and further concentrated using Centricon filter units (MWCO 10 kDa) and subsequently lyophilized. The obtained polyethylene glycol/peptide polymers (HO-PEG 5000-S-(S-CGH₅R₄H₅GC-S-)7-S-PEG 5000-OH), dissolved in water, were used to prepare RNA formulations. RNA was complexed with the polymer at the 1:2 ratio (W/W). Two different 3-C12-C3-OH lipidoid concentrations were used (CVCM® 1 and 2). Formulated RNA was lyophilized and stored at −80° C.

1.4. Preparation of Lipid-Based Carriers Encapsulating the mRNA:

An ethanolic lipid solution is prepared by solubilizing a cationic lipid, a neutral lipid, cholesterol, and an aggregation reducing lipid in ethanol. An aqueous RNA solution is prepared by adjusting the purified RNA (obtained according to Example 1.2) to a target concentration in 50 mM citrate buffer, pH 4.0.

Lipid-based carriers are prepared essentially according to the procedures described in WO2015199952, WO2017004143 and WO2017075531. In short, lipid-based carriers are prepared at a ratio of RNA to total Lipid of about 0.03-0.04 w/w. Pumps are used to combine the ethanolic lipid solution with a flow rate F1 and the RNA aqueous solution with a flow rate F2 at a ratio of about 1:5 to 1:3 (vol/vol) in a T-piece system. F1 and/or F2 are adjusted to flow rates above 15 ml/min to allow the formation of LNPs encapsulating the RNA that have a Z-average size in a range from about 50 nm to about 120 nm. After formulation, the ethanol is removed by at least one TFF step and at least one clarifying filtration step. After clarifying filtration, the filtrate is adjusted to a desired concentration (typically 1 g/l RNA) using a buffer comprising sucrose, sodium chloride, and a buffering agent. Subsequently, the resulting formulation is filtered through sterilizing filters to reduce bioburden as used for in vitro and in vivo studies essentially according to Example 2 to Example 6.

The herein tested 315 LNPs were composed of 40.9 mol % cholesterol, 10 mol % DSPC, 47.4 mol % ALC-0315, and 1.7 mol % ALC-0159 (N: P ratio of 6).

The herein tested SM-102 LNPs were composed of 38.5 mol % cholesterol, 10 mol % DSPC, 50 mol % SM-102, and 1.5 mol % DMG-PEG 2000 (N: P ratio of 6).

### Example 2: Methods Used in the Experiments

The present example provides methods used for testing the nucleic acid-based transcription factor inhibitor formulations (obtained according to Example 1).

### 2.1. Western Blotting:

Protein concentration was calculated using the BCA assay. Subsequently 10 μg of protein was prepared in 10 μl RIPA buffer (Cell Signaling Technology), 4 μl DTT (1 M, Sigma Aldrich) and 10 μl Laemmli buffer (Boston Bioproducts) to a final volume of 40 μl and denatured 5 min at 90° C. Samples were separated electrophoretically for 1 h at 70 V using 4-20% pre cast gradient gels (Mini-PROTEAN TGX, Bio-Rad) and SDS-Tris-Glycine buffer. Proteins were transferred to nitrocellulose membranes (VWR; 27376-991) for 1 h at 70 V. Membranes were blocked for 1 h with Odyssey Blocking Buffer (LI-COR Biosciences), and probed overnight at 4° C. with primary antibody and 1 h at room temperature with IRDye 800CW donkey anti-rabbit (1:10,000, LI-COR Biosciences) antibodies. Immunoreactive bands were visualized using the Odyssey Infrared Imaging System and visualized on the Image Studio version 2.1 (LI-COR Biosciences).

### 2.2. Luciferase Assay:

Frozen rabbit eyes were dissected and the vitreous and retina were collected and homogenized in lysis buffer, subsequently 20 μl of lysate are added to each well of a 96 well plate and before the plate was read using the Luminometer, 100 μl of luciferase assay reagent was added to each well and the plate was read immediately. Statistical analysis was carried out using a one-way ANOVA with Tukey's multiple comparison correction.

### 2.3. C-PVR Cell Culture:

Human PVR membranes obtained after the surgery were immediately processed for single cell isolation according to previous published protocol (Delgado-Tirado et al 2020, Scientific Reports. 2020; 10 (1): 20554). C-PVR were seeded with 30×10³/well density in 48 well plates. 24 h after seeding, the cells were treated with the nucleic acid formulation or PVR media alone (control) in triplicates for each condition and incubated for 2 h. The cells were washed with PBS and cultured in PVR media with the growth factors for 24 h then collected for further analysis.

### 2.4. Proliferation and Cell Viability Assays:

C-PVR cells were cultured and maintained in 96 well plates. Cells were incubated with the formulations for 2 h in serum free media with the formulations. After 2 h, the media was switched to complete media and cells incubated for 24 h. 24 h post treatment, the proliferation rate was measured using CyQUANT Direct Cell Proliferation Assay (ThermoFisher, Waltham, MA) according to the manufacturer's recommendation. The proliferation result was validated using a Ki67 ELISA method (Abcam, ab253221) according to the manufacturer's guidelines. C-PVR cells were cultured in 24 well plate to form a confluent monolayer. Following the proliferation assay, a lactate dehydrogenase (LDH) assay (Promega, G1780) was used to assess toxicity of the formulations according to the manufacturer's guidelines.

### 2.5. PVR Induction in Rabbits and RNA Formulation Administration:

PVR induction in rabbits was carried out as previously described (see Delgado-Tirado et al 2020, Scientific Reports. 2020; 10 (1): 20554). Briefly, animal experiments were performed in accordance with the guidelines for the Use of Animals in Ophthalmic and Vision Research of the Association for Research in Vision and Ophthalmology (ARVO). This study was also approved by Institutional Animal Care and Use Committee (IACUC) of the Schepens Eye Research Institute of Mass Eye and Ear. Only male New Zealand White rabbits (2.3 kg of weight, 6-8 week-old) purchased from Charles River (Charles River Laboratories, Inc., Wilmington, MA) were used. A PVR score grading system was developed to assess severity of disease as previously published (Delgado-Tirado et al 2020; Scientific Reports. 2020; 10 (1): 20554). The score was determined by combination of the most severe phenotypes identified by indirect ophthalmoscopy, fundus imaging and OCT. OCT, fundus and histology were performed as previously described (Delgado-Tirado et al 2020; Scientific Reports. 2020; 10 (1): 20554).

### 2.6. Immunocytochemistry Protocol:

C-PVR cells were seeded on glass slides (MatTek), 40,000 cells per well then after 24 h, cells were treated with 5 μg in 500 μl of serum free media and incubated for 2 h, the media was then exchanged with 500 μl growth media and incubated for 24 h at 37° C. Cells were washed with ice cold PBS, then fixed in PFA 4% for 5 min, washed in PBS, then incubated in PB (Permeabilization and Blocking buffer) consisting of 1×PBS, 1% Donkey Serum and 0.05% Triton X overnight. Subsequently, cells were incubated with primary antibody (see Table 6) diluted in blocking buffer overnight at 4° C. Samples were then washed in PBS and incubated with the corresponding secondary antibody for 2 h at room temperature, and nuclei were stained with DAPI (1:1000) for 15 min. Finally, samples were mounted with vectashield mouting media (Vector Laboratories). Images were acquired with a Leica SP8 confocal microscope (Leica, Wetzlar, Germany).

TABLE 6
Summary of primary and secondary antibody dilutions

| Target | Antibody | Company-Cat No. | Dilution |
|---|---|---|---|
| CBFbeta | Sheep Polyclonal | Fischer Scientific (AF73349SP) | 1:200 |
| N-Cadherin | Mouse Monoclonal | Santa Cruz (sc-53488) | 1:500 |
| RUNX1 | Rabbit Polyclonal | Lifespan Bioscience (LSB13948) | 1:500 |

| Secondary Antibody | Company-Cat No. | Dilution |
|---|---|---|
| IRDye 800CW-Anti Mouse | Licor | 1:2000 |
| IRDye 800CW-Anti Rabbit | Licor | 1:2000 |
| Donkey Anti-Goat AlexaFluor 488 | ThermoFisher (A-11057) | 1:200 |
| Donkey Anti- Mouse Alexa Fluor 594 | ThermoFisher (A10037) | 1:200 |
| Donkey Anti-Rabbit AlexaFluor 647 | ThermoFisher (A32795) | 1:200 |

### 2.7. Sample Preparation and Single Cell RNA Sequencing:

C-PVR cells were treated in culture with 5 μg of RNA encoding for Luciferase or RUNX inhibitor for 24 h prior to cell harvesting. Single cells suspension of C-PVR primary culture was prepared according to the protocol recommended by the 10× Genomics sample preparation guide (CG00053) and scRNAseq was performed using 10× Genomics Chromium Single Cell 3′ Reagent Kits version 2. Size distribution and molarity of resulting cDNA libraries were assessed via Bioanalyzer High Sensitivity DNA Assay (Agilent Technologies, USA). The libraries were then pooled and sequenced on an Illumina NextSeq 500 instrument according to Illumina and 10× Genomics guidelines with 1.4-1.8 pM input and 1% PhiX control library spike-in (Illumina, USA). The data was processed using 10× Genomics' Cell Ranger pipeline, which generates feature/barcode matrices from the raw counts data. The full dataset contained 27,215 features and 7,379 cells. Before clustering, the counts matrix was filtered to only include the top 5,000 variable features. If the dataset was smaller than the specified number of variable genes, all the features were kept for analysis. The final dataset contained 27,215 genes and 6,585 cells. The single cell sequencing data were then analyzed using the open-source Seurat kit with Louvain clustering and visualized by Uniform Manifold Approximation and Projection (UMAP). The cell percentage calculation was done at clustering resolution of 0.2. Differential expression genes analysis was used to identify the cell types and compared each cluster to all others using the Wilcoxon method in Seurat with each retained marker expressed at a minimum log fold change threshold of 0.25 for cluster-specific marker genes identification.

2.8. Identification of C-PVR Cell Types and Transcription Factors Enrichment Analysis of scRNAseq Data:

Cells were clustered using the recursiveSplitModule and recursiveSplitCellfunctions, testing for different numbers of cell clusters and feature modules. Feature module numbers L were tested from 10 to 150. Since there is no clear “elbow” in the resulting perplexity plot, the selected number of modules was chosen to be L=100. To complete the clustering, the recursiveSplitCell function was applied to the selected celda model with a range of K values from 10 to 40. Differential expression genes analysis was used to identify the cell types and compared each cluster to all others using the Wilcoxon method in Seurat with each retained marker expressed at a minimum log fold change threshold of 0.25 for cluster-specific marker genes identification.

### Example 3: Primary Cultures from Human PVR Contain Cells Representative of the EMT Continuum

The goal of the experiment was to test whether human primary cell cultures obtained from surgically removed PVR membranes (C-PVRs) can serve as a model for testing the effect of nucleic acid encoded transcription factor inhibitors. Methods used herein are further described in Example 2.

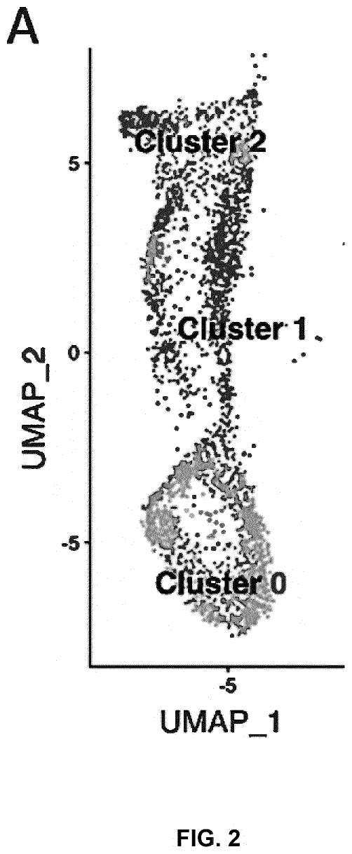

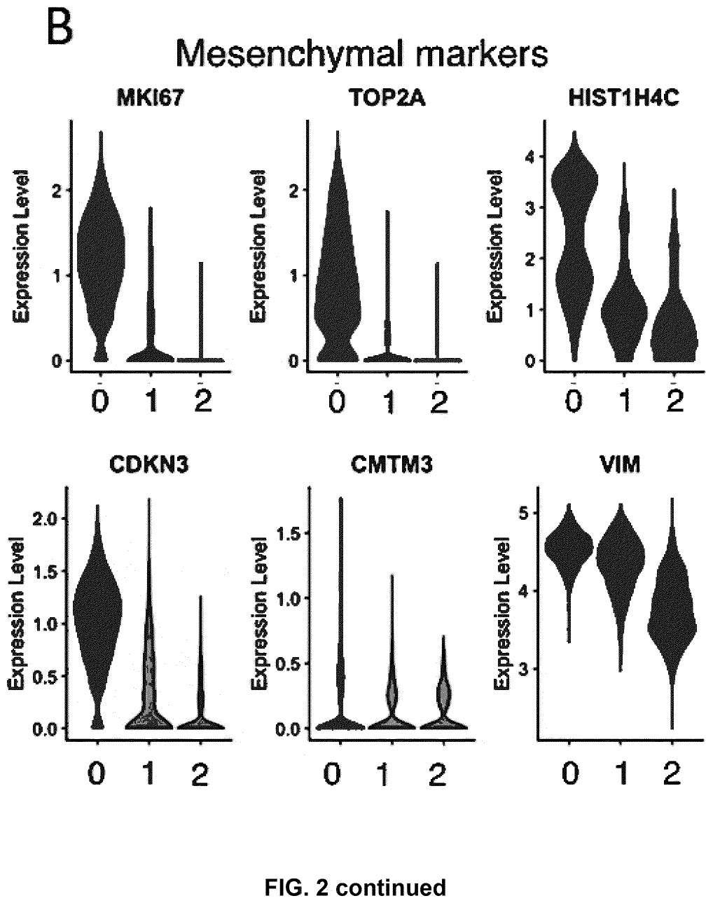

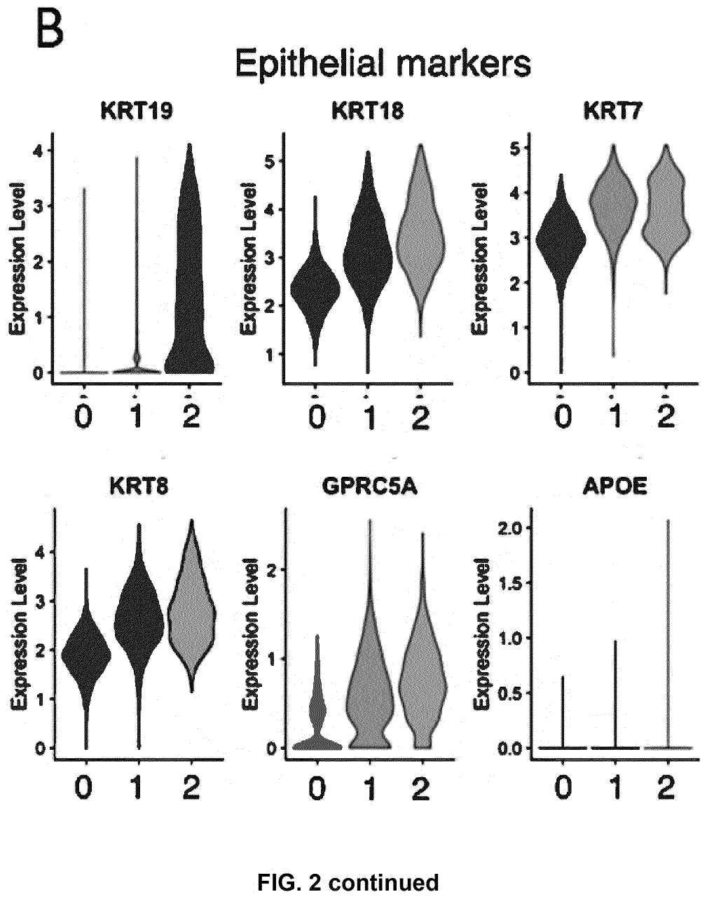

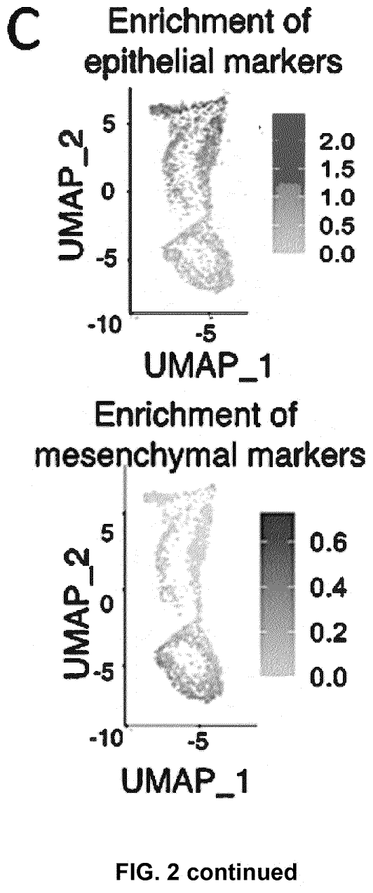

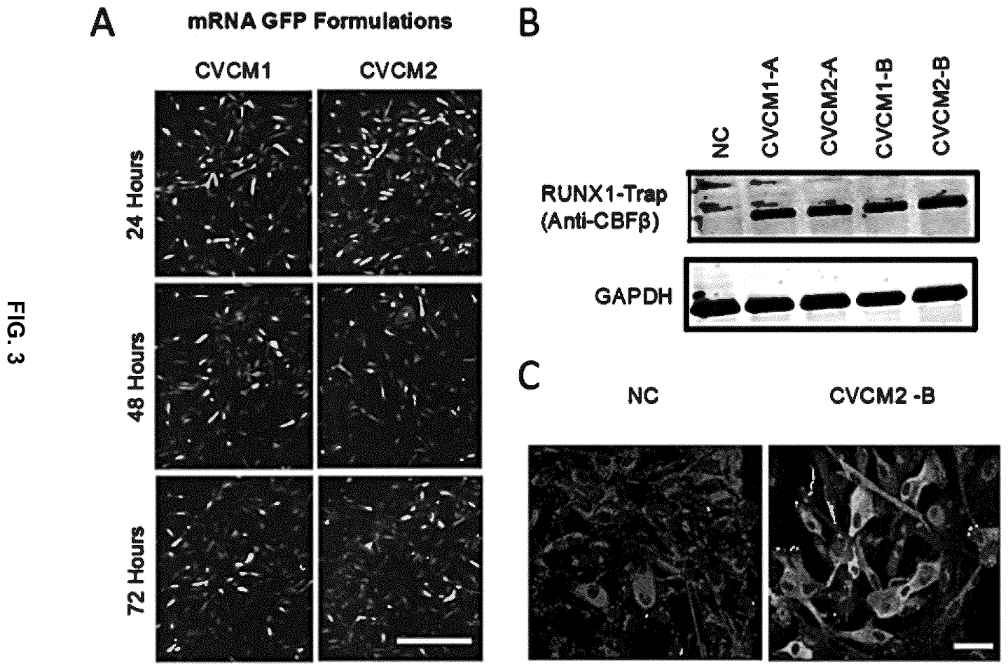

EMT is a dynamic and continuous biological process that contributes to organogenesis and disease. During EMT, cells fluctuate from extremes of differentiated epithelium to undifferentiated mesenchyme with a multitude of potential phenotypes in between. Single cell RNA sequencing (scRNA-seq) was carried out to characterize the baseline status of EMT within human primary cell cultures obtained from surgically removed PVR membranes (named C-PVRs) (see FIG. 2A). UMAP dimensionality reduction projection plot displays the trajectories of expression of epithelial and mesenchymal markers in C-PVR primary culture (see FIG. 2B). A gradual loss of epithelial markers was observed such in clusters 2>1> and 0, respectively. This correlated with a gain of mesenchymal genes or EMT-associated markers in cluster 0>1> and 2, indicating a shift toward mesenchymal phenotypes in cluster 0 from epithelial phenotypes in cluster 3 (see FIG. 2C).

The data of the experiment demonstrates that the used C-PVR primary cultures display a continuum of cell clusters undergoing various stages of EMT and can therefore be used as an informative model for testing and developing of nucleic acid-based transcription factor inhibitors.

### Example 4: RNA Encoded RUNX Trap Sequestered RUNX1 in the Cytoplasm and Reduced Proliferation and TGFbeta2-Induced EMT of C-PVRs In Vitro

The goal of the experiment was to test whether an exemplary nucleic acid encoded transcription factor inhibitor, namely encoding a RUNX inhibitor according to protein SEQ ID NO: 323, displayed the expected cytosolic localization. Further, the effect of the produced a RUNX inhibitor on EMT was explored. RNA formulations used in the experiment were prepared according to Example 1, methods used herein are described in Example 2.

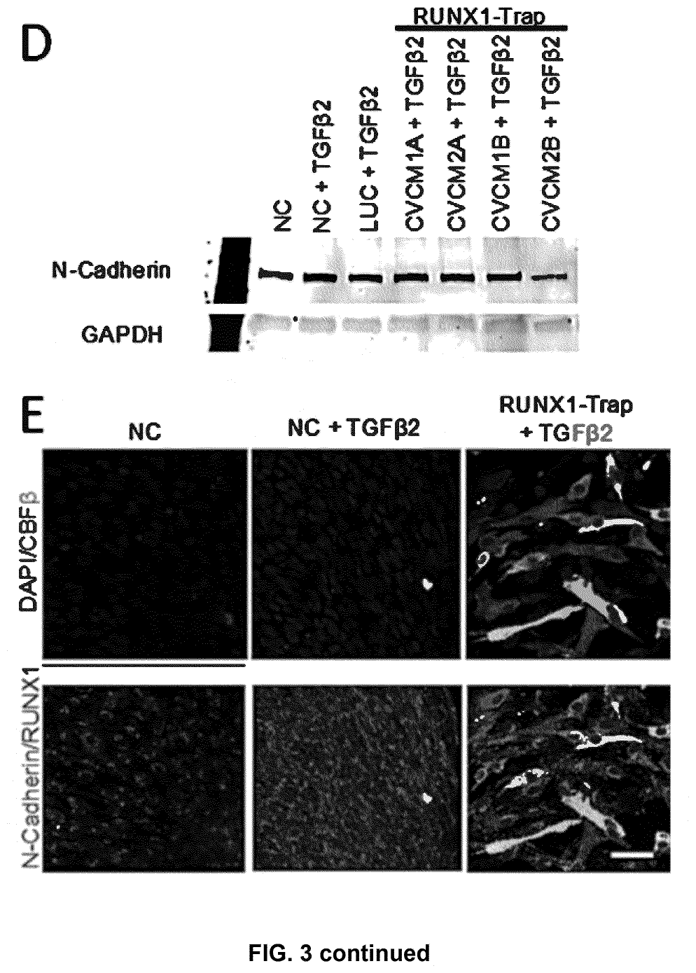

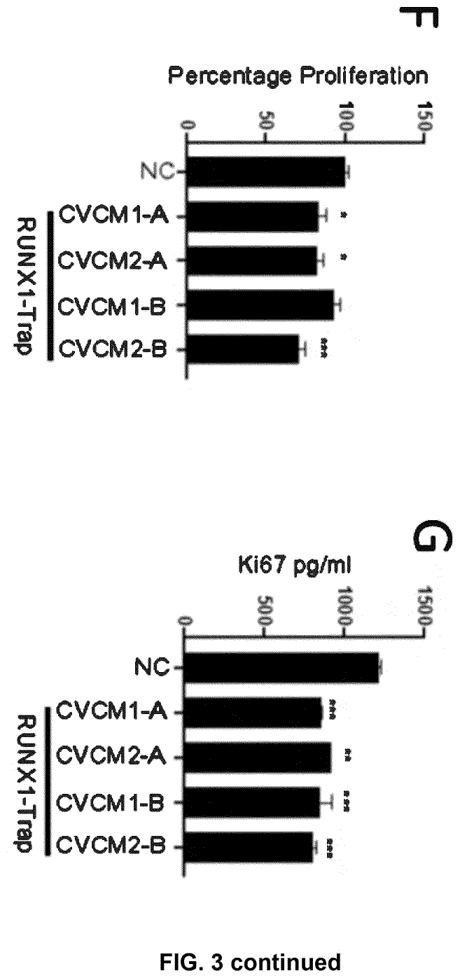

C-PVR cells (that have been analyzed in Example 3) were treated with 10 μg RNA encoding green fluorescent protein (GFP; RNA SEQ ID NO: 1519) using delivery carriers consisting of a cationic polymer in the core and a polyethylene glycol (PEG) layer on the outside to examine effectiveness and duration of protein production in that specific cell type ((CVCM® 1 and CVCM® 2, generated according to Example 1). The data shows that both tested formulations showed stronger mRNA delivery capabilities which resulted in a strong GFP expression. Cells transfected with both formulations continued to display GFP positive signal at 72 hours (FIG. 3A).

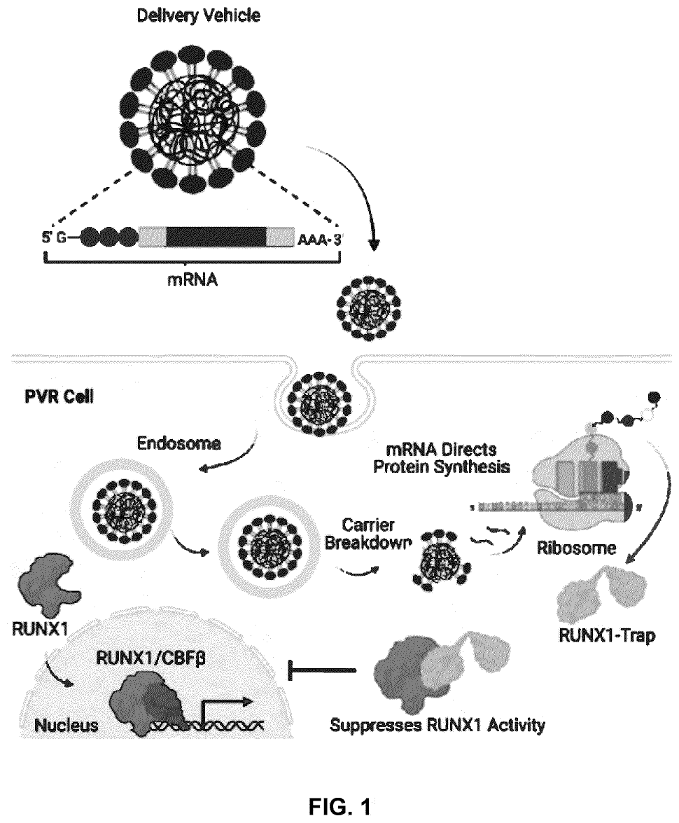

Accordingly, RNAs encoding for CBFbeta-SMMHC (RUNX trap; RNA sequence SEQ ID NO: 819; RNA sequence SEQ ID NO: 820) were formulated in these polymer-based carrier formulations and used to treat C-PVR. A robust production of CBFbeta-SMMHC was observed upon administration of 10 μg RNA after 24 hours as determined by Western blot (FIG. 3B). Cytosolic localization of CBFbeta-SMMHC and RUNX1 was confirmed by immunocytochemistry and confocal microscopy (FIG. 3C) which was consistent with the proposed sequestering mechanism (illustrated in FIG. 1). As shown in FIG. 3C, the produced RUNX trap CBFbeta-SMMHC co-localized with the target cellular RUNX1 in the cytosol which shows that the produced RUNX trap bound to the target transcription factor to captured RUNX1 in the cytosol. RUNX1 expression was reduced in cells with high levels of CBFbeta-SMMHC.

Stimulation of C-PVR cells with TGFbeta2 promotes EMT. C-PVR cells were stimulated with TGFbeta2 for 24 hours to assess the effect of the provided RUNX trap in EMT by measuring the expression of the mesenchymal marker N-Cadherin (FIG. 3D). Treatment with the nucleic acid encoding the RUNX trap blunted the ability of TGFbeta2 to induce N-Cadherin expression and reduced cell numbers as shown on confocal images (FIG. 3E).

A significant reduction in C-PVR proliferation upon treatment with the nucleic acid encoding the RUNX trap was confirmed using CyQuant Direct Cell Proliferation kit and an ELISA for Ki67 proliferation marker (FIG. 3F,G). The results show a clear effect on EMT and proliferation for all tested formulations and RNA constructs. The RNA construct comprising an optimized coding sequence was more efficient (SEQ ID NO: 820). Importantly, the treatments had no significant cellular toxicity measured by the lactate dehydrogenase (LDH) assay FIG. 3H). We directly confirmed the presence of the RNA encoding the RUNX1 trap by scRNA-Seq of C-PVRs after treatment. Dot plots showed that the levels of SMMHC (amino acid element B) was very high in cells treated with the RNA, while RUNX1 expression was significantly reduced compared with controls (FIG. 3I). The data of the experiment prove high RNA delivery efficacy and its translation into the encoded transcription factor inhibitor protein. Moreover, the data demonstrates that sequestering of RUNX1 by the provided RUNX trap further reduces RUNX1 expression, indicating that the produced RUNX trap protein was biologically active. The data is consistent with previous findings of a self-regulatory feedback loop for RUNX1 expression.

### Example 5: RNA-Encoded RUNX Trap Reduced PVR Membrane Growth and Eye Pathology in a Rabbit Model

The goal of the experiment was to test whether the nucleic acid encoded RUNX inhibitor is also effective in a clinically relevant rabbit model for PVR. RNA formulations used in the experiment were prepared according to Example 1, and methods used herein are further described in Example 2.

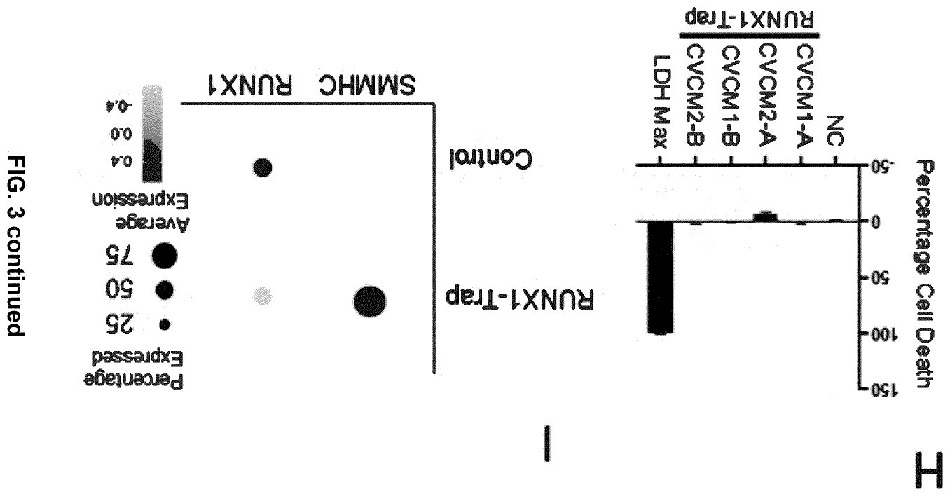

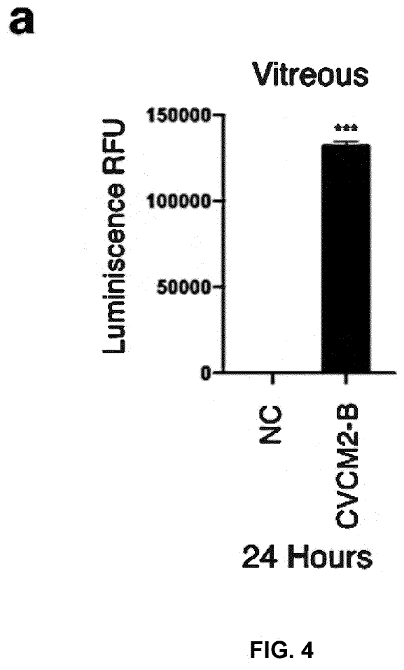

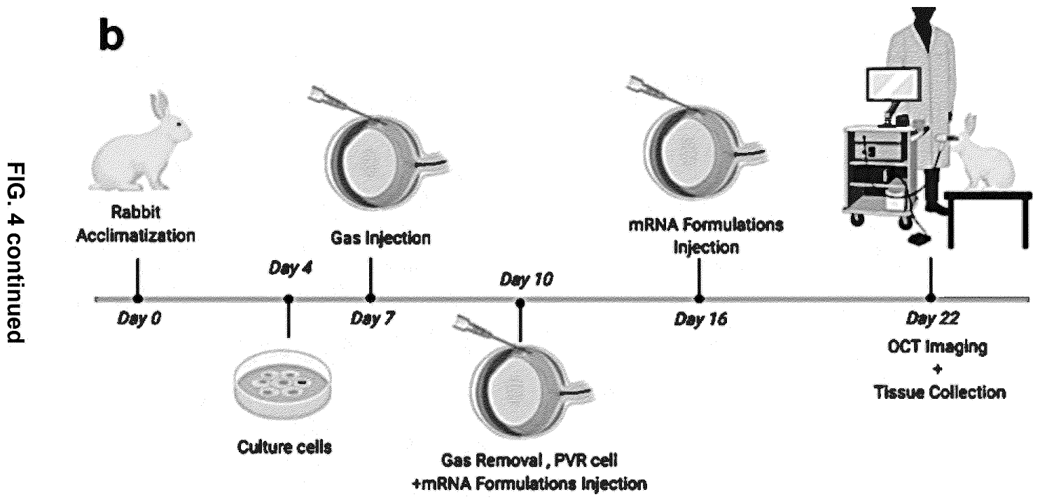

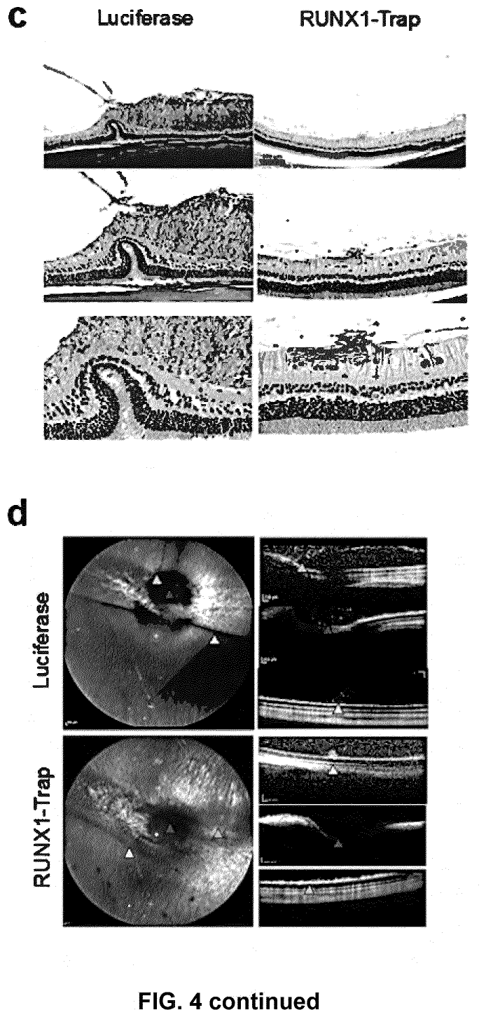

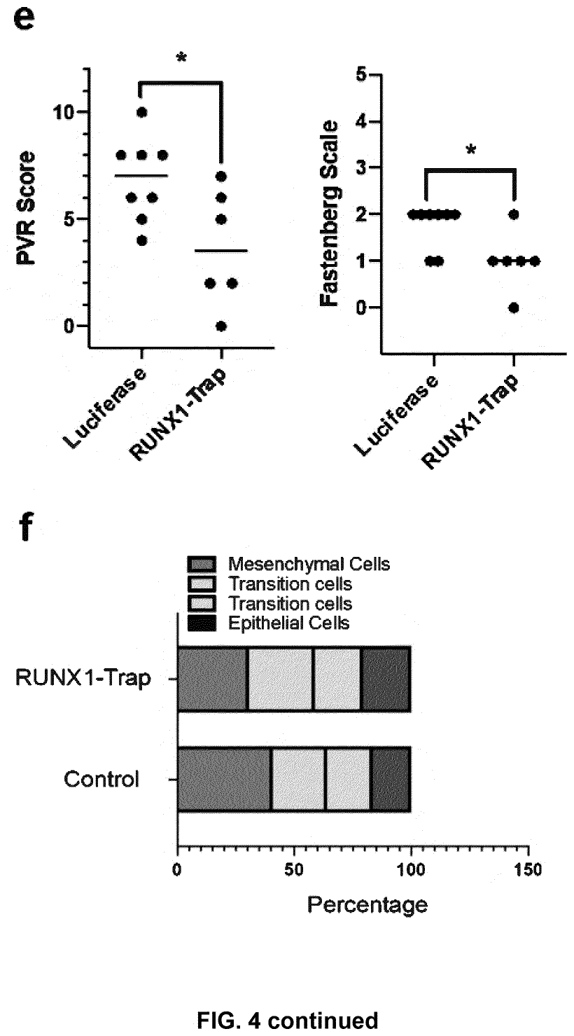

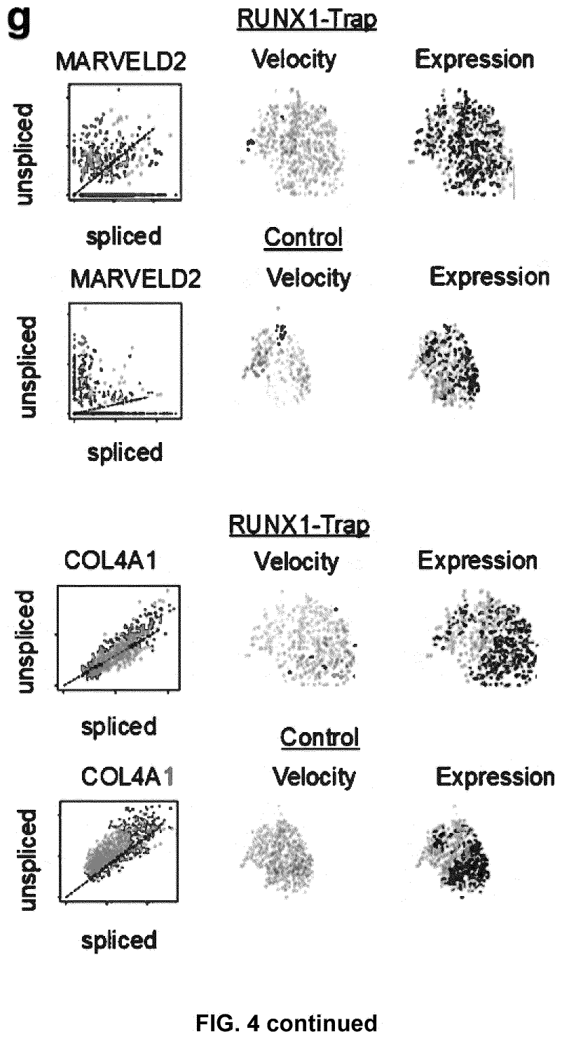

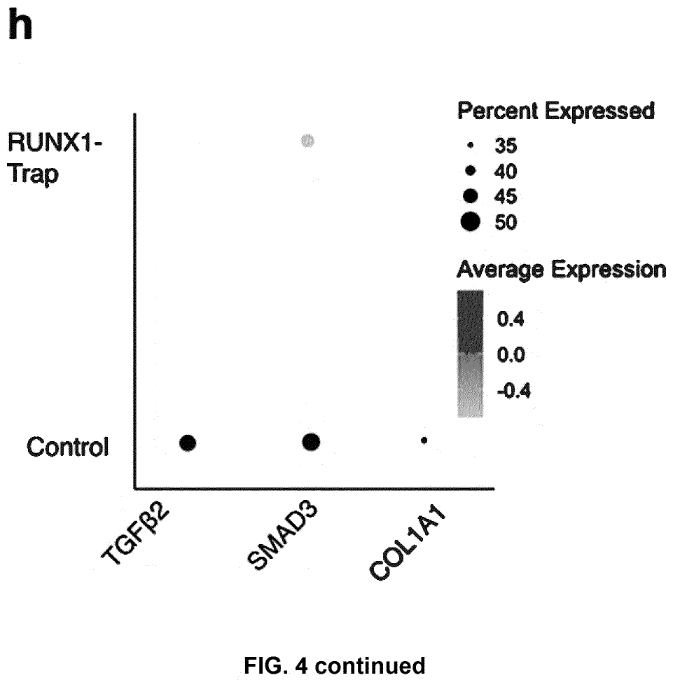

First, the delivery capabilities of the cationic polymer carrier was tested in vivo using RNA encoding for luciferase by injection of 36.5 μg RNA formulation (in 50 μl injection volume) into the vitreous cavity of rabbit eyes previously injected with C-PVR cells. A robust increase in luciferase activity in the vitreous containing human C-PVR cells was observed after administration of the formulation, showing that the formulation is suitable for in vivo applications (FIG. 4A). Next, the RUNX trap RNA formulation SEQ ID NO: 820 was tested in the in vivo PVR rabbit model. Rabbits received two injections of the RUNX trap RNA formulations (36.5 μg RNA in 50 μl injection volume) administered one week apart for a total duration of treatment of two weeks starting immediately after injection of C-PVRs (FIG. 4B).

The vehicle injected eyes developed PVR epiretinal membranes and had an average PVR severity score of 6.9±0.67 S.E.M whereas eyes that received the RNA encoded RUNX trap had less severe manifestations of disease with an average PVR score of 3.67±1.1 S.E.M (p<0.05) and less extracellular matrix deposition surrounding the injected C-PVR cells (FIG. 4C-E).

Interestingly, no PVR-related pathology has been observed in one of the RUNX trap treated eyes (PVR score=0). Measures of severity with our PVR scoring system comprising optical coherence tomography (OCT) data and clinical assessment agreed with more traditional scoring based on the Fastenberg scale to show less PVR pathology in RUNX trap treated eyes (FIG. 4E). Accordingly, histopathologic examination of rabbit eyes treated with the RUNX trap confirms a reduction in cellular proliferation and extracellular matrix deposition

Accordingly, the results show that intraocular administration of the RNA encoded RUNX trap led to a reduction in cellular proliferation and extracellular matrix deposition and strongly reduced severity of PVR in human PVR cells in a rabbit eye.

### Example 6: RNA-Encoded RUNX Trap Treatment Reversed EMT in C-PVR

Methods used herein are further described in Example 2.
scRNA-seq was used to assess gene expression changes related to EMT in C-PVR treated with the RNA encoded RUNX trap and controls. A higher percentages of cells with epithelial phenotypes (4%) and transitioning cells (6%) have been found after treatment with the RNA encoded RUNX trap compared with control. In contrast, the number of mesenchymal cells was lower by 10% after treatment with the RNA encoded RUNX trap compared with control (FIG. 4F).

This finding suggested that treatment with the RNA encoded RUNX trap triggered a mesenchymal to epithelial transition within these cells (MET). scRNA-seq data was used to predict cell evolution trajectories on a time scale, or RNA velocity, to further confirm the effect of the RNA encoded RUNX trap in regulating MET. An increase in splicing ratio molecule counts transcribed from the gene MARVELD2 was observed, an epithelial gene marker, after treatment with RNA encoded RUNX trap compared with control, indicative of cell differentiation towards epithelial phenotypes (FIG. 4G).

Differential gene expression analysis showed that treatment with the RNA encoded RUNX trap significantly reduced the expression of EMT related genes including TGFbeta2, SMAD3 and COL1A1 (p<0.05). (FIG. 4H). Accordingly, Treatment of C-PVR cells with the RNA encoded RUNX trap led to a significant reduction in EMT-associated genes including TGFbeta2, SMAD3 and COL1A1, genes that are directly involved in extracellular matrix deposition.

These results indicate the presence of a positive feedback loop between TGFbeta2 signalling and RUNX1 inducing EMT that was effectively inhibited and reversed by the RUNX trap (FIG. 4I).

### Summary of the Findings of Example 1 to 6

As demonstrated in the present Examples, an artificial nucleic acid encoding a transcription factor inhibitor according to the invention was effective in reducing the activity of the target transcription factor. As exemplified for a RUNX trap, a fusion protein comprising fragments of CBFbeta and SMMHC, the provided nucleic acid encoding said RUNX trap efficiently sequestered RUNX1 from the cell nucleus by binding and trapping the target transcription factor in the cytosol. That strongly reduced proliferation in primary human cell cultures derived from surgically excised membranes from eyes of patients with proliferative vitreoretinopathy (PVR). In addition, the expression of cellular RUNX1 was reduced and gene expression in the treated cells was shifted from a mesenchymal phenotype towards an epithelial profile across the EMT continuum (e.g. cellular expression of TGFbeta2, SMAD3, COL1A1 was reduced and). Furthermore, intravitreal administration of the RNA encoded RUNX trap strongly reduced proliferation and ocular pathology triggered by injection of human PVR cells in a rabbit eye. Mechanistically, treatment with the RNA encoded RUNX trap reversed EMT. This was illustrated by an increase of the epithelial cell clusters at the expense of mesenchymal cell clusters and by an increase in the transcription rate of MARVELD2, a tight junction associated epithelial marker, as a predictor of the future state of the cell.

In conclusion, the RNA encoded RUNX trap therapeutically targeting RUNX1 can effectively inhibit and reverse EMT in C-PVR cells and significantly reduce the severity of PVR in a rabbit model inter alia through the downregulation of key mediators of the TGFbeta/Smad3 pathway, effectively reducing cellular proliferation.

The data clearly demonstrates that the RNA encoded RUNX trap is suitable for medical uses and methods of treatment for diseases associated with RUNX, for example disorder, or condition associated with or caused by pathological epithelial to mesenchymal transition (EMT), pathological cell proliferation, aberrant angiogenesis, aberrant neovascularization, degeneration, and/or fibrosis.

Moreover, the data exemplifies how the present invention could be leveraged to develop a plethora of therapeutic artificial nucleic acid constructs for providing transcription factor inhibitors against transcription factors that are overexpressed and/or overactive in a disease, disorder, or condition.

### Example 7: RUNX Trap Variants

The goal of the experiment was to test whether different RUNX trap variants (Table 7) reduce proliferation of C-PVRs in vitro without having an impact on cell viability. RNA formulations encoding RUNX trap variants used in the experiment were prepared according to Example 1, methods used herein are described in Example 2.

Briefly, all resulting mRNAs have been formulated in CVCMs. Proliferation and cell viability assays (Example 2.4) have been performed to validate their functional activity. Hereby, C-PVR cells (10,000 cells/well) were cultured and maintained in 96-well plates. Cells were incubated with the RUNX variant formulations for 4 hours in serum free media. The proliferation rate was measured using CyQUANT Direct Cell Proliferation Assay (ThermoFisher, Waltham, MA) according to the manufacturer's recommendation. Cell viability was measured using a lactate dehydrogenase (LDH) assay (Promega, G1780).

TABLE 7
RUNX trap variant constructs used in Example 7

| | | | SEQ | SEQ | SEQ |
|---|---|---|---|---|---|
| | | | ID NO: | ID NO: | ID NO: |
| RNA ID | Variant | Name | Protein | CDS | RNA |
| R11353 | 1 | CBFbeta(1-165)-SMMHC(1527-1877) | 232 | 418 | 1582 |
| R11345 | 2 | CBFbeta(1-165)-HIF1alpha(549-575) | 234 | 420 | 822 |
| R11346 | 3 | RUNX1(1-177)-RUNX1T1a(31-604) | 236 | 422 | 824 |
| R11349 | 4 | RUNX1(K83E, R174Q) | 197 | 383 | 818 |
| R11302 | 5 | CBFbeta(1-165)-SMMHC(1527-1877) | 232 | 418 | 820 |
| R11344 | 6 | CBFbeta(1-141)-SMMHC(1527-1877) | 1545 | 1553 | 1563 |
| R9744 | 7 | CBFbeta(1-165)-Linker-LifeAct | 1548 | 1556 | 1566 |

### Summary of the Findings

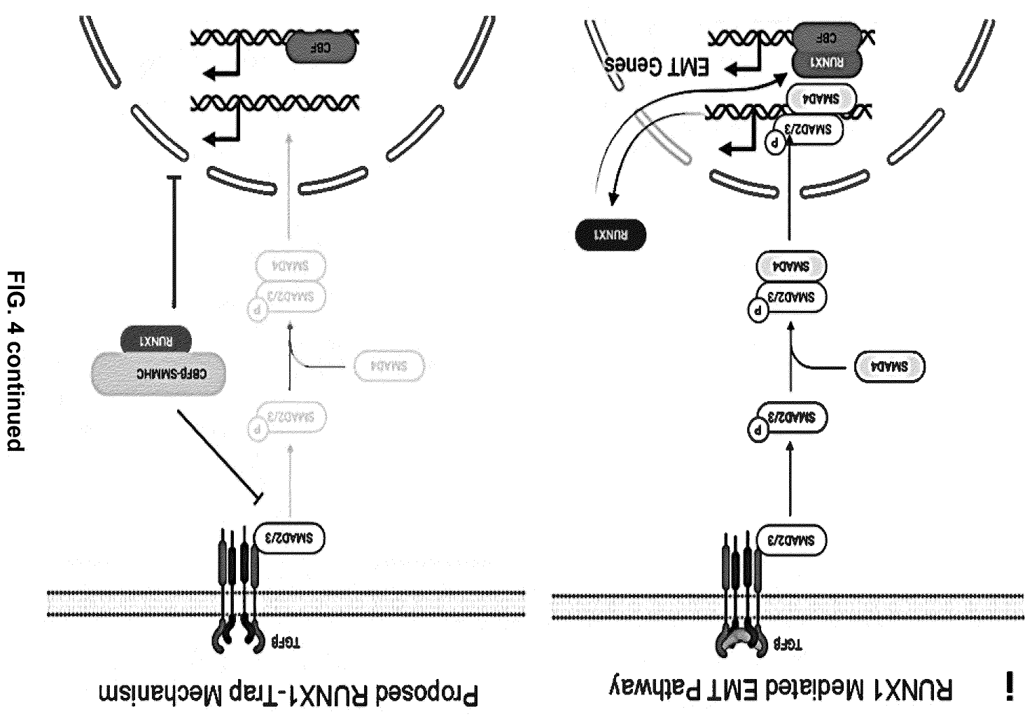

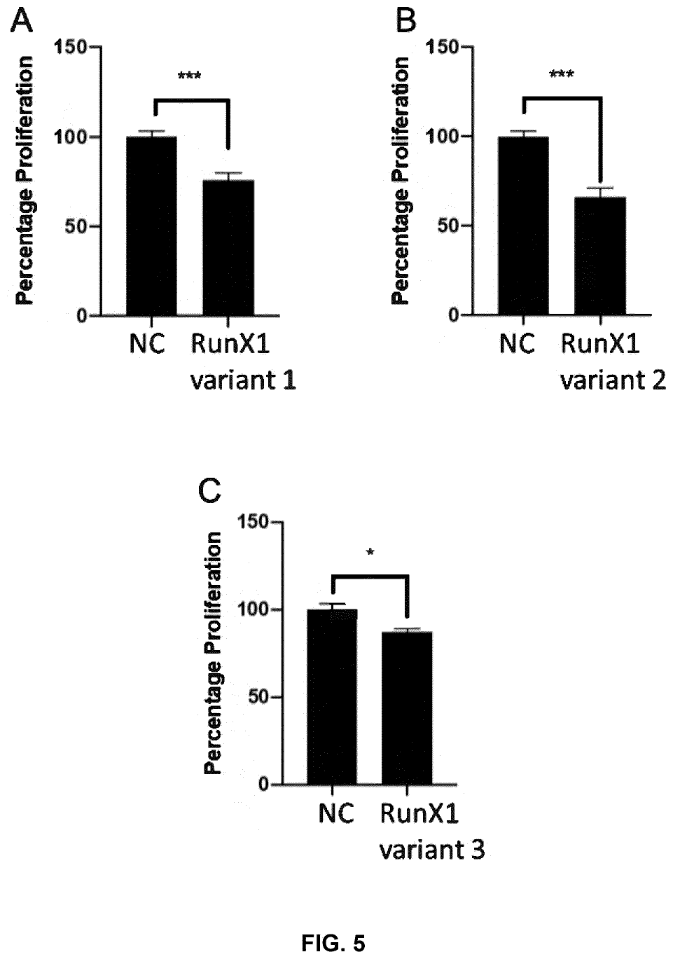

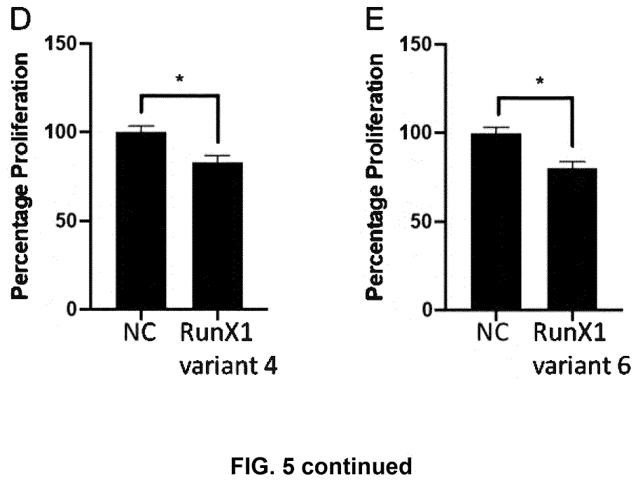

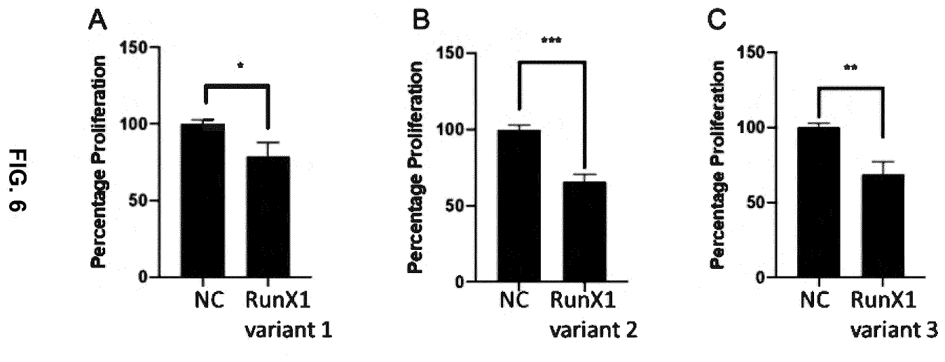

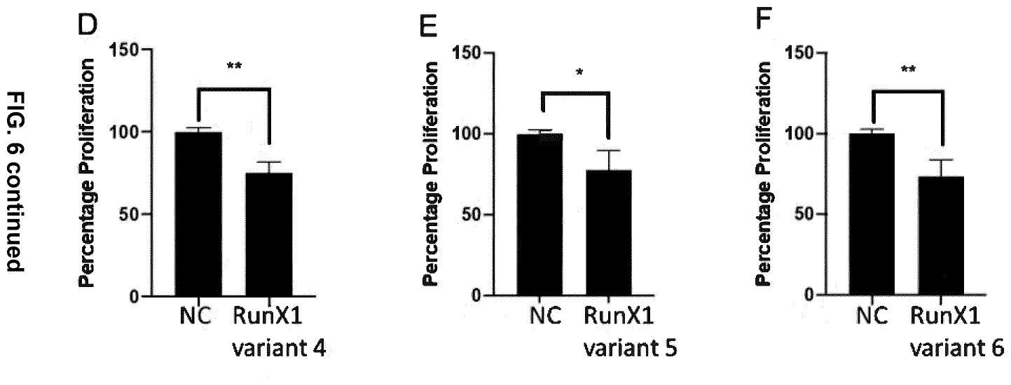

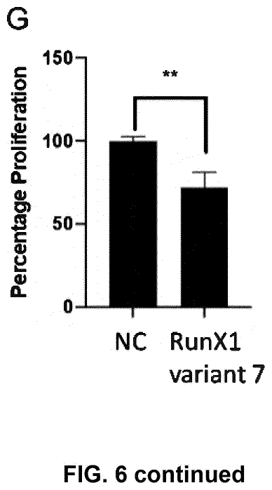

RUNX trap variants 1-4 and 6 already show a reduced proliferation of C-PVRs in vitro 24 h after transfection (FIG. 5). All RUNX trap mRNA variants reduce proliferation of C-PVRs in vitro 48 h after transfection (FIG. 6) and did not have an impact on cell viability.

### Example 8: SM-102 LNP-Formulated mRNA Encoding RUNX Trap Tested in ARPE-19 Cells

The goal of the experiment was to analyse the transfection efficiency (intracellular levels of CBFbeta) of eye cells using an LNP formulation. In the present example, SM-102 LNP-formulated mRNA encoding RUNX trap was used to transfect ARPE-19 cells. ARPE-19 is a spontaneously arising retinal pigment epithelia (RPE) cell line derived from the normal eyes of a 19-year-old male.

ARPE-19 cells were transfected with 200 ng of SM-102 LNP-formulated mRNA encoding RUNX trap (LNP1: R11302-unmodified and LNP2: R11316-N1-methylpseudouridine (m1w) modified). RNA formulations encoding RUNX trap used in the experiment were prepared according to Example 1, Western Blot analysis used herein is described in Example 2. Briefly, the samples were heated to 90° C. for 5 min and 30 μg RNA/sample was loaded on the gel. CBFbeta and actin were stained.

TABLE 8
LNP formulations and RUNX trap constructs used in Example 8

| | mRNA | | |
|---|---|---|---|
| LNP | TYPE | MW | SEQ ID NO: |
| LNP1:SM-102 | R11302 | 593114.6 | 820 |
| | non-modified | | |
| LNP2:SM-102 | R11316 | 595075.8 | 1580 |
| | m1ψ modified | | |

### Summary of the Findings

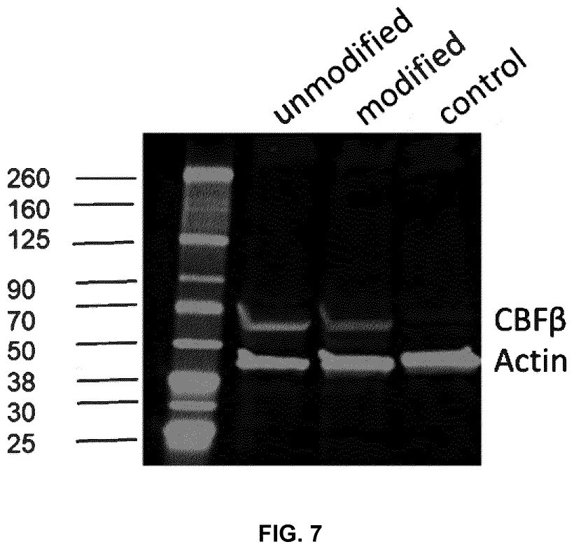

The data shows that LNP formulations (here SM-102 LNP) are effective and suitable for transfecting eye cells. CBFbeta could be detected in cells transfected with SM-102 LNPs for both mRNA constructs. In that experimental setting, levels of CBFbeta detected in cells transfected with non-modified mRNAs were slightly higher than those determined in cells transfected with N1-methylpseudouridine-modified mRNA (FIG. 7).

### Example 9: 315 LNP-Formulated mRNA Encoding Optimized RUNX Trap Tested in ARPE-19 Cells

The goal of the experiment was to analyse the transfection efficiency (intracellular levels of CBFbeta) of eye cells using an alternative LNP formulation. In the present example, 315 LNP-formulated mRNA encoding RUNX trap was used to transfect ARPE-19 cells.

Different sequence optimized RUNX trap mRNAs were compared (Table 9). ARPE cells were seeded 75.000 cell per well 24h prior transfection. 200 ng of RNA (each LNP-formulated RNA in 5 replicates) were transfected per well. Cells were harvested after 24 h after transfection and stained with 1st antibody (anti-CBFbeta) and secondary antibody (CF633) for FACS analysis. RNA formulations used in the experiment were prepared according to Example 1, methods used herein are described in Example 2.

Different codon-optimized and LNP-formulated mRNAs encoding RUNX trap were analyzed using FACS analysis:

TABLE 9
Different GC-optimized mRNAs encoding RUNX trap used in Example 9

| Formulation | mRNA | SEQ ID NO: | Optimization | LNP |
|---|---|---|---|---|
| F1 | R11350 | 830 | opt1 | L315C_R11602 |
| F2 | R11352 | 850 | opt3 | L315C_R11558 |
| F3 | R11302 | 820 | opt1 | L315C_R11641 |
| F4 (luciferase) | R8730 | 1520 | none | L315C_R11285 |

### Summary of the Findings

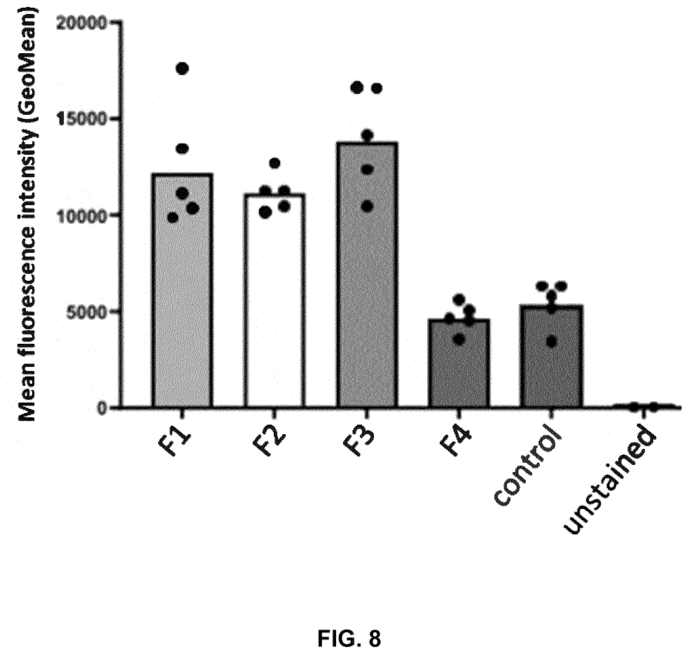

The data shows that 315 LNP formulations are effective and suitable for transfecting eye cells. Levels of protein production (CBFbeta) were comparable between the three tested codon optimizations (FIG. 8).

### Example 10:315 LNP-Formulated mRNA Encoding RUNX Trap Tested in C-PVR Cells

Different modified mRNAs encoding RUNX trap have been formulated in LNPs and tested in cells deriving form patients with PVR. RNA formulations used in the experiment were prepared according to Example 1, methods used herein are described in Example 2.

CBFbeta expression from RUNX trap formulations (Table 10) was analyzed using Western Blot (data not shown). Proliferation and cell viability assays (Example 2.4) have been performed to validate their functional activity. Hereby, C-PVR cells (10,000 cells/well) were cultured and maintained in 96-well plates. Cells were incubated with the RUNX variant formulations for 4 hours in serum free media. The proliferation rate was measured using CyQUANT Direct

Cell Proliferation Assay (ThermoFisher, Waltham, MA) according to the manufacturer's recommendation. Cell viability was measured using a lactate dehydrogenase (LDH) assay (Promega, G1780).

For IFNα analysis, C-PVR cells were cultured and maintained in 96-well plates. Cells were incubated with the RUNX trap-LNP formulation F5, F6, and F7 for 4 hours in complete media with the different concentrations. After 4 hours the media was switched to complete media without formulations and cells incubated for 20 hours. 24 hours post treatment, the cell supernatants were collected for detection and measurement of human pan interferon-alpha using Human pan IFNalpha ELISA Kit (Catalog #02000) of Stemcell following the manufacturer's protocol.

For LDL toxicity assay, C-PVR cells were cultured and maintained in 96-well plates. The cells were incubated with the formulations in media with serum. After exchanging the media the cells were further incubated and an LDH toxicity assay was performed according to the manufacturer's instructions.

Levels of cyclin D, a proliferation marker, were measured according to the manufacturer's instructions.

In brief, protein concentration was determined by Pierce bicinchoninic acid protein assay kit (ThermoFisher, Waltham, MA; 23227), according to the manufacturer's instructions. A total of 20 μg of total cell lysates was prepared in 4 μl 1 mol/L 1,4-dithiothreitol (Sigma Aldrich) and 10 μl Laemmli buffer (Boston Bioproducts, Ashland, MA) to a final volume of 40 μl and denatured for 5 minutes at 90° C. Samples were separated electrophoretically for 1 hour at 70 V using 4% to 20% precast gradient gels (MiniPROTEAN TGX; Bio-Rad, Hercules, CA) and SDS-Tris-Glycine buffer (Bio-Rad). Proteins were transferred to 0.45 μm nitrocellulose membranes for 1 hour at 70 V in ice-cold 20% Methanol Tris-Glycine buffer (Bio-Rad). Membranes were blocked for 1 hour with Odyssey Blocking Buffer (LI-COR Biosciences, Lincoln, NE). The membrane was then probed with Cyclin D1 Monoclonal Antibody (Thermo Fisher, Cat No 2G3G5) concentration (1:2000) followed by anti-mouse secondary antibody for 1 hour and washed 3× with Tris-buffered saline with Tween 20. Immunoreactive bands were detected using the Odyssey Infrared Imaging System and visualized on the Image Studio version 2.1 software (LI-COR Biosciences).

TABLE 10
RUNX trap formulations used in Example 10

| | | SEQ | | |
|---|---|---|---|---|
| Formulation | mRNA | ID NO: | Modification | LNP |
| F5 | R11353 | 1582 | pseudouridine | L315C_R11544 |
| F6 | R11316 | 1580 | N1-methylpseudouridine | L315C_R11545 |
| F7 | R11315 | 910 | none | L315C_R11546 |

### Summary of the Findings

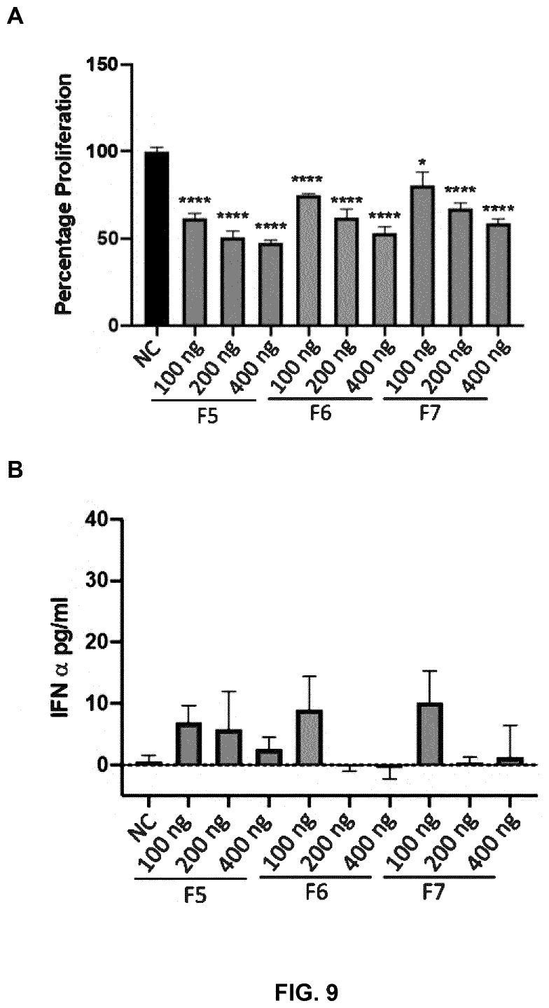

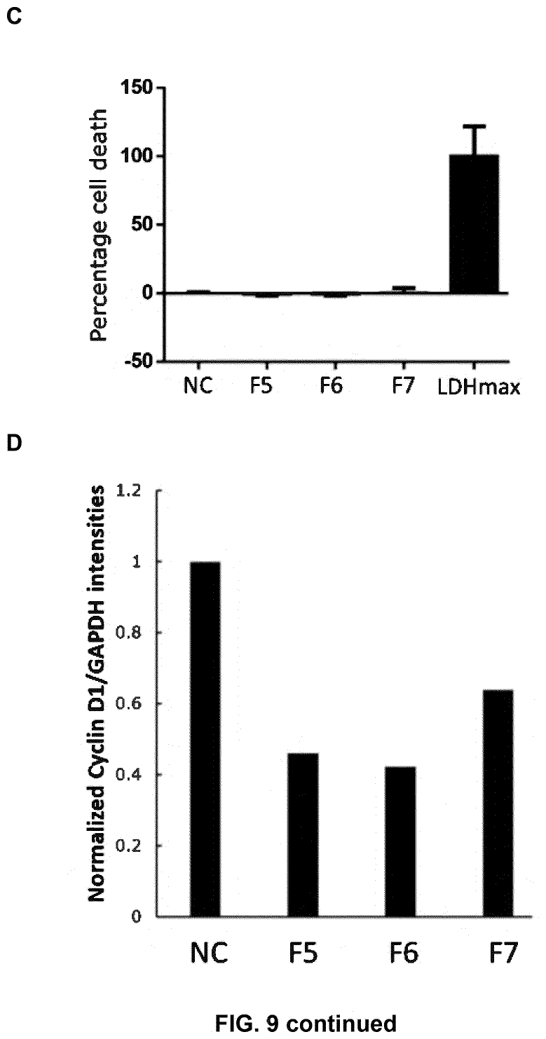

All formulations (F5-F7) show a clear dose dependent effect on proliferation upon transfection with mRNAs encoding RUNX traps (FIG. 9A). Low levels of IFNalpha could be detected and no clear differences between different mRNAs could be detected. The levels were the lowest in cells transfected with highest concentrations of mRNA, suggesting RUNX trap might have an anti-inflammatory effect (FIG. 9B). The transfection of C-PVR cells with RUNX trap formulations does not impair cell viability (FIG. 9C). Levels of cyclin D, a proliferation marker, were decreased in cells transfected with LNPs carrying mRNAs encoding RUNX trap (FIG. 9D).

### Example 11: Dose Effect of RUNX Trap 315 LNPs in a Laser-CNV Mice Model

315 LNPs carrying mRNA encoding RUNX trap have been tested in an in vivo CNV model in mice. For the laser-induced choroidal neovascularization (CNV) model, laser photocoagulation was performed using the Micron image-guide system (Pheonix, Oregon) and a 532 nm laser; 4 laser spots were administered at the 3-, 6-, 9-, and 12-o'clock meridians at 2-to-3-disc diameters of distance from the optic nerve head. Then, 1 μl of each treatment was injected via intravitreal administration. After 7 days of treatment, fundus fluorescein angiography was recorded under general anesthesia. 0.1 ml of 2% sodium fluorescein (AKORN, IL) was administered intraperitoneally, and serial photographs from the early (0 to 60 seconds) and late phases (6 minutes) were captured using the Micron IV imaging system (Pheonix, OR). Light source intensity and gain were standardized and maintained in all experiments. Animals were treated with vehicle, Eylea (40 μg/μl), or 1 μl of an LNP formulation (R11302, L315_R11641 non-modified mRNA, SEQ ID NO:820) at two concentrations (25 ng/μl and 50 ng/μl; ng referring to RNA). Leakage was quantified using ImageJ V13, recording the difference between the early and late phases of the lesions. Experimental groups were compared by a Kruskal-Wallis followed by a Dunn's test. The lesion sizes were also quantified on choroidal flat-mounts after ILB4 (Isolectin-IB 4) vascular staining which was performed according to manufacturer's description. Eylea (also known as Zaltrap) was used as a positive control and is an FDA-approved, anti-VEGF (anti-vascular endothelial growth factor) medication to treat wet age-related macular degeneration (AMD), impaired vision due to macular oedema (central retinal vein occlusion (CRVO) or branch retinal vein occlusion (BRVO)), impaired vision due to macular oedema caused by diabetes and impaired vision due to myopic choroidal neovascularization. Eylea contains the active substance aflibercept.

### Summary of the Findings

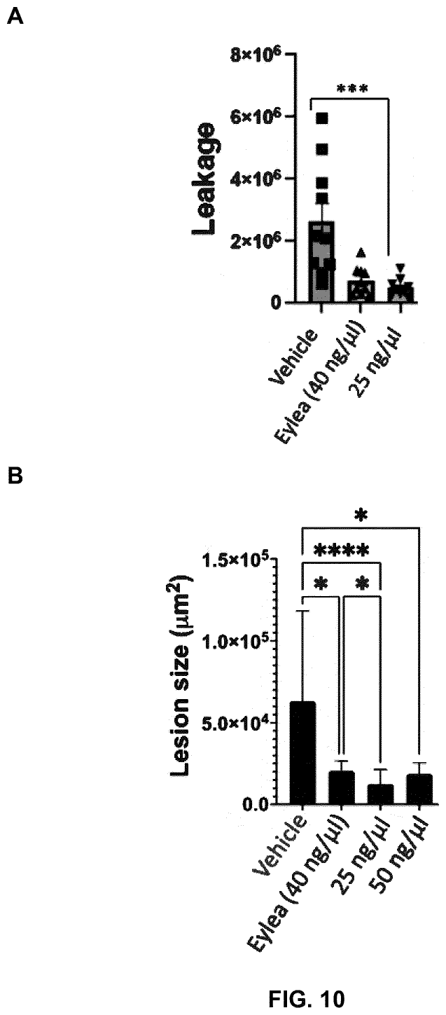

A dose of 25 ng RUNX trap mRNA (formulated in 315 LNPs) was sufficient to reduce leakage after 7 days of treatment (FIG. 10A). After 7 days of treatment, LNP-formulated RUNX trap reduces lesion size in a laser-CNV mouse model (FIG. 10B). A dose of 50 ng RUNX trap mRNA (formulated in 315 LNPs) was equally effective to Eylea and doses of 25 ng were even more effective in reducing lesion size compared to Eylea. Moreover, the data shows that the used LNP formulation is effective for ocular administration (intravitreal injection) of RNA, in particular mRNA encoding RUNX inhibitors.

### Example 12 LNP-Formulated mRNA Encoding RUNX Trap in a PVR Rabbit Disease Model

The goal of this study is to confirm efficacy of LNP-formulated mRNA encoding RUNX trap in a PVR rabbit disease model. The experiments are performed essentially as described in Example 5.

315 LNPs formulated RUNX trap mRNA (R11316, SEQ ID NO:1580 N1-methylpseudouridine (m1w) modified) or CVCM formulated RUNX trap mRNA (R9717, SEQ ID NO:820, non-modified) is injected intravitreally in eyes of a PVR rabbit disease model.

As a control, a small molecule RUNX1 inhibitor Ro5-335 is used (Delgado-Tirado et al., 2020). Details regarding the experimental setup are provided in Table 11.

TABLE 11
Study groups used in Example 12

| | | Administration | | Number of |
|---|---|---|---|---|
| Group | Administration | route | Dose | rabbits |
| 1 | NaCl | Intravitreal injection | 50 μl | N = 8 rabbits |
| 2 | RUNX trap mRNA in CVMCs | Intravitreal injection | 36 μg in 50 μl volume | N = 8 rabbits |
| 3 | RUNX trap mRNA in LNPs | Intravitreal injection | 3 μg in 50 μl volume | N = 8 rabbits |
| 4 | RUNX trap mRNA in LNPs | Intravitreal injection | 1 μg in 50 μl volume | N = 8 rabbits |
| 5 | RUNX trap mRNA in LNPs | Intravitreal injection | 0.3 μg in 50 μl volume | N = 8 rabbits |
| 6 | Small molecule inhibitor Ro5-335 | Eye drops | 3×/day | N = 16 rabbits |

1. An artificial nucleic acid comprising at least one coding sequence encoding at least one RUNX transcription factor inhibitor for reducing or inhibiting the activity of a RUNX target transcription factor in a cell, wherein the nucleic acid comprises at least one heterologous untranslated region (UTR).

2. The artificial nucleic acid of claim 1, wherein the RUNX transcription factor inhibitor is produced in the cytosol upon administration of the artificial nucleic acid to a cell, tissue, or subject.

3. The artificial nucleic acid of claim 1 or 2, wherein the RUNX transcription factor inhibitor is a dominant negative inhibitor of the target transcription factor and/or its transcription co-factor.

4. The artificial nucleic acid of claims 1 to 3, wherein the RUNX target transcription factor is selected from RUNX1, RUNX2, RUNX3, preferably RUNX1.

5. The artificial nucleic acid of claims 1 to 4, wherein the RUNX transcription factor inhibitor comprises at least one amino acid sequence element A that is configured to bind to the target transcription factor or its transcription co-factor.

6. The artificial nucleic acid of any one of the preceding claims, wherein the RUNX transcription factor inhibitor comprises at least one amino acid sequence element A that comprises or consists of an amino acid sequence selected or derived from the target transcription factor, an interaction partner of the target transcription factor, a binding partner of the target transcription factor, a transcription co-factor of the target transcription factor, an antibody moiety, an intrabody moiety, a peptide-based aptamer, or a fragment or variant of any of these.

7. The artificial nucleic acid of claim 5 or 6, wherein the at least one amino acid sequence element A comprises or consists of an amino acid sequence selected or derived from a transcription co-factor of RUNX, or a fragment or variant thereof.

8. The artificial nucleic acid of claim 7, wherein the transcription co-factor of RUNX is selected or derived from CBFbeta, for example CBFbeta1 or CBFbeta2, or a fragment or variant thereof.

9. The artificial nucleic acid of claim 8, wherein the CBFbeta amino acid sequence is an N-terminal fragment of a human CBFbeta, preferably a fragment comprising amino acid 1 to amino acid 141 of CBFbeta, more preferably a fragment comprising amino acid 1 to amino acid 162 of CBFbeta.

10. The artificial nucleic acid of any one of the preceding claims, wherein the RUNX transcription factor inhibitor comprises at least one amino acid sequence element A that comprises or consists of an amino acid sequence selected or derived from CBFbeta being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 178-183, or fragments or variants of any of these.

11. The artificial nucleic acid of claim 5 or 6, wherein the at least one amino acid sequence element A comprises or consists of an amino acid sequence selected or derived from the RUNX target transcription factor, or a fragment or variant thereof.

12. The artificial nucleic acid of claim 11, wherein the amino acid sequence selected or derived from the RUNX target transcription factor comprises at least one amino acid substitution or deletion that reduces or prevents binding to its target DNA and/or at least one amino acid substitution or deletion that reduces or prevents nuclear translocation and/or at least one amino acid substitution or deletion that reduces or prevents homodimerization or heterodimerization.

13. The artificial nucleic acid of claim 11 or 12, wherein the at least one amino acid sequence element A comprises or consists of an amino acid sequence selected or derived from RUNX or a fragment or variant thereof, preferably wherein the RUNX amino acid sequence is an N-terminal fragment of a human RUNX1, e.g. a fragment comprising the Runt homology domain (RHD).

14. The artificial nucleic acid of claim 13, wherein at least one amino acid substitution in the RUNX1 amino acid sequence is selected from R80A, K83A, K83E, R135A, R139A, R142A, K167A, T169A, D171A, R174A, or R177A, or any functionally equivalent amino acid substitution at position R80, K83, R135, R139, R142, K167, T169, D171, R174, or R177, preferably wherein at least one amino acid substitution in the RUNX1 amino acid sequence is selected from R174Q and/or K83E, or any functionally equivalent amino acid substitution at position R174 and/or K83.

15. The artificial nucleic acid of claims 11 to 14, wherein the RUNX transcription factor inhibitor comprises at least one amino acid sequence element A that comprises or consists of an amino acid sequence selected or derived from RUNX1 being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 197-215, or fragments or variants of any of these.

16. The artificial nucleic acid of any one of the preceding claims, wherein the RUNX transcription factor inhibitor comprises at least one amino acid sequence element B that is configured to prevent or reduce the interaction of the target transcription factor with its target DNA.

17. The artificial nucleic acid of any one of the preceding claims, wherein the RUNX transcription factor inhibitor comprises at least one amino acid sequence element B that is configured to prevent or reduce interaction of the RUNX target transcription factor with at least one transcription co-factor of the RUNX target transcription factor.

18. The artificial nucleic acid of any one of the preceding claims, wherein the RUNX transcription factor inhibitor comprises at least one amino acid sequence element B that is configured to prevent or reduce nuclear translocation of the RUNX target transcription factor or its transcription co-factor.

19. The artificial nucleic acid of any one of the preceding claims, wherein the RUNX transcription factor inhibitor comprises at least one amino acid sequence element B that is configured to bind the RUNX target transcription factor or its transcription co-factor preferably in the cytosol.

20. The artificial nucleic acid of any one of the preceding claims, wherein the RUNX transcription factor inhibitor comprises at least one amino acid sequence element B that comprises or consists of an amino acid sequence selected or derived from a cytoplasmic protein, a protein that is associated with or binds to a cytoplasmic protein, or a fragment or variant thereof.

21. The artificial nucleic acid of claims 16 to 20, wherein the amino acid sequence element B is selected or derived from a cytoskeletal protein or a protein that is associated with or binds to a cytoskeletal protein, or a fragment or variant of any of these.

22. The artificial nucleic acid of claims 16 to 21, wherein the amino acid sequence element B is selected or derived from a myofibrillar protein, a microtubule protein, an intermediate filament protein, or a fragment or variant of any of these or selected or derived from a peptide or protein that is associated with or binds to a myofibrillar protein, a microtubule protein, an intermediate filament protein, or a fragment or variant of any of these.

23. The artificial nucleic acid of claims 16 to 22, wherein the amino acid sequence element B is selected or derived from a protein that comprises an myofibrillar binding domain (e.g. actin), a microtubule binding domain, an intermediate filament binding domain, preferably wherein the amino acid sequence element B is selected or derived from a protein that comprises an actin binding domain.

24. The artificial nucleic acid of claims 16 to 23, wherein the amino acid sequence element B is selected from smooth muscle myosin heavy chain (SMMHC), or a fragment or variant thereof.

25. The artificial nucleic acid of claim 24, wherein the amino acid sequence selected or derived from SMMHC comprises at least one of a high-affinity binding domain (HABD) and/or an assembly competent domain (ACD) and/or a transcriptional repression domain (TRD), or fragments or variants of any of these.

26. The artificial nucleic acid of claim 24 or 25, wherein the SMMHC amino acid sequence is a C-terminal fragment of a human SMMHC, for example an SMMHC fragment comprising amino acid 1527 to amino acid 1972.

27. The artificial nucleic acid of claims 24 to 26, wherein the SMMHC amino acid sequence comprises a deletion in the C-terminus of about 95aa (SMMHCAC95), for example an SMMHC fragment comprising amino acid 1527 to amino acid 1877.

28. The artificial nucleic acid of any one of the preceding claims, wherein the RUNX transcription factor inhibitor comprises at least one amino acid sequence element B that comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 184-193, 1521, 1522, or fragments or variants of any of these.

29. The artificial nucleic acid of claims 16 to 19, wherein the at least one element B comprises or consists of an amino acid sequence selected or derived from a transcriptional repressor of the RUNX target transcription factor, or a fragment or variant thereof, preferably wherein the transcriptional repressor of the RUNX target transcription factor is selected or derived from RUNX1T1a, RUNX1T1b, or a fragment or variant of any of these.

30. The artificial nucleic acid of claim 29, wherein the RUNX transcription factor inhibitor comprises at least one amino acid sequence element B that comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 216-230, or fragments or variants of any of these.

31. The artificial nucleic acid of claims 16 to 19, wherein the at least one element B comprises or consists of an amino acid sequence selected or derived from a peptide or protein that promotes degradation of the target transcription factor, or a fragment or variant thereof.

32. The artificial nucleic acid of claim 31, wherein the peptide or protein that promotes degradation may be selected from a protein that binds to E3 ligase, preferably a protein selected from HIF1alpha, MDM2, or CRBN, or a fragment or variant of any of these, preferably wherein the peptide or protein that promotes degradation is selected or derived from HIF1alpha, preferably a HIF1alpha fragment comprising amino acid 549 to amino acid 575.

33. The artificial nucleic acid of claim 31 or 32, wherein the RUNX transcription factor inhibitor comprises at least one amino acid sequence element B that comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 194 or 195, or fragments or variants of any of these.

34. The artificial nucleic acid of any one of the preceding claims, wherein the RUNX transcription factor inhibitor is a fusion protein that comprises or consists of at least one amino acid sequence element A and at least one amino acid sequence element B, preferably wherein the at least one element A is located at the N-terminus of the transcription inhibitor and the at least one element B is located at the C-terminus of the transcription inhibitor.

35. The artificial nucleic acid of claim 34, wherein the RUNX transcription factor inhibitor comprises at least one further amino acid sequence element selected from a linker sequence, preferably a flexible linker, more preferably a flexible GGS linker according to SEQ ID NO: 196, or a variant thereof, optionally wherein the linker sequence is positioned between amino acid sequence element A and amino acid sequence element B.

36. The artificial nucleic acid of any one of the preceding claims, wherein the RUNX transcription factor inhibitor is a RUNX trap, preferably a RUNX1 trap.

37. The artificial nucleic acid of any one of the preceding claims, wherein the RUNX transcription factor inhibitor, preferably the RUNX trap, comprises or consists of a fusion protein comprising a CBFbeta amino acid sequence element and an SMMHC amino acid sequence element.

38. The artificial nucleic acid of any one of the preceding claims, wherein the RUNX transcription factor inhibitor, preferably the RUNX trap, comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 231-233, 1541-1548, or fragments or variants of any of these.

39. The artificial nucleic acid of any one of the preceding claims, wherein the RUNX transcription factor inhibitor, preferably the RUNX trap, comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 232, or a fragment or variant thereof.

40. The artificial nucleic acid of any one of the preceding claims, wherein the RUNX transcription factor inhibitor, preferably the RUNX trap, sequesters cellular RUNX by binding to RUNX in the cytosol and preferably trapping RUNX1 in the cytosol.

41. The artificial nucleic acid of claims 1 to 35, wherein the RUNX transcription factor inhibitor comprises or consists of a fusion protein comprising a CBFbeta amino acid sequence element and an HIF1alpha amino acid sequence element.

42. The artificial nucleic acid of claim 41, wherein the RUNX transcription factor inhibitor comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 234 or 235, or fragments or variants thereof.

43. The artificial nucleic acid of 41 or 42, wherein the RUNX transcription factor inhibitor degrades cellular RUNX.

44. The artificial nucleic acid of claims 1 to 35, wherein the RUNX transcription factor inhibitor comprises or consists of a fusion protein comprising a RUNX1 amino acid sequence element and a RUNX1T1a or RUNX1T1b amino acid sequence element.

45. The artificial nucleic acid of claim 44, wherein the RUNX transcription factor inhibitor comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 236-239, or fragments or variants thereof.

46. The artificial nucleic acid of claims 1 to 35, wherein the RUNX transcription factor inhibitor comprises or consists of an amino acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 197, or a fragment or a variant thereof.

47. The artificial nucleic acid of claims 44 to 46, wherein the RUNX transcription factor inhibitor drives transcriptional repression of genes that are under control of RUNX.

48. The artificial nucleic acid of any one of the preceding claims, wherein the RUNX transcription factor inhibitor, preferably the RUNX trap, reduces or prevents the interaction of cellular RUNX with cellular CBFbeta.

49. The artificial nucleic acid of any one of the preceding claims, wherein the RUNX transcription factor inhibitor, preferably the RUNX trap, reduces cellular RUNX-CBFbeta complex formation and/or activity.

50. The artificial nucleic acid of any one of the preceding claims, wherein the RUNX transcription factor inhibitor, preferably the RUNX trap, reduces the cellular expression of RUNX controlled or regulated gene products.

51. The artificial nucleic acid of any one of the preceding claims, wherein the RUNX transcription factor inhibitor, preferably the RUNX trap, reduces the cellular expression of RUNX, TGFbeta2, SMAD3, and/or COL1A1.

52. The artificial nucleic acid of any one of the preceding claims, wherein the RUNX transcription factor inhibitor, preferably the RUNX trap, reduces or prevents pathological epithelial to mesenchymal transition (EMT), pathological cell proliferation, aberrant angiogenesis, aberrant neovascularization, degeneration, and/or fibrosis.

53. The artificial nucleic acid of any one of the preceding claims, wherein the at least one coding sequence is a codon modified coding sequence, preferably wherein codon modified coding sequence is selected from a C maximized coding sequence, a CAI maximized coding sequence, human codon usage adapted coding sequence, a G/C content modified coding sequence, and a G/C optimized coding sequence, or any combination thereof, preferably wherein the at least one codon modified coding sequence is a G/C optimized coding sequence.

54. The artificial nucleic acid of any one of the preceding claims, wherein the at least one coding sequence comprises a nucleic acid sequence encoding an amino acid sequence element A, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 240-245, 302-307, 364-369, 426-431, 488-493, 550-555, 612-617, 674-679, 736-741, or a fragment or a variant of any of these.

55. The artificial nucleic acid of any one of the preceding claims, wherein the at least one coding sequence comprises a nucleic acid sequence encoding an amino acid element B, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 246-255, 308-317, 370-379, 432-441, 494-503, 556-565, 618-627, 680-689, 742-751, 1523-1540, or a fragment or a variant of any of these.

56. The artificial nucleic acid of any one of the preceding claims, wherein the at least one coding sequence comprises or consists of a nucleic acid sequence encoding a RUNX transcription factor inhibitor, preferably a RUNX trap, that is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 293-295, 355-357, 417-419, 479-481, 541-543, 603-605, 665-667, 727-729, 789-791, 1549-1558, or a fragment or a variant of any of these.

57. The artificial nucleic acid of any one of the preceding claims, wherein the at least one coding sequence comprises or consists of a nucleic acid sequence encoding a RUNX transcription factor inhibitor, preferably a RUNX trap CBFbeta-SMMHC, that is identical or at least 70%, 80%, 90%, 91%, 92%, 93%, 94%, 95%, 96%, 97%, 98%, or 99% identical to SEQ ID NO: 418, or a fragment or a variant thereof.

58. The artificial nucleic acid of claims 1 to 54, wherein the at least one coding sequence comprises a nucleic acid sequence encoding an amino acid sequence element B, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 257, 319, 381, 443, 505, 567, 629, 691, 753, or a fragment or a variant of any of these.

59. The artificial nucleic acid of claim 58, wherein the at least one coding sequence comprises or consists of a nucleic acid sequence encoding a RUNX transcription factor inhibitor, preferably a RUNX inhibitor CBFbeta-HIF1alpha, that is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 296-297, 358-359, 420-421, 482-483, 544-545, 606-607, 668-669, 730-731, 792-793, or a fragment or a variant of any of these.

60. The artificial nucleic acid of claims 1 to 53, wherein the at least one coding sequence comprises a nucleic acid sequence encoding an amino acid element A, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 259-277, 321-339, 383-401, 445-463, 507-525, 569-587, 631-649, 693-711, 755-773, or a fragment or a variant of any of these.

61. The artificial nucleic acid of claim 60, wherein the at least one coding sequence comprises a nucleic acid sequence encoding an amino acid element B, wherein the nucleic acid sequence is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 278-292, 340-354, 402-416, 464-478, 526-540, 588-602, 650-664, 712-726, 774-788, or a fragment or a variant of any of these.

62. The artificial nucleic acid of claims 60 to 61, wherein the at least one coding sequence comprises or consists of a nucleic acid sequence encoding a RUNX transcription factor inhibitor, preferably a RUNX inhibitor RUNX1-RUNX1T1, that is identical or at least 70%, 80%, 85%, 86%, 87%, 88%, 89%, 90%, 91%, 92%, 93%, 94%, 95%, 96%, 97%, 98%, or 99% identical to any one of the nucleic acid sequences SEQ ID NOS: 298-301, 360-363, 422-425, 484-487, 546-549, 608-611, 670-673, 732-735, 794-797, or a fragment or a variant of any of these.

63. The artificial nucleic acid of claims 1 to 53, wherein the at least one coding sequence comprises or consists of a nucleic acid sequence encoding a RUNX transcription factor inhibitor, preferably a RUNX inhibitor RUNX1 (K83E, R174Q), that is identical or at least 70%, 80%, 85%, 86%, 87%, 88%, 89%, 90%, 91%, 92%, 93%, 94%, 95%, 96%, 97%, 98%, or 99% identical to any one of the nucleic acid sequences SEQ ID NOs: 259, 321, 383, 445, 507, 569, 631, 693, 755, or a fragment or a variant of any of these.

64. The artificial nucleic acid of any one of the preceding claims, wherein the at least one heterologous untranslated region (UTR) is selected from at least one heterologous 5′-UTR and/or at least one heterologous 3′-UTR.

65. The artificial nucleic acid of claim 64, wherein the at least one heterologous 3′-UTR comprises or consists of a nucleic acid sequence derived from a 3′-UTR of a gene selected from PSMB3, ALB7, alpha-globin, CASP1, COX6B1, GNAS, NDUFA1 and RPS9, or from a homolog, a fragment or a variant of any one of these genes, preferably wherein the at least one heterologous 3′-UTR comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 67-90, 109-120, or a fragment or a variant of any of these.

66. The artificial nucleic acid of claim 64 or 65, wherein the at least one heterologous 5′-UTR comprises or consists of a nucleic acid sequence derived from a 5′-UTR of a gene selected from HSD17B4, RPL32, ASAH1, ATP5A1, MP68, NDUFA4, NOSIP, RPL31, SLC7A3, TUBB4B and UBQLN2, or from a homolog, a fragment or variant of any one of these genes, preferably selected from HSD17B4, preferably wherein the at least one heterologous 3′-UTR comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 1-32, 65-66, or a fragment or a variant of any of these.

67. The artificial nucleic acid of claims 64 to 66, wherein the at least one heterologous 5′-UTR is selected from HSD17B4 and the at least one heterologous 3′ UTR is selected from PSMB3, preferably wherein.

68. The artificial nucleic acid of claims 64 to 67, wherein the at least one heterologous 3′-UTR comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 68, or a fragment or a variant thereof.

69. The artificial nucleic acid of claims 64 to 68, wherein the at least one heterologous 5′-UTR comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 2, or a fragment or a variant thereof.

70. The artificial nucleic acid of any one of the preceding claims, additionally comprising at least one poly(A) sequence, preferably wherein the at least one poly(A) sequence comprises about 40 to about 500 adenosine nucleotides, preferably about 60 to about 250 adenosine nucleotides, more preferably about 60 to about 150 adenosine nucleotides.

71. The artificial nucleic acid of claim 70, wherein the at least one poly(A) sequence comprises about 100 adenosine nucleotides.

72. The artificial nucleic acid of claim 70 or 71, wherein the at least one poly(A) sequence is located at the 3′ terminus, optionally, wherein the 3′ terminal nucleotide is an adenosine.

73. The artificial nucleic acid of any one of the preceding claims, additionally comprising at least one poly(C) sequence and/or at least one miRNA binding site and/or histone stem-loop sequence, preferably a histone stem-loop sequence.

74. The artificial nucleic acid of any one of the preceding claims, wherein the nucleic acid a DNA vector, preferably an AAV vector, or an RNA.

75. The artificial nucleic acid of any one of the preceding claims, wherein the nucleic acid is an RNA selected from mRNA, circular RNA, replicon RNA, or viral RNA.

76. The artificial nucleic acid of any one of the preceding claims, wherein the nucleic acid is an mRNA.

77. The artificial nucleic acid of claim 75 or 76, wherein the RNA comprises at least one modified nucleotide, preferably selected from pseudouridine (ψ) or N1-methylpseudouridine (m1ψ).

78. The artificial nucleic acid of claim 77, wherein the RNA comprises N1-methylpseudouridine (m1ψ).

79. The artificial nucleic acid of any one of the preceding claims, wherein the nucleic acid is a modified RNA wherein each uracil is substituted by N1-methylpseudouridine (m1ψ).

80. The artificial nucleic acid of any one of the preceding claims, wherein the nucleic acid is an RNA that comprises a 5′-cap structure.

81. The artificial nucleic acid of claim 80, wherein the 5′-cap structure is selected from a cap1 structure or a modified cap1 structure.

82. The artificial nucleic acid of any one of the preceding claims, wherein the nucleic acid is an in vitro transcribed RNA, preferably wherein RNA in vitro transcription has been performed in the presence of a sequence optimized nucleotide mixture.

83. The artificial nucleic acid of any one of the preceding claims, wherein the nucleic acid is a purified RNA, preferably wherein the RNA has been purified by RP-HPLC, AEX, SEC, hydroxyapatite chromatography, TFF, filtration, precipitation, core-bead flow through chromatography, oligo(dT) purification, cellulose-based purification, or any combination thereof.

84. The artificial nucleic acid of claim 83, wherein the at least one step of purification is selected from RP-HPLC and/or TFF.

85. The artificial nucleic acid of any one of the preceding claims, wherein the nucleic acid, preferably the RNA has an integrity of at least about 50%, preferably of at least about 60%, more preferably of at least about 70%, most preferably of at least about 80%.

86. The artificial nucleic acid of any one of the preceding claims, wherein the nucleic acid, preferably the RNA is suitable for use in treatment or prevention of a disease, disorder or condition, preferably an ocular disease, disorder or condition.

87. The artificial nucleic acid of any one of the preceding claims, wherein the nucleic acid, preferably the RNA comprises the following sequence elements preferably in 5′- to 3′-direction:
A) a 5′-cap structure;
B) a 5′-UTR preferably selected or derived from a 5′-UTR of a HSD17B4 gene;
C) a coding sequence encoding a RUNX inhibitor;
D) a 3′-UTR preferably selected or derived from a 3′-UTR of a PSMB3 gene;
E) optionally, a histone stem-loop; and
F) a poly(A) sequence preferably comprising about 100 A nucleotides.

88. The artificial nucleic acid of any one of the preceding claims, wherein the nucleic acid, preferably the RNA comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 798-1517, 1559-1582, or a fragment or variant of any of these sequences.

89. The artificial nucleic acid of any one of the preceding claims, wherein nucleic acid, preferably the RNA comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 799-801, 809-811, 819-821, 829-831, 839-841, 849-851, 859-861, 869-871, 879-881, 889-891, 899-901, 909-911, 919-921, 929-931, 939-941, 949-951, 959-961, 969-971, 979-981, 989-991, 999-1001, 1009-1011, 1019-1021, 1029-1031, 1039-1041, 1049-1051, 1059-1061, 1069-1071, 1079-1081, 1089-1091, 1099-1101, 1109-1111, 1119-1121, 1129-1131, 1139-1141, 1149-1151, 1159-1161, 1169-1171, 1179-1181, 1189-1191, 1199-1201, 1209-1211, 1219-1221, 1229-1231, 1239-1241, 1249-1251, 1259-1261, 1269-1271, 1279-1281, 1289-1291, 1299-1301, 1309-1311, 1319-1321, 1329-1331, 1339-1341, 1349-1351, 1359-1361, 1369-1371, 1379-1381, 1389-1391, 1399-1401, 1409-1411, 1419-1421, 1429-1431, 1439-1441, 1449-1451, 1459-1461, 1469-1471, 1479-1481, 1489-1491, 1499-1501, 1509-1511, 1559-1582, or a fragment or variant of any of these sequences.

90. The artificial nucleic acid of any one of the preceding claims, wherein the nucleic acid, preferably the RNA comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 800, 810, 820, 830, 840, 850, 860, 870, 880, 890, 900, 910, 920, 930, 940, 950, 960, 970, 1567, 1568, 1577, 1578-1582, 980, 990, 1000, 1010, 1020, 1030, 1040, 1050, 1060, 1070, 1080,1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, or a fragment or variant of any of these sequences.

91. The artificial nucleic acid of any one of the preceding claims, wherein the nucleic acid, preferably the RNA comprises or consists of a nucleic acid sequence which is identical or at least 70%, 80%, 90%, 91%, 92%, 93%, 94%, 95%, 96%, 97%, 98%, or 99% identical to a nucleic acid sequence selected from SEQ ID NOs: 820, 1579, 1581, 910, 1580 or 1582 or a fragment or variant of that sequence.

92. The artificial nucleic acid of claim 91, wherein the nucleic acid, preferably the RNA, comprises or consists of a nucleic acid sequence which is identical to the nucleic acid sequence SEQ ID NO: 1580 or 1579.

93. A pharmaceutical composition comprising at least one artificial nucleic acid comprising at least one coding sequence encoding at least one RUNX transcription factor inhibitor as defined in any one of the claims 1 to 92.

94. The pharmaceutical composition of claim 93, comprising at least one pharmaceutically acceptable carrier or excipient.

95. The pharmaceutical composition of claim 93 or 94, wherein the at least one artificial nucleic acid, preferably the RNA is formulated in at least one cationic or polycationic compound.

96. The pharmaceutical composition of claim 95, wherein the at least one cationic or polycationic compound is selected from a cationic or polycationic polymer, a cationic or polycationic polysaccharide, a cationic or polycationic lipid, a cationic or polycationic protein, a cationic or polycationic peptide, or any combinations thereof.

97. The pharmaceutical composition of claim 96, wherein the one or more cationic or polycationic peptides are selected from SEQ ID NOs: 173-177, or any combinations thereof.

98. The pharmaceutical composition of claim 97, wherein the cationic or polycationic polymer is selected from a polyethylene glycol/peptide polymer, preferably comprising HO-PEG5000-S-(S-CHHHHHHRRRRHHHHHHC-S-)7-S-PEG5000-OH (SEQ ID NO: 176 as peptide monomer), HO-PEG5000-S-(S-CHHHHHHRRRRHHHHHHC-S-) 4-S-PEG5000-OH (SEQ ID NO: 176 as peptide monomer), HO-PEG5000-S-(S-CGHHHHHRRRRHHHHHGC-S-) 7-S-PEG5000-OH (SEQ ID NO: 177 as peptide monomer), or HO-PEG5000-S-(S-CGHHHHHRRRRHHHHHGC-S-) 4-S-PEG5000-OH (SEQ ID NO: 177 as peptide monomer).

99. The pharmaceutical composition of claims 96 to 98, wherein the cationic or polycationic polymer additionally comprises at least one lipidoid component.

100. The pharmaceutical composition of claim 99, wherein the at least one lipidoid component is selected from 3-C12-OH, 3-C12-OH-cat, or 3-C12-C3-OH.

101. The pharmaceutical composition of claims 93 to 96, wherein the at least one artificial nucleic acid, preferably the RNA is formulated in lipid-based carriers.

102. The pharmaceutical composition of claim 101, wherein the lipid-based carriers are selected from liposomes, lipid nanoparticles, lipoplexes, solid lipid nanoparticles, lipo-polylexes, and/or nanoliposomes.

103. The pharmaceutical composition of claim 101 or 102, wherein the lipid-based carriers are lipid nanoparticles, preferably wherein the lipid nanoparticles encapsulate the artificial nucleic acid.

104. The pharmaceutical composition of claims 101 to 103, wherein the lipid-based carriers comprise at least one aggregation-reducing lipid, at least one cationic lipid or ionizable lipid, at least one neutral lipid or phospholipid, and at least one steroid or steroid analog.

105. The pharmaceutical composition of claim 104, wherein the aggregation reducing lipid is selected from a polymer conjugated lipid.

106. The pharmaceutical composition of claim 105, wherein the polymer conjugated lipid is selected from a PEG-conjugated lipid or a PEG-free lipid.

107. The pharmaceutical composition of claim 105 or 106, wherein the polymer conjugated lipid is selected or derived from DMG-PEG 2000, C10-PEG2K, Cer8-PEG2K, POZ-lipid, or ALC-0159.

108. The pharmaceutical composition of claims 105 to 107, wherein the polymer conjugated lipid is a PEG-free lipid selected from a POZ-lipid.

109. The pharmaceutical composition of claims 104 to 108, wherein the cationic lipid or ionizable lipid is selected from an amino lipid, preferably wherein the amino lipid comprises a tertiary amine group.

110. The pharmaceutical composition of claims 104 to 109, wherein the at least one cationic or ionizable lipid is a lipid selected or derived from formula (III-1) 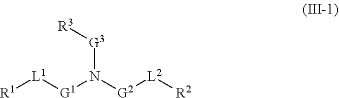
preferably, wherein one of L1 or L2 is —O(C═O)—, —(C═O)O—, —C(═O)—, —O—, —S(O)x-, —S—S—, —C(═O)S—, SC(═O)—, —NRaC(═O)—, —C(═O)NRa—, —NRaC(═O)NRa—, —OC(═O)NRa— or —NRaC(═O)O—, and the other of L1 or L2 is —O(C═O)—, —(C═O)O—, —C(═O)—, —O—, —S(O)x-, —S S—, —C(═O)S—, SC(═O)—, —NRaC(═O)—, —C(═O)NRa—, —NRaC(═O)NRa—, —OC(═O)NRa— or —NRaC(═O)O— or a direct bond; G1 and G2 are each independently unsubstituted C1-C12 alkylene or C1-C12 alkenylene; G3 is C1-C24 alkylene, C1-C24 alkenylene, C3-C8 cycloalkylene, C3-C8 cycloalkenylene; Ra is H or C1-C12 alkyl; R1 and R2 are each independently C6-C24 alkyl or C6-C24 alkenyl; R3 is H, OR5, CN, C(═O)OR4, OC(═O)R4 or —NR5C(═O)R4; R4 is C1-C12 alkyl; R5 is H or C1-C6 alkyl; and x is 0, 1 or 2.

111. The pharmaceutical composition of claims 104 to 110, wherein the at least one cationic or ionizable lipid is selected or derived from a lipid according to formula III-3:

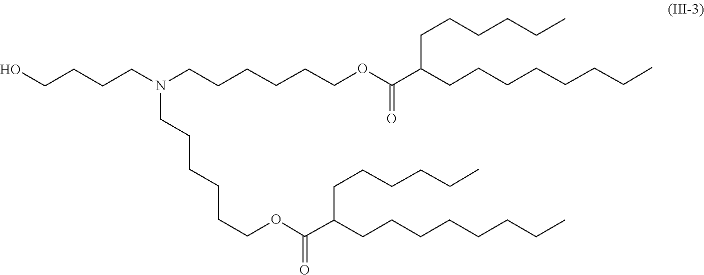 [SMILES: CCCCCCCCC(CCCCCC)C(=O)OCCCCCCN(CCCCO)CCCCCCOC(=O)C(CCCCCC)CCCCCCCC]

112. The pharmaceutical composition of claims 101 to 111, wherein the lipid-based carriers comprise a cationic lipid selected or derived from ALC-0315, SM-102, SS-33/4PE-15, HEXA-C5DE-PipSS, or compound C26.

113. The pharmaceutical composition of claims 101 to 112, wherein the lipid-based carriers comprise a neutral lipid selected or derived from DSPC, DHPC, or DphyPE.

114. The pharmaceutical composition of claims 101 to 113, wherein the lipid-based carriers comprise a steroid or steroid analog selected or derived from cholesterol, cholesteryl hemisuccinate (CHEMS), preferably cholesterol.

115. The pharmaceutical composition of claims 101 to 114, wherein the lipid-based carriers comprise
(i) at least one cationic lipid, preferably as defined in claims 109 to 112;
(ii) at least one neutral lipid, preferably as defined in claim 113;
(iii) at least one steroid or steroid analogue, preferably as defined in claim 114; and
(iv) at least one aggregation reducing lipid, preferably as defined in claims 105 to 108.

116. The pharmaceutical composition of claims 101 to 115, wherein the lipid-based carriers comprise
(i) at least one cationic lipid selected from ALC-0315;
(ii) at least one neutral lipid selected from DSPC;
(iii) at least one steroid or steroid analogue selected from cholesterol; and
(iv) at least one aggregation reducing lipid selected from ALC-0159.

117. The pharmaceutical composition of claims 101 to 115, wherein the lipid-based carriers comprise
(i) at least one cationic lipid selected from SM-102;
(ii) at least one neutral lipid selected from DSPC;
(iii) at least one steroid or steroid analogue selected from cholesterol; and
(iv) at least one aggregation reducing lipid selected from DMG-PEG 2000.

118. The pharmaceutical composition of claims 101 to 117, wherein the lipid-based carriers comprise about 20-60% cationic lipid, about 5-25% neutral lipid, about 25-55% steroid or steroid analogue, and about 0.5-15% aggregation reducing lipid.

119. The pharmaceutical composition of claims 101 to 118, wherein the wt/wt ratio of lipid to nucleic acid in the lipid-based carrier is from about 10:1 to about 60:1, preferably from about 20:1 to about 30:1.

120. The pharmaceutical composition of claims 101 to 119, wherein the N/P ratio of the lipid-based carriers encapsulating the nucleic acid, preferably the RNA is in a range from about 1 to about 10, preferably in a range from about 5 to about 7.

121. The pharmaceutical composition of claims 101 to 120, wherein the lipid-based carriers have a Z-average size in a range of about 50 nm to about 120 nm.

122. The pharmaceutical composition of claims 93 to 122, additionally comprising at least one antagonist of at least one RNA sensing pattern recognition receptor selected from a Toll-like receptor, preferably a TLR7 antagonist and/or a TLR8 antagonist.

123. The pharmaceutical composition of claims 93 to 123, additionally comprising at least one small molecule inhibitor an inhibitory nucleic acid (siRNA) of the RUNX target transcription factor, preferably a small molecule inhibitor of RUNX or an inhibitory nucleic acid (siRNA) of RUNX.

124. The pharmaceutical composition of claims 93 to 124, wherein the composition is a liquid composition or a dried composition.

125. The pharmaceutical composition or the artificial nucleic acid of any one of the preceding claims, wherein upon intramuscular, intranasal, transdermal, intradermal, intralesional, intracranial, subcutaneous, intracardial, intratumoral, intravenous, intrapulmonal, intraarticular, sublingual, pulmonary, intrathecal, or ocular administration of the composition or nucleic acid to a cell, tissue, or subject, the RUNX transcription factor inhibitor is produced.

126. The pharmaceutical composition or the artificial nucleic acid of any one of the preceding claims, wherein upon local administration of the composition or artificial nucleic acid to a cell, tissue, or subject, the RUNX transcription factor inhibitor is produced.

127. The pharmaceutical composition or the artificial nucleic acid of claim 125 or 126, wherein the administration is an ocular administration.

128. The pharmaceutical composition or the artificial nucleic acid of claim 127, wherein the ocular administration is selected from topical, intravitreal, intracameral, subconjunctival, subretinal, subtenon, retrobulbar, topical, orbital, suprachoroidal, posterior juxtascleral, or intraoperative administration.

129. The pharmaceutical composition or the artificial nucleic acid of claim 127 or 128, wherein the ocular administration is intravitreal or intraoperative administration.

130. The pharmaceutical composition or the artificial nucleic acid of any one of the preceding claims, wherein the administration is performed into a tamponade agent-filled human eye.

131. The pharmaceutical composition or the artificial nucleic acid of claim 130, wherein the tamponade agent is a gas agent or a silicone agent.

132. The pharmaceutical composition or the artificial nucleic acid of any one of the preceding claims, wherein the ocular administration is performed using lipid-based carriers, preferably lipid nanoparticles (LNPs) comprising lipids as defined in claim 116 or 117.

133. The pharmaceutical composition or the artificial nucleic acid of any one of the preceding claims, wherein ocular administration of the composition or the nucleic acid leads to a production of the RUNX transcription factor inhibitor in cells and/or tissues of the eye, preferably in cells and/or tissues selected from cornea, lens, ciliary body, vitreous, sclera, choroid, retina, optic nerve, macula, scleral cells, choroid plexus epithelial cells, retinal cells, inflammatory cells, retinal pigment epithelium (RPE), Bruch's membrane, and retinal or choroidal blood vessels.

134. The pharmaceutical composition or the artificial nucleic acid of any one of the preceding claims, wherein ocular administration of the composition or the nucleic acid leads to a production of the RUNX transcription factor inhibitor in retinal pigment epithelial (RPE) cells or cells derived from them.

135. The pharmaceutical composition or the artificial nucleic acid of any one of the preceding claims, wherein ocular administration of the composition or the nucleic acid leads to a production of the RUNX transcription factor inhibitor in retinal cells selected from photoreceptor cells, bipolar cells, ganglion cells, horizontal cells, Muller cells, mural cells, microglia, and amacrine cells.

136. A pharmaceutical composition comprising at least one RNA encoding a therapeutic protein formulated in a lipid nanoparticle (LNP), wherein the LNP comprise an aggregation reducing lipid, a cationic lipid selected or derived from formula (III), a neutral lipid or phospholipid and a steroid or steroid analog, for use in treatment or prevention of an ophthalmic disease, disorder or condition, wherein said composition is administered via intravitreal administration to a subject in need thereof.

137. A Kit or kit of parts comprising at least one artificial nucleic acid of any one of claims 1 to 92, and/or at least one pharmaceutical composition of any one of claims 93 to 135, optionally comprising a liquid vehicle for solubilising, and optionally comprising technical instructions providing information on administration and dosage of the components.

138. An artificial nucleic acid of any one of claims 1 to 92, or a pharmaceutical composition of any one of claims 93 to 135, or a kit or kit of parts of claim 137, for use as a medicament in treating or preventing a disease, disorder, or condition in a subject.

139. An artificial nucleic acid of any one of claims 1 to 92, or a pharmaceutical composition of any one of claims 93 to 135, or a kit or kit of parts of claim 137, for use as a medicament in treating or preventing an ocular disease, disorder, or condition in a subject.

140. The artificial nucleic acid of any one of claims 1 to 92, or the pharmaceutical composition of any one of claims 93 to 135, or the kit or kit of parts of claim 137, for use as a medicament of claim 138 or 139, wherein the disease, disorder, or condition is associated with or caused by an overexpressed and/or an overactive RUNX transcription factor.

141. The artificial nucleic acid of any one of claims 1 to 92, or the pharmaceutical composition of any one of claims 93 to 135, or the kit or kit of parts of claim 137, for use as a medicament of claims 138 to 140, wherein the disease, disorder, or condition is associated with or caused by pathological epithelial to mesenchymal transition (EMT), pathological cell proliferation, aberrant angiogenesis, aberrant neovascularization, degeneration, fibrosis and/or solid tumors.

142. The artificial nucleic acid of any one of claims 1 to 92, or the pharmaceutical composition of any one of claims 93 to 135, or the kit or kit of parts of claim 137 for use as a medicament of claims 139 to 141, wherein the ocular disease, disorder, or condition is associated with or caused by aberrant proliferation or migration of retinal pigment epithelial (RPE) cells in a subject.

143. The artificial nucleic acid of any one of claims 1 to 92, or the pharmaceutical composition of any one of claims 93 to 135, or the kit or kit of parts of claim 137 for use as a medicament of claims 139 to 142, wherein the ocular disease, disorder, or condition is selected from proliferative diabetic retinopathy (PDR), macular edema, non-proliferative diabetic retinopathy, age-related macular degeneration, geographic atrophy, ocular neovascularization, retinopathy of prematurity (ROP), a retinal vein occlusion, ocular ischemic syndrome, neovascular glaucoma, a retinal hemangioma, Coats' disease, FEVR, or Norrie disease, persistent hyperplastic primary vitreous (PHPV), thyroid eye disease, epiretinal membrane, small vessel disease, or proliferative vitreoretinopathy (PVR).

144. The artificial nucleic acid of any one of claims 1 to 92, or the pharmaceutical composition of any one of claims 93 to 135, or the kit or kit of parts of claim 137 for use as a medicament of claims 138 to 143, wherein the ocular disease, disorder, or condition is PVR, preferably the prevention of PVR.

145. The artificial nucleic acid of any one of claims 1 to 92, or the pharmaceutical composition of any one of claims 93 to 135, or the kit or kit of parts of claim 137 for use as a medicament of claim 144, wherein cell proliferation and/or cell growth is reduced in eyes with PVR.

146. The artificial nucleic acid of any one of claims 1 to 92, or the pharmaceutical composition of any one of claims 93 to 135, or the kit or kit of parts of claim 137 for use as a medicament of claims 138 to 145, wherein the subject has suffered a trauma to the eye, comprises a retinal hole, a retinal tear, a retinal detachment disorder, or has undergone an ocular surgery.

147. The artificial nucleic acid of any one of claims 1 to 92, or the pharmaceutical composition of any one of claims 93 to 135, or the kit or kit of parts of claim 137 for use as a medicament of claim 146, wherein the retinal detachment disorder is selected from rhegmatogenous retinal detachment, exudative retinal detachment, or tractional retinal detachment.

148. The artificial nucleic acid of any one of claims 1 to 92, or the pharmaceutical composition of any one of claims 93 to 135, or the kit or kit of parts of claim 137 for use as a medicament of claims 138 to 147, wherein the use comprises administration of the artificial nucleic acid, the pharmaceutical composition, or the kit or kit of parts by local administration, preferably by ocular administration.

149. The artificial nucleic acid of any one of claims 1 to 92, or the pharmaceutical composition of any one of claims 93 to 135, or the kit or kit of parts of claim 137 for use as a medicament of claims 138 to 148, wherein the use comprises administration of the artificial nucleic acid, the pharmaceutical composition, or the kit or kit of parts by intravitreal administration, by administration prior to an ocular surgery, during an ocular surgery, or after an ocular surgery.

150. The artificial nucleic acid of any one of claims 1 to 92, or the pharmaceutical composition of any one of claims 93 to 135, or the kit or kit of parts of claim 137 for use as a medicament of claims 138 to 149, wherein the disease, disorder, or condition is associated with or caused by overexpressed and/or overactive RUNX, preferably RUNX1.

151. A method of treating or preventing a disease, disorder or condition, wherein the method comprises applying or administering to a subject in need thereof an effective amount of the artificial nucleic acid of any one of claims 1 to 92, or the pharmaceutical composition of any one of claims 93 to 135, or the kit or kit of parts of claim 137.

152. The method of treating or preventing a disease, disorder or condition of claim 151, wherein the disease, disorder, or condition is an ocular disease, disorder, or condition.

153. The method of treating or preventing a disease, disorder or condition of claim 151 or 152, wherein the disease, disorder, or condition is associated with or caused by an overexpressed and/or an overactive RUNX transcription factor.

154. The method of treating or preventing a disease, disorder or condition of claims 151 to 153, wherein the disease, disorder, or condition associated with or caused by pathological epithelial to mesenchymal transition (EMT), pathological cell proliferation, aberrant angiogenesis, aberrant neovascularization, degeneration, solid tumors, and/or fibrosis.

155. The method of treating or preventing a disease, disorder or condition of claims 151 to 154, wherein the ocular disease, disorder, or condition is associated with or caused by aberrant proliferation or migration of retinal pigment epithelial (RPE) cells in a subject.

156. The method of treating or preventing a disease, disorder or condition of claims 151 to 155, wherein the ocular disease, disorder, or condition is selected from proliferative diabetic retinopathy (PDR), macular edema, non-proliferative diabetic retinopathy, age-related macular degeneration, ocular neovascularization, retinopathy of prematurity (ROP), a retinal vein occlusion, ocular ischemic syndrome, neovascular glaucoma, a retinal hemangioma, Coats' disease, FEVR, or Norrie disease, persistent hyperplastic primary vitreous (PHPV), thyroid eye disease, epiretinal membrane, small vessel disease, or proliferative vitreoretinopathy (PVR).

157. The method of treating or preventing a disease, disorder or condition of claims 151 to 156, wherein the ocular disease, disorder, or condition is PVR, preferably the prevention of PVR.

158. The method of treating or preventing a disease, disorder or condition of claim 157, wherein cell proliferation and/or cell growth is reduced in eyes with PVR.

159. The method of treating or preventing a disease, disorder or condition of claims 151 to 158, wherein the subject has suffered a trauma to the eye, comprises a retinal hole, a retinal tear, a retinal detachment disorder, or has undergone an ocular surgery.

160. The method of treating or preventing a disease, disorder or condition of claim 159, wherein the retinal detachment disorder is selected from rhegmatogenous retinal detachment, exudative retinal detachment, or tractional retinal detachment.

161. The method of treating or preventing a disease, disorder or condition of claims 151 to 160, wherein the applying or administering is performed more than once, for example two times, three times, or four times, for example periodically.

162. The method of treating or preventing a disease, disorder or condition of claims 151 to 161, wherein the applying or administering is performed by local administration, preferably by ocular administration.

163. The method of treating or preventing a disease, disorder or condition of claims 151 to 162, wherein the applying or administering is performed by intravitreal administration, by administration prior to an ocular surgery, during an ocular surgery, and/or after an ocular surgery.

164. The method of treating or preventing a disease, disorder or condition of claims 151 to 163, wherein the disease, disorder, or condition is associated with or caused by overexpressed and/or overactive RUNX, preferably RUNX1.

165. A method of treating or preventing or preventing an ocular disease in a subject comprising, administering to the subject an effective amount of a composition comprising an artificial nucleic acid comprising at least one coding sequence encoding the RUNX inhibitor, the RUNX inhibitor comprising:
at least one amino acid element A selected or derived from CBFbeta configured to bind to the target transcription factor RUNX in the cytosol, preferably wherein element A is further characterized by any of claims 5 to 10; and
at least one amino acid element B selected or derived from SMMHC configured to trap the target transcription factor RUNX in the cytosol, preferably wherein element B is further characterized by any of claims 17 to 28.

166. The method of claim 165, wherein the composition is administered by ocular administration.

167. The method of claim 166, wherein the ocular administration is selected from intravitreal or intraoperative administration.

168. The method of claims 165 to 157, wherein the artificial nucleic acid is an mRNA as defined by any of claims 76 to 92.

169. The method of claims 165 to 157, wherein the artificial nucleic acid is comprised in a viral vector, preferably an AAV vector.

170. The method of claims 165 to 169, wherein the artificial nucleic acid is formulated in lipid-based carriers, preferably as defined by any of claims 101 to 121.

171. The method of claims 165 to 170, wherein the ocular disease is associated with or caused by overexpressed RUNX and/or overactive RUNX, preferably, wherein the ocular disease is PVR.

172. A method of reducing the activity of a RUNX transcription factor in a cell or a subject, wherein the method comprises
a) applying or administering an artificial nucleic acid comprising at least one coding sequence encoding at least one RUNX transcription factor inhibitor of claims 1 to 92; or
b) applying or administering a pharmaceutical composition comprising the artificial nucleic acid comprising at least one coding sequence encoding at least one RUNX transcription factor inhibitor of claims 93 to 135
to a cell, tissue, or subject, wherein the RUNX transcription factor inhibitor is produced in the cell, tissue, or subject after administration or application to said cell, tissue, or subject.

173. The method of claim 172, wherein the produced RUNX transcription factor inhibitor is a RUNX trap and the target transcription factor is RUNX.

174. The method of claim 173, wherein the produced RUNX transcription factor inhibitor or RUNX trap
sequesters cellular RUNX by binding to RUNX in the cytosol or sequesters cellular CBFbeta by binding to CBFbeta in the nucleus
reduces or prevents the interaction of cellular RUNX with cellular CBFbeta; and/or
reduces cellular RUNX-CBFbeta complex formation and/or activity; and/or
reduces the cellular expression of RUNX controlled or regulated gene products; and/or
reduces the cellular expression of TGFβ2, SMAD3, and/or COL1A1; and/or
reduces the cellular expression of RUNX; and/or
reduces or prevents pathological epithelial to mesenchymal transition (EMT), pathological cell proliferation, aberrant angiogenesis, aberrant neovascularization, degeneration, and/or fibrosis; and/or
reduces or prevents cell proliferation and/or cell growth in eyes with PVR.

175. The method of claims 172 to 174, wherein an ocular administration leads to a production of the RUNX transcription factor inhibitor in cells and/or tissues of the eye, preferably in retinal pigment epithelial (RPE) cells.

176. The method of claims 172 to 175, wherein the reduction of the activity of a RUNX transcription factor is a transient reduction of the activity of a RUNX transcription factor.

177. The method of claims 172 to 176, wherein the RUNX transcription factor inhibitor is a dominant negative inhibitor of the RUNX target transcription factor.
